(19) United States
(12) Patent Application Publication
(10) Pub. No.: US 2003/0204865 A1
(43) Pub. Date: Oct. 30, 2003

(54) CaaX prenyl protease nucleic acids and polypeptides and methods of use thereof

(76) Inventors: Jiangxin Wan, Kingston (CA); Yafan Huang, Kingston (CA); Delina Mary-Jane Melo, Inverary (CA); Monika Maria Kuzma, Glenburnie (CA); Angela Patricia Gilley Sample, Inverary (CA)

Correspondence Address: Ivor R. Elrifi, Ph.D., Mintz, Levin, Cohn, Ferris,, Glovsky and Popeo, P.C., One Financial Center, Boston, MA, 02111, US

(21) Appl. No.: 10/210,760

(22) Filed: Aug. 1, 2002

Related U.S. Application Data
(60) Provisional application No. 60/309,396, filed on Aug. 1, 2001.
(60) Provisional application No. 60/337,084, filed on Dec. 4, 2001.

(51) Int. Cl. A01H 1/00; C12N 15/82; C12N 9/50

(52) U.S. Cl. 800/278; 435/219

(57) ABSTRACT

The present invention provides novel isolated prenyl protease polynucleotides and polypeptides encoded by the prenyl protease polynucleotides. Also provided are the antibodies that immunospecifically bind to a prenyl protease polypeptide or any derivative, variant, mutant or fragment of the prenyl protease polypeptide, polynucleotide or antibody. The invention additionally provides methods of constructing transgenic plants that have altered levels of prenyl protease polynucleotides and polypeptides. Methods for identifying prenyl protease enzymes substrates and inhibitors are also provided.

### RELATED APPLICATIONS

This application claims priority to U.S. S No. 60/309,396 filed Aug. 1, 2001 and U.S. S No. 60/337,084 filed Dec. 4, 2001 each of which are incorporated herein by reference in their entireties.

### FIELD OF THE INVENTION

The invention relates to novel plant CaaX prenyl protease polynucleotides and polypeptides. Also included are transgenic plants expressing the novel polynucleotides and polypeptides. Also included are transgenic plant cells, tissues and plants having novel phenotypes resulting from the expression of these polynucleotides in either the sense or antisense orientation.

### BACKGROUND OF THE INVENTION

Most higher plants encounter at least transient decreases in relative water content at some stage of their life cycle and, as a result, have evolved a number of desiccation protection mechanisms. If however, the change in water deficit is prolonged the effects on the plants growth and development can be profound. Decreased water content due to drought, cold or salt stress can irreparably damage plant cells which in turn limits plant growth and crop productivity in agriculture.

Plants respond to adverse conditions of drought, salinity and cold with a variety of morphological and physiological changes. Although our understanding of plant tolerance mechanisms to these stresses is incomplete, the plant hormone abscisic acid (ABA) is believed to be an essential mediator between environmental stimulus and plant responses. ABA levels increase in response to water deficits and exogenously applied ABA mimics many of the responses induced by water-stress. Once ABA is synthesized it causes the closure of the leaf stomata thereby decreasing water loss through transpiration.

The identification of genes that transduce ABA into a cellular response opens the possibility of exploiting these regulators to enhance desiccation tolerance in crop species. In principle, these ABA signaling genes can be coupled with the appropriate controlling elements to allow optimal plant growth, development and productivity. Thus, not only would these genes allow the genetic tailoring of crops to withstand transitory environmental stresses, but they should also broaden the environments where traditional crops can be grown.

The recent isolation of an Arabidopsis mutant, era1, is hypersensitive to ABA and has been shown to also be tolerant to conditions of water deprivation. ERA1 has been identified as a D subunit of farnesyl transferase knockout mutant in. Farnesyl transferase is a heterodimeric enzyme that provides the specific addition of a farnesyl pyrophosphate moiety onto the substrate target sequence. The target sequence is defined as a sequence of four amino acids which are present at the carboxy terminus of the protein and is referred to as a CaaX motif in which the “C” is cysteine, “a” is any aliphatic amino acid and “X” is any amino acid. The α subunit is common with a second prenylation enzyme, geranylgeranyl transferase, that has a different β subunit and adds a geranylgeranyl isoprenyl pyrophosphate moiety to the target sequence.

Prenylation is a multistep pathway which includes prenylation of the cysteine residue of the CaaX site, cleavage of the -aaX tripeptide and methylation of the prenyl-cysteine residue. Potentially, each of these steps could represent a target for genetic manipulation of the prenylation process to generate a desired phenotype such as stress tolerance.

In plants, prenylation has been linked to cell cycle control, meristem development, and phytohormone signal transduction, however, few details of the role of prenylation, the substrate proteins or the extent to which the plant system will be analogous to the mammalian and yeast systems are known. The most characterized substrates for CaaX modification are the Ras and a-factor proteins of yeast. Although there are three steps to complete protein maturation, abolition or modification of any one step does not necessarily result in cessation of target biological activities. Ras function is attenuated if the -aaX tripeptide is not cleaved but not abolished and some proteins retain the -aaX tripeptide after farnesylation.

In Arabidopsis, more than 600 proteins contain a CaaX motif, suggesting a role for the post-translational modification by prenylation in numerous cellular processes. In Arabidopsis, it has been demonstrated that the loss-of-function of the β-subunit of farnesyl transferase will result in a ABA-hypersensitive phenotype. Although it is still not clear why plants lacking the functional β-subunit of farnesyl transferase become more sensitive to ABA, it clearly suggests that protein prenylation is involved in regulation of the homeostasis of ABA sensitivity. The balance of ABA cellular responses, whether more sensitive or less sensitive to ABA, is possibly regulated by the relative activities of prenylated proteins. The changes in AtCPP expression and gene activity may affect the activity of two pools of genes, one pool acting as positive regulators (pool A) and the second pool (pool B) as negative regulators, which require prenylation in order to function properly. Pool A may contain genes that can promote ABA sensitivity, and pool B genes that may reduce ABA sensitivity. The homeostasis of ABA sensitivity may therefore governed by the ratio of activity of pool A to pool B. For example, in the case of up-regulation of AtCPP in Arabidopsis, the activity ratio of pool A over pool B may be increased due to difference in substrate affinity of pool A proteins toward AtCPP, thus the homeostasis of ABA sensitivity is changed, and the AtCPP over-expression plants are more sensitive to ABA.

This invention is directed at the manipulation of the CaaX prenyl protease enzyme (CPP), which catalyses the proteolytic cleavage of the -aaX tripeptide in the second step of the prenylation process. Included in this invention are vector constructs containing CPP sequence under the control of appropriate regulatory sequences to produce a water-stress tolerant phenotype.

### SUMMARY OF THE INVENTION

The present invention is based in part upon the discovery of novel CaaX prenyl protease (CPP) nucleic acid sequences and polypeptides isolated from Arabidopsis thaliana, Brassica napus and Glycine max. The nucleic acids, polynucleotides, proteins and polypeptides, or fragments thereof described herein are collectively referred to as CPP nucleic acids and polypeptides.

Accordingly, in one aspect, the invention provides an isolated nucleic acid molecule that includes the sequence of SEQ ID NO:1, SEQ ID NO:14, or SEQ ID NO:17 or fragment, homolog, analog or derivative thereof. The nucleic acid can include, e.g., a nucleic acid sequence encoding a polypeptide at least 99% identical to a polypeptide that includes the amino acid sequences of SEQ ID NO:2, SEQ ID NO:15, or SEQ ID NO:18 or a nucleic acid sequence encoding a polypeptide at least 96% identical to a polypeptide that includes the amino acid sequences of SEQ ID NO:15. In yet another aspect, the invention provides a nucleic acid that includes the sequence of SEQ ID NO: 68, 70, 72 or 74. The nucleic acid can be, e.g., a genomic DNA fragment, or a cDNA molecule. Preferably, the nucleic acid is naturally occurring. The invention also provides a nucleic acid sequence that is complementary to the nucleic acid sequence of SEQ ID NO:1, SEQ ID NO:14, or SEQ ID NO:17. For example, SEQ ID NO:16, 19 or 20.

Also included in the invention is a vector containing one or more of the nucleic acids described herein, and a cell containing the vectors or nucleic acids described herein. In various aspects the vector comprises the nucleic acid sequences of SEQ ID NO: 4, 5, 36-53.

The invention is also directed to host cells transformed with a vector comprising any of the nucleic acid molecules described above.

The invention is also directed to plants and cells transformed with a CPP nucleic acid or a vector comprising a CPP nucleic acid. Also included in the invention is the seed, and progeny of the transformed plants or cells.

In a further aspect, the invention includes a substantially purified CPP polypeptide, e.g., any of the CPP polypeptides encoded by an CPP nucleic acid, and fragments, homologs, analogs, and derivatives thereof. Accordingly, in one aspect, the invention provides an isolated polypeptide molecule that includes the sequence of SEQ ID NO:2, SEQ ID NO:15, or SEQ ID NO:18.

In yet another aspect the invention provides a polypeptides that includes the sequence of SEQ ID NO: 69, 71, 73 or 75.

In still a further aspect, the invention provides an antibody that binds specifically to an CPP polypeptide. The antibody can be, e.g., a monoclonal or polyclonal antibody, and fragments, homologs, analogs, and derivatives thereof. The invention is also directed to isolated antibodies that bind to an epitope on a polypeptide encoded by any of the nucleic acid molecules described above.

The invention also includes a method of producing a transgenic plant which has an altered phenotype such as, but not limited to, increased tolerance to stress, delayed senescence, increased ABA sensitivity, increased yield, increased productivity and increased biomass compared to a wild type plant by introducing into one or more cells of a plant a compound that alters (e.g., increases or decreases) CPP expression or activity in the plant. In one aspect the compound is a CPP nucleic acid or polypeptide. In one emodiment the nucleic acid is an inhibitor or farnesylation. For example, the compound comprises SEQ ID NO: 1, 14, 17, 68, 70, 72, 74, 21, 23, 25, 27, 29, 31, 33, 2, 15, 18, 22, 24, 26, 28, 30, 32, 34, 69, 71, 73, or 75. Alternatively, the compound is a CPP double stranded RNA-inhibition hair-pin nucleic acid or CPP antisense nucleic acid, such as for example, SEQ ID NO: 16, 19, 20, 5, 35, 37, 42, 45, 46, 48, 49, 51 or 51.

The invention further provides a method for producing a CPP polypeptide by providing a cell containing an CPP nucleic acid, e.g., a vector that includes a CPP nucleic acid, and culturing the cell under conditions sufficient to express the CPP polypeptide encoded by the nucleic acid. The expressed CPP polypeptide is then recovered from the cell. Preferably, the cell produces little or no endogenous CPP polypeptide. The cell can be, e.g., a prokaryotic cell or eukaryotic cell.

The invention is also directed to methods of identifying a CPP polypeptide or nucleic acid in a sample by contacting the sample with a compound that specifically binds to the polypeptide or nucleic acid, and detecting complex formation, if present. The invention further provides methods of identifying a compound that modulates the activity of a CPP polypeptide by contacting a CPP polypeptide with a compound and determining whether the CPP polypeptide activity is modified.

The invention is also directed to compounds that modulate CPP polypeptide activity identified by contacting a CPP polypeptide with the compound and determining whether the compound modifies activity of the CPP polypeptide, binds to the CPP polypeptide, or binds to a nucleic acid molecule encoding a CPP polypeptide.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1. is a schematic representation of the vector constructs; A) pBI121-AtCPP, B) pBI121-antisense-AtCPP, C) pBI121-HP-AtCPP.

FIG. 2. is an illustration of (A) nucleic acid and (B) amino acid sequence identities as determined by ClustalW analysis.

FIG. 3. is a scan of a typical Southern blot of transgenic Arabidopsis T1 lines carrying the pBI121-AtCPP construct.

FIG. 4. is a scan of a typical Southern blot of transgenic Arabidopsis T3 lines carrying the pBI121-HP-AtCPP construct.

FIG. 5. is a scan of a typical Southern blot of transgenic Arabidopsis lines carrying the pRD29A-AtCPP construct.

FIG. 6. is a scan of a typical Southern blot of transgenic Arabidopsis lines carrying the pRD29A-HP-AtCPP construct.

FIG. 7 is an illustration showing the relative expression of AtCPP mRNA transcript (solid bars) and AtCPP protein levels (stippled bars) in several pBI121-AtCPP transgenic lines.

FIG. 8. is a histogram showing the percentage of lines which were categorized as ABA sensitive, moderately ABA sensitive or ABA insensitive. Seedlings were assessed on agar plates containing 1 μM ABA and scored at 21 days growth. Thirty-six lines of the pBI121-AtCPP over-expression construct were assessed at 21 days by leaf and seedling development. Thirty-two lines of the ³⁵S-HP-AtCPP down-regulation construct were assessed at 21 days for leaf and seedling development. Each line was assessed by plating approximately 100 seeds per plate and the seedlings scored and recorded as the percent insensitive seedlings per plate. Each line was then expressed as a percent of wild type (Wt). Lines were categorized as sensitive (less than 1% of Wt) solid bars, intermediate (1-50% of Wt) diagonally lined or insensitive (greater than 50% of Wt) stippled, based on their relationship to Wt and the percentage of each category plotted as a histogram.

FIG. 9. is an illustration showing the response of wild type and a pRD29A-HP-AtCPP transgenic line to various concentrations of ABA in two week old seedlings.

FIG. 10. is a histogram showing the analysis of transgenic plants containing the pBI121-AtCPP over-expression construct, (SEQ ID NO:4). Water loss per gram shoot dry weight after four days of water stress treatment. Lines that are marked with a star are those which were strongly ABA sensitive. Lines marked with a triangle are moderately ABA sensitive. Bars represent means of eight replicates. Lines marked with a filled dot above the bar represents lines which were significantly different from control at a p=0.05 value.

FIG. 11. is a histogram showing seed yield in grams of transgenic Arabidopsis lines of pBI121-AtCPP grown under optimal water conditions

FIG. 12. is a bar chart howing growth and yield of transgenic Arabidopsis lines of pBI121-AtCPP grown under optimal watering conditions plus a biotic stress condition. Tields as a % of wild type, rosette leaf number, rosette leaf fresh weight and shoot dry weight are plotted.

FIG. 13. are photographs showing rowth of transgenic Arabidopsis lines of pBI121At-CPP grown on agar plates. Changes to root growth visible.

FIG. 14. is a bar chart showing rowth of transgenic Arabidopsis lines of pRD29A-HP—AtCPP grown under optimal watering conditions. Rosette leaf number, rosette leaf dry weight and shoot dry weight are plotted.

### DETAILED DESCRIPTION OF INVENTION

The present invention provides novel CaaX prenyl protease (CPP) nucleic acid sequences (SEQ ID No:1, SEQ ID NO:14 and SEQ ID NO:17) the encoded polypeptides: SEQ ID NO:2, SEQ ID NO:15 and SEQ ID NO:18) isolated from Arabidopsis thaliana (At) Brassica napus (Bn) and Glycine Max (Gm) respectively. The invention also provides CaaX prenyl protease antisense nucleic acids. (SEQ ID NO:16, SEQ ID NO:19 and SEQ ID NO:20). The sequences are collectively-referred to as “CPP nucleic acids”, CPP nucleotides” or “CPP antisense nucleic acids” and the corresponding encoded polypeptide is referred to as a “CPP polypeptide” or “CPP protein”. Unless indicated otherwise, “CPP” is meant to refer to any of the novel sequences disclosed herein. Table A below summarizes the nucleic acids and polypeptides according to the invention

TABLE A

| SEQ | | | | Species |
|---|---|---|---|---|
| ID NO. | SEQ | Type | Transformed | |
| 1 | AtCPP | NA | PCR | |
| 2 | AtCPP | AA | Translation | |
| 3 | At-AFCl | AA | Ref. | |
| 4 | pBI121-AtCPP | NA | Construct | At, Bn |
| 5 | pBI121-HP-AtCPP | NA | Construct | At |
| 6 | AtCPP BamFW | NA | Primer | |
| 7 | AtCPP SmaRV | NA | Primer | |
| 8 | AtCPP-HP-SacFW | NA | Primer | |
| 9 | AtCPP-HP-SacRV | NA | Primer | |
| 10 | pBI121-AtCPP Forward | NA | Primer | |
| 11 | pBI121-antiAtCPP-SmaFW | NA | Primer | |
| 12 | pBI121-antiAtCPP-BamRV | NA | Primer | |
| 13 | p35S-HP-AtCPP Reverse | NA | Primer | |
| 14 | BnCPP | NA | PCR | |
| 15 | BnCPP | AA | Translation | |
| 16 | BnCPP antisense | NA | PCR | |
| 17 | GmCPP | NA | PCR | |
| 18 | GmCPP | AA | Translation | |
| 19 | GmCPP antisense | NA | PCR | |
| 20 | AtCPP antisense | NA | PCR | |
| 21 | BASF-AT1 | NA | Ref. | |
| 22 | BASF-AT1 | AA | Ref. | |
| 23 | BASF-AT2 | NA | Ref. | |
| 24 | BASF-AT2 | AA | Ref. | |
| 25 | BASF-Corn | NA | Ref. | |
| 26 | BASF-Corn | AA | Ref. | |
| 27 | BASF-Soy | NA | Ref. | |
| 28 | BASF-Soy | AA | Ref. | |
| 29 | AFC1 | NA | Ref. | |
| 30 | AFC1 | AA | Ref | |
| 31 | AT4g01320 | NA | Ref. | |
| 32 | AT4g01320 | AA | Ref. | |
| 33 | AF007269 | NA | Ref. | |
| 34 | AF007269 | AA | Ref. | |
| 35 | pBI121-antisense-AtCPP | NA | Construct | |
| 36 | pRD29A-AtCPP | NA | Construct | At, Bn |
| 37 | pRD29A-HP-AtCPP | NA | Construct | At |
| 38 | pRD29A-antisense-AtCPP | NA | Construct | |
| 39 | MuA-AtCPP | NA | Construct | Gm, Zm |
| 40 | MuA-GmCPP | NA | Construct | |
| 41 | pBI121-GmCPP | | Construct | |
| 42 | pBI121-HP-GmCPP | | Construct | |
| 43 | pBI121-antisense-GmCPP | | Construct | |
| 44 | pRD29A-GmCPP | | Construct | |
| 45 | pRD29A-HP-GmCPP | | Construct | |
| 46 | pRD29A-antisense-GmCPP | | Construct | |
| 47 | pBI121-BnCPP | | Construct | |
| 48 | pBI121-HP-BnCPP | | Construct | |
| 49 | pBI121-antisense-BnCPP | | Construct | |
| 50 | pRD29A-BnCPP | | Construct | |
| 51 | pRD29A-HP-BnCPP | | Construct | |
| 52 | pRD29A-antisense-BnCPP | | Construct | |
| 53 | MuA-BnCPP | | Construct | |
| 54 | GmCPP SmaFW | | Primer | |
| 55 | GmCPP SacRV | | Primer | |
| 56 | BnCPP-anti-SmaFW | | Primer | |
| 57 | BnCPP-anti-BamRV | | Primer | |
| 58 | BnCPP-HP-Sac-FW | | Primer | |
| 59 | BnCPP-HP-Sac-RV | | Primer | |
| 60 | BnCPP-HP-BamFW | | Primer | |
| 61 | BnCPP-HP-XbaRV | | Primer | |
| 62 | GmCPP-HP-Sac-FW | | Primer | |
| 63 | GmCPP-HP-Sac-RV | | Primer | |
| 64 | GmCPP-HP-BamFW | | Primer | |
| 65 | GmCPP-HP-XbaRV | | Primer | |
| 66 | pRD29AP | | Primer | |
| 67 | Nosterm-RV | | Primer | |
| 68 | Consensus-BASF | NA | | |
| 69 | Consensus-BASF | AA | | |
| 70 | Consensus- Generic | NA | | |
| 71 | Consensus- Generic | AA | | |
| 72 | Consensus- PPI | NA | | |
| 73 | Consensus- PPI | AA | | |
| 74 | Consensus- PPI/Generic | NA | | |
| 75 | Consensus- PPI/Genreric | AA | | |

In a BLAST search of public sequence databases, it was found, for example, that the Arabidopsis thaliana nucleic acid sequence has 99.5% identity to an Arabidopsis thaliana CaaX processing zinc-metallo endoprotease (AFC 1) mRNA (Genbank Accesion No.: AF353722). The full amino acid sequence of the protein of the invention was found to be 98% identical to Arabidopsis thaliana CaaX processing zinc-metallo endoprotease (AFC1) polypeptide (Genbank Accesion No.:AAK39514). A ClustalW alignment of the Arabidopsis thaliana CPP polypeptide (SEQ ID NO:2), the Brassica napus CPP polypeptide (SEQ ID NO:15), the Glycine max CPP polypeptide (SEQ ID NO:18) and seven other published CPP sequences is illustrated in Table 6B. ClustalW alignment of these polypeptides indicate that SEQ ID NO:2, SEQ ID NO:15 and SEQ ID NO:18 are 99%, 93% and 83% identical to the published AFC sequence (SEQ ID NO:30) respectively. The Glycine max CPP polypeptide (SEQ ID NO:18) is 99% identical to the published sequence shown as SEQ ID NO:28. Similarly, ClustalW alignment of the Arabidopsis thaliana CPP polynucleotide (SEQ ID NO:1), the Brassica napus CPP polynucleotide (SEQ ID NO:14), the Glycine max CPP polynucleotide (SEQ ID NO:17) and seven other published CPP sequences is illustrated in Table 6a indicate that SEQ ID NO:1, SEQ ID NO:14 and SEQ ID NO:17 are 99%, 93% and 77% identical to the published AFC sequence (SEQ ID NO:30) respectively. The Glycine max CPP polynucleotide (SEQ ID NO:17) is 93% identical to the published sequence shown as SEQ ID NO:27.

CaaX prenyl proteases belong to a family of putative membrane-bound proteins that are involved in protein and/or peptide modification (i.e., prenylation) and secretion. Prenylation is a post translational modification of specific proteins and is required for the proper localization of these polypeptides to the correct cellular site for functionality. Prenylation is a three step process involving the addition of either a C 15 farnesyl, or C20 geranylgeranyl group to the cysteine residue of the target 3′ terminal CaaX sequence, where “C” is a cysteine, “a” is any aliphatic amino acid and “X” is any amino acid. Secondly, a CaaX prenyl protease (CPP) cleaves the -aaX tripeptide from the protein and thirdly the exposed a-carboxyl group of the cysteine is methylated by a prenyl-cysteine methyltransferase.

Protein farnesylation, the addition of a C-terminal, 15 carbon chain to protein and subsequent processing is a three step enzymatic reaction including farnesylation, proteolytic cleavage and methylation. First, a farnesyltransferase adds the C-terminal 15 carbon chain to a conserved cysteine residue of the CaaX terminal motif, where “C” is a Cystine, “a” is an aliphatic amino acid and “X” is any amino acid. Second, the last three amino acid residues (aaX) are cleaved by a prenyl protease. Lastly, the modified cysteine is methylated by a methylase to create the final active product of the protein farnesylation pathway. The Applicant's have shown previously that over expression and down-regulation of the alpha or the beta farnesyl transferase gene in plant cells (i.e, the first step in farnesylation) results in plants with an altered phenotype such as but not limited to drought tolerance and delayed senescence. The present invention shows that over expression and down-regulation of the prenyl protease gene (i.e, the second step in farnesylation) in plant cells also results in a plant displaying an altered phenotype including for example but not limited to drought tolerance and increased resistance to biotic and abiotic stress. These results taken together support the hypothesis that modification of the expression of any of the enzymes in the farnnesylation pathway in a plant cell will result in a plant displaying an altered phenotype

Based on their structural and functional relatedness to known CaaX prenyl protease proteins, the CPP proteins are novel members of the CaaX prenyl protease family of proteins. CPP nucleic acids, and their encoded polypeptides, according to the invention are useful in a variety of applications and contexts. For example, the nucleic acids (i.e., sense or antisense CPP nucleic acids) can be used produce transgenic plants that have an increase resistance to biotic and abiotic stresses, e.g., chilling stress, salt stress, water stress, wound healing, pathogen challenge, or herbicides. Additionally, the transgenic plants have an increased productivity during both optimal and suboptimal growth conditions, increased yield, or increased biomass. Alternatively, the transgenic plants have an increased sensitivity to the phytohormone abscisic acid (ABA).

This invention includes methods to up-regulate the CPP enzyme activity in transgenic plants, cells and tissue cultures by using an over-expression vector construct and methods to down-regulate the CPP enzyme activity in transgenic plants, cells and tissue cultures by using a double stranded RNA-inhibition, hairpin vector constructs or antisense constructs. Alteration (i.e., upregulation or downregulation) of CPP enzyme activity or expression results in transgenic plants with altered phenotypes as described below. These methods are by way of example to produce the up-regulation or down-regulation effects and are not meant to be limiting as to the method of achieving this outcome.

Additionally, the nucleic acids and polypeptides according to the invention may be used as targets for the identification of small molecules that modulate or inhibit, CPP activity. Alternatively, the CPP nucleic acids and polypeptides can be used to identify proteins that are members of the CaaX prenyl protease family of proteins.

Additional utilities for CPP nucleic acids and polypeptides according to the invention are disclosed herein.

CPP Nucleic Acids

The nucleic acids of the invention include those that encode a CPP polypeptide or protein. As used herein, the terms polypeptide and protein are interchangeable.

In some embodiments, a CPP nucleic acid encodes a mature CPP polypeptide. As used herein, a “mature” form of a polypeptide or protein described herein relates to the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps that may take place within the cell in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

Among the CPP nucleic acids is the nucleic acid whose sequence is provided in SEQ ID NO:1, SEQ ID NO:14 OR SEQ ID NO:17 or a fragment thereof. Additionally, the invention includes mutant or variant nucleic acids of SEQ ID NO:1, SEQ ID NO:14 OR SEQ ID NO:17 or a fragment thereof, any of whose bases may be changed from the corresponding base shown in SEQ ID NO:1, SEQ ID NO:14 or SEQ ID NO:17, while still encoding a protein that maintains at least one of its CPP-like activities and physiological functions. The invention further includes the complement of the nucleic acid sequence of SEQ ID NO:1, SEQ ID NO:14 or SEQ ID NO:17, including fragments, derivatives, analogs and homologs thereof. Complement nucleic acid CPP sequences include SEQ ID NO:16, 19 or 20. The invention additionally includes nucleic acids or nucleic acid fragments, or complements thereto, whose structures include chemical modifications.

One aspect of the invention pertains to isolated nucleic acid molecules that encode CPP proteins or biologically active portions thereof. Also included are nucleic acid fragments sufficient for use as hybridization probes to identify CPP-encoding nucleic acids (e.g., CPP mRNA) and fragments for use as polymerase chain reaction (PCR) primers for the amplification or mutation of CPP nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated CPP nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO:1, SEQ ID NO:14, or SEQ ID NO:17 or a complement of any of this nucleotide sequence, can be isolated using standard molecular biology techniques and the sequence information provided herein. Using all or a portion of the nucleic acid sequence of SEQ ID NO:1, SEQ ID NO:14 or SEQ ID NO:17 as a hybridization probe, CPP nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to CPP nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of SEQ ID NO:1, 14 or 17, or a complement thereof. Oligonucleotides may be chemically synthesized and may be used as probes.

In another embodiment, an isolated nucleic acid molecule of the invention includes a nucleic acid molecule that is a complement of the nucleotide sequence shown in SEQ ID NO:1, SEQ ID NO:14 or SEQ ID NO:17. In another embodiment, an isolated nucleic acid molecule of the invention comprises a nucleic acid molecule that is a complement of the nucleotide sequence shown in SEQ ID NO:1, SEQ ID NO:14 or SEQ ID NO:17, or a portion of these nucleotide sequence. A nucleic acid molecule that is complementary to the nucleotide sequence shown in SEQ ID NO:1, SEQ ID NO:14 or SEQ ID NO:17 is one that is sufficiently complementary to the nucleotide sequence shown in SEQ ID NO:1, SEQ ID NO:14 or SEQ ID NO:17 that it can hydrogen bond with little or no mismatches to the nucleotide sequence shown in SEQ ID NO:1, SEQ ID NO:14 or SEQ ID NO:17, thereby forming a stable duplex. Exemplary complement nucleic acid sequences include the sequences of SEQ ID NO: 16, 19 or 20.

As used herein, the term “complementary” refers to Watson-Crick or Hoogsteen base pairing between nucleotide units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of SEQ ID NO: 1, SEQ ID NO:14 or SEQ ID NO:17, e.g., a fragment that can be used as a probe or primer, or a fragment encoding a biologically active portion of CPP. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which is incorporated herein by reference in its entirety). A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a CPP polypeptide. Isoforms can be expressed in different tissues of the same organism as a result of, for example, alternative splicing of RNA. Alternatively, isoforms can be encoded by different genes. Homologous nucleotide sequences also include, but are not limited to, naturally occurring allelic variations and mutations of the nucleotide sequences set forth herein. Exemplary homologous nucleic acid sequences include the nucleic acid sequences of SEQ ID NO: 68, 70, 72 and 74. Homologous nucleic acid sequences include those nucleic acid sequences that encode conservative amino acid substitutions (see below) in SEQ ID NO:2, SEQ ID NO:15 and SEQ ID NO:18, as well as a polypeptide having CPP activity, e.g substrate binding.

The nucleotide(sequence determined from the cloning of the Arabidopsis thaliana, Brassica napus or Glycine max CPP gene allows for the generation of probes and primers designed for use in identifying and/or cloning CPP homologues in other cell types, e.g., from other tissues, as well as CPP homologues from other plants. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NO: 1, SEQ ID NO:14 or SEQ ID NO:17; or an anti-sense strand nucleotide sequence of SEQ ID NO: 1, SEQ ID NO:14 or SEQ ID NO:17; or of a naturally occurring mutant of SEQ ID NO: 1, SEQ ID NO:14 or SEQID NO:17.

Probes based on the Arabidopsis thaliana, Brassica napus or Glycine max CPP nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a CPP protein, such as by measuring a level of a CPP-encoding nucleic acid in a sample of cells from a subject e.g., detecting CPP mRNA levels or determining whether a genomic CPP gene has been mutated or deleted.

A “polypeptide having a biologically active portion of CPP” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of CPP” can be prepared by isolating a portion of SEQ ID NO:1, SEQ ID NO:14 or SEQ ID NO:17 that encodes a polypeptide having a CPP biological activity (biological activities of the CPP proteins are described below), expressing the encoded portion of CPP protein (e.g., by recombinant expression in vitro) and assessing the activity of the encoded portion of CPP. In another embodiment, a nucleic acid fragment encoding a biologically active portion of CPP includes one or more regions.

CPP Variants

The invention further encompasses nucleic acid molecules that differ from the nucleotide sequences shown in SEQ ID NO: 1, SEQ ID NO:14 or SEQ ID NO:17 due to the degeneracy of the genetic code. These nucleic acids thus encode the same CPP protein as that encoded by the nucleotide sequence shown in SEQ ID NO: 1, SEQ ID NO:14 or SEQ ID NO:17, e.g., the polypeptide of SEQ ID NO: 2, SEQ ID NO:15, SEQ ID NO: 18. In another embodiment, an isolated nucleic acid molecule of the invention has a nucleotide sequence encoding a protein having an amino acid sequence shown in SEQ ID NO: 2, SEQ ID NO:15, SEQ ID NO: 18.

In addition to the Arabidopsis thaliana, Brassica napus or Glycine max CPP nucleotide sequence shown in SEQ ID NO:1, SEQ ID NO:14 or SEQ ID NO:17, it will be appreciated by those skilled in the art that DNA sequence polymorphisms that lead to changes in the amino acid sequences of CPP may exist within a population (e.g., the plant). Such genetic polymorphism in the CPP gene may exist among individuals within a population due to natural allelic variation. As used herein, the terms “gene” and “recombinant gene” refer to nucleic acid molecules comprising-an open reading frame encoding a CPP protein, preferably a plant CPP protein. Such natural allelic variations can typically result in 1-5% variance in the nucleotide sequence of the CPP gene. Any and all such nucleotide variations and resulting amino acid polymorphisms in CPP that are the result of natural allelic variation and that do not alter the functional activity of CPP are intended to be within the scope of the invention.

Moreover, nucleic acid molecules encoding CPP proteins from other species, and thus that have a nucleotide sequence that differs from the sequence of SEQ ID NO:1, SEQ ID NO:14 or SEQ ID NO:17 are intended to be within the scope of the invention. Nucleic acid molecules corresponding to natural allelic variants and homologues of the CPP cDNAs of the invention can be isolated based on their homology to the Arabidopsis thaliana, Brassica napus or Glycine max CPP nucleic acids disclosed herein using the cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:1, SEQ ID NO:14 or SEQ ID NO:17. In another embodiment, the nucleic acid is at least 10, 25, 50, 100, 250, 500 or 750 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding CPP proteins derived from species other than Arabidopsis thaliana, Brassica napus or Glycine max) or other related sequences (e.g., paralogs) can be obtained by low, moderate or high stringency hybridization with all or a portion of the particular sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different depending upon circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS TN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of SEQ ID NO:1, SEQ ID NO:14 or SEQ ID NO:17 corresponds to a naturally occurring nucleic acid molecule. As used herein, a “inaturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 1, SEQ ID NO:14 or SEQ ID NO:17, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of SEQ ID NO: 1 or SEQ ID NO:14, or SEQ ID NO: 17 or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

Conservative Mutations

In addition to naturally-occurring allelic variants of the CPP sequence that may exist in the population, the skilled artisan will further appreciate that changes can be introduced by mutation into the nucleotide sequence of SEQ ID NO: 1, SEQ ID NO:14 or SEQ ID NO: 17, thereby leading to changes in the amino acid sequence of the encoded CPP protein, without altering the functional ability of the CPP protein. For example, nucleotide substitutions leading to amino acid substitutions at “non-essential” amino acid residues can be made in the sequence of SEQ ID NO: 1, SEQ ID NO:14 or SEQ ID NO: 17. A “non-essential” amino acid residue is a residue that can be altered from the wild-type sequence of CPP without altering the biological activity, whereas an “essential” amino acid residue is required for biological activity. For example, amino acid residues that are conserved among the CPP proteins of the present invention, are predicted to be particularly unamenable to alteration.

Another aspect of the invention pertains to nucleic acid molecules encoding CPP proteins that contain changes in amino acid residues that are not essential for activity. Such CPP proteins differ in amino acid sequence from SEQ ID NO: 2, SEQ ID NO:15 or SEQ ID NO:18, yet retain biological activity. In one embodiment, the isolated nucleic acid molecule comprises a nucleotide sequence encoding a protein, wherein the protein comprises an amino acid sequence at least about 75% homologous to the amino acid sequence of SEQ ID NO: 2, SEQ ID NO:15 or SEQ ID NO:18. Preferably, the protein encoded by the nucleic acid is at least about 80% homologous to S SEQ ID NO: 2, SEQ ID NO:15 or SEQ ID NO:18 more preferably at least about 90%, 95%, 98%, and most preferably at least about 99% homologous to SEQ ID NO: 2, SEQ ID NO:15 or SEQ ID NO:18.

An isolated nucleic acid molecule encoding a CPP protein homologous to the protein of SEQ ID NO: 2, SEQ ID NO:15 or SEQ ID NO:18 can be created by introducing one or more nucleotide substitutions, additions or deletions into the nucleotide sequence of SEQ ID NO:1 or SEQ ID NO:14, or SEQ ID NO:17 such that one or more amino acid substitutions, additions or deletions are introduced into the encoded protein.

Mutations can be introduced into the nucleotide sequence of SEQ ID NO:1, SEQ ID NO:14 or SEQ ID NO:17 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in CPP is replaced with another amino acid residue from the same side chain family. Alternatively, in another embodiment, mutations can be introduced randomly along all or part of a CPP coding sequence, such as by saturation mutagenesis, and the resultant mutants can be screened for CPP biological activity to identify mutants that retain activity. Following mutagenesis of SEQ ID NO:1, SEQ ID NO:14 or SEQ ID NO:17 the encoded protein can be expressed by any recombinant technology known in-the art and the activity of the protein can be determined.

In one embodiment, a mutant CPP protein can be assayed for (1) the ability to form protein:protein interactions with other CPP proteins, other cell-surface proteins, or biologically active portions thereof, (2) complex formation between a mutant CPP protein and a CPP receptor; (3) the ability of a mutant CPP protein to bind to an intracellular target protein or biologically active portion thereof; (e.g., avidin proteins); (4) the ability to bind CPP protein; or (5) the ability to specifically bind an anti-CPP protein antibody. Antisense CPP Nucleic Acids Another aspect of the invention pertains to isolated antisense nucleic acid molecules that are hybridizable to or complementary to the nucleic acid molecule comprising the nucleotide sequence of SEQ ID NO: 1 or SEQ ID NO:14 or SEQ ID NO:17, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire CPP coding strand, or to only a portion thereof. Nucleic acid molecules encoding fragments, homologs, derivatives and analogs of a CPP protein of SEQ ID NO: 2 or SEQ ID NO:15 or SEQ ID NO:18 or antisense nucleic acids complementary to a CPP nucleic acid sequence of SEQ ID NO: 1, SEQ ID NO:14 or SEQ ID NO:17 are additionally provided. Exemplary CPP anti-sense nucleic acid include the nucleic acid sequences of SEQ ID NO:16, 19, and 20.

In one embodiment, an antisense nucleic acid molecule is antisense to a “coding region” of the coding strand of a nucleotide sequence encoding CPP. The term “coding region” refers to the region of the nucleotide sequence comprising codons which are translated into amino acid residues (e.g., the protein coding region of Arabidopsis thaliana, Brassica napus or Glycine max CPP corresponds to SEQ ID NO: 2 or SEQ ID NO:15 or SEQ ID NO:18). In another embodiment, the antisense nucleic acid molecule is antisense to a “noncoding region” of the coding strand of a nucleotide sequence encoding CPP. The term “noncoding region” refers to 5′ and 3′ sequences which flank the coding region that are not translated into amino acids (i.e., also referred to as 5′ and 3′ untranslated regions).

Given the coding strand sequences encoding CPP disclosed herein (e.g., SEQ ID NO:1 or SEQ ID NO:14 or SEQ ID NO:17), antisense nucleic acids of the invention can be designed according to the rules of Watson and Crick or Hoogsteen base pairing. The antisense nucleic acid molecule can be complementary to the entire coding region of CPP mRNA, but more preferably is an oligonucleotide that is antisense to only a portion of the coding or noncoding region of CPP mRNA. For example, the antisense oligonucleotide can be complementary to the region surrounding the translation start site of CPP mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in, an antisense orientation (i.e., RNA transcribed from the inserted nucleic acid will be of an antisense orientation to a target nucleic acid of interest, described further in the following subsection).

The antisense nucleic acid molecules of the invention are generated in situ such that they hybridize with or bind to cellular mRNA and/or genomic DNA encoding a CPP protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an a-anomeric nucleic acid molecule. An o:-anomeric nucleic acid molecule forms specific double-stranded hybrids with complementary RNA in which, contrary to the usual P-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Lett 215: 327-330).

Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in applications.

Double Stranded RNA Inhibition (RNAi) by Hairpin Nucleic Acids

Another aspect of the invention pertains to the use of post transcriptional gene silencing (PTGS) to repress gene expression. Double stranded RNA can initiate the sequence specific repression of gene expression in plants and animals. Double stranded RNA is processed to short duplex oligomers of 21-23 nucleotides in length. These small interfering RNA's suppress the expression of endogenous and heterologous genes in a sequence specific manner (Fire et al. Nature 391:806-811, Carthew, Curr. Opin. in Cell Biol., 13:244-248, Elbashir et al., Nature 411:494-498). A RNAi suppressing construct can be designed in a number of ways, for example, transcription of a inverted repeat which can form a long hair pin molecule, inverted repeats separated by a spacer sequence that could be an unrelated sequence such as GUS or an intron sequence. Transcription of sense and antisense strands by opposing promoters or cotranscription of sense and antisense genes.

CPP Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as a mRNA, to which they have a complementary region. Thus, ribozymes (e.g., hammerhead ribozymes (described in Haselhoff and Gerlach (1988) Nature 334:585-591)) can be used to catalytically cleave CPP mRNA transcripts to thereby inhibit translation of CPP mRNA. A ribozyme having specificity for a CPP-encoding nucleic acid can be designed based upon the nucleotide sequence of a CPP DNA disclosed herein (i.e., SEQ ID NO: 1, SEQ ID NO:14 or SEQ ID NO:17). For example, a derivative of a Tetrahymena L-19 IVS RNA can be constructed in which the nucleotide sequence of the active site is complementary to the nucleotide sequence to be cleaved in a CPP-encoding mRNA. See, e.g., Cech et al. U.S. Pat. No. 4,987,071; and Cech et al. U.S. Pat. No. 5,116,742. Alternatively, CPP mRNA can be used to select a catalytic RNA having a specific ribonuclease activity from a pool of RNA molecules. See, e.g., Bartel et al., (1993) Science 261:1411-1418.

Alternatively, CPP gene expression can be inhibited by targeting nucleotide sequences complementary to the regulatory region of the CPP (e.g., the CPP promoter and/or enhancers) to form triple helical structures that prevent transcription of the CPP gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

In various embodiments, the nucleic acids of CPP can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of CPP can be used in therapeutic and diagnostic applications. For example, PNAs can be used as antisense or antigene agents for sequence-specific modulation of gene expression by, e.g., inducing transcription or translation arrest or inhibiting replication. PNAs of CPP can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of CPP can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of CPP can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl) amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

CPP Polypeptides

A CPP polypeptide of the invention includes the protein whose sequence is provided in SEQ ID NO: 2, SEQ ID NO:15 or SEQ ID NO:18. The invention also includes a mutant or variant protein any of whose residues may be changed from the corresponding residue shown in SEQ ID NO: 2, SEQ ID NO:15 or SEQ ID NO:18 while still encoding a protein that maintains its CPP-like activities and physiological functions, or a functional fragment thereof. In some embodiments, up to 20% or more of the residues may be so changed in the mutant or variant protein. In some embodiments, the CPP polypeptide according to the invention is a mature polypeptide.

In general, a CPP-like variant that preserves CPP-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated CPP proteins, and biologically active portions thereof, or derivatives, fragments, analogs or homologs thereof. Also provided are polypeptide fragments suitable for use as immunogens to raise anti-CPP antibodies. In one embodiment, native CPP proteins can be isolated from cells or tissue sources by an appropriate purification scheme using standard protein purification techniques. In another embodiment, CPP proteins are produced by recombinant DNA techniques. Alternative to recombinant expression, a CPP protein or polypeptide can be synthesized chemically using standard peptide synthesis techniques.

An “isolated” or “purified” protein or biologically active portion thereof is substantially free of cellular material or other contaminating proteins from the cell or tissue source from which the CPP protein is derived, or substantially free from chemical precursors or other chemicals when chemically synthesized. The language “substantially free of cellular material” includes preparations of CPP protein in which the protein is separated from cellular components of the cells from which it is isolated or recombinantly produced. In one embodiment, the language “substantially free of cellular material” includes preparations of CPP protein having less than about 30% (by dry weight) of non-CPP protein (also referred to herein as a “contaminating protein”), more preferably less than about 20% of non-CPP protein, still more preferably less than about 10% of non-CPP protein, and most preferably less than about 5% non-CPP protein. When the CPP protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of CPP protein in which the protein is separated from chemical precursors or other chemicals that are involved in the synthesis of the protein. In one embodiment, the language “substantially free of chemical precursors or other chemicals” includes preparations of CPP protein having less than about 30% (by dry weight) of chemical precursors or non-CPP chemicals, more preferably less than about 20% chemical precursors or non-CPP chemicals, still more preferably less than about 10% chemical precursors or non-CPP chemicals, and most preferably less than about 5% chemical precursors or non-CPP chemicals.

Biologically active portions of a CPP protein include peptides comprising amino acid sequences sufficiently homologous to or derived from the amino acid sequence of the CPP protein, e.g., the amino acid sequence shown in SEQ ID NO: 2 that include fewer amino acids than the full length CPP proteins, and exhibit at least one activity of a CPP protein, e.g. substrate binding. Typically, biologically active portions comprise a domain or motif with at least one activity of the CPP protein. A biologically active portion of a CPP protein can be a polypeptide which is, for example, 10, 25, 50, 100 or more amino acids in length.

A biologically active portion of a CPP protein of the present invention may contain at least one of the above-identified domains conserved between the CPP proteins, e.g. Moreover, other biologically active portions, in which other regions of the protein are deleted, can be prepared by recombinant techniques and evaluated for one or more of the functional activities of a native CPP protein.

A biologically active portion or a CPP protein can be the N-terminal domain of the CPP polypeptide. Alternatively, a biologically active portion or a CPP protein can be the C-terminal domain of the CPP polypeptide. Preferably, the biologically active portion comprises at least 75 amino acids of the C-terminal domain. More preferably, the biologically active portion comprises at least 25 amino acids of the C-terminal domain. Most preferably, the biologically active portion comprises at least 10 amino acids of the C-terminal.

In an embodiment, the CPP protein has an amino acid sequence shown in SEQ ID NO: 2, SEQ ID NO:15 or SEQ ID NO:18. In other embodiments, the CPP protein is substantially homologous to SEQ ID NO: 2, SEQ ID NO:15 or SEQ ID NO:18 and retains the functional activity of the protein of SEQ ID NO: 2, SEQ ID NO:15 or SEQ ID NO:18, yet differs in amino acid sequence due to natural allelic variation or mutagenesis, as described in detail below. Accordingly, in another embodiment, the CPP protein is a protein that comprises an amino acid sequence at least about 45% homologous to the amino acid sequence of S SEQ ID NO: 2, SEQ ID NO:15 or SEQ ID NO:18 and retains the functional activity of the CPP proteins of SEQ ID NO: 2, SEQ ID NO:15 or SEQ ID NO:18.

Exemplary homologous CPP polypeptides include for example the polypeptide sequences of SEQ ID NO: 69, 71, 73 and 75.

Determining Homology Between Two or More Sequence

To determine the percent homology of two amino acid sequences or of two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO:1 or SEQ ID NO:14 or SEQ ID NO:17.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

Chimeric and Fusion Proteins

The invention also provides CPP chimeric or fusion proteins. As used herein, a CPP “chimeric protein” or “fusion protein” comprises a CPP polypeptide operatively linked to a non-CPP polypeptide. An “CPP polypeptide” refers to a polypeptide having an amino acid sequence corresponding to CPP, whereas a “non-CPP polypeptide” refers to a polypeptide having an amino acid sequence corresponding to a protein that is not substantially homologous to the CPP protein, e.g., a protein that is different from the CPP protein and that is derived from the same or a different organism. Within a CPP fusion protein the CPP polypeptide can correspond to all or a portion of a CPP protein. In one embodiment, a CPP fusion protein comprises at least one biologically active portion of a CPP protein. In another embodiment, a CPP fusion protein comprises at least two biologically active portions of a CPP protein. Within the fusion protein, the term “operatively linked” is intended to indicate that the CPP polypeptide and the non-CPP polypeptide are fused in-frame to each other. The non-CPP polypeptide can be fused to the N-terminus or C-terminus of the CPP polypeptide.

A CPP chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A CPP-encoding nucleic acid can be cloned into such an expression vector such that the fusion moiety is linked in-frame to the CPP protein.

CPP Agonists and Antagonists

The present invention also pertains to variants of the CPP proteins that function as either CPP agonists (mimetics) or as CPP antagonists. Variants of the CPP protein can be generated by mutagenesis, e.g., discrete point mutation or truncation of the CPP protein. An agonist of the CPP protein can retain substantially the same, or a subset of, the biological activities of the naturally occurring form of the CPP protein. An antagonist of the CPP protein can inhibit one or more of the activities of the naturally occurring form of the CPP protein by, for example, competitively binding to a downstream or upstream member of a cellular signaling cascade which includes the CPP protein. Thus, specific biological effects can be elicited by treatment with a variant of limited function.

Variants of the CPP protein that function as either CPP agonists (mimetics) or as CPP antagonists can be identified by screening combinatorial libraries of mutants, e.g., truncation mutants, of the CPP protein for CPP protein agonist or antagonist activity. In one embodiment, a variegated library of CPP variants is generated by combinatorial mutagenesis at the nucleic acid level and is encoded by a variegated gene library. A variegated library of CPP variants can be produced by, for example, enzymatically ligating a mixture of synthetic oligonucleotides into gene sequences such that a degenerate set of potential CPP sequences is expressible as individual polypeptides, or alternatively, as a set of larger fusion proteins (e.g., for phage display) containing the set of CPP sequences therein. There are a variety of methods which can be used to produce libraries of potential CPP variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential CPP sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide Libraries

In addition, libraries of fragments of the CPP protein coding sequence can be used to generate a variegated population of CPP fragments for screening and subsequent selection of variants of a CPP protein. In one embodiment, a library of coding sequence fragments can be generated by treating a double stranded PCR fragment of a CPP coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the CPP protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of CPP proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique that enhances the frequency of functional mutants in the libraries, can be used in combination with the screening assays to identify CPP variants (Arkin and Yourvan (1992) PNAS 89:7811-7815; Delgrave et al. (1993) Protein Engineering 6:327-331).

CPP Antibodies

CPP polypeptides, including chimeric polypeptides, or derivatives, fragments, analogs or homologs thereof, may be utilized as immunogens to generate antibodies that immunospecifically-bind these peptide components. Such antibodies include, e.g., polyclonal, monoclonal, chimeric, single chain, Fab fragments and a Fab expression library. In a specific embodiment, fragments of the CPP polypeptides are used as immunogens for antibody production. Various procedures known within the art may be used for the production of polyclonal or monoclonal antibodies to a CPP polypeptides, or derivative, fragment, analog or homolog thereof. For the production of polyclonal antibodies, various host animals may be immunized by injection with the native peptide, or a synthetic variant thereof, or a derivative of the foregoing. Various adjuvants may be used to increase the immunological response and include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.) and human adjuvants such as Bacille Calmette-Guerin and Corynebacterium parvum.

For preparation of monoclonal antibodies directed towards a CPP polypeptides, or derivatives, fragments, analogs or homologs thereof, any technique that provides for the production of antibody molecules by continuous cell line culture may be utilized. Such techniques include, but are not limited to, the hybridoma technique (see, Kohler and Milstein, 1975. Nature 256: 495-497); the trioma technique; the human B-cell hybridoma technique (see, Kozbor, et al., 1983. Immunol Today 4: 72) and the EBV hybridoma technique to produce human monoclonal antibodies (see, Cole, et al., 1985. In: Monoclonal Antibodies and Cancer Therapy, Alan R. Liss, Inc., pp. 77-96). Human monoclonal antibodies may be utilized in the practice of the present invention and may be produced by the use of human hybridomas (see, Cote, et al., 1983. Proc Natl Acad Sci USA 80: 2026-2030) or by transforming human B-cells with Epstein Barr Virus in vitro (see, Cole, et al., 1985. In: Monoclonal Antibodies and Cancer Therapy (Alan R. Liss, Inc., pp. 77-96).

According to the invention, techniques can be adapted for the production of single-chain antibodies specific to a CPP polypeptides (see, e.g., U.S. Pat. No. 4,946,778). In addition, methodologies can be adapted for the construction of Fab expression libraries (see, e.g., Huse, et al., 1989. Science 246: 1275-1281) to allow rapid and effective identification of monoclonal Fab fragments with the desired specificity for a CPP polypeptides or derivatives, fragments, analogs or homologs thereof. Antibody fragments that contain the idiotypes to a CPP polypeptides may be produced by techniques known in the art including, e.g., (i) an F(ab′)₂ fragment produced by pepsin digestion of an antibody molecule; (ii) an Fab fragment generated by reducing the disulfide bridges of an F(ab′)₂ fragment; (iii) an Fab fragment generated by the treatment of the antibody molecule with papain and a reducing agent and (iv) Fv fragments. In one embodiment, methodologies for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a CPP polypeptides is facilitated by generation of hybridomas that bind to the fragment of a CPP polypeptides possessing such a domain. Antibodies that are specific for a domain within a CPP polypeptides, or derivative, fragments, analogs or homologs thereof, are also provided herein. The anti-CPP polypeptide antibodies may be used in methods known within the art relating to the localization and/or quantitation of a CPP polypeptide(e.g., for use in measuring levels of the peptide within appropriate physiological samples, for use in diagnostic methods, for use in imaging the peptide, and the like).

CPP Recombinant Expression Vectors and Host Cells

Another aspect of the invention pertains to vectors, preferably expression vectors, containing a nucleic acid encoding a CPP protein, or derivatives, fragments, analogs or homologs thereof. As used herein, the term “vector” refers to a nucleic acid molecule capable of transporting another nucleic acid to which it has been linked. Exemplary expression vector constructs include for example the constructs of SEQ ID NO: 4, 5, 36, 37, 39, 40, 441, 42, 44, 45, 47, 48, 50, 51 and 53. Additional exemplary expression vector constructs include contructs comprising CPP anti-sense nucleic acid such as SEQ ID NO: 38. 43., 46, 49, 52. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication). Other vectors are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors or plant transformation vectors, binary or otherwise, which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). Examples of suitable promoters include for example constitutive promoters, ABA inducible promoters, tissue specific promters or guard cell specific promoters. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., CPP proteins, mutant forms of CPP proteins, fusion proteins, etc.).

The recombinant expression vectors of the invention can be designed for expression of CPP proteins in prokaryotic or eukaryotic cells. For example, CPP proteins can be expressed in bacterial cells such as Escherichia coli, insect cells (using baculovirus expression vectors) yeast cells, plant cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the CPP expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). Alternatively, CPP can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In yet another embodiment, a nucleic acid of the invention is expressed in plants cells using a plant expression vector. Examples of plant expression vectors systems include tumor inducing (Ti) plasmid or portion thereof found in Agrobacterium, cauliflower mosaic virus (CAMV) DNA and vectors such as pBI121.

For expression in plants, the recombinant expression cassette will contain in addition to the CPP nucleic acids, a plant promoter region, a transcription initiation site (if the coding sequence to transcribed lacks one), and a transcription termination/polyadenylation sequence. The termination/polyadenylation region may be obtained from the same gene as the promoter sequence or may be obtained from different genes. Unique restriction enzyme sites at the 5′ and 3′ ends of the cassette are typically included to allow for easy insertion into a pre-existing vector.

Examples of suitable promotors include promoters from plant viruses such as the ³⁵S promoter from cauliflower mosaic virus (CaMV). Odell, et al., Nature, 313: 810-812 (1985). and promoters from genes such as rice actin (McElroy, et al., Plant Cell, 163-171 (1990)); ubiquitin (Christensen, et al., Plant Mol. Biol., 12: 619-632 (1992); and Christensen, et al., Plant Mol. Biol., 18: 675-689 (1992)); pEMU (Last, et al., Theor. Appl. Genet., 81: 581-588 (1991)); MAS (Velten, et al., EMBO J., 3: 2723-2730 (1984)); maize H3 histone (Lepetit, et al., Mol. Gen. Genet., 231: 276-285 (1992); and Atanassvoa, et al., Plant Journal, 2(3): 291-300 (1992)), the 5′- or 3′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, ALS promoter, (WO 96/30530), a synthetic promoter, such as, Rsyn7, SCP and UCP promoters, ribulose-1,3-diphosphate carboxylase, fruit-specific promoters, heat shock promoters, seed-specific promoters and other transcription initiation regions from various plant genes, for example, include the various opine initiation regions, such as for example, octopine, mannopine, and nopaline. Additional regulatory elements that may be connected to a CPP encoding nucleic acid sequence for expression in plant cells include terminators, polyadenylation sequences, and nucleic acid sequences encoding signal peptides that permit localization within a plant cell or secretion of the protein from the cell. Such regulatory elements and methods for adding or exchanging these elements with the regulatory elements CPP gene are known, and include, but are not limited to, 3′ termination and/or polyadenylation regions such as those of the Agrobacterium tumefaciens nopaline synthase (nos) gene (Bevan, et al., Nucl. Acids Res., 12: 369-385 (1983)); the potato proteinase inhibitor II (PINII) gene (Keil, et al., Nucl. Acids Res., 14: 5641-5650 (1986) and hereby incorporated by reference); and An, et al., Plant Cell, 1: 115-122 (1989)); and the CaMV 19S gene (Mogen, et al., Plant Cell, 2: 1261-1272 (1990)).

Plant signal sequences, including, but not limited to, signal-peptide encoding DNA/RNA sequences which target proteins to the extracellular matrix of the plant cell (Dratewka-Kos, et al., J. Biol. Chem., 264: 4896-4900 (1989)) and the Nicotiana plumbaginifolia extension gene (DeLoose, et al., Gene, 99: 95-100 (1991)), or signal peptides which target proteins to the vacuole like the sweet potato sporamin gene (Matsuka, et al., Proc. Nat'l Acad. Sci. (USA), 88: 834 (1991)) and the barley lectin gene (Wilkins, et al., Plant Cell, 2: 301-313 (1990)), or signals which cause proteins to be secreted such as that of PRIb (Lind, et al., Plant Mol. Biol., 18: 47-53 (1992)), or those which target proteins to the plastids such as that of rapeseed enoyl-ACP reductase (Verwaert, et al., Plant Mol. Biol., 26: 189-202 (1994)) are useful in the invention.

In another embodiment, the recombinant expression vector is capable of directing expression of the nucleic acid preferentially in a particular cell type (e.g., tissue-specific regulatory elements are used to express the nucleic acid). Tissue-specific regulatory elements are known in the art. Especially useful in connection with the nucleic acids of the present invention are expression systems which are operable in plants. These include systems which are under control of a tissue-specific promoter, as well as those which involve promoters that are operable in all plant tissues.

Organ-specific promoters are also well known. For example, the patatin class I promoter is transcriptionally activated only in the potato tuber and can be used to target gene expression in the tuber (Bevan, M., 1986, Nucleic Acids Research 14:4625-4636). Another potato-specific promoter is the granule-bound starch synthase (GBSS) promoter (Visser, R. G. R, et al., 1991, Plant Molecular Biology 17:691-699).

Other organ-specific promoters appropriate for a desired target organ can be isolated using known procedures. These control sequences are generally associated with genes uniquely expressed in the desired organ. In a typical higher plant, each organ has thousands of mRNAs that are absent from other organ systems (reviewed in Goldberg, P., 1986, Trans. R. Soc. London B314:343).

For in situ production of the antisense mRNA of GST, those regions of the GST gene which are transcribed into GST mRNA, including the untranslated regions thereof, are inserted into the expression vector under control of the promoter system in a reverse orientation. The resulting transcribed mRNA is then complementary to that normally produced by the plant.

The resulting expression system or cassette is ligated into or otherwise constructed to be included in a recombinant vector which is appropriate for plant transformation. The vector may also contain a selectable marker gene by which transformed plant cells can be identified in culture. Usually, the marker gene will encode antibiotic resistance. These markers include resistance to G418, hygromycin, bleomycin, kanamycin, and gentamicin. After transforming the plant cells, those cells having the vector will be identified by their ability to grow on a medium containing the particular antibiotic. Replication sequences, of bacterial or viral origin, are generally also included to allow the vector to be cloned in a bacterial or phage host, preferably a broad host range prokaryotic origin of replication is included. A selectable marker for bacteria should also be included to allow selection of bacterial cells bearing the desired construct. Suitable prokaryotic selectable markers also include resistance to antibiotics such as kanamycin or tetracycline.

Other DNA sequences encoding additional functions may also be present in the vector, as is known in the art. For instance, in the case of Agrobacterium transformations, T-DNA sequences will also be included for subsequent transfer to plant chromosomes.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell.

A host cell of the invention, such as a prokaryotic or eukaryotic host cell in culture, can be used to produce (i.e., express) a polypeptide of the invention encoded in a an open reading frame of a polynucleotide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

A number of types of cells may act as suitable host cells for expression of a polypeptide encoded by an open reading frame in a polynucleotide of the invention. Plant host cells include, for example, plant cells that could function as suitable hosts for the expression of a polynucleotide of the invention include epidermal cells, mesophyll and other ground tissues, and vascular tissues in leaves, stems, floral organs, and roots from a variety of plant species, such as Arabidopsis thaliana, Nicotiana tabacum, Brassica napus, Zea mays, Oryza sativa, Gossypium hirsutum and Glycine max.

Alternatively, it may be possible to produce a polypeptide in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous polypeptides. If the polypeptide is made in yeast or bacteria, it may be necessary to modify the polypeptide produced therein, for example by phosphorylation or glycosylation of the appropriate sites, in order to obtain a functional polypeptide, if the polypeptide is of sufficient length and conformation to have activity. Such covalent attachments may be accomplished using known chemical or enzymatic methods.

A polypeptide may be prepared by culturing transformed host cells under culture conditions suitable to express the recombinant protein. The resulting expressed polypeptide or protein may then be purified from such culture (e.g., from culture medium or cell extracts) using known purification processes, such as gel filtration and ion exchange chromatography. The purification of the polypeptide or protein may also include an affinity column containing agents which will bind to the protein; one or more column steps over such affinity resins as concanavalin A-agarose, heparin-toyopearlo or Cibacrom blue 3GA SepharoseOR; one or more steps involving hydrophobic interaction chromatography using such resins as phenyl ether, butyl ether, or propyl ether; or immunoaffinity chromatography.

Alternatively, a polypeptide or protein may also be expressed in a form which will facilitate purification. For example, it may be expressed as a fusion protein containing a six-residue histidine tag. The histidine-tagged protein will then bind to a Ni-affinity column. After elution of all other proteins, the histidine-tagged protein can be eluted to achieve rapid and efficient purification. One or more reverse-phase high performance liquid chromatography (RP-HPLC) steps employing hydrophobic RP-HPLC media, e.g., silica gel having pendant methyl or other aliphatic groups, can be employed to further purify a polypeptide. Some or all of the foregoing purification steps, in various combinations, can also be employed to provide a substantially homogeneous isolated recombinant polypeptide. The protein or polypeptide thus purified is substantially free of other plant proteins or polypeptides and is defined in accordance with the present invention as “isolated.”

Transformed Plants Cells and Transgenic Plants

The invention includes protoplast, plants cells, plant tissue and plants (e.g., monocots and dicots transformed with a CPP nucleic acid (i.e, sense or anti sense), a vector containing a CPP nucleic acid (i.e, sense or antisense)or an expression vector containing a CPP nucleic acid (i.e, sense or antisense). As used herein, “plant” is meant to include not only a whole plant but also a portion thereof (i.e., cells, and tissues, including for example, leaves, stems, shoots, roots, flowers, fruits and seeds).

The plant can be any plant type including, for example, species from the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Gossypium, Picea, Caco, and Populus.

In some aspects of the invention, the transformed plant is resistant to biotic and abiotic stresses, e.g., chilling stress, salt stress, water stress (e.g., drought), disease, grazing pests and wound healing. Additionally, the invention also includes a transgenic plant that is resistant to pathogens such as for example fungi, bacteria, nematodes, viruses and parasitic weeds. Alternatively, the transgenic plant is resistant to herbicides or has delayed senesence. The transgenic plant has an increase in yield, productivity, biomass or ABA sensitivity. By resistant is meant the plant grows under stress conditions (e.g., high salt, decreased water, low temperatures) or under conditions that normally inhibit, to some degree, the growth of an untransformed plant. Methodologies to determine plant growth or response to stress include for example, height measurements, weight meaurements, leaf area, ability to flower, water use, transpiration rates and yield.

The invention also includes cells, tissues, including for example, leaves, stems, shoots, roots, flowers, fruits and seeds and the progeny derived from the tranformed plant.

Numerous methods for introducing foreign genes into plants are known and can be used to insert a gene into a plant host, including biological and physical plant transformation protocols. See, for example, Miki et al., (1993) “Procedure for Introducing Foreign DNA into Plants”, In: Methods in Plant Molecular Biology and Biotechnology, Glick and Thompson, eds., CRC Press, Inc., Boca Raton, pages 67-88 and Andrew Bent in, Clough SJ and Bent AF, 1998. Floral dipping: a simplified method for Agrobacterium-mediated transformation of Arabidopsis thaliana. The methods chosen vary with the host plant, and include chemical transfection methods such as calcium phosphate, polyethylene glycol (PEG) transformation, microorganism-mediated gene transfer such as Agrobacterium (Horsch, et al., Science, 227: 1229-31 (1985)), electroporation, protoplast transformation, micro-injection, flower dipping and biolistic bombardment.

Agrobacterium-Mediated Transformation

The most widely utilized method for introducing an expression vector into plants is based on the natural transformation system of Agrobacterium. A. tumefaciens and A. rhizogenes are plant pathogenic soil bacteria which genetically transform plant cells. The Ti and Ri plasmids of A. tumefaciens and A. rhizogenes, respectfully, carry genes responsible for genetic transformation of plants. See, for example, Kado, Crit. Rev. Plant Sci., 10: 1-32 (1991). Descriptions of the Agrobacterium vector systems and methods for Agrobacterium-mediated gene transfer are provided in Gruber et al., supra; and Moloney, et al, Plant Cell Reports, 8: 238-242 (1989).

Transgenic Arabidopsis plants can be produced easily by the method of dipping flowering plants into an Agrobacterium culture, based on the method of Andrew Bent in, Clough S J and Bent A F, 1998. Floral dipping: a simplified method for Agrobacterium-mediated transformation of Arabidopsis thaliana. Wild type plants are grown until the plant has both developing flowers and open flowers. The plant are inverted for 1 minute into a solution of Agrobacterium culture carrying the appropriate gene construct. Plants are then left horizontal in a tray and kept covered for two days to maintain humidity and then righted and bagged to continue growth and seed development. Mature seed is bulk harvested.

Direct Gene Transfer

A generally applicable method of plant transformation is microprojectile-mediated transformation, where DNA is carried on the surface of microprojectiles measuring about 1 to 4 mu.m. The expression vector is introduced into plant tissues with a biolistic device that accelerates the microprojectiles to speeds of 300 to 600 m/s which is sufficient to penetrate the plant cell walls and membranes. (Sanford, et al., Part. Sci. Technol., 5: 27-37 (1987); Sanford, Trends Biotech, 6: 299-302 (1988); Sanford, Physiol. Plant, 79: 206-209 (1990); Klein, et al., Biotechnology, 10: 286-291 (1992)).

Another method for physical delivery of DNA to plants is sonication of target cells as described in Zang, et al., BioTechnology, 9: 996-996 (1991). Alternatively, liposome or spheroplast fusions have been used to introduce expression vectors into plants. See, for example, Deshayes, et al., EMBO J., 4: 2731-2737 (1985); and Christou, et al., Proc. Nat'l. Acad. Sci. (USA), 84: 3962-3966 (1987). Direct uptake of DNA into protoplasts using CaCl.sub.2 precipitation, polyvinyl alcohol or poly-L-omithine have also been reported. See, for example, Hain, et al., Mol. Gen. Genet., 199: 161 (1985); and Draper, et al., Plant Cell Physiol., 23: 451-458 (1982).

Electroporation of protoplasts and whole cells and tissues has also been described. See, for example, Donn, et al., (1990) In: Abstracts of the VIIth Int;l. Congress on Plant Cell and Tissue Culture IAPTC, A2-38, page 53; D'Halluin et al., Plant Cell, 4: 1495-1505 (1992); and Spencer et al., Plant Mol. Biol., 24: 51-61 (1994).

Particle Wounding/Agrobacterium Delivery

Another useful basic transformation protocol involves a combination of wounding by particle bombardment, followed by use of Agrobacterium for DNA delivery, as described by Bidney, et al., Plant Mol. Biol., 18: 301-31 (1992). Useful plasmids for plant transformation include Bin 19. See Bevan, Nucleic Acids Research, 12: 8711-8721 (1984), and hereby incorporated by reference.

In general, the intact meristem transformation method involves imbibing seed for 24 hours in the dark, removing the cotyledons and root radical, followed by culturing of the meristem explants. Twenty-four hours later, the primary leaves are removed to expose the apical meristem. The explants are placed apical dome side up and bombarded, e.g., twice with particles, followed by co-cultivation with Agrobacterium. To start the co-cultivation for intact meristems, Agrobacterium is placed on the meristem. After about a 3-day co-cultivation period the meristems are transferred to culture medium with cefotaxime plus kanamycin for the NPTII selection.

The split meristem method involves imbibing seed, breaking of the cotyledons to produce a clean fracture at the plane of the embryonic axis, excising the root tip and then bisecting the explants longitudinally between the primordial leaves. The two halves are placed cut surface up on the medium then bombarded twice with particles, followed by co-cultivation with Agrobacterium. For split meristems, after bombardment, the meristems are placed in an Agrobacterium suspension for 30 minutes. They are then removed from the suspension onto solid culture medium for three day co-cultivation. After this period, the meristems are transferred to fresh medium with cefotaxime plus kanamycin for selection.

Transfer by Plant Breeding

Alternatively, once a single transformed plant has been obtained by the foregoing recombinant DNA method, conventional plant breeding methods can be used to transfer the gene and associated regulatory sequences via crossing and backcrossing. Such intermediate methods will comprise the further steps of: (1) sexually crossing the transgenic plant with a plant from a second taxon; (2) recovering reproductive material from the progeny of the cross; and (3) growing transgenic plants from the reproductive material. Where desirable or necessary, the agronomic characteristics of the second taxon can be substantially preserved by expanding this method to include the further steps of repetitively: (1) backcrossing the transgenic progeny with non-transgenic plants from the second taxon; and (2) selecting for expression of an associated marker gene among the progeny of the backcross, until the desired percentage of the characteristics of the second taxon are present in the progeny along with the gene or genes imparting marker gene trait.

By the term “taxon” herein is meant a unit of botanical classification. It thus includes, genus, species, cultivars, varieties, variants and other minor taxonomic groups which lack a consistent nomenclature.

Regeneration of Transformants

The development or regeneration of plants from either single plant protoplasts or various explants is well known in the art (Weissbach and Weissbach, 1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a polypeptide of interest introduced by Agrobacterium from leaf explants can be achieved by methods well known in the art such as described (Horsch et al., 1985). In this procedure, transformants are cultured in the presence of a selection agent and in a medium that induces the regeneration of shoots in the plant strain being transformed as described (Fraley et al., 1983). In particular, U.S. Pat. No. 5,349,124 (specification incorporated herein by reference) details the creation of genetically transformed lettuce cells and plants resulting therefrom which express hybrid crystal proteins conferring insecticidal activity against Lepidopteran larvae to such plants.

This procedure typically produces shoots within two to four months and those shoots are then transferred to an appropriate root-inducing medium containing the selective agent and an antibiotic to prevent bacterial growth. Shoots that rooted in the presence of the selective agent to form plantlets are then transplanted to soil or other media to allow the production of roots. These procedures vary depending upon the particular plant strain employed, such variations being well known in the art.

Preferably, the regenerated plants are self-pollinated to provide homozygous transgenic plants, or pollen obtained from the regenerated plants is crossed to seed-grown plants of agronomically important, preferably inbred lines. Conversely, pollen from plants of those important lines is used to pollinate regenerated plants. A transgenic plant of the present invention containing a desired polypeptide is cultivated using methods well known to one skilled in the art.

A preferred transgenic plant is an independent segregant and can transmit the CPP gene and its activity to its progeny. A more preferred transgenic plant is homozygous for the gene, and transmits that gene to all of its offspring on sexual mating. Seed from a transgenic plant may be grown in the field or greenhouse, and resulting sexually mature transgenic plants are self-pollinated to generate true breeding plants. The progeny from these plants become true breeding lines that are evaluated for increased expression of the CPP transgene.

Method of Producing Transgenic Plants

Also included in the invention are methods of producing a transgenic plant. The method includes introducing into one or more plant cells a compound that alters CaaX prenyl protease expression or activity in the plant to generate a transgenic plant cell and regenerating a transgenic plant from the transgenic cell. In some aspects the compound increases alters CaaX prenyl protease expression or activity. Alternatively, the compound decrease alters CaaX prenyl protease expression or activity. The compound can be, e.g., (i) a CaaX prenyl protease polypeptide; (ii) a nucleic acid encoding a CaaX prenyl protease polypeptide; (iii) a nucleic acid that increases expression of a nucleic acid that encodes a CaaX prenyl protease polypeptide; (iv) a nucleic acid that decreases the expression of a nucleic acid that encodes a CaaX prenyl protease polypeptide; (v) a CaaX prenyl protease antisense nucleic acid and derivatives, fragments, analogs and homologs thereof. A nucleic acid that increases expression of a nucleic acid that encodes a CaaX prenyl protease polypeptide includes, e.g., promoters, enhancers. The nucleic acid can be either endogenous or exogenous. Preferably, the compound is a CaaX prenyl protease polypeptide or a nucleic acid encoding a CaaX prenyl protease polypeptide. For example the compound comprises the nucleic acid sequence of SEQ ID NO:1, 14, or 17 or fragement thereof. Alternatively, the compound is a CaaX prenyl protease antisence nucleic acid. For example the compound comprises the nucleic acid sequence of SEQ ID NO: 16, 19 or 20.

In various aspects the transgenic plant has an altered phenotype as compared to a wild type plant (i.e., untransformed). By altered phenotype is meant that the plant has a one or more characteristic that is different from the wild type plant. For example, the transgenic plant has an increased resistence to stress. Increased stress resistance is meant that the transgenic plant can grow under stress conditions (e.g., high salt, decreased water, low temperatures, high temperatures) or under conditions that normally inhibit the growth of an untransformed Stresses include, for example, chilling stress, heat stress, heat shock, salt stress, water stress (i.e, drought), nutritional stress, disease, grazing pests, wound healing, pathogens such as for example fungi, bacteria, nematodes, viruses or parasitic weed and herbicides. Methodologies to determine plant growth or response to stress include for example, height measurements, weight or biomass measurements, leaf area or number, ability to flower, water use, transpiration rates and yield. Alternatively, the transformed plant has an increased (i.e., enhanced) ABA sensitivity. The enhanced ABA sensitivity is at the seedling growth stage. Alternatively, the enhanced ABA sensitivity is at the mature plant stage. Additional altered phenotypes include for example, enhanced vegetative growth (e.g., increased leaf number, thickness and overall biomass), delayed reproductive growth (e.g., flowering later); enhanced seedling vigor (e.g.,increased root biomass and length), enhanced lateral root formation and therefore soil penetration more extensive vascular system resulting in an enhanced transport system.

The plant can be any plant type including, for example, species from the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Gossypium, Picea, Caco, and Populus.

Screening Methods

The isolated nucleic acid molecules of the invention can be used to express CPP protein (e.g., via a recombinant expression vector in a host cell), to detect CPP mRNA (e.g., in a biological sample) or a genetic lesion in a CPP gene, and to modulate CPP activity, as described further, below. In addition, the CPP proteins can be used to screen compounds that modulate the CPP protein activity or expression. In addition, the anti-CPP antibodies of the invention can be used to detect and isolate CPP proteins and modulate CPP activity.

The invention provides a method (also referred to herein as a “screening assay”) for identifying modulators, i.e., candidate or test compounds or agents (e.g., peptides, peptidomimetics, small molecules or other drugs) that bind to CPP proteins or have a stimulatory or inhibitory effect on, e.g., CPP protein expression or CPP protein activity. The invention also includes compounds identified in the screening assays described herein.

In one embodiment, the invention provides assays for screening candidate or test compounds which bind to a CPP protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. SmaIl molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention. Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. US.A. 87: 6378-6382; Felici, 1991. J. Mol. Biol. 222: 301-310; Ladner, U.S. Pat. No. 5,233,409.). In one embodiment, an assay is a cell-based assay in which a cell which expresses a CPP protein, or a biologically-active portion thereof, is contacted with a test compound and the ability of the test compound to bind to a CPP protein determined. The cell, for example, can be of mammalian origin, plant cell or a yeast cell. Determining the ability of the test compound to bind to the CPP protein can be accomplished, for example, by coupling the test compound with a radioisotope or enzymatic label such that binding of the test compound to the CPP protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a CPP protein, or a biologically-active portion thereof, with a known compound which binds CPP to form an assay mixture, contacting the assay mixture with a test compound, and determining the ability of the test compound to interact with a CPP protein, wherein determining the ability of the test compound to interact with a CPP protein comprises determining the ability of the test compound to preferentially bind to CPP protein or a biologically-active portion thereof as compared to the known compound.

In another embodiment, an assay is a cell-based assay comprising contacting a cell expressing a CPP protein, or a biologically-active portion thereof, with a test compound and determining the ability of the test compound to modulate (e.g., stimulate or inhibit) the activity of the CPP protein or biologically-active portion thereof. Determining the ability of the test compound to modulate the activity of CPP or a biologically-active portion thereof can be accomplished, for example, by determining the ability of the CPP protein to bind to or interact with a CPP target molecule. As used herein, a “target molecule” is a molecule with which a CPP protein binds or interacts in nature, for example, a molecule on the surface of a cell which expresses a CPP interacting protein, a molecule on the surface of a second cell, a molecule in the extracellular milieu, a molecule associated with the internal surface of a cell membrane or a cytoplasmic molecule. A CPP target molecule can be a non-CPP molecule or a CPP protein or polypeptide of the invention In one embodiment, a CPP target molecule is a component of a signal transduction pathway that facilitates transduction of an extracellular signal (e.g. a signal generated by binding of a compound to a membrane-bound molecule) through the cell membrane and into the cell. The target, for example, can be a second intercellular protein that has catalytic activity or a protein that facilitates the association of downstream signaling molecules with CPP. Determining the ability of the CPP protein to bind to or interact with a CPP target molecule can be accomplished by one of the methods described above for determining direct binding. In one embodiment, determining the ability of the CPP protein to bind to or interact with a CPP target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a CPP-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a CPP protein or biologically-active portion thereof with a test compound and determining the ability of the test compound to bind to the CPP protein or biologically-active portion thereof. Binding of the test compound to the CPP protein can be determined either directly or indirectly as described above. In one such embodiment, the assay comprises contacting the CPP protein or biologically-active portion thereof with a known compound which binds CPP to form an assay mixture, contacting the assay mixture with a test compound, and determining the ability of the test compound to interact with a CPP protein, wherein determining the ability of the test compound to interact with a CPP protein comprises determining the ability of the test compound to preferentially bind to CPP or biologically-active portion thereof as compared to the known compound.

In still another embodiment, an assay is a cell-free assay comprising contacting CPP protein or biologically-active portion thereof with a test compound and determining the ability of the test compound to modulate (e.g. stimulate or inhibit) the activity of the CPP protein or biologically-active portion thereof. Determining the ability of the test compound to modulate the activity of CPP can be accomplished, for example, by determining the ability of the CPP protein to bind to a CPP target molecule by one of the methods described above for determining direct binding. In an alternative embodiment, determining the ability of the test compound to modulate the activity of CPP protein can be accomplished by determining the ability of the CPP protein further modulate a CPP target molecule. For example, the catalytic/enzymatic activity of the target molecule on an appropriate substrate can be determined as described above.

In yet another embodiment, the cell-free assay comprises contacting the CPP protein or biologically-active portion thereof with a known compound which binds CPP protein to form an assay mixture, contacting the assay mixture with a test compound, and determining the ability of the test compound to interact with a CPP protein, wherein determining the ability of the test compound to interact with a CPP protein comprises determining the ability of the CPP protein to preferentially bind to or modulate the activity of a CPP target molecule.

The cell-free assays of the invention are amenable to use of both the soluble form or the membrane-bound form of CPP protein. In the case of cell-free assays comprising the membrane-bound form of CPP protein, it may be desirable to utilize a solubilizing agent such that the membrane-bound form of CPP protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Tritone X-100, Tritono X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either CPP protein or its target molecule to facilitate separation of complexed from uncomplexed forms of one or both of the proteins, as well as to accommodate automation of the assay. Binding of a test compound to CPP protein, or interaction of CPP protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-CPP fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or CPP protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of CPP protein binding or activity determined using standard techniques.

Other techniques for immobilizing proteins on matrices can also be used in the screening assays of the invention. For example, either the CPP protein or its target molecule can be immobilized utilizing conjugation of biotin and streptavidin. Biotinylated CPP protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with CPP protein or target molecules, but which do not interfere with binding of the CPP protein to its target molecule, can be derivatized to the wells of the plate, and unbound target or CPP protein trapped in the wells by antibody conjugation. Methods for detecting such complexes, in addition to those described above for the GST-immobilized complexes, include immunodetection of complexes using antibodies reactive with the CPP protein or target molecule, as well as enzyme-linked assays that rely on detecting an enzymatic activity associated with the CPP protein or target molecule.

In another embodiment, modulators of CPP protein expression are identified in a method wherein a cell is contacted with a candidate compound and the expression of CPP mRNA or protein in the cell is determined. The level of expression of CPP mRNA or protein in the presence of the candidate compound is compared to the level of expression of CPP mRNA or protein in the absence of the candidate compound. The candidate compound can then be identified as a modulator of CPP mRNA or protein expression based upon this comparison. For example, when expression of CPP mRNA or protein is greater (i.e., statistically significantly greater) in the presence of the candidate compound than in its absence, the candidate compound is identified as a stimulator of CPP mRNA or protein expression. Alternatively, when expression of CPP mRNA or protein is less (statistically significantly less) in the presence of the candidate compound than in its absence, the candidate compound is identified as an inhibitor of CPP mRNA or protein expression. The level of CPP mRNA or protein expression in the cells can be determined by methods described herein for detecting CPP mRNA or protein.

In yet another aspect of the invention, the CPP proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with CPP (“CPP-binding proteins” or “CPP-bp”) and modulate CPP activity. Such CPP-binding proteins are also likely to be involved in the propagation of signals by the CPP proteins as, for example, upstream or downstream elements of the CPP pathway.

The two-hybrid system is based on the modular nature of most transcription factors, which consist of separable DNA-binding and activation domains. Briefly, the assay utilizes two different DNA constructs. In one construct, the gene that codes for CPP is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a CPP-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with CPP.

In yet another aspect of the invention are methods which utilize the transgenic plants of the invention to identify CPP-interacting components via genetic screening protocols. These components can be for example, regulatory elements which modify CPP-gene expression, interacting proteins which directly modify CPP activity or interacting proteins which modify components of the same signal transduction pathway and thereby exert an effect on the expression or activity of CPP. Briefly, genetic screening protocols are applied to the transgenic plants of the invention and in so doing identify related genes which are not identified using a wild type background for the screen. For example an activation tagged library (Weigel, et al., 2000. Plant Physiol. 122: 1003-1013), can be produced using the transgenic plants of the invention as the genetic background. Plants are then screened for altered phenotypes from that displayed by the parent plants. Alternative methods of generating libraries from the transgenic plants of the invention can be used, for example, chemical or irradiation induced mutations, insertional inactivation or insertional activation methods.

The invention further pertains to novel agents identified by the aforementioned screening assays and uses thereof.

### EXAMPLES

### Example 1

### RT-PCR Amplification and Cloning of CaaX Prenyl Proteases

Total RNA was isolated from leaf tissue of Arabidopsis thaliana, Brassica napus and Glycine max, using the Qiagen RNeasy kit and used as template to amplify the CPP genes by RT-PCR. Reaction conditions were as follows; IX reaction buffer (10 mM Tris-HCl.PH 8.8, 1.5 mM MgCl₂, 50 mM KCl), dNTP's at 200 μM, IpM AtCPP BamFW and AtCPP SmaRV primers, 2.5U. Pfu DNA polymerase, and template plus water to a final volume of 100 μL. Reactions were run at 1 minute 94° C., 1 minute 60° C., 1 minute 72° C., for 30 cycles. Primers used to PCR amplify Arabidopsis and Brassica sequences were those identified by SEQ ID NO:6 and SEQ ID NO:7. Primers used to PCR amplify the Glycine sequence were those identified by SEQ ID NO:54 and SEQ ID NO:55. PCR products were separated from the RT-PCR reaction mixture using the Qiagen PCR column spin kit and ligated into the prepared cloning vector, pBluescript KS+. The vector had been prepared by digestion with EcoRV and treated with Taq polymerase in the presence of dTTP to produce a 3′ overhand suitable for ligation with the PCR products. The ligation products were transformed into E. coli DH5α cells, positive colonies selected and the resulting inserts sequenced. The above methodology is applicable to obtain homologous sequences and may require alternative primers.

TABLE 1

AtCPP BamFW:

(SEQ ID NO:6)

5′-AAAGGATCCATGGCGATTCCTTTCATGG-3′
AtCPP SmaRV:

(SEQ ID NO:7)

5′-AAACCCGGGTTAATCTGTCTTCTTGTCTTCTCCA-3′
GmCPP SmaFW:

(SEQ ID NO:54)

5′-AAACCCGGGATGGCGTTTCCCTACATGGAAGCC-3′
GmCPP SacRV:

(SEQ ID NO:55)

5′-AAAGAGCTCTTAGTCTTCCTTCTTATCCGGTTCG-3′

### Example 2

### Vector Construction

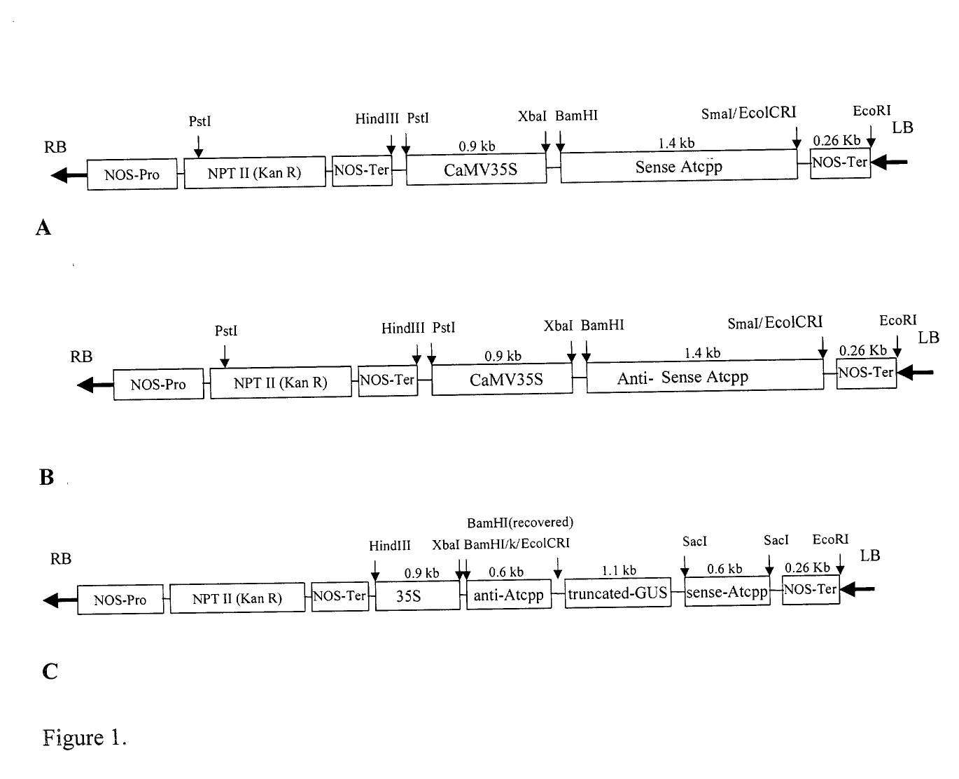

Construction of the pBI121-AtCPP construct (SEQ ID NO: 4) was prepared as follows. The pBI121 vector was digested with BamHI and SmaI. The AtCPP, 1.4 kb DNA fragment from RT-PCR (SEQ ID NO:1) was digested with BamHI and SmaI and ligated into the pBI121 vector. The GUS sequence was then removed by digestion with SmaI and EcolCRI and the vector ligated after purification of the vector from the GUS insert to produce the pBI121-AtCPP vector (FIG. 1A). This construct was used to further generate constructs expressing the CPP gene from Brassica and Glycine. To produce the pBI121-BnCPP construct (SEQ ID NO:47) primer pairs identified by SEQ ID NO:6 and SEQ ID NO:7 are used to PCR amplify the appropriate fragment which is ligated into the prepared parent vector. To produce the pBI121-GmCPP construct (SEQ ID NO:41) primer pairs identified by SEQ ID NO:54 and SEQ ID NO:55 are used to PCR amplify the appropriate fragment which is ligated into the prepared parent vector.

Construction of the pBI121-antisense-AtCPP construct (SEQ ID NO:35). The antisense fragment was produced using PCR amplification with SEQ ID NO:1 as template and primers identified as SEQ ID NO:11 and SEQ ID NO:12, listed in Table 2. This fragment was digested with BamHI and SmaI and used to replace the sense fragment of the pBI121-AtCPP construct (SEQ ID NO: 4), to yield SEQ ID NO:35 (FIG. 1B). This construct, SEQ ID NO:35, was used to further generate constructs expressing the antisense CPP gene from Brassica and Glycine. To produce the pBI121-antisense-BnCPP construct (SEQ ID NO:49) primer pairs identified by SEQ ID NO:56 and SEQ ID NO:57 are used to PCR amplify the appropriate fragment which is ligated into the prepared parent vector. To produce the pBI121-antisense-GmCPP construct (SEQ ID NO:43) primer pairs identified by SEQ ID NO:58 and SEQ ID NO:59 are used to PCR amplify the appropriate fragment which is ligated into the prepared parent vector.

Construction of the pBI121-HP-AtCPP construct (SEQ ID NO: 5). The cloning strategy involved truncating the GUS gene of pBI121 and flanking the GUS sequence with a AtCPP fragment in the antisense orientation upstream of the GUS and in the sense orientation on the downstream side of GUS. The pBI121 vector was digested with SmaI and SacI, the GUS sequence and the vector fragments were purified from one another. The isolated GUS fragment was digested using EcoRV and the 1079 bp. blunt ended EcoRVISacI fragment isolated. This was ligated back into the digested parent vector at the SmaI/SacI sites. This intermediate vector was used in the subsequent production of the hair-pin vectors. The AtCPP fragment to be used as the gene specific hair-pin sequence was isolated by PCR. Primers identified as SEQ ID NO:8 and SEQ ID NO:9, listed in Table 2, were used to generate a 596 bp fragment. Cloning of the sense orientation fragment was achieved by digesting the PCR AtCPP fragment with SacI and ligation into the SacI site at the 3′ end of GUS. To insert the same fragment upsteam of GUS, the BamHI site was opened and the ends blunted with Klenow. The PCR amplified AtCPP fragment was digested with EcolCRI, which is an isoschizomer of SacI but leaves blunt ends, and ligated into the blunted BamHI site of the vector to yield the final construct (FIG. 1C). The intermediate construct used to produce SEQ ID NO:5 above contained only the truncated GUS gene and no CPP sequences this intermediate vector was used to further generate constructs expressing hair-pin CPP gene constructs from Brassica and Glycine. To produce the pBI121-HP-BnCPP construct (SEQ ID NO:48) primer pairs identified by SEQ ID NO:58 and SEQ ID NO:59 are used to PCR amplify the sense fragment and primer pairs identified by SEQ ID NO:60 and SEQ ID NO:61 are used to PCR ampliify the antisense fragment. These fragments are cloned into the prepared intermediate vector described above. To produce the pBI121-HP-GmCPP construct (SEQ ID NO:42) primer pairs identified by SEQ ID NO:62 and SEQ ID NO:63 are used to PCR amplify the sense fragment and primer pairs identified by SEQ ID NO:64 and SEQ ID NO:65 are used to PCR amplify the antisense fragment. These fragments are cloned into the prepared intermediate vector described above. The above vector constructs were modified to place the genes under the control of alternative promoters, such as, but not limited to, the RD29A or MuA. This was accomplished by excising the ³⁵S promoter sequence and replacing it with an appropriate promoter sequence. In this way SEQ ID NO's:39 and 40 were generated and SEQ ID NO's:38, 41-53 can be constructed.

TABLE 2

AtCPP-HP-SacFW

(SEQ ID NO:8)

5′-CTGGAGCTCTTTTACCGAGGTTGGGCCTTGATCC-3′
AtCPP-HP-SacRV

(SEQ ID NO:9)

5′-ATTGAGCTCCCAATGTCCAAGCTCGTGTGCAATA-3′
AtCPP-anti-SmaFW

(SEQ ID NO:11)

5′-AAACCCGGGATGGCGATTCCTTTCATGG-3′
AtCPP-anti-BamRV

(SEQ ID NO:12)

5′-AAAGGATCCTTAATCTGTCTTCTTGTCTTCTCCA-3′
BnCPP-anti-SmaFW

(SEQ ID NO:56)

5′-AAACCCGGGATGGCGATTCCTTTCATGG-3′
BnCPP-anti-BamRV

(SEQ ID NO:57)

5′-AAAGGATCCTTAATCTGTCTTCTTGTCTTCTCC-3′
BnCPP-HP-Sac-FW

(SEQ ID NO:58)

5′-AAAGAGCTCTTCTACCAATGGTGGGACTCG-3′
BnCPP-HP-Sac-RV

(SEQ ID NO:59)

5′-AAAGAGCTCCCAGTGTCCCAGCTCGTGTG-3′
BnCPP-HP-BamFW

(SEQ ID NO:60)

5′-AAAGGATCCTTCTACCAATGGTGGGACTCG-3′
BnCPP-HP-XbaRV

(SEQ ID NO:61)

5′-AAATCTAGACCAGTGTCCCAGCTCGTGTG-3′
GmCPP-HP-Sac-FW

(SEQ ID NO:62)

5′-GATGAGCTCACAAGATCAAGTCACAGCAATGCCT-3′
GmCPP-HP-Sac-RV

(SEQ ID NO:63)

5′-AAAGAGCTCCCGGTTCGTCCAGCGCGGCC-3′
GmCPP-HP-BamFW

(SEQ ID NO:64)

5′-GATGGATCCACAAGATCAAGTCACAGCAATGCCT-3′
GmCPP-HP-XbaRV

(SEQ ID NO:65)

5′-CCTTCTAGACCGGTTCGTCCAGCGCGGCC-3′

### Example 3

### Sequence Analysis

Arabidopsis thaliana CPP (AtCPP)

A disclosed nucleic acid of 1275 nucleotides (SEQ ID NO:1) and also referred to as AtCPP, is shown in Table 3.

TABLE 3A

AtCPP Nucleotide Sequence.

| | |
|---|---|
| ATGGCGATTCCTTTCATGGAAACCGTCGTGGGTTTTATGATAGTGATGTACATTTTTGAG | (SEQ ID NO:1) |
| ACGTATTTGGATCTGAGGCAACTCACTGCTCTCAAGCTTCCAACTCTCCCGAAAACCTTG | |
| GTTGGTGTAATTAGCCAAGAGAAGTTTGAGAAATCACGAGCATACAGTCTTGACAAAAGC | |
| TATTTTCACTTTGTTCATGAGTTTGTAACTATACTTATGGACTCTGCAATTTTGTTCTTT | |
| GGGATCTTGCCTTGGTTTTGGAAGATGTCTGGAGCTGTTTTACCGAGGTTGGGCCTTGAT | |
| CCGGAGAATGAAATACTGCATACTCTTTCATTCTTGGCTGGTGTTATGACATGGTCACAG | |
| ATCACTGATTTGCCATTTTCTTTGTACTCAACTTTCGTGATCGAGTCTCGGCATGGGTTC | |
| AACAAACAAACAATATGGATGTTCATTAGGGACATGATCAAAGGAACATTCCTCTCTGTC | |
| ATACTAGGCCCACCCATTGTTGCTGCGATAATTTTCATAGTCCAGAAAGGAGGTCCTTAT | |
| CTTGCCATCTATCTGTGGGCATTCATGTTTATCCTGTCTCTAGTGATGATGACTATATAC | |
| CCGGTCTTGATAGCACCGCTCTTCAACAAATTCACTCCTCTTCCAGATGGAGACCTCCGG | |
| GAGAAGATTGAGAAACTTGCTTCTTCCCTAAAGTTTCCTTTGAAGAAGCTGTTTGTTGTC | |
| GATGGATCTACAAGGTCAAGCCATAGCAATGCTTACATGTATGGTTTCTTTAAGAACAAA | |
| AGGATTGTTCTTTATGATACGTTGATTCAGCAGTGCAAGAATGAGGATGAAATTGTGGCG | |
| GTTATTGCACACGAGCTTGGACATTGGAAACTGAATCACACTACATACTCGTTCATTGCA | |
| GTTCAAATCCTTGCCTTCTTACAATTTGGAGGATACACTCTTCTCAGAAACTCCACTGAT | |
| CTCTTCAGGAGTTTCGGATTTGATACACAGCCTGTTCTCATTGGTTTGATCATATTTCAG | |
| CACACTGTAATACCACTGCAACATCTAGTAAGCTTTGGCCTGAACCTCGTTAGTCGAGCG | |
| TTTGAGTTTCAGGCTGATGCTTTTGCTGTGAAGCTTGACTATGCAAAAGATCTTCGTCCT | |
| GCTCTAGTGAAACTACAGGAAGAGAACTTATCAACAATGAACACTGATCCATTGTACTCA | |
| GCTTATCACTACTCACATCCTCCTCTTGTTGAAAGGCTTCGAGCCACTGATGGAGAAGAC | |
| AAGAAGACAGATTAA | |

A disclosed CPP polypeptide (SEQ ID NO:2) encoded by SEQ ID NO:1 has 424 amino acid residues and is presented in Table 3B using the one-letter amino acid code.

TABLE 3B

Encoded CPP protein sequence.

| | |
|---|---|
| MAIPFMETVVGFMIVMYIFETYLDLRQLTALKLPTLPKTLVGVISQEKFEKSRAYSLDKS | (SEQ ID NO:2) |
| YFHFVHEFVTILMDSAILFFGILPWFWKMSGAVLPRLGLDPENEILHTLSFLAGVMTWSQ | |
| ITDLPFSLYSTFVIESRHGFNKQTIWMFIRDMIKGTFLSVILGPPIVAAIIFIVQKGGPY | |
| LAIYLWAFMFILSLVMMTIYPVLIAPLFNKFTPLPDGDLREKIEKLASSLKFPLKKLFVV | |
| DGSTRSSHSNAYMYGFFKNKRIVLYDTLIQQCKNEDEIVAVIAHELGHWKLNHTTYSFIA | |
| VQILAFLQFGGYTLLRNSTDLFRSFGFDTQPVLIGLIIFQHTVIPLQHLVSFGLNLVSRA | |
| FEFQADAFAVKLDYAKDLRPALVKLQEENLSTMNTDPLYSAYHYSHPPLVERLRATDGED | |
| KKTD | |

The present invention also includes a nucleic acid sequence complimentary to the Arabidopsis thaliana CaaX prenyl protease of SEQ ID NO:1. The disclosed complimentary sequence is shown as SEQ ID NO:20.

| | |
|---|---|
| TTAATCTGTCTTCTTGTCTTCTCCATCAGTGGCTCGAAGCCTTTCAACAAGAGGAGGAT | SEQ ID NO:20 |
| GTGAGTAGTGATAAGCTGAGTACAATGGATCAGTGTTCATTGTTGATAAGTTCTCTTCC | |
| TGTAGTTTCACTAGAGCAGGACGAAGATCTTTTGCATAGTCAAGCTTCACAGCAAAAGC | |
| ATCAGCCTGAAACTCAAACGCTCGACTAACGAGGTTCAGGCCAAAGCTTACTAGATGTT | |
| GCAGTGGTATTACAGTGTGCTGAAATATGATCAAACCAATGAGAACAGGCTGTGTATCA | |
| AATCCGAAACTCCTGAAGAGATCAGTGGAGTTTCTGAGAAGAGTGTATCCTCCAAATTG | |
| TAAGAAGGCAAGGATTTGAACTGCAATGAACGAGTATGTAGTGTGATTCAGTTTCCAAT | |
| GTCCAAGCTCGTGTGCAATAACCGCCACAATTTCATCCTCATTCTTGCACTGCTGAATC | |
| AACGTATCATAAAGAACAATCCTTTTGTTCTTAAAGAAACCATACATGTAAGCATTGCT | |
| ATGGCTTGACCTTGTAGATCCATCGACAACAAACAGCTTCTTCAAAGGAAACTTTAGGG | |
| AAGAAGCAAGTTTCTCAATCTTCTCCCGGAGGTCTCCATCTGGAAGAGGAGTGAATTTG | |
| TTGAAGAGCGGTGCTATCAAGACCGGGTATATAGTCATCATCACTAGAGACAGGATAAA | |
| CATGAATGCCCACAGATAGATGGCAAGATAAGGACCTCCTTTCTGGACTATGAAAATTA | |
| TCGCAGCAACAATGGGTGGGCCTAGTATGACAGAGAGGAATGTTCCTTTGATCATGTCC | |
| CTAATGAACATCCATATTGTTTGTTTGTTGAACCCATGCCGAGACTCGATCACGAAAGT | |
| TGAGTACAAAGAAAATGGCAAATCAGTGATCTGTGACCATGTCATAACACCAGCCAAGA | |
| ATGAAAGAGTATGCAGTATTTCATTCTCCGGATCAAGGCCCAACCTCGGTAAAACAGCT | |
| CCAGACATCTTCCAAAACCAAGGCAAGATCCCAAAGAACAAAATTGCAGAGTCCATAAG | |
| TATAGTTACAAACTCATGAACAAAGTGAAAATAGCTTTTGTCAAGACTGTATGCTCGTG | |
| ATTTCTCAAACTTCTCTTGGCTAATTACACCAACCAAGGTTTTCGGGAGAGTTGGAAGC | |
| TTGAGAGCAGTGAGTTGCCTCAGATCCAAATACGTCTCAAAAATGTACATCACTATCAT | |
| AAAACCCACGACGGTTTCCATGAAAGGAATCGCCAT | |

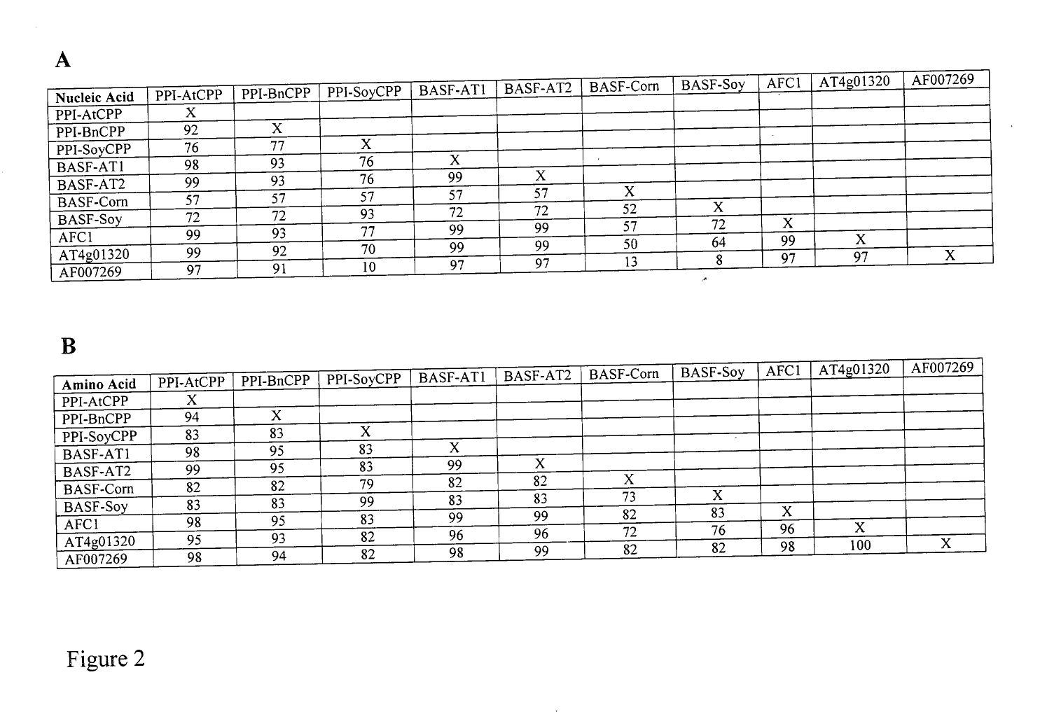

Due to the nature of the cloning strategy the sequence presented is not full length but is missing the 5′ and 3′ non-translated regions. The percent identities of the Arabidopsis thaliana nucleotide sequence and its encoded amino acid sequence to that of other CPP sequences as determined by ClustalW analysis are shown in FIG. 2. Using the sequences disclosed herein as hybridization probes, one is able to screen and isolate full length sequences from cDNA or genomic libraries or use the rapid amplification of cDNA ends (RACE) technology or other such PCR techniques.

Brassica napus CPP (BnCPP)

A disclosed nucleic acid of 1275 nucleotides (SEQ ID NO:14) and also referred to as BnCPP, is shown in Table 4.

TABLE 4A

BnCPP Nucleotide Sequence.

| | |
|---|---|
| ATGGCGATTCCTTTCATGGAAACCGTCGTTGGTTTTATGATAGTGATGTACGTTTTTGAGACGTA | (SEQ ID NO:14) |
| TTTGGATCTGAGGCAACATACTGCTCTCAAGCTTCCCACTCTCCCAAAGACTTTGGTTGGAGTCA | |
| TTAGCCAAGAGAAGTTTGAGAAATCTCGAGCTTACAGTCTTGACAAAAGCCATTTTCACTTTGTT | |
| CATGAGTTTGTTACTATACTTATGGACTCTGCGATTCTGTTCTTTGGGATCTTGCCTTGGTTTTG | |
| GAAGATATCTGGCGGCTTTCTACCAATGGTGGGACTCGATCCAGAGAATGAAATCCTGCACACTC | |
| TTTCATTCTTGGCTGGTCTTATGACATGGTCACAGATCACTGATTTGCCATTTTCTTTGTACTCA | |
| ACTTTCGTGATCGAGTCTCGGCATGGGTTCAACAAACAAACAATATGGATGTTCATTAGGGACAT | |
| GATCAAAGGAATACTCCTCTCTGTCATACCTGCCCCTCCTATCGTTGCCGCAATTATTGTTATAG | |
| TTCAGAAAGGAGGTCCTTACCTCGCCATCTATCTGTGGGCATTCATGTTTATCCTGTCTCTAGTG | |
| ATGATGACTATATACCCTGTTTTGATTGCACCTCTTTTCAACAAGTTCACTCCTCTTCCTGATGG | |
| AGACCTCCGGGAGAAGATTGAGAAACTTGCTTCTTCTCTAAAGTTTCCTCTGAAGAAGCTGTTTG | |
| TTGTCGATGGATCTACAAGGTCAAGCCATAGTAATGCTTACATGTATGGTTTCTTCAAGAACAAA | |
| AGGATTGTTCTTTATGACACATTGATTCAGCAGTGCCAGAATGAGAATGAAATTGTGGCGGTTAT | |
| TGCACACGAGCTGGGACACTGGAAGCTGAATCACACTACATACTCGTTCATTGCTGTTCAAATCC | |
| TTGCCTTCTTGCAATTTGGAGGATACACTCTTGTCAGAAACTCCACTGATCTCTTCAGGAGTTTT | |
| GGTTTTGATACACAACCAGTTCTCATTGGTTTGATCATATTTCAGCACACTGTAATACCACTTCA | |
| ACACCTAGTAAGCTTTGACCTCAACCTTGTTAGTCGAGCGTTTGAGTTTCAGGCTGATGCTTTTG | |
| CAGTGAATCTTGGTTATGCAAAGGATCTACGTCCTGCCCTAGTGAAGCTACAGGAAGAGAACTTA | |
| TCAGCGATGAACACAGACCCATTGTACTCAGCTTATCACTACTCACACCCTCCTCTTGTAGAGAG | |
| GCTTCGAGCCATTGATGGAGAAGACAAGAAGACAGATTAA | |

A disclosed CPP polypeptide (SEQ ID NO:15) encoded by SEQ ID NO:14 has 424 amino acid residues and is presented in Table 4B using the one-letter amino acid code.

TABLE 4B

Encoded CPP protein sequence.

(SEQ ID NO:15)

MAIPFMETVVGFMIVMYVFETYLDLRQHTALKLPTLPKTLVGVISQEKFE
KSRAYSLDKSHFHFVHEFVTILMDSAILFFGILPWFWKISGGFLPMVGLD
PENEILHTLSFLAGLMTWSQITDLPFSLYSTFVIESRHGFNKQTIWMFIR
DMIKGILLSVIPAPPIVAAIIVIVQKGGPYLAIYLWAFMFILSLVMMTIY
PVLIAPLFNKFTPLPDGDLREKIEKLASSLKFPLKKLFVVDGSTRSSHSN
AYMYGFFKNKRIVLYDTLIQQCQNENEIVAVIAHELGHWKLNHTTYSFIA
VQILAFLQFGGYTLVRNSTDLFRSFGFDTQPVLIGLIIFQHTVIPLQHLV
SFDLNLVSRAFEFQADAFAVNLGYAKDLRPALVKLQEENLSAMNTDPLYS
AYHYSHPPLVERLRAIDGEDKKTD

The present invention also includes a nucleic acid sequence complimentary to the Brassica napus CaaX prenyl protease of SEQ ID NO:14. The disclosed complimentary sequence is shown as SEQ ID NO:16.

| | |
|---|---|
| TTAATCTGTCTTCTTGTCTTCTCCATCAATGGCTCGAAGCCTCTCTACAAGAGGAGGGT | SEQ ID NO:16 |
| GTGAGTAGTGATAAGCTGAGTACAATGGGTCTGTGTTCATCGCTGATAAGTTCTCTTCC | |
| TGTAGCTTCACTAGGGCAGGACGTAGATCCTTTGCATAACCAAGATTCACTGCAAAAGC | |
| ATCAGCCTGAAACTCAAACGCTCGACTAACAAGGTTGAGGTCAAAGCTTACTAGGTGTT | |
| GAAGTGGTATTACAGTGTGCTGAAATATGATCAAACCAATGAGAACTGGTTGTGTATCA | |
| AAACCAAAACTCCTGAAGAGATCAGTGGAGTTTCTGACAAGAGTGTATCCTCCAAATTG | |
| CAAGAAGGCAAGGATTTGAACAGCAATGAACGAGTATGTAGTGTGATTCAGCTTCCAGT | |
| GTCCCAGCTCGTGTGCAATAACCGCCACAATTTCATTCTCATTCTGGCACTGCTGAATC | |
| AATGTGTCATAAAGAACAATCCTTTTGTTCTTGAAGAAACCATACATGTAAGCATTACT | |
| ATGGCTTGACCTTGTAGATCCATCGACAACAAACAGCTTCTTCAGAGGAAACTTTAGAG | |
| AAGAAGCAAGTTTCTCAATCTTCTCCCGGAGGTCTCCATCAGGAAGAGGAGTGAACTTG | |
| TTGAAAAGAGGTGCAATCAAAACAGGGTATATAGTCATCATCACTAGAGACAGGATAAA | |
| CATGAATGCCCACAGATAGATGGCGAGGTAAGGACCTCCTTTCTGAACTATAACAATAA | |
| TTGCGGCAACGATAGGAGGGGCAGGTATGACAGAGAGGAGTATTCCTTTGATCATGTCC | |
| CTAATGAACATCCATATTGTTTGTTTGTTGAACCCATGCCGAGACTCGATCACGAAAGT | |
| TGAGTACAAAGAAAATGGCAAATCAGTGATCTGTGACCATGTCATAAGACCAGCCAAGA | |
| ATGAAAGAGTGTGCAGGATTTCATTCTCTGGATCGAGTCCCACCATTGGTAGAAAGCCG | |
| CCAGATATCTTCCAAAACCAAGGCAAGATCCCAAAGAACAGAATCGCAGAGTCCATAAG | |
| TATAGTAACAAACTCATGAACAAAGTGAAAATGGCTTTTGTCAAGACTGTAAGCTCGAG | |
| ATTTCTCAAACTTCTCTTGGCTAATGACTCCAACCAAAGTCTTTGGGAGAGTGGGAAGC | |
| TTGAGAGCAGTATGTTGCCTCAGATCCAAATACGTCTCAAAAACGTACATCACTATCAT | |
| AAAACCAACGACGGTTTCCATGAAAGGAATCGCCAT | |

Due to the nature of the cloning strategy the sequence presented is not full length but is missing the 5′ and 3′ non-translated regions. The percent identities of the Brassica napus nucleotide sequence and its encoded amino acid sequence to that of other CPP sequences as determined by ClustalW analysis are shown in FIG. 2. Using the sequences disclosed herein as hybridization probes, one is able to screen and isolate full length sequences from cDNA or genomic libraries or use the rapid amplification of cDNA ends (RACE) technology or other such PCR techniques.

Glycine max CPP (GmCPP)

A disclosed nucleic acid of 1275 nucleotides (SEQ ID NO:17) and also referred to as GmCPP, is shown in Table 5.

TABLE 5A

GmCPP Nucleotide Sequence.

| | |
|---|---|
| ATGGCGTTTCCCTACATGGAAGCCGTTGTCGGATTTATGATATTAATGTACATTTTTGAAACTTA | (SEQ ID NO:17) |
| CTTGGATGTGCGACAACATAGGGCCCTCAAACTTCCTACTCTTCCAAAGACTTTAGAGGGTGTTA | |
| TCAGCCAAGAGAAATTTGAGAAATCTAGAGCCTATAGTCTTGATAAAAGCCACTTCCATTTTGTT | |
| CACGAGTTTGTGACAATAGTGACAGACTCTACAATTTTGTACTTTGGGGTATTGCCCTGGTTTTG | |
| GAAGAAATCAGGAGATTTTATGACAATAGCTGGTTTCAATGCTGAGAATGAAATACTGCATACCC | |
| TTGCCTTCTTAGCAGGGCTGATGATTTGGTCACAGATAACAGATTTGCCCTTTTCTCTGTACTCA | |
| ACTTTTGTGATTGAGGCCCGTCATGGTTTTAATAAGCAAACACCATGGTTATTCTTTAGGGACAT | |
| GCTTAAAGGAATTTTCCTTTCTGTAATAATTGGTCCACCTATTGTGGCTGCAATCATTGTAATAG | |
| TACAGAAAGGAGGTCCATACTTGGCCATCTATCTTTGGGTTTTTACGTTTGGTCTTTCTATTGTG | |
| ATGATGACCCTTTATCCAGTACTAATAGCTCCACTCTTCAATAAGTTCACTCCACTTCCAGATGG | |
| TCAACTCAGGGAGAAAATCGAGAAACTTGCTTCCTCCCTCAACTATCCGTTAAAGAAACTATTTG | |
| TTGTCGATGGATCCACAAGATCAAGTCACAGCAATGCCTATATGTATGGATTCTTCAAGAACAAG | |
| AGGATTGTCCCTTATGACACATTAATTCAACAGTGCAAAGACGATGAGGAAATTGTTGCTGTTAT | |
| TGCCCATGAGTTGGGACACTGGAAGCTCAACCATACTGTGTACACATTTGTTGCTATGCAGATTC | |
| TTACACTTCTACAATTTGGAGGATATACACTAGTGCGAAATTCAGCTGATCTGTATCGAAGCTTT | |
| GGGTTTGATACGCAGCCAGTCCTCATTGGGCTCATCATATTTCAGCATACTGTAATCCCACTTCA | |
| GCAATTGGTCAGCTTTGGTCTGAACCTAGTCAGCCGATCATTTGAATTTCAGGCTGATGGCTTTG | |
| CCAAGAAGCTTGGATATGCATCTGGATTACGCGGTGGTCTTGTGAAACTACAGGAGGAGAATCTG | |
| TCAGCTATGAATACAGATCCTTGGTACTCTGCTTATCACTATTCTCATCCTCCCCTTGTTGAAAG | |
| ATTGGCCGCGCTGGACGAACCGGATAAGAAGGAAGACTAA | |

A disclosed CPP polypeptide (SEQ ID NO:18) encoded by SEQ ID NO:17 has 424 amino acid residues and is presented in Table 5B using the one-letter amino acid code.

TABLE 5B

Encoded CPP protein sequence.

| | |
|---|---|
| MAFPYMEAVVGFMILMYIFETYLDVRQHRALKLPTLPKTLEGVISQEKFEKSRAYSLDKS | (SEQ ID NO:18) |
| HFHFVHEFVTIVTDSTILYFGVLPWFWKKSGDFMTIAGFNAENEILHTLAFLAGLMIWSQ | |
| ITDKPFSLYSTFVIEARHGFNKQTPWLFFRDMLKGIFLSVIIGPPIVAAIIVIVQKGGPY | |
| LAIYLWVFTFGLSIVMMTLYPVLIAPLFNKFTPLPDGQLREKIEKLASSLNYPLKKLFVV | |
| DGSTRSSHSNAYMYGFFKNKRIVPYDTLIQQCKDDEEIVAVIAHELGHWKLNHTVYTFVA | |
| MQILTLLQFGGYTLVRNSADLYRSFGFDTQPVLIGLIIFQHTVIPLQQLVSFGLNLVSRS | |
| FEFQADGFAKKLGYASGLRGGLVKLQEENLSAMNTDPWYSAYHYSHPPLVERLAALDEPD | |
| KKED | |

The present invention also includes a nucleic acid sequence complimentary to the Glycine max CaaX prenyl protease of SEQ ID NO:17. The disclosed complimentary sequence is shown as SEQ ID NO:19.

| | |
|---|---|
| TTAGTCTTCCTTCTTATCCGGTTCGTCCAGCGCGGCCAATCTTTCAACAAGGGGAGGAT | SEQ ID NO:19 |
| GAGAATAGTGATAAGCAGAGTACCAAGGATCTGTATTCATAGCTGACAGATTCTCCTCC | |
| TGTAGTTTCACAAGACCACCGCGTAATCCAGATGCATATCCAAGCTTCTTGGCAAAGCC | |
| ATCAGCCTGAAATTCAAATGATCGGCTGACTAGGTTCAGACCAAAGCTGACCAATTGCT | |
| GAAGTGGGATTACAGTATGCTGAAATATGATGAGCCCAATGAGGACTGGCTGCGTATCA | |
| AACCCAAAGCTTCGATACAGATCAGCTGAATTTCGCACTAGTGTATATCCTCCAAATTG | |
| TAGAAGTGTAAGAATCTGCATAGCAACAAATGTGTACACAGTATGGTTGAGCTTCCAGT | |
| GTCCCAACTCATGGGCAATAACAGCAACAATTTCCTCATCGTCTTTGCACTGTTGAATT | |
| AATGTGTCATAAGGGACAATCCTCTTGTTCTTGAAGAATCCATACATATAGGCATTGCT | |
| GTGACTTGATCTTGTGGATCCATCGACAACAAATAGTTTCTTTAACGGATAGTTGAGGG | |
| AGGAAGCAAGTTTCTCGATTTTCTCCCTGAGTTGACCATCTGGAAGTGGAGTGAACTTA | |
| TTGAAGAGTGGAGCTATTAGTACTGGATAAAGGGTCATCATCACAATAGAAAGACCAAA | |
| CGTAAAAACCCAAAGATAGATGGCCAAGTATGGACCTCCTTTCTGTACTATTACAATGA | |
| TTGCAGCCACAATAGGTGGACCAATTATTACAGAAAGGAAAATTCCTTTAAGCATGTCC | |
| CTAAAGAATAACCATGGTGTTTGCTTATTAAAACCATGACGGGCCTCAATCACAAAAGT | |
| TGAGTACAGAGAAAAGGGCAAATCTGTTATCTGTGACCAAATCATCAGCCCTGCTAAGA | |
| AGGCAAGGGTATGCAGTATTTCATTCTCAGCATTGAAACCAGCTATTGTCATAAAATCT | |
| CCTGATTTCTTCCAAAACCAGGGCAATACCCCAAAGTACAAAATTGTAGAGTCTGTCAC | |
| TATTGTCACAAACTCGTGAACAAAATGGAAGTGGCTTTTATCAAGACTATAGGCTCTAG | |
| ATTTCTCAAATTTCTCTTGGCTGATAACACCCTCTAAAGTCTTTGGAAGAGTAGGAAGT | |
| TTGAGGGCCCTATGTTGTCGCACATCCAAGTAAGTTTCAAAAATGTACATTAATATCAT | |
| AAATCCGACAACGGCTTCCATGTAGGGAAACGCCAT | |

Due to the nature of the cloning strategy the sequence presented is not full length but is missing the 5′ and 3′ non-translated regions. The percent identities of the Glycine max nucleotide sequence and its encoded amino acid sequence to that of other CPP sequences as determined by ClustalW analysis are shown in FIG. 2.

Using the sequences disclosed herein as hybridization probes, one is able to screen and isolate full length sequences from cDNA or genomic libraries or use the rapid amplification of cDNA ends (RACE) technology or other such PCR techniques. The CPP nucleic acids and amino acids disclosed above have homology to other disclosed CPP sequences (GenBank ID NOs: AL161491 (AT4g01320), AF007269 and AF353722; WO 02/16625 A2). The homology between these and other sequences is shown in the ClustalW alignment analysis shown in Tables 6A-6B.

TABLE 6A

```
ClustalW Nucleic Acid Analysis of CaaX Prenyl Protease
  1: PPI-AtCPP SEQ ID NO:1
  2: PPI-BnCPP SEQ ID NO:14
  3: PPI-GmCPP SEQ ID NO:17
  4: BASF_AT1 SEQ ID NO:21
  5: BASF_AT2 SEQ ID NO:23
  6: BASF-Com SEQ ID NO:25
  7: BASF-Gm SEQ ID NO:27
  8: AFC1 SEQ ID NO:29
  9: AT4g01320 SEQ ID NO:31
10: AF007269 SEQ ID NO:33
```

CLUSTAL W (1.81) multiple sequence alignment

| | |
|---|---|
| PPI-GmCPP | ------------------------------------------------------------ |
| BASF-Gm | ------------------------------------------------------------ |
| AT4901320 | ------------------------------------------------------------ |
| AF007269 | ATGGCGATTCCTTTCATGGAAACCGTCGTGGGTAAGCTTCAAAACCTTTTTCTGAGACAT |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| ppI-BnCPP | ------------------------------------------------------------ |
| BASF-Corn | ------------------------------------------------------------ |
| PPI-GmCPP | ------------------------------------------------------------ |
| BASF-Gm | ------------------------------------------------------------ |
| AT4g01320 | ------------------------------------------------------------ |
| AF007269 | TTTACTATCCTGTTTCACTCATCGTATTTCGTTTTTGTTTGGGTTTTGCTTTCTGTGTTG |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | ------------------------------------------------------------ |
| BASF-Gm | ------------------------------------------------------------ |
| AT4g01320 | ------------------------------------------------------------ |
| AF007269 | TGTGTGTTGAGATTCCATGACTCGTTTGTTTCATATACCATCGTCTCTGCTTCTCGTTTC |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | ------------------------------------------------------------ |
| BASF-Gm | ------------------------------------------------------------ |
| AT4g01320 | ------------------------------------------------------------ |
| AF007269 | TAAATTTTGTTCTTTTCTAATAGTGCGTACCTTGATCTGAGGTTTTATTACTCCTACTAG |
| PPI-AtCPP | ------------------------------------------------------------ |
| BASF AT2 | ------------------------------------------------------------ |
| atc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | ------------------------------------------------------------ |
| BASF-Gm | ------------------------------------------------------------ |
| AT4g01320 | ------------------------------------------------------------ |
| AF007269 | TTTCTTGTCTTACTCGTGCGTTTGATTTGATTTGAGCTTATGTGATTTCATCATCTCTTC |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | ------------------------------------------------------------ |
| BASF-Gm | ------------------------------------------------------------ |
| AT4g01320 | ------------------------------------------------------------ |
| AE007269 | CTCGGTTTTAGAATGTACGGAGCTTCTCTGTTAACCAAAATCTAGGATTTGGGAAGAAAA |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | ------------------------------------------------------------ |
| BASF-Gm | ------------------------------------------------------------ |
| AT4901320 | ------------------------------------------------------------ |
| AE007269 | GTCGGAGTCTTTTTTTTCCTCATTCCCGATTGGAAATTGAG~TCTTG~TTTTTCTTT |
| PPI-AtCPP | ------------------------------------------------------------ |
| BASF_AT2 | ------------------------------------------------------------ |
| atc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | ------------------------------------------------------------ |
| BASF-Gm | ------------------------------------ CTAATACGACTCACTATAGGGC |
| AT4g01320 | ------------------------------------------------------------ |
| AF007269 | GTTCAAGTCATACAGCTTGAGGTTTTGGGTTTTCTTGTCAGGGTATTATTATGTTCGTGA |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | ------------------------------------------------------------ |
| BASF-Gm | AAGCAGTGGTAACAACGCAGAGTACGCGGGGGGAGACGCATGGTTCTGAACTAATTGTTA |
| AT4g01320 | ------------------------------------------------------------ |
| AF007269 | CTGCAACTAGAGTTTTCTGGAGTTTTTTGAAATGGGTTTTGTGTTGTGGAACCGTATGTG |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | ------------------------------------------------------------ |
| BASF-Gm | TAAATAATACCTAAAATTTTGAGTTGTCCTAAACATTGGGGTTTAAACAAATCCAATCTC |
| AT4g01320 | ------------------------------------------------------------ |
| AF007269 | AATGTTGCATCAAAACTCTTTCAGTGCTCCAATGTTTCCATCAGTAGTAAGCACAAGAGA |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | ------------------------------------------------------------ |
| BASF-Gm | TCAATATAAAACCCAATGATCTCACC--CTCACTCCGTTTCTGATTTCTCACTCTTCGTT |
| AT4g01320 | ------------------------------------------------------------ |
| AF007269 | TCTTTTTATATCTGGTTGATCAAAAAAGTAGATGATGTTATTGAATTTTAAGTGATGGAG |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | --------------------------------ATGGCGTTTCCC--TACATGGAAGCCG |
| BASF-Gm | TCTCGTTCGGTTCATCAGCGTGTGTCTCAGC-CATGCCGTTTCCC--TACATGGAAGCCG |
| AT4g01320 | --------------------------------ATGGCGATTCCT--TTCATGGAAACCG |
| AF007269 | TATCTGTTGTTGTGGCATTTAGAGTAGATTCGTATTTCATCTTCTGTTTTATTCTTTTTC |
| PPI-AtCPP | --------------------------------ATGGCGATTCCT--TTCATGGAAACCG |
| BASF_AT2 | -------------------------ATGGCGATTCCT--TTCATGGAAACCG |
| afc1 | ----------------------------------ATGGCGATTCCT--TTCATGGAAACCG |
| BASF_AT1 | ---------------------------ATGGCGATTCCT--TTCATGGAAACCG |
| PPI-BnCPP | --------------------------------ATGGCGATTCCT--TTCATGGAAACCG |
| BASF-Corn | ------------------------------------------------------------ |
| PPI-GmCPP | TTGTCGGATTTATGATATTAATGTACATTTTTGAAACTTACTTGGATGTGCGACAACATA |
| BASF-Gm | TTGTCGGATTTATGATATTAATGTACATTTTTGAAACTTACTTGGATGTGCGACAACATA |
| AT4g01320 | TCGTGGGTTTTATGATAGTGATGTACATTTTTGAGACGTATTTGGATCTGAGGCAAACTA |
| AF007269 | TTACAGGTTTTATGATAGTGATGTACATTTTTGAGACGTATTTGGATCTGAGGCAACTCA |
| PPI-AtCPP | TCGTGGGTTTTATGATAGTGATGTACATTTTTGAGACGTATTTGGATCTGAGGCAACTCA |
| BASF_AT2 | TCGTGGGTTTTATGATAGTGATGTACATTTTTGAGACGTATTTGGATCTGAGGCAACTCA |
| afc1 | TCGTGGGTTTTATGATAGTGATGTACATTTTTGAGACGTATTTGGATCTGAGGCAACTCA |
| BASF_AT1 | TCGTGGGTTTTATGATAGTGATGTACATTTTTGAGACGTATTTGGATCTGAGGCAACTCA |
| PPI-BnCPP | TCGTTGGTTTTATGATAGTGATGTACGTTTTTGAGACGTATTTGGATCTGAGGCAACATA |
| BASF-Corn | ------------------------------------------------------------ |
| PPI-GmCPP | GGGCCCTCAAACTTCCTACTCTTCCAAAGACTTTAGAGGGTGTTATCAGCCAAGAGAAAT |
| BASF-Gm | GGGCCCTCAAACTTCCTACTCTTCCAAAGACTTTAGAGGGTGTTATCAGCCAAGAGAAAT |
| AT4g01320 | CTGCTCTCAAGCTTCCAACTCTCCCGAAAACCTTGGTTGGTGTAATTAGCCAAGAGAAGT |
| AF007269 | CTGCTCTCAAGCTTCCAACTCTCCCGAAAACCTTGGTTGGTGTAATTAGCCAAGAGAAGT |
| PPI-AtCPP | CTGCTCTCAAGCTTCCAACTCTCCCGAAAACCTTGGTTGGTGTAATTAGCCAAGAGAAGT |
| BASF_AT2 | CTGCTCTCAAGCTTCCACTCTCCCGAAACCTTGGTTGGTGTAATTAGCCAAGAGAAGT |
| afc1 | CTGCTCTCAAGCTTCCACTCTCCCGAAACCTTGGTTGGTGTAATTAGCCAAGAGAAGT |
| BASF_AT1 | CTGCTCTCAAGCTTCCACTCTCCCGAAACCTTGGTTGGTGTAATTAGCCAAGAGAAGT |
| PPI-BnCPP | CTGCTCTCAAGCTTCCACTCTCCCGAAACCTTGGTTGGTGTAATTAGCCAAGAGAAGT |
| BASF-Corn | ------------------------------------------------------------ |
| PPI-GmCPP | TTGAGAAATCTAGAGCCTATAG------------------------------------- |
| BASF-Gm | TTGAGAAATCTAGAGCCTATAG------------------------------------- |
| AT4g01320 | TTGAGAAATCACGAGCATACAG------------------------------------- |
| AF007269 | TTGAGAAATCACGAGCATACAGTCTTGACAAAAGGTTTCGTCTTGATCATATTTATATCA |
| PPI-AtCPP | TTGAGAAATCACGAGCATACAG------------------------------------- |
| BASF_AT2 | ----------------------------TTGAGAAATCACGAGCATACAG |
| afc1 | ------------------------------------TTGAGAAATCACGAGCATACAG |
| BASF_AT1 | ----------------------------TTGAGAAATCACGAGCATACAG |
| PPI-BnCPP | ------------------------------------TTGAGAAATCTCGAGCTTACAG |
| BASF-Corn | ------------------------------------------------------------ |
| PPI-GmCPP | ------------------------------------------TCTTGATAAA---AGCCA |
| BASF-Gm | ------------------------------------------TCTTGATAAA---AGCCA |
| AT4g01320 | ------------------------GGATATCATCACTGAGAACTTTAATATATGCAGCTA |
| AF007269 | TTTTAGTTTTTTATAATTGCCAGGGGATATCATCACTGAGAACTTTAATATATGCAGCTA |
| PPI-AtCPP | ------------------------------------------TCTTGACAAA---AGCTA |
| BASF_AT2 | ---------------------------------TCTTGACAAA---AGCTA |
| afc1 | -------------------------------------------CTTGACAAA---AGCTA |
| BASF_AT1 | ---------------------------------TCTTGACAAA---AGCTA |
| PPI-BnCPP | ------------------------------------------TCTTGACAAA---AGCCA |
| BASF-Corn | ------------------------------------------------------------ |
| PPI-GmCPP | CTTCCATTTTGTTCACGAGTTTGTGACAATAGTGACAGACTCTACAATTTTGTACTTTGG |
| BASF-Gm | CTTCCATTTTGTTCACGAGTTTGTGACAATAGTGACAGACTCTACAATTTTGTACTTTGG |
| AT4g01320 | TTTTCACTTTGTTCATGAGTTTGTAACTATACTTATGGACTCTGCAATTTTGTTCTTTGG |
| AF007269 | TTTTCACTTTGTTCATGAGTTTGTAACTATACTTATGGACTCTGCAATTTTGTTCTTTGG |
| PPI-AtCPP | TTTTCACTTTGTTCATGAGTTTGTAACTATACTTATGGACTCTGCAATTTTGTTCTTTGG |
| BASF_AT2 | TTTTCACTTTGTTCATGAGTTTGTAACTATACTTATGGACTCTGCAATTTTGTTCTTTGG |
| afc1 | TTTTCACTTTGTTCATGAGTTTGTAACTATACTTATGGACTCTGCAATTTTGTTCTTTGG |
| BASF_AT1 | TTTTCACTTTGTTCATGAGTTTGTAACTATACTTATGGACTCTGCAATTTTGTTCTTTGG |
| PPI-BnCPP | TTTTCACTTTGTTCATGAGTTTGTTACTATACTTATGGACTCTGCGATTCTGTTCTTTGG |
| BASF-Corn | ------------------------------------------------------------ |
| PPI-GmCPP | GGTATTGCCCTGGTTTTGGAAG------------------------------------- |
| BASF-Gm | GGTATTGCCCTGGTTTTGGAAG------------------------------------- |
| AT4g01320 | GATCTTGCCTTGGTTTTGGAAG------------------------------------- |
| AF007269 | GATCTTGCCTTGGTTTTGGAAGGTACATATCTGGTTTCGGTATACAGTATCTCATTTTGA |
| PPI-AtCPP | GATCTTGCCTTGGTTTTGGAAG------------------------------------- |
| BASF_AT2 | GATCTTGCCTTGGTTTTGGAAG--------------------------- |
| afc1 | GATCTTGCCTTGGTTTTGGAAG------------------------------------- |
| BASF_AT1 | GATCTTGCCTTGGTTTTGGAAG--------------------------- |
| PPI-BnCPP | GATCTTGCCTTGGTTTTGGAAG------------------------------------- |
| BASF-Corn | ------------------------------------------------------------ |
| PPI-GmCPP | ------------------------------------------------AAATCAGGAGAT |
| BASF-Gm | ------------------------------------------------AAATCAGGAGAT |
| AT4g01320 | ------------------------------------------------ATGTCTGGAGCT |
| AF007269 | ATATAGAGTTGTTACATTACAATTGTAAAGTTTTCATTTTTACCTTAGATGTCTGGAGCT |
| PPI-AtCPP | ------------------------------------------------ATGTCTGGAGCT |
| BASF_AT2 | ---------------------------------------ATGTCTGGAGCA |
| afc1 | -----------------------------------------------------ATGTCTGGAGCT |
| BASF_AT1 | ---------------------------------------ATGTCTGGAGCT |
| PPI-BnCPP | ------------------------------------------------ATATCTGGCGGC |
| BASF-Corn | ------------------------------------------------------------ |
| PPI-GmCPP | TTTATGACAATAGCTGGTTTCAATGCTGAGAATGAAATACTGCATACCCTTGCCTTCTTA |
| BASF-Gm | TTTATGACAATAGCTGGTTTCAATGCTGAGAATGAAATACTGCATACCCTTGCCTTCTTA |
| AT4g01320 | GTTTTACCGAGGTTGGGCCTTGATCCAGAGAATGAAATACTGCATACTCTTTCATTCTTG |
| AF007269 | GTTTTACCGAGGTTGGGCCTTGATCCAGAGAATGAAATACTGCATACTCTTTCATTCTTG |
| PPI-AtCPP | GTTTTACCGAGGTTGGGCCTTGATCCGGAGAATGAAATACTGCATACTCTTTCATTCTTG |
| BASF_AT2 | GTTTTACCGAGGTTGGGCCTTGATCCAGAGAATGAAATACTGCATACTCTTTCATTCTTG |
| afc1 | GTTTTACCGAGGTTGGGCCTTGATCCAGAGAATGAAATACTGCATACTCTTTCATTCTTG |
| BASF_AT1 | GTTTTACCGAGGTTGGGCCTTGATCCAGAGAATGAAATACTGCATACTCTTTCATTCTTG |
| PPI-BnCPP | TTTCTACCAATGGTGGGACTCGATCCAGAGAATGAAATCCTGCACACTCTTTCATTCTTG |
| BASF-Corn | --------- ACGAGGCTGAGTGCTGAGAATGAGATAATACACACCCTTGCTTTCTTA |
| | * * * * ******** ** * ** ** *** * ***** |
| PPI-GmCPP | GCAGGGCTGATGATTTGGTCACAG---------------------------------- |
| BASF-Gm | GCAGGGCTGATGATTTGGTCACAG---------------------------------- |
| AT4g01320 | GCTGGTGTTATGACATGGTCACAG---------------------------------- |
| AF007269 | GCTGGTGTTATGACATGGTCACAGGTGTTCCAAATAAACCCCTTCATATAGTCCTATACG |
| PPI-AtCPP | GCTGGTGTTATGACATGGTCACAG---------------------------------- |
| BASF_AT2 | GCTGGTGTTATGACATGGTCACAG------------------------ |
| afc1 | GCTGGTGTTATGACATGGTCACAG---------------------------------- |
| BASF_AT1 | GCTGGTGTTATGACATGGTCACAC---------------------------- |
| PPI-BnCPP | GCTGGTCTTATCACATGGTCACAG---------------------------------- |
| BASF-Corn | GCTGGTTCCATGGTTTGGTCGCAG---------------------------------- |
| | ** ** *** ***** ** |
| PPI-GmCPP | ------------------------------------------------------------ |
| BASF-Gm | ------------------------------------------------------------ |
| AT4g01320 | ------------------------------------------------------------ |
| AF007269 | TTTAGCATCAAAATATCTATTTTCTTAAGATAATAATATTTCTTTTATATTCTGATGCAG |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | ATAACAGATTTGCCCTTTTCTCTGTACTCAACTTTTGTGATTGAGGCCCGTCATGGTTTT |
| BASF-Gm | ATAACAGATTTGCCCTTTTCTCTGTACTCAACTTTTGTGATTGAGGCCCGTCATGGTTTT |
| AT4g01320 | ATCACTGATTTGCCATTTTCTTTGTACTCAACTTTCGTGATCGAGTCTCGGCATGGGTTC |
| AF007269 | ATCACTGATTTGCCATTTTCTTTGTACTCAACTTTCGTGATCGAGTCTCGGCATGGGTTC |
| PPI-AtCPP | ATCACTGATTTGCCATTTTCTTTGTACTCAACTTTCGTGATCGAGTCTCGGCATGGGTTC |
| BASF_AT2 | ATCACTGATTTGCCATTTTCTTTGTACTCAACTTTCGTGATCGAGTCTCGGCATGGGTTC |
| afc1 | ATCACTGATTTGCCATTTTCTTTGTACTCAACTTTCGTGATCGAGTCTCGGCATGGGTTC |
| BASF_AT1 | ATCACTGATTTGCCATTTTCTTTGTACTCAACTTTCGTGATCGAGTCTCGGCATGGGTTC |
| PPI-BnCPP | ATCACTGATTTGCCATTTTCTTTGTACTCAACTTTCGTGATCGAGTCTCGGCATGGGTTC |
| BASF-Corn | ATTACAGACTTGCCGTTCTCTCTCTATTCAACTTTTGTTATAGAGGCTCGACATGGTTTT |
| | ** ** ** ***** ** *** * ** ******** ** ** *** * ** ***** ** |
| PPI-GmCPP | AATAAG-------------------------------------------------------- |
| BASF-Gm | AATAAG-------------------------------------------------------- |
| AT4g01320 | AACAAA-------------------------------------------------------- |
| AE007269 | AACAAAGTATGTCGTATTTCCAACACTACCTTGTGACTTACGTTTTTTTATCAGAGATGT |
| PPI-AtCPP | AACAAA-------------------------------------------------------- |
| BASF_AT2 | AACAAA---------------------------------------------- |
| afc1 | AACAAA-------------------------------------------------------- |
| BASF_AT1 | AACAAA------------------------------------------------ |
| PPI-BnCPP | AACAAA--------------------------------------------------------- |
| BASF-Corn | AACAAG-------------------------------------------------------- |
| PPI-GmCPP | ---------------------------------CAAACACCATGGTTATTCTTTAGGGACA |
| BASF-Gm | ------------------------------------CAAACACCATGGTTATTCTTTAGGGACA |
| AT4g01320 | ----------------------------------CAAACAATATGGATGTTCATTAGGGACA |
| AF007269 | GGATTAAATTTGCTTCTAAATTCTGTTGACAGCAAACAATATGGATGTTCATTAGGGACA |
| PPI-AtCPP | ----------------------------------CAAACAATATGGATGTTCATTAGGGACA |
| BASF_AT2 | ------------------------CAAACAATATGGATGTTCATTAGGGACA |
| afc1 | ---------------------------------------CAAACAATATGGATGTTCATTAGGGACA |
| BASF_AT1 | -------------------------CAAACAATATGGATGTTCATTAGGGACA |
| PPI-BnCPP | ----------------------------------CAAACAATATGGATGTTCATTAGGGACA |
| BASF-Corn | ----------------------------------CAAACTATATGGCTCTTCATTAGGGATA |
| | ******* **** * *** ******** * |
| PPI-GmCPP | TGCTTAAAGGAATTTTCCTTTCTGTAATAATTGGTCCACCTATTGTGGCTGCAATCATTG |
| BASF-Gm | TGCTTAAAGGAATTTTCCTTTCCGTAATAATTGGTCCACCTATTGTGGCTGCAATCATTG |
| AT4g01320 | TGATCAAAGGAACATTCCTCTCTGTCATACTAGGCCCACCCATTGTTCCTGCGATAATTT |
| AF007269 | TGATCAAAGGAACATTCCTCTCTGTCATACTAGGCCCACCCATTGTTGCTGCGATAATTT |
| PPI-AtCPP | TGATCAAAGGAACATTCCTCTCTGTCATACTAGGCCCACCCATTGTTGCTGCGATAATTT |
| BASF_AT2 | TGATCAAAGGAACATTCCTCTCTGTCATACTAGGCCCACCCATTGTTGCTGCGATAATTT |
| afc1 | TGATCAAAGGAACATTCCTCTCTGTCATACTAGGCCCACCCATTGTTGCTGCGATAATTT |
| BASF_AT1 | TGATCAAAGGAACATTCCTCTCTGTCATACTAGGCCCACCCATTGTTGCCGCGATAATTT |
| PPI-BnCPP | TGATCAAAGGAATACTCCTCTCTGTCATACCTGCCCCTCCTATCGTTGCCGCAATTATTG |
| BASF-Corn | TGATCAAAGGAATTTTACTATCCATGATATTGGGGCCACCAATCGTGGCTGCTATCATCT |
| | ** * ******* * ** ** * ** ** ** ** ** ** ** ** |
| PPI-GmCPP | TAATAGTACAG------------------------------------------------- |
| BASF-Gm | TAATAGTACAG------------------------------------------------- |
| AT4g01320 | TCATAGTCCAG------------------------------------------------- |
| AF007269 | TCATAGTCCAGGTTTGATGATTCTGGATTCATCTTATTTCTGAGTTTTTCACATGGATGA |
| PPI-AtCPP | TCATAGTCCAG------------------------------------------------- |
| BASF_AT2 | TCATAGTCCAG--------------------------------------- |
| afc1 | TCATAGTCCAG------------------------------------------------- |
| BASF_AT1 | TCATAGTCCAG--------------------------------------- |
| PPI-BnCPP | TTATAGTTCAG------------------------------------------------- |
| BASF-Corn | ACATAGTACAG------------------------------------------------- |
| PPI-GmCPP | ------------------------------------------------------------ |
| BASF-Gm | ------------------------------------------------------------ |
| AT4g01320 | ------------------------------------------------------------ |
| AF007269 | CTATTCTCCATTGAGTGTGAGCTTCAAAGTTTTTAGTTTTCGTGTTAAAAATTTAAAATT |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | ---------------------------------AAAGGAGGTCCATACTTGGCCATC |
| BASF-Gm | ---------------------------------AAAGGAGGTCCATACTTGGCCATC |
| AT4g01320 | ---------------------------------AAAGGAGGTCCTTATCTTGCCATC |
| AF007269 | TGCTTCTCTGAGCATGAAGTTTCTATCTTTTTCCAGAAAGGAGGTCCTTATCTTGCCATC |
| PPI-AtCPP | ---------------------------------AAAGGAGGTCCTTATCTTGCCATC |
| BASF_AT2 | ------------------------AAAGGAGGTCCTTATCTTGCCATC |
| afc1 | ---------------------------------------AAAGGAGGTCCTTATCTTGCCATC |
| BASF_AT1 | ------------------------AAAGGAGGTCCTTATCTTGCCATC |
| PPI-BnCPP | ----------------------------------AAAGGAGGTCCTTACCTCGCCATC |
| BASF-Corn | ----------------------------------ATTGGAGGACCTTACCTGGCTATA |
| | * ***** ** ** * ** ** |
| PPI-GmCPP | TATCTTTGGGTTTTTACGTTTGGTCTTTCTATTGTGATGATGACCCTTTATCCAGTACTA |
| BASF-Gm | TATCTTTGGGTTTTTACGTTTGGTCTTTCTATTGTGATGATGACCCTTTATCCAGTACTA |
| AT4g01320 | TATCTGTGGGCATTCATGTTTATCCTGTCTCTAGTGATGATGACTATATACCCGGTCTTG |
| AF007269 | TATCTGTGGGCATTCATGTTTATCCTGTCTCTAGTGATGATGACTATATACCCGGTCTTG |
| PPI-AtCPP | TATCTGTGGGCATTCATGTTTATCCTGTCTCTAGTGATGATGACTATATACCCGGTCTTG |
| BASF_AT2 | TATCTGTGGGCATTCATGTTTATCCTGTCTCTAGTGATGATGACTATATACCCGGTCTTG |
| afc1 | TATCTGTGGGCATTCATGTTTATCCTGTCTCTAGTGATGATGACTATATACCCGGTCTTG |
| BASF_AT1 | TATCTGTGGGCATTCATGTTTATCCTGTCTCTAGTCATGATGACTATATACCCGGTCTTG |
| PPI-BnCPP | TATCTGTGGGCATTCATGTTTATCCTGTCTCTAGTGATCATGACTATATACCCTGTTTTG |
| BASF-Corn | TATCTCTGGGGTTTTATGTTTGTATTAGCTCTACTGATGATGATGACAATATACCCCATTGTG |
| | ***** **** ** * **** * ** * ********** * ** ** * * |
| PPI-GmCPP | ATAGCTCCACTCTTCAATAAGTTCACTCCA---------------------------------- |
| BASF-Gm | ATAGCTCCACTCTTCAATAAGTTCACTCCA---------------------------------- |
| AT4g01320 | ATAGCACCGCTCTTCAACAAGTTCACTCCT---------------------------------- |
| AF007269 | ATAGCACCGCTCTTCAAcAGTTCACTCCTGTGTGTATTTCTGTCATGGCCATTTTACAA |
| PPI-AtCPP | ATAGCACCGCTCTTCAACAAATTCACTCCT---------------------------------- |
| BASF_AT2 | ATAGCACCGCTCTTCAACAAGTTCACTCCT------------------------ |
| afc1 | ATAGCACCGCTCTTCAACAAGTTCACTCCT---------------------------------- |
| BASF_AT1 | ATAGCACCGCTCTTCAACAAGTTCACTCCT------------------------ |
| PPI-BnCPP | ATTGCACCTCTTTTCAACAACTTCACTCCT---------------------------------- |
| BASF-Corn | ATAGCTCCTCTGTTCAACAAGTTCACTCCT---------------------------------- |
| | ** ** ** ** ***** ** ******** |
| PPI-GmCPP | ------------------------------------------------------------ |
| BASF-Gm | ------------------------------------------------------------ |
| AT4g01320 | ------------------------------------------------------------ |
| AF007269 | TTCACTGCTTGTTTGCATATGTTGTTACCAGACAATATAATCTCCCGCTTTTTTATGGCT |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | ----CTTCCAGATGGTCAACTCAGGGAGAAAATCGAGAAACTTGCTTCCTCCCTCAACTA |
| BASF-Gm | ----CTTCCAGATGGTCAACTCAGGGAGAAAATCCAGAAACTTGCTTCCTCCCTCAACTA |
| AT4g01320 | ----CTTCCAGATGGAGACCTCCGGGAGAAGATTGAGAAACTTGCTTCTTCTCTAAAGTT |
| AF007269 | ATAGCTTCCAGATGGAGACCTCCGGGAGAAGATTGAGAAACTTGCTTCTTCTCTAAAAGTT |
| PPI-AtCPP | ----CTTCCAGATGCAGACCTCCGGGACAAGATTGAGAAACTTGCTTCTTCCCTAAAGTT |
| BASF_AT2 | ----CTTCCAGATGGAGACCTCCGGGAGAAGATTGAGAAACTTGCTTCTTCTCTAAAGTT |
| atc1 | ----CTTCCAGATGGAGACCTCCGGGAGAAGATTGAGAAACTTCCTTCTTCTCTAAAGTT |
| BASF_AT1 | ----CTTCCAGATGGAGACCTCCGGGAGAGATTGAGAAACTTGCTTCTTCTCTAAAGTT |
| PPI-BnCPP | ----CTTCCTGATGGAGACCTCCGGGAGAGATTCAGACTTGCTTCTTCTCTAAAGTT |
| BASF-Corn | ----CTTCCTGAAGGAGTCCTCAGGGAAAAAATAGAGAAGCTGGCAGCTTCCCTAAAGTT |
| | ***** ** ** *** **** ** ** ***** ** ** * ** ** ** * |
| PPI-CmCPP | TCCGTTAAAGAACTATTTGTTGTCGATGGATCCACGATCAAGTCACAGCAATG----- |
| BASF-Gm | TCCGTTAAAGAACTATTTGTTGTCGATGGATCCACGATCAAGTCACAGCAATG----- |
| AT4g01320 | TCCTTTGAGAAGCTGTTTGTTGTCGATGGATCTACAAGGTCAAGCCATAGATG----- |
| AF007269 | TCCTTTGAGAAGCTGTTTGTTGTCGATGGATCTACAAGGTCAAGCCATAGCAATGTGAG |
| PPI-AtCPP | TCCTTTGAAGAAGCTGTTGTTGTCGATGGATCTACAAGGTCAAGCCATAGCAATG--- |
| BASF_AT2 | TCCTTTGAAGAAGCTGTTGTTGTCGATGGATCTACAAGGTCAAGCCATAGCAATG |
| afc1 | TCCTTTGAAGAAGCTGTTTGTTGTCGATGGATCTACAAGGTCAAGCCATAGCAATG--- |
| BASF_AT1 | TCCTTTGAAGAAGCTGTTTGTTGTCGATGGATCTACAAGGTCAAGCCATAGCAATG |
| PPI-BnCPP | TCCTCTGAAGAAGCTGTTTGTTGTCGATGGATCTACAAGGTCAAGCCATAGTAATG--- |
| BASF-Corn | TCCTTTGAAAAAGCTTTTCGTGGTAGATGGGTCTACCAGATCAAGCCACAGTAATG--- |
| | *** * ** ** ** ** ** ** ***** ** ** ** ***** ** ** **** |
| PPI-GmCPP | ------------------------------------------------------------ |
| BASF-Gm | ------------------------------------------------------------ |
| AT4g01320 | ------------------------------------------------------------ |
| AF007269 | AAGCTTGAGATCTCTTCCTACCTACTTTACTCTAGTTTACCATTAGAAGCTTACGTATCT |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | ------CCTATATGTATGGATTCTTCAAGAACAAGAGGATTGTCCCTTAT |
| BASF-Gm | ------CCTATATGTATGGATTCTTCAAGAACAAGAGGATTGTCCTTTAT |
| AT4g01320 | ------CTTACATGTATGGTTTCTTTAAGAACAAAAGGATTGTTCTTTAT |
| AF007269 | TGTTACATCATACAGGCTTACATGTATGGTTTCTTTAAGAACAAAAGGATTGTTCTTTAT |
| PPI-AtCPP | ------CTTACATGTATGGTTTCTTTAAGAACAAAAGGATTGTTCTTTAT |
| BASF_AT2 | -------CTTACATGTATGGTTTCTTTAAGAACAAAAGGATTGTTCTTTAT |
| afc1 | ------CTTACATGTATGGTTTCTTTAAGAACAAAAGGATTGTTCTTTAT |
| BASF_AT1 | -----CTTACATGTATGGTTTCTTTAAGAACAAAAGGATTGTTCTTTAT |
| PPI-BnCPP | -----CTTACATGTATGGTTTCTTCAAGAACAAAAGGATTGTTCTTTAT |
| BASF-Corn | -----CCTACATGTATGGTTTTTTCAAGAACAAGCGCATAGTACTCTAT |
| PPI-GmCPP | GACACATTAATTCAACAG---------------------------------- |
| BASF-Gm | GACACATTAATTCAACAG----------------------------------- |
| AT4g01320 | GATACGTTGATTCAGCAG---------------------------------- |
| AF007269 | GATACGTTGATTCAGCAGGTACTGTGACTCTTGATGCTTCAAACGAGCTATACGTATCT |
| PPI-AtCPP | GATACGTTGATTCAGCAG---------------------------------- |
| BASF_AT2 | GATACGTTGATTCAGCAG------------------------- |
| atc1 | GATACGTTGATTCAGCAG--------------------------------------- |
| BASF_AT1 | GATACGTTGATTCAGCAG------------------------- |
| PPI-BnCPP | GACACATTGATTCAGCAG---------------------------------- |
| BASF-Corn | GACACATTGATTCAGCAG------------------------------------------------------------ |
| | ** ** ** ***** *** |
| PPI-GmCPP | ------------------------------------------------TGCAAAGACGATGAGG |
| BASF-Gm | --------------------------------------------------TGCAAAGACGATGAGG |
| AT4501320 | ------------------------------------------------TGCAAGAATGAGGATG |
| AF007269 | TCTGTTTCTGGTTCTGAAACATAACATAATCTTCTATTGTGCAGTGCAAGAATGAGGATG |
| PPI-AtCPP | ------------------------------------------------TGCAAGAATGAGGATG |
| BASF_AT2 | ---------------------------------------TGCAAGAATGAGGATG |
| afc1 | -----------------------------------------------------TGCAAGAATGAGGATG |
| BASF_AT1 | ---------------------------------------TGCAAGAATGAGGATG |
| PPI-BnCPP | ------------------------------------------------TGCCAGAATGAGAATG |
| BASF-Corn | ------------------------------------------------TGTAGCAATGAGGATG |
| | ** * ** * * |
| PPI-GmCPP | AAATTGTTGCTGTTATTGCCCATGAGTTGGGACACTGGAAGCTCAACCATACTGTGTACA |
| BASF-Gm | AAATTGTTGCTGTTATTGCCCATGAGTTGGGACACTGGAAGCTCAACCATACTGTGTACA |
| AT4g01320 | AAATTGTGGCGGTTATTGCACACGAGCTTGGACATTGGAAACTGAATCACACTACATACT |
| AE007269 | AAATTGTGGCGGTTATTGCACACGAGCTTGGACATTGGAAACTGAATCACACTACATACT |
| PPI-AtCPP | AAATTGTGGCGGTTATTGCACACGAGCTTGGACATTGGAAACTGAATCACACTACATACT |
| BASF_AT2 | AAATTGTGGCGGTTATTGCACACGAGCTTGGACATTGGAAACTGAATCACACTACATACT |
| atc1 | AAATTGTGGCGGTTATTGCACACGAGCTTGGACATTGGAAACTGAATCACACTACATACT |
| BASF_AT1 | AAATTGTGGCGGTTATTGCACACGAGCTTGGACATTGGAAACTGAATCACACTACATACT |
| PPI-BnCPP | AAATTGTGGCGGTTATTGCACACGAGCTGGGACACTGGAAGCTGAATCACACTACATACT |
| BASF-Corn | AGATAGTTTCTGTTATAGCACATGAACTTGGACACTGGAAACTCAATCATACTGTCTATT |
| | * ** ** * **** ** ** ** * ***** ***** ** ** ** *** ** |
| PPI-GmCPP | CATTTGTTGCTATGCAG------------------------------------------- |
| BASF-Gm | CATTTGTTGCTATGCAG--------------------------------------------- |
| AT4901320 | CGTTCATTGCAGTTCAA------------------------------------------- |
| AE007269 | CGTTCATTGCAGTTCAAGTGAGGCTCAACCGACAGTTCAAAAACTTACTCACATCTAAT |
| PPI-AtCPP | CGTTCATTGCAGTTCAA------------------------------------------- |
| BASF_AT2 | CGTTCATTGCAGTTCAA---------------------------------- |
| atc1 | CGTTCATTGCAGTTCAA------------------------------------------------ |
| BASF_AT1 | CGTTCATTGCAGTTCAA---------------------------------- |
| PPI-BnCPP | CGTTCATTGCTGTTCAA------------------------------------------- |
| BASF-Corn | CCTTTGTAGCTGTCCAG------------------------------------------- |
| | * ** * ** * ** |
| PPI-GmCPP | ---------------------------------------------------------ATTCTTACA |
| BASF-Gm | -----------------------------------------------------------ATTCTTACA |
| AT4901320 | ---------------------------------------------------------ATCCTTGCC |
| AE007269 | TTCACTTAAGAAATCATGTCTTATGACCCTCTCTCAATGTTTTGCTTGCAGATCCTTGCC |
| PPI-AtCPP | ---------------------------------------------------------ATCCTTGCC |
| BASF_AT2 | -----------------------------------------------ATCCTTGCC |
| afc1 | --------------------------------------------------------------ATCCTTGCC |
| BASF_AT1 | ------------------------------------------------ATCCTTGCC |
| PPI-BnCPP | ---------------------------------------------------------ATCCTTGCC |
| BASF-Corn | ---------------------------------------------------------CTGCTTATG |
| | * *** |
| PPI-GmCPP | CTTCTACAATTTGGAGGATATACACTAGTGCGAAATTCAGCTGATCTGTATCGAAGCTTT |
| BASF-Gm | CTTCTACAATTTGGAGGATATACACTAGTGCGAAATTCAGCTGATCTGTATCGAAGCTTT |
| AT4g01320 | TTCTTACAATTTGGAGGATACACTCTTCTCAGAAACTCCACTGATCTCTTCAGGAGTTTC |
| AF007269 | TTCTTACAATTTGGAGGATACACTCTTCTCAGAAACTCCACTGATCTCTTCAGGAGTTTC |
| PPI-AtCPP | TTCTTACAATTTGGAGGATACACTCTTCTCAGAAACTCCACTGATCTCTTCAGGAGTTTC |
| BASF_AT2 | TTCTTACAATTTGGAGGATACACTCTTGTCAGATCTCCACTGATCTCTTCAGGAGTTTC |
| afc1 | TTCTTACAATTTGGAGGATACACTCTTGTCAGAAACTCCACTGATCTCTTCAGGAGTTTC |
| BASF_AT1 | TTCTTACATTTGGAGGATACACTCTTGTCAGAAATCCACTGATCTCTTCAGGAGTTTC |
| PPI-BnCPP | TTCTTGCAATTTGGAGGATACACTCTTGTCAGAAACTCCACTGATCTCTTCAGGAGTTTT |
| BASF-Corn | TTTCTTCAATTTGGAGGATATACTCTAGTAAGGAGCTCCACTGATCTCTTCAGGAGTTTT |
| | * * ************** * ** * * * ** ***** * * ** ** |
| PPI-GmCPP | GGGTTTGATACGCAGCCAGTCCTCATTGGGCTCATCATATTTCAG |
| BASF-Gm | GGGTTTGATACGCAGCCAGTCCTCATTGGGCTCATCATATTTCAG |
| AT4g01320 | GGATTTGATACACAGCCTGTTCTCATTGGTTTGATCATATTTCAG |
| AF007269 | GGATTTGATACACAGCCTGTTCTCATTGGTTTGATCATATTTCAGGTTTGTTATTTTTGC |
| PPI-AtCPP | GGATTTGATACACAGCCTGTTCTCATTGGTTTGATCATATTTCAG |
| BASF_AT2 | GGATTTGATACACAGCCTGTTCTCATTGGTTTGATCATATTTCAG |
| afc1 | GGATTTGATACACAGCCTGTTCTCATTGGTTTGATCATATTTCAG |
| BASF_AT1 | GGATTTGATACACAGCCTGTTCTCATTGGTTTGATCATATTTCAG |
| PPI-BnCPP | GGTTTTGATACACAACCAGTTCTCATTGGTTTGATCATATTTCAG |
| BASF-Corn | GGCTTCAAGGACCAGCCAGTAATAATTGGATTGATCATTTTCCCG |
| | ** ** * ** ** ** * ***** * ***** ** * * |
| PPI-GmCPP | ------------------------------------------------------------ |
| BASF-Gm | ------------------------------------------------------------ |
| AT4g01320 | ------------------------------------------------------------ |
| AF007269 | CTTTTGACACTAATCTAATGAATCAAGGATGGATTAAGAAAAAAAAACTCTAAACCTTTG |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | -------------------------CATACTGTAATCCCACTTCAGCAATTGGTCAGC |
| BASF-Gm | -------------------------CATACTGTAATCCCACTTCAGCAATTGGTCAGC |
| AT4g01320 | -------------------------CACACTGTAATACCACTGCAACATCTAGTAAGC |
| AE007269 | GTTATATCTCCTGTCTGATTATCACAGCACACTGTAATACCACTGCAACATCTAGTAAGC |
| PPI-AtCPP | -------------------------CACACTGTAATACCACTGCAACATCTAGTAAGC |
| BASF_AT2 | ----------------CACACTGTAATACCACTGCAAC |
| afc1 | -----------------------------CACACTGTAATACCACTGCAACATCTAGTAAGC |
| BASF_AT1 | ---------------CACACTGTAATACCACTGCAACATCCAGTAAGC |
| PPI-BnCPP | -----------------------CACACTGTAATACCACTTCAACACCTAGTAAGC |
| BASF-Corn | ------------------------CACACCATAATACCCATCCAACACCTTCTGAGC |
| | *** ** ***** ** ** ** * ***** ******* |
| PPI-GmCPP | TTTGGTCTGAACCTAGTCAGCCGATCATTTGAATTTCAGG---------------- |
| BASF-Gm | TTTGGTCTGAACCTAGTCAGCCGATCATTTGAATTTCAGG------------------ |
| AT4g01320 | TTTGGCCTGAACCTCGTTAGTCGAGCGTTTGAGTTTCAGG--------------- |
| AF007269 | TTTGGCCTGAACCTCGTTAGTCGAGCGTTTGAGTTTCAGGTACCATCTTACAATCCCTCA |
| PPI-AtCPP | TTTGGCCTGAACCTCGTTAGTCGAGCGTTTGAGTTTCAGG---------------- |
| BASF_AT2 | TTTGGCCTGAACCTCGTTAGTCGAGCGTTTGAGTTTCAGG------- |
| afc1 | TTTGGCCTGAACCTCGTTAGTCGAGCGTTTGAGTTTCAGG--------------------- |
| BASF_AT1 | TTTGGCCTCAACCTTGTTAGTCGAGCGTTTGAGTTTCAGG------- |
| PPI-BnCPP | TTTGACCTCAACCTTGTTAGTCGAGCGTTTGAGTTTCAGG---------------- |
| BASF-Corn | TTTCGCCTGAACCTTGTCAGCAGAGCATTTGAATTTCAGG---------------- |
| | *** ** ***** ** ** ** * ***** ******* |
| PPI-GmCPP | ------------------------------------------------------------ |
| BASF-Gm | ------------------------------------------------------------ |
| AT4g01320 | ------------------------------------------------------------ |
| AF007269 | AGATCCACCATAGTTTCTTTATTGCATGGCCCCTCATCTACTAATCTGAGTTAAGT |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | ------------------------------------------------------------ |
| PPI-GmCPP | -------------CTGATGGCTTTGCCAAGAAGCTTGGATATGCATCTGGATTACGCGGTG |
| BASF-Gm | -------------CTGATGGCTTTGCCAAGAAGCTTGGATATGCATCTGGATTACGCGGTG |
| AT4g01320 | -------------CTGATGCTTTTGCTGTGAAGCTTGGCTATGCAAAAAGATCTTCGTCCTG |
| AF007269 | TCCTTTTGCAGGCTGATGCTTTTGCTGTGAAGCTTGGCTATGCAAAAGATCTTCGTCCTG |
| BASF_AT2 | ----------CTGATGCTTTTGCTGTGAAGCTTGGCTATGCAAGATCTTCGTCCTG |
| afc1 | --------------CTGATGCTTTTGCCGTGAAGCTTGCCTATGCAAAAGATCTTCGTCCTG |
| BASF_AT1 | --------------CTGATGCTTTTGCTGTGAAGCTTGGCTATGCAAAAGATCTTCGTCCTA |
| PPI-BnCPP | --------------CTGATGCTTTTGCAGTGAATCTTGGTTATGCAAAGGATCTACGTCCTG |
| BASF-Corn | --------------CTGATCCCTTTGCCAAGAACCTTGGATATGCCCCTCAGCTCCGAGCAG |
| PPI-GmCPP | GTCTTGTGAAACTACAGG--------------------------------------------- |
| BASF-Gm | GTCTTGTGAAACTACAGG-------------------------------------------- |
| AT4g001320 | CTCTAGTGAACTACAGGTCAGAGAACATAACAACAGAACAAACTGTTACCTCAATTT |
| AF007269 | CTCTAGTGAACTACAGGTCAGAGAAGATAACAACAGAACAAACTGTTACCTCAATTT |
| PPI-AtCPP | CTCTAGTGAAACTACAGG------------------------------------------- |
| BASF_AT2 | CTCTAGTGAAACTACAGG--------------------------------- |
| afc1 | CTCTAGTGAAACTACAGG------------------------------------------- |
| BASF_AT1 | CTCTAGTGAAACTACAGG--------------------------------- |
| PPI-BnCPP | CCCTAGTGAACCTACAGG------------------------------------------- |
| BASF-Corn | CCCTTGTTAAACTACAGG------------------------------------------- |
| | ** ** ** ******* |
| PPI-GmCPP | -------------------------------------------AGGAGAATCTGTCAGCTA |
| BASF-Gm | --------------------------------------------AGGAGAATCTGTCAGCTA |
| AT4g01320 | GTGTCACACACTTAATGGATTTTTTGTTGGGATTTTGAAGAAGAGAACTTATCAGCAAA |
| AF007269 | GTGTCACACACTTAATGGATTTTTTGTTGGGATTTTGAAGAAGAGAACTTATCAGCAAA |
| PPI-AtCPP | --------------------------------------------AAGAGAACTTATCAACAA |
| BASF_AT2 | -----------------------------------AAGAGAACTTATCAGCAA |
| afc1 | -------------------------------------------------AAGAGAACTTATCAGCAA |
| BASF_AT1 | -----------------------------------AAGAGAACTTATCAGCAA |
| PPI-BnCPP | --------------------------------------------AGGAGAACTTATGTGCGA |
| BASF-Corn | --------------------------------------------AGGAGAACTTGTCTGCGA |
| PPI-GmCPP | TGAATACAGATCCTTGGTACTCTGCTTATCACTATTCTATTCCTCCCCTTGTTGAAAGAT |
| BASF-Gm | TGAATACAGATCCTTGCT--CGTGCCG---------------------------------- |
| AT4g01320 | TGAACACTGATCAATTGTACTCAGCTTATCACTACTCAATCCTCCTCTTGTTGAAAAGGC |
| AF007269 | TGAACACTGATCCATTGTACTCAGCTTATCACTACTAAAATCCTCCTCTTGTTGAAAGGC |
| PPI-AtCPP | TGAACACTGATCCATTGTACTCAGCTTATCACTACTCACATCCTCCTCTTGTTGAAAGGC |
| BASF_AT2 | TGAAACTGATCTATTGTACTCAGCTTATCACTACTCACATCCTCCTCTTGTTGAAAAC |
| afc1 | TGAACACTGATCCATTGCACTCAGCTTATCACTACTAAATCCTCCTCTTGTTGAAAGGC |
| BASF_AT1 | TGAATACTGATCCATTGTACTCAGCTTATCACTACTCAAATCCTCCTCTTGTTGAAAGGC |
| PPI-BnCPP | TGAACACAGACCCATTGTACTCAGCTTATCACTACTCACACCCTCCTCTTGTAGAGAGGC |
| BASF-Corn | TGAACACCGATCCTTGGTATTCGGCATATCACTACTCCCACCAAACCACTCGTCGAGAGGC |
| PPI-GmCPP | TGGCCGCGCTGGACGA---ACCGGATAAGAAGGAAGACTAA--------------------- |
| BASF-Gm | ------------------------------------------------------------ |
| AT4g01320 | TTCGAGCCATTGATGG---AGAAGACAAGAAGACAGATTAA----------------- |
| AF007269 | TTCGAGCCATTGATGG---AGAAGACAAGAAGACAGATTAA----------------- |
| PPI-AtCPP | TTCGAGCCACTGATGG---AGAAGACAAGAAGACAGATTAA----------------- |
| BASF_AT2 | TTCGAGCCATTGATGG---AGAGACAAGAAGACAGATTAA---------- |
| atc1 | TTCGAGCCATTGATGG---AGAAGACAAGAAGACAGATTAA------------------------- |
| BASF_AT1 | TTCGAGCCATTGATGG---AGAAGACAAGAAGACAGATTAA----------- |
| PPI-BnCPP | TTCGAGCCATTGATGG---AGAAGACAAGAAGACAGATTAA-------------------- |
| BASF-Corn | TGCAAGCTTTGGAAGATTCAGACGACAAAAAAGAAGATTAGTCGATCCTTGTATGAGGTT |
| PPI-GmCPP | ------------------------------------------------------------ |
| BASF-Gm | ------------------------------------------------------------ |
| AT4g01320 | ------------------------------------------------------------ |
| AF007269 | ------------------------------------------------------------ |
| PPI-AtCPP | ------------------------------------------------------------ |
| BASF_AT2 | ------------------------------------------------------------ |
| afc1 | ------------------------------------------------------------ |
| BASF₁₃ AT1 | ------------------------------------------------------------ |
| PPI-BnCPP | ------------------------------------------------------------ |
| BASF-Corn | TACATATGGATTTTTCCCTGCCACATGCACACCGATTCAGTGCTTGGATGGTGAGGGTTT |
| PPI-GmCPP | ------------------------------------------------------------ |
| BASF-Gm | ------------------------------------------------------------ |
| AT4g01320 | ------------------------------------------------------------ |
| AF007269 | ------------------------------------------------------------ |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | TGACATAGGAGTGTTGTCAAAGCTTTAGAGTGCATCTTTCGGTCAGGTGCAACAGCCTTT |
| PPI-GmCPP | ------------------------------------------------------------ |
| BASP-Gm | ------------------------------------------------------------ |
| AT4g01320 | ------------------------------------------------------------ |
| AF007269 | ------------------------------------------------------------ |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | CGGTCATTGAGACATATAAGCGAATTAGCTATTAAAAAAAACAGAACTGTTGCATCAAAA |
| PPI-GmCPP | ------------------------------------------------------------ |
| BASF-Gm | ------------------------------------------------------------ |
| AT4901320 | ------------------------------------------------------------ |
| AF007269 | ------------------------------------------------------------ |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |
| BASF-Corn | AAAAAAAAAAAAAAGAAACAAAAAAAAAAAGAAAAAAAAAAAAAAAAAAAAAAAAAAAA |
| PPI-GmCPP | ------------------------------------------------------------ |
| BASF-Gm | ------------------------------------------------------------ |
| AT401320 | ------------------------------------------------------------ |
| AF007269 | ------------------------------------------------------------ |
| PPI-AtCPP | ------------------------------------------------------------ |
| BASF_AT2 | ------------------------------------------------------------ |
| afc1 | ------------------------------------------------------------ |
| BASF_AT1 | ------------------------------------------------------------ |
| PPI-BnCPP | ------------------------------------------------------------ |

BASF-Corn AAAAAGTGCTCTGCGTTGTTACCACTGCTTGCCCTATAGTGATCGTATCAGA

TABLE 6B

```
ClustalW Amino Acid Analysis of CaaX Prenyl Protease
  1: PPI-AtCPP SEQ ID NO:2
  2: PPI-BnCPP SEQ ID NO:15
  3: PPI-GmCPP SEQ ID NO:18
  4: BASF_AT1 SEQ ID NO:22
  5: BASF_AT2 SEQ ID NO:24
  6: BASF-Corn SEQ ID NO:26
  7: BASF-Gm SEQ ID NO:28
  8: AFC1 SEQ ID NO:30
  9: AT4g01320 SEQ ID NO:32
10: AF007269 SEQ ID NO:34
```

| | |
|---|---|
| PPI-CmCPP | MAFPYMEAVVGFMILMYIFETYLDVRQHRALKLPTLPKTLEG--------VISQEKFEKSR |
| BASF-Gm | MAFPYMEAVVGFMILMYIFETYLDVRQHRALKLPTLPKTLEG--------VISQEKFEKSR |
| AF007269 | MAIPFMETVVGFMIVMYIFETYLDLRQLTALKLPTLPKTLI-------------------- |
| AT4g-AtCPP | MAIPFMETVVGFMIVMYIFETYLDLRQLTALKLPTLPKTLVG----------VISQEKFEKSRAYRDIIT |
| BASF_AT2 | MAIPFMETVVCFMIVNYIFETYLDLRQLTALKLPTLPKTLVG----------VISQEKFEKSR |
| AFC1 | MAIPFMETVVGFMIVDAYIFETYLDLRQLTALKLPTLPKTLVG-----------VISQEKFEKSR |
| BASF_AT1 | MAIPFMETVVGFMIVMYIFETYLDLRQLTALKLPTLPKTLVG--------VISQEKFEKSR |
| PPI-AtCPP | MAIPFMETVVGFMIVMYIFETYLDLRQLTALKLPTLPKTLVG-------------VISQEKFEKSR |
| PPI-BnCPP | MAIPFMETVVGFMIVMYVFETYLDLRQHTALKLPTLPKTLVG-----------VISQEKFEKSR |
| BASF-Corn | ----------------------------------------------------------- |
| PPI-GmCPP | AYSLDKSHFNFVHEFVTIVTDSTILYFGVLPWFWKKSGDFMTIAGFNAENEILHTLAFLA |
| BASF-Gm | AYSLDKSHFHFVHEFVTIVTDSTILYFGVLPWFWKKSGDFMTIAGFNAENEILHTLAFLA |
| AF007269 | ----------------------------------------------------------- |
| AT4g-AtCPP | ENFNICSYFHFVHEFVTILMDSAILFFGILPWFWKMSGAVLPRLGLDPENEILHTLSFLA |
| BASF_AT2AYSLDKSYFHFVHEFVTILMDSAILFFGTLPWFWKMSGAVLPRLGLDPENEILHTLSFT-IA | |
| AFC1 | AYSLDKSYFHFVHEFVTILMDSAILFFGILPWFWKMSGAVLPRLGLDPENEILHTLSFLA |
| BASF_AT1 | AYSLDKSYFHFVHEFVTILMDSAILFFGILPWFWKMSGAVLPRLGLDPENEILHTLSFLA |
| PPI-AtCPP | AYSLDKSYFHFVHEFVTILMDSAILFFGILPWFWKMSGAVLPRLGLDPENEILHTLSFLA |
| PPI-BnCPP | AYSLDKSHFHFVHEFVTILMDSAILFFGILPWFWKISGGFLPMVGLDPENEILHTLSFLA |
| BASF-Corn | -------------------------------------------TRLSAENEIIHTLAFLA |
| PPI-GmCPP | GLMIWSQITDLPFSLYSTFVIEARNGFNKQTPWLFFRDMLKGIFLSVIIGPPIVAAIIVI |
| BASF-Gm | GLMIWSQITDLPFSLYSTFVIEARNGFNKQTPWLFFRDMLKGIFLSVIIGPPIVAAIIVI |
| AF007269 | --------TDLPFSLYSTFVIESRHGFNKQTIWMFIRDMIKGTFLSVILGPPIVAAIIFI |
| AT4g-AtCPP | GVMTWSQITDLPFSLYSTFVIESRNGFNKQTIWMFIRDMIKGTFLSVILGPPIVAAIIFI |
| BASF_AT2 | GVMTWSQITDLPFSLYSTFVIESRHGFNKQTIWMFIRDMIKGTFLSVILGPPIVAAIIFI |
| AFC1 | GVMTWSQITDLPFSLYSTFVIESRIIGFNKQTIWMFIRDMIKGTFLSVILGPPIVAAIIFI |
| BASF_AT1 | GVMTWSQITDLPFSLYSTFVIESRHGFNKQTIWMFIRDMTKGTFLSVILGPPIVAATIFI |
| PPI-AtCPP | GVMTWSQITDLPFSLYSTFVIESRHGFNKQTIWMFIRDMIKGTFLSVILGPPIVAAIIFI |
| PPI-BnCPP | GLMTWSQITDLPFSLYSTFVIESRHGFNKQTIWMFIRDMIKGILLSVIPAPPIVAAIIVI |
| BASF-Corn | GSMVWSQITDLPFSLYSTFVIEARHGFNKQTIWLFIRDMIKGILLSMILGPPIVAATIYI |
| | *********** ********* * * *** ** ** ******* * |
| PPI-GmCPP | VQKGGPYLAIYLWVFTFGLSIVMMTLYPVLIAPLFNKFTPLPDCQLREKIEKLASSLNYP |
| BASF-Gm | VQKGGPYLATYLWVFTFGLSIVMMTLYPVLIAPLFNKFTPLPDGQLREKIEKIASSLNYP |
| AF007269 | VQKGGPYLATYLWAFMFILSLVMMTIYPVLIAPLFNKFTPLPDGDLREKIEKLASSLKFP |
| AT4g-AtCPP | VQKGGPYLAIYLWAFMFILSLVMMTIYPVLIAPLFNKFTPLPDGDLREKIEKLASSLKFP |
| BASF_AT2 | VQKGGPYLAIYLWAFMFILSLVMMTIYPVLIAPLFNKFTPLPDGDLREKIEKLASSLKFP |
| AFC1 | VQKGGPYLAIYLWAFMFILSLVMMTIYPVLIAPLFNKFTPLPDGDLREKIEKLASSLKFP |
| BASF_AT1 | VQKGGPYLAIYLWAFMFILSLVMMTIYPVLIAPLFNKFTDLPDGDLREKIEKLASSLKFP |
| PPI-AtCPP | VQKCGPYLAIYLWAFMFILSLVMMTIYPVLIAPLFNKFTPLPDGDLREKIEKLASSLKFP |
| PPI-BnCPP | VQKCGPYLAIYLWAFMFILSLVMMTIYPVLIAPLFNKFTPLPDGDLREKIEKLASSLKFP |
| BASF-Corn | VQKGGPYLAIYLWGFMFVLALLMMTIYPIVIAPLFNKFTPLPEGVLREKIEKLAASLKFP |
| | ** ******** * * * *** ** *********** * ************* |
| PPI-CmCPP | LKKLFVVDGSTRSSHSNAYMYCFFKNKRIVPYDTLIQQCKDDEEIVAVIAHELGHWKLNH |
| BASF-Gm | LKKLFVVDCSTRSSHSNAYMYGFFKNKRIVLYDTLIQQCKDDEEIVAVIAHELGHWKLNH |
| AF007269 | LKKLFVVDCSTRSSHSNAYMYGFFKNKRIVLYDTLTQQCKNEDEIVAVIAHELGHWKLNH |
| AT4g-AtCPP | LKKLFVVDGSTRSSHSNAYMYGEEKNKRIVLYDTLIQQCKNEDEIVAVIAHELGWKKLNH |
| BASF_AT2 | LKKLFVVDGSTRSSUSNAYMYGEFKNKRIVLYDTLIQQCKNEDEIVAVIAHELGWKLNH |
| AFC1 | LKKLFVVDGSTRSSHSNAYMYGFFKNKRIVLYDTLIQQCKNEDEIVAVTAHELGHWKLNH |
| BASF_AT1 | LKKLEVVDGSTRSSHSNAYMYGFFKNKRIVLYDTLIQQCKNEDEIVAVIAHELGHWKLNH |
| PPI-AtCPP | LKKLEVVDGSTRSSHSNAYMYGEFKNKRIVLYDTLIQQCKNEDEIVAVIAHELGHWKLNH |
| PPI-BnCPP | LKKLFVVDCSTRSSHSNAYMYGEFKNKRIVLYDTLIQQCQNENEIVAVIAHELGHWKLNH |
| BASF-Corn | LKKLEVVDGSTRSSHSNAYMYGFEKNKRIVLYDTLIQQCSNEDEIVSVIAHELGBWKLNH |
| | **************** ********* *** ************* |
| PPI-GmCPP | TVYTEVANQILTLLQFGGYTLVRNSADLYRSEGEDTQPVLIGLIIFQHTVIPLQQLVSEG |
| BASF-Gm | TVYTEVANQILTLLQFGGYTLVRNSADLYRSEGEDTQPVLIGLIIEQHTVIPLQQLVSFG |
| AE007269 | TTYSEIAV--------------------------------------QHTVIPLQHLVSEG |
| AT4g-AtCPP | TTYSFIAVQILAFLQEGGYTLVRNSTDLFRSEGFDTQPVLIGLITEQHTVIPLQHLVSFG |
| BASF_AT2 | TTYSEIAVQILAELQFGGYTLVRNSTDLERSEGEDTQPVLIGLIIEQHTVIPLQHLVSFG |
| AFC1 | TTYSEIAVQILAFLQFGGYTLVRNSTDLERSEGFDTQPVLIGLIIEQHTVIPLQHLVSFG |
| BASF_AT1 | TTYSFIAVQILAFLQEGGYTLVRNSTDLFRSEGEDTQPVLIGLIIFQHTVIPLQHPVSFG |
| PPI-AtCPP | TTYSEIAVQILAFLQEGGYTLLRNSTDLERSEGEDTQPVLIGLIIEQHTVIPLQHLVSFG |
| PPI-BnCPP | TTYSFIAVQILAELQEGGYTLVRNSTDLERSEGEDTQPVLIGLIIFQHTVIPLQHLVSFD |
| BASF-Corn | TVYSFVAVQLLMELQEGGYTLVRSSKDLEGSEGEKDQPVIIGLIIFPHTIIPIQHLLSER |
| PPI-GmCPP | LNLVSRSEEEQADGEAKKLGYASGLRG--------------------------------- |
| BASF-Gm | LNLVSRSEEEQADGEAKKLGYASGLRG--------------------------------- |
| AE007269 | LNTNSRAEEEQADAFAVKLGYAKDLR-------PALV---KLQVREDNNRTQ-------- |
| AT4g-AtCPP | LNLVSRAEEEQADAFAVKLGYAKDLR-------PALV---KLQVREDNNRTQTVTSICV |
| BASF_AT2 | LNLVSRAEEEQADAEAVKLGYAKDL--------PALV----KLQE----- |
| AFC1 | LNLVSRAEEFQADAEAVKLGYAKDLR-------- PALVKLQE----------------- |
| BASF_AT1 | LNLVSPAEEFQADAEAVKLGYAKDLRPTLVKLQ----------------- |
| PPI-AtCPP | LNLVSRAFEEQADAEAVKLDYAKDLRPALVKLQ--------------------------- |
| PPI-BnCPP | LNLVSRAEEEQADAFAVNLGYAKDLRP--------------------------------- |
| BASF-Corn | LNLVSRAEEEQADAFAKNLGYAPQLR---------------------------------- |
| | ****** ****** ** * ** ** |
| PPI-GmCPP | -----------GLVKLQEENLSAMNTDPWYSAYHYSHPPLVERLAALDEPDKKED |
| BASF-Gm | -----------GLVKLQEENLSANNTDPCSC------------------------ |
| AE007269 | -----------TEENLSAMNTDPLYSAYHYSHPPLVERLRAIDGEDKKTD- |
| AT4g-AtCPP | -----------THLNGEEVGILQEENLSAMNTDPLYSAYHYSHPPLVERLRAIDGEDKKTD- |
| BASF_AT2 | ------------ENLSANNTDPLYSAYHYSHPPLVERLRAIDGEDKKTD- |
| AFC1 | -----------ENLSAMNTDPLHSAYHYSHPPLVERLRAIDGEDKKTD- |
| BASF_AT1 | ---------EENLSAMNTDPLYSAYHYSHPPLVERLRAIDGEDKKTD- |
| PPI-AtCPP | ------------------EENLSTMNTDPLYSAYBYSHPPLVERLRATDGEDKKTD- |
| PPI-BnCPP | ------------------ALVKLQEENLSAMNTDPLYSAYHYSHPPLVERLRAIDGEDKKTD- |
| BASF-Corn | ------------------AALVKLQEENLSANNTDPWYSAYBYSHPPLVERLQALEDSDDKKED |

### Example 4

### Plant Transformation

Arabidopsis transgenic plants were made by the method of dipping flowering plants into an Agrobacterium culture, based on the method of Andrew Bent in, Clough S J and Bent A F, 1998. Floral dipping: a simplified method for Agrobacterium-mediated transformation of Arabidopsis thaliana. Wild type plants were grown under standard conditions until the plant has both developing flowers and open flowers. The plant was inverted for 2 minutes into a solution of Agrobacterium culture carrying the appropriate gene construct. Plants were then left horizontal in a tray and kept covered for two days to maintain humidity and then righted and bagged to continue growth and seed development. Mature seed was bulk harvested.

Transformed T1 plants were selected by germination and growth on MS plates containing 50 μg/ml kanamycin. Green, kanamycin resistant (Kan^{R}) seedlings were identified after 2 weeks growth and transplanted to soil. Plants were bagged to ensure self fertilization and the T2 seed of each plant harvested separately. During growth of T1 plants leaf samples were harvested, DNA extracted and Southern blot and PCR analysis performed.

T2 seeds were analysed for Kan^{R} segregation. From those lines that showed a 3:1 resistant phenotype, surviving T2 plants were grown, bagged during seed set, and T3 seed harvested from each line. T3 seed was again used for Kan^{R} segregation analysis and those lines showing 100% Kan^{R}phenotype were selected as homozygous lines. Further molecular and physiological analysis was done using T3 seedlings.

Transgenic Brassica napus, Glycine max and Zea maize plants were produced using Agrobacterium mediated transformation of cotyledon petiole tissue. Seeds were sterilized as follows. Seeds were wetted with 95% ethanol for a short period of time such as 15 seconds. Approximately 30 ml of sterilizing solution I was added (70% Javex, 100 μl Tween20) and left for approximately 15 minutes. Solution I was removed and replaced with 30 ml of solution 11 (0.25% mecuric chloride, 1001 Tween20) and incubated for about 10 minutes. Seeds were rinsed with at least 500 ml double distilled sterile water and stored in a sterile dish. Seeds were germinated on plates of ½ MS medium, pH 5.8, supplemented with 1% sucrose and 0.7% agar. Fully expanded cotyledons were harvested and placed on Medium I (Murashige minimal organics (MMO), 3% sucrose, 4.5 mg/L benzyl adenine (BA), 0.7% phytoagar, pH 5.8). An Agrobacterium culture containing the nucleic acid construct of interest was grown for 2 days in AB Minimal media. The cotyledon explants were dipped such that only the cut portion of the petiole is contacted by the Agrobacterium solution. The explants were then embedded in Medium I and maintained for 5 days at 24° C., with 16,8 hr light dark cycles.

Explants were transferred to Medium II (Medium 1,300 mg/L timentin,) for a further 7 days and then to Medium III (Medium II, 20 mg/L kanamycin). Any root or shoot tissue which had developed at this time was dissected away. Transfer explants to fresh plates of Medium III after 14-21 days. When regenerated shoot tissue developed the regenerated tissue was transferred to Medium IV (MMO, 3% sucrose, 1.0% phytoagar, 300 mg/L timentin, 20 mg/L 20 mg/L kanamycin). Once healthy shoot tissue developed shoot tissue dissected from any callus tissue was dipped in 10×IBA and transferred to Medium V (Murashige and Skooge (MS), 3% sucrose, 0.2 mg/L indole butyric acid (IBA), 0.7% agar, 300 mg/L timentin, 20 mg/L 20 mg/L kanamycin) for rooting. Healthy plantlets were transferred to soil. The above method, with or without modifications, is suitable for the transformation of numerous plant species including Glycine max, Zea maize and cotton.

Transgenic Glycine max, Zea maize and cotton can be produced using Agrobacterium-based methods which are known to one of skill in the art. Alternatively one can use a particle or non-particle biolistic bombardment transformation method. An example of non-particle biolistic transformation is given in U.S. patent application 20010026941. This method has been used to produce transgenic Glycine max and Zea maize plants. Viable plants are propagated and homozygous lines are generated. Plants are tested for the presence of drought tolerance, physiological and biochemical phenotypes as described elsewhere.

The following table identifies the constructs and the species which they have been transformed.

TABLE 7
Transformation List

| SEQ ID NO: | Construct | Species Transformed |
|---|---|---|
| 4 | pBII121-AtCPP | A. thaliana, B. napus |
| 5 | pBII121-HP-AtCPP | A. thaliana |
| 36 | pRD29A-AtCPP | A. thaliana, B. napus |
| 37 | pRD29A-HP-AtCPP | A. thaliana |
| 39 | MuA-AtCPP | Glycine max, Zea mays |

Non-limiting examples of vector constructs suitable for plant transformation are given in SEQ ID NO: 4, 5, 35-53.

| | |
|---|---|
| gtttacccgccaatatatcctgtcaaacactgatagtttaaactgaaggcgggaaacga | SEQ ID NO:4 |
| caatctgatcatgagcggagaattaagggagtcacgttatgacccccgccgatgacgcg | |
| ggacaagccgttttacgtttggaactgacagaaccgcaacgttgaaggagccactcagc | |
| cgcgggtttctggagtttaatgagctaagcacatacgtcagaaaccattattgcgcgtt | |
| caaaagtcgcctaaggtcactatcagctagcaaatatttcttgtcaaaaatgctccact | |
| gacgttccataaattcccctcggtatccaattagagtctcatattcactctcaatccaa | |
| ataatctgcaccggatctggatcgtttcgcatgattgaacaagatggattgcacgcagg | |
| ttctccggccgcttgggtggagaggctattcggctatgactgggcacaacagacaatcg | |
| gctgctctgatgccgccgtgttccggctgtcagcgcaggggcgcccggttctttttgtc | |
| aagaccgacctgtccggtgccctgaatgaactgcaggacgaggcagcgcggctatcgtg | |
| gctggccacgacgggcgttccttgcgcagctgtgctcgacgttgtcactgaagcgggaa | |
| gggactggctgctattgggcgaagtgccggggcaggatctcctgtcatctcaccttgct | |
| cctgccgagaaagtatccatcatggctgatgcaatgcggcggctgcatacgcttgatcc | |
| ggctacctgcccattcgaccaccaagcgaaacatcgcatcgagcgagcacgtactcgga | |
| tggaagccggtcttgtcgatcaggatgatctggacgaagagcatcaggggctcgcgcca | |
| gccgaactgttcgccaggctcaaggcgcgcatgcccgacggcgatgatctcgtcgtgac | |
| ccatggcgatgcctgcttgccgaatatcatggtggaaaatggccgcttttctggattca | |
| tcgactgtggccggctgggtgtggcggaccgctatcaggacatagcgttggctacccgt | |
| gatattgctgaagagcttggcggcgaatgggctgaccgcttcctcgtgctttacggtat | |
| cgccgctcccgattcgcagcgcatcgccttctatcgccttcttgacgagttcttctgag | |
| cgggactctggggttcgaaatgaccgaccaagcgacgcccaacctgccatcacgagatt | |
| tcgattccaccgccgccttctatgaaaggttgggcttcggaatcgttttccgggacgcc | |
| ggctggatgatcctccagcgcggggatctcatgctggagttcttcgcccacgggatctc | |
| tgcggaacaggcggtcgaaggtgccgatatcattacgacagcaacggccgacaagcaca | |
| acgccacgatcctgagcgacaatatgatcgggcccggcgtccacatcaacggcgtcggc | |
| ggcgactgcccaggcaagaccgagatgcaccgcgatatcttgctgcgttcggatatttt | |
| cgtggagttcccgccacagacccggatgatccccgatcgttcaaacatttggcaataaa | |
| gtttcttaagattgaatcctgttgccggtcttgcgatgattatcatataatttctgttg | |
| aattacgttaagcatgtaataattaacatgtaatgcatgacgttatttatgagatgggt | |
| ttttatgattagagtcccgcaattatacatttaatacgcgatagaaaacaaaatatagc | |
| gcgcaaactaggataaattatcgcgcgcggtgtcatctatgttactagatcgggcctcc | |
| tgtcaatgctggcggcggctctggtggtggttctggtggcggctctgagggtggtggct | |
| ctgagggtggcggttctgagggtggcggctctgagggaggcggttccggtggtggctct | |
| ggttccggtgattttgattatgaaaagatggcaaacgctaataagggggctatgaccga | |
| aaatgccgatgaaaacgcgctacagtctgacgctaaaggcaaacttgattctgtcgcta | |
| ctgattacggtgctgctatcgatggtttcattggtgacgtttccggccttgctaatggt | |
| aatggtgctactggtgattttgctggctctaattcccaaatggctcaagtcggtgacgg | |
| tgataattcacctttaatgaataatttccgtcaatatttaccttccctccctcaatcgg | |
| ttgaatgtcgcccttttgtctttggcccaatacgcaaaccgcctctccccgcgcgttgg | |
| ccgattcattaatgcagctggcacgacaggtttcccgactggaaagcgggcagtgagcg | |
| caacgcaattaatgtgagttagctcactcattaggcaccccaggctttacactttatgc | |
| ttccggctcgtatgttgtgtggaattgtgagcggataacaatttcacacaggaaacagc | |
| tatgaccatgattacgccaagcttgcatgcctgcagcccacagatggttagagaggctt | |
| acgcagcaggtctcatcaagacgatctacccgagcaataatctccaggaaatcaaatac | |
| cttcccaagaaggttaaagatgcagtcaaaagattcaggactaactgcatcaagaacac | |
| agagaaagatatatttctcaagatcagaagtactattccagtatggacgattcaaggct | |
| tgcttcacaaaccaaggcaagtaatagagattggagtctctaaaaaggtagttcccact | |
| gaatcaaaggccatggagtcaaagattcaaatagaggacctaacagaactcgccgtaaa | |
| gactggcgaacagttcatacagagtctcttacgactcaatgacaagaagaaaatcttcg | |
| tcaacatggtggagcacgacacacttgtctactccaaaaatatcaaagatacagtctca | |
| gaagaccaaagggcaattgagacttttcaacaaagggtaatatccggaaacctcctcgg | |
| attccattgcccagctatctgtcactttattgtgaagatagtggaaaaggaaggtggct | |
| cctacaaatgccatcattgcgataaaggaaaggccatcgttgaagatgcctctgccgac | |
| agtggtcccaaagatggacccccacccacgaggagcatcgtggaaaaagaagacgttcc | |
| aaccacgtcttcaaagcaagtggattgatgtgatatctccactgacgtaagggatgacg | |
| cacaatcccactatccttcgcaagacccttcctctatataaggaagttcatttcatttg | |
| gagagaacacgggggactctagaggatccatggcgattcctttcatggaaaccgtcgtg | |
| ggttttatgatagtgatgtacatttttgagacgtatttggatctgaggcaactcactgc | |
| tctcaagcttccaactctcccgaaaaccttggttggtgtaattagccaagagaagtttg | |
| agaaatcacgagcatacagtcttgacaaaagctattttcactttgttcatgagtttgta | |
| actatacttatggactctgcaattttgttctttgggatcttgccttggttttggaagat | |
| gtctggagctgttttaccgaggttgggccttgatccggagaatgaaatactgcatactc | |
| tttcattcttggctggtgttatgacatggtcacagatcactgatttgccattttctttg | |
| tactcaactttcgtgatcgagtctcggcatgggttcaacaaacaaacaatatggatgtt | |
| cattagggacatgatcaaaggaacattcctctctgtcatactaggcccacccattgttg | |
| ctgcgataattttcatagtccagaaaggaggtccttatcttgccatctatctgtgggca | |
| ttcatgtttatcctgtctctagtgatgatgactatatacccggtcttgatagcaccgct | |
| cttcaacaaattcactcctcttccagatggagacctccgggagaagattgagaaacttg | |
| cttcttccctaaagtttcctttgaagaagctgtttgttgtcgatggatctacaaggtca | |
| agccatagcaatgcttacatgtatggtttctttaagaacaaaaggattgttctttatga | |
| tacgttgattcagcagtgcaagaatgaggatgaaattgtggcggttattgcacacgagc | |
| ttggacattggaaactgaatcacactacatactcgttcattgcagttcaaatccttgcc | |
| ttcttacaatttggaggatacactcttctcagaaactccactgatctcttcaggagttt | |
| cggatttgatacacagcctgttctcattggtttgatcatatttcagcacactgtaatac | |
| cactgcaacatctagtaagctttggcctgaacctcgttagtcgagcgtttgagtttcag | |
| gctgatgcttttgctgtgaagcttgactatgcaaaagatcttcgtcctgctctagtgaa | |
| actacaggaagagaacttatcaacaatgaacactgatccattgtactcagcttatcact | |
| actcacatcctcctcttgttgaaaggcttcgagccactgatggagaagacaagaagaca | |
| gattaacccctcgaatttccccgatcgttcaaacatttggcaataaagtttcttaagat | |
| tgaatcctgttgccggtcttgcgatgattatcatataatttctgttgaattacgttaag | |
| catgtaataattaacatgtaatgcatgacgttatttatgagatgggtttttatgattag | |
| agtcccgcaattatacatttaatacgcgatagaaaacaaaatatagcgcgcaaactagg | |
| ataaattatcgcgcgcggtgtcatctatgttactagatcgggaattcactggccgtcgt | |
| tttacaacgtcgtgactgggaaaaccctggcgttacccaacttaatcgccttgcagcac | |
| atccccctttcgccagctggcgtaatagcgaagaggcccgcaccgatcgcccttcccaa | |
| cagttgcgcagcctgaatggcgcccgctcctttcgctttcttcccttcctttctcgcca | |
| cgttcgccggctttccccgtcaagctctaaatcgggggctccctttagggttccgattt | |
| agtgctttacggcacctcgaccccaaaaaacttgatttgggtgatggttcacgtagtgg | |
| gccatcgccctgatagacggtttttcgccctttgacgttggagtccacgttctttaata | |
| gtggactcttgttccaaactggaacaacactcaaccctatctcgggctattcttttgat | |
| ttataagggattttgccgatttcggaaccaccatcaaacaggattttcgcctgctgggg | |
| caaaccagcgtggaccgcttgctgcaactctctcagggccaggcggtgaagggcaatca | |
| gctgttgcccgtctcactggtgaaaagaaaaaccaccccagtacattaaaaacgtccgc | |
| aatgtgttattaagttgtctaagcgtcaatttgtttacaccacaatatatcctgcca | |

SEQ ID NO:4 is the nucleic acid sequence of pBI121-AtCPP. Italicized sequences are the right and left border repeats. Underlined sequence is the ³⁵S promoter and bolded sequence is the AtCPP sense sequence.

| | |
|---|---|
| gtttacccgccaatatatcctgtcaaacactgatagtttaaactgaaggcgggaaacga | SEQ ID NO:5 |
| caatctgatcatgagcggagaattaagggagtcacgttatgacccccgccgatgacgcg | |
| ggacaagccgttttacgtttggaactgacagaaccgcaacgttgaaggagccactcagc | |
| cgcgggtttctggagtttaatgagctaagcacatacgtcagaaaccattattgcgcgtt | |
| caaaagtcgcctaaggtcactatcagctagcaaatatttcttgtcaaaaatgctccact | |
| gacgttccataaattcccctcggtatccaattagagtctcatattcactctcaatccaa | |
| ataatctgcaccggatctggatcgtttcgcatgattgaacaagatggattgcacgcagg | |
| ttctccggccgcttgggtggagaggctattcggctatgactgggcacaacagacaatcg | |
| gctgctctgatgccgccgtgttccggctgtcagcgcaggggcgcccggttctttttgtc | |
| aagaccgacctgtccggtgccctgaatgaactgcaggacgaggcagcgcggctatcgtg | |
| gctggccacgacgggcgttccttgcgcagctgtgctcgacgttgtcactgaagcgggaa | |
| gggactggctgctattgggcgaagtgccggggcaggatctcctgtcatctcaccttgct | |
| cctgccgagaaagtatccatcatggctgatgcaatgcggcggctgcatacgcttgatcc | |
| ggctacctgcccattcgaccaccaagcgaaacatcgcatcgagcgagcacgtactcgga | |
| tggaagccggtcttgtcgatcaggatgatctggacgaagagcatcaggggctcgcgcca | |
| gccgaactgttcgccaggctcaaggcgcgcatgcccgacggcgatgatctcgtcgtgac | |
| ccatggcgatgcctgcttgccgaatatcatggtggaaaatggccgcttttctggattca | |
| tcgactgtggccggctgggtgtggcggaccgctatcaggacatagcgttggctacccgt | |
| gatattgctgaagagcttggcggcgaatgggctgaccgcttcctcgtgctttacggtat | |
| cgccgctcccgattcgcagcgcatcgccttctatcgccttcttgacgagttcttctgag | |
| cgggactctggggttcgaaatgaccgaccaagcgacgcccaacctgccatcacgagatt | |
| tcgattccaccgccgccttctatgaaaggttgggcttcggaatcgttttccgggacgcc | |
| ggctggatgatcctccagcgcggggatctcatgctggagttcttcgcccacgggatctc | |
| tgcggaacaggcggtcgaaggtgccgatatcattacgacagcaacggccgacaagcaca | |
| acgccacgatcctgagcgacaatatgatcgggcccggcgtccacatcaacggcgtcggc | |
| ggcgactgcccaggcaagaccgagatgcaccgcgatatcttgctgcgttcggatatttt | |
| cgtggagttcccgccacagacccggatgatccccgatcgttcaaacatttggcaataaa | |
| gtttcttaagattgaatcctgttgccggtcttgcgatgattatcatataatttctgttg | |
| aattacgttaagcatgtaataattaacatgtaatgcatgacgttatttatgagatgggt | |
| ttttatgattagagtcccgcaattatacatttaatacgcgatagaaaacaaaatatagc | |
| gcgcaaactaggataaattatcgcgcgcggtgtcatctatgttactagatcgggcctcc | |
| tgtcaatgctggcggcggctctggtggtggttctggtggcggctctgagggtggtggct | |
| ctgagggtggcggttctgagggtggcggctctgagggaggcggttccggtggtggctct | |
| ggttccggtgattttgattatgaaaagatggcaaacgctaataagggggctatgaccga | |
| aaatgccgatgaaaacgcgctacagtctgacgctaaaggcaaacttgattctgtcgcta | |
| ctgattacggtgctgctatcgatggtttcattggtgacgtttccggccttgctaatggt | |
| aatggtgctactggtgattttgctggctctaattcccaaatggctcaagtcggtgacgg | |
| tgataattcacctttaatgaataatttccgtcaatatttaccttccctccctcaatcgg | |
| ttgaatgtcgcccttttgtctttggcccaatacgcaaaccgcctctccccgcgcgttgg | |
| ccgattcattaatgcagctggcacgacaggtttcccgactggaaagcgggcagtgagcg | |
| caacgcaattaatgtgagttagctcactcattaggcaccccaggctttacactttatgc | |
| ttccggctcgtatgttgtgtggaattgtgagcggataacaatttcacacaggaaacagc | |
| tatgaccatgattacgccaagcttgcatgcctgcagcccacagatggttagagaggctt | |
| acgcagcaggtctcatcaagacgatctacccgagcaataatctccaggaaatcaaatac | |
| cttcccaagaaggttaaagatgcagtcaaaagattcaggactaactgcatcaagaacac | |
| agagaaagatatatttctcaagatcagaagtactattccagtatggacgattcaaggct | |
| tgcttcacaaaccaaggcaagtaatagagattggagtctctaaaaaggtagttcccact | |
| gaatcaaaggccatggagtcaaagattcaaatagaggacctaacagaactcgccgtaaa | |
| gactggcgaacagttcatacagagtctcttacgactcaatgacaagaagaaaatcttcg | |
| tcaacatggtggagcacgacacacttgtctactccaaaaatatcaaagatacagtctca | |
| gaagaccaaagggcaattgagacttttcaacaaagggtaatatccggaaacctcctcgg | |
| attccattgcccagctatctgtcactttattgtgaagatagtggaaaaggaaggtggct | |
| cctacaaatgccatcattgcgataaaggaaaggccatcgttgaagatgcctctgccgac | |
| agtggtcccaaagatggacccccacccacgaggagcatcgtggaaaaagaagacgttcc | |
| aaccacgtcttcaaagcaagtggattgatgtgatatctccactgacgtaagggatgacg | |
| cacaatcccactatccttcgcaagacccttcctctatataaggaagttcatttcatttg | |
| gagagaacacgggggactctagaggatcctcccaatgtccaagctcgtgtgcaataacc | |
| gccacaatttcatcctcattcttgcactgctgaatcaacgtatcataaagaacaatcct | |
| tttgttcttaaagaaaccatacatgtaagcattgctatggcttgaccttgtagatccat | |
| cgacaacaaacagcttcttcaaaggaaactttagggaagaagcaagtttctcaatcttc | |
| tcccggaggtctccatctggaagaggagtgaatttgttgaagagcggtgctatcaagac | |
| cgggtatatagtcatcatcactagagacaggataaacatgaatgcccacagatagatgg | |
| caagataaggacctcctttctggactatgaaaattatcgcagcaacaatgggtgggcct | |
| agtatgacagagaggaatgttcctttgatcatgtccctaatgaacatccatattgtttg | |
| tttgttgaacccatgccgagactcgatcacgaaagttgagtacaaagaaaatggcaaat | |
| cagtgatctgtgaccatgtcataacaccagccaagaatgaaagagtatgcagtatttca | |
| ttctccggatcaaggcccaacctcggtaaaagaggatccccatctacccgcttcgcgtc | |
| GGCATCCGGTCAGTGGCAGTGAAGGGCGAACAGTTCCTGATTAACCACAAACCGTTCTA | |
| CTTTACTGGCTTTGGTCGTCATGAAGATGCGGACTTGCGTGGCAAAGGATTCGATAACG | |
| TGCTGATGGTGCACGACCACGCATTAATGGACTGGATTGGGGCCAACTCCTACCGTACC | |
| TCGCATTACCCTTACGCTGAAGAGATGCTCGACTGGGCAGATGAACATGGCATCGTGGT | |
| GATTGATGAAACTGCTGCTGTCGGCTTTTCGCTCTCTTTAGGCATTGGTTTCGAAGCGG | |
| GCAACAAGCCGAAAGAACTGTACAGCGAAGAGGCAGTCAACGGGGAAACTCAGCAAGCG | |
| CACTTACAGGCGATTAAAGAGCTGATAGCGCGTGACAAAAACCACCCAAGCGTGGTGAT | |
| GTGGAGTATTGCCAACGAACCGGATACCCGTCCGCAAGGTGCACGGGAATATTTCGCGC | |
| CACTGGCGGAAGCAACGCGTAAACTCGACCCGACGCGTCCGATCACCTGCGTCAATGTA | |
| ATGTTCTGCGACGCTCACACCGATACCATCAGCGATCTCTTTGATGTGCTGTGCCTGAA | |
| CCGTTATTACGGATGGTATGTCCAAAGCGGCGATTTGGAAACGGCAGAGAAGGTACTGG | |
| AAAAAGAACTTCTGGCCTGGCAGGAGAAACTGTACACCGACATGTGGAGTGAAGAGTAT | |
| CAGTGTGCATGGCTGGATATGTATCACCGCGTCTTTGATCGCGTCAGCGCCGTCGTCGG | |
| TGAACAGGTATGGAATTTCGCCGATTTTGCGACCTCGCAAGGCATATTGCGCGTTGGCG | |
| GTAACAAGAAAGGGATCTTCACTCGCGACCCCAAACCGAAGTCGGCGGCTTTTCTGCTG | |
| CAAAAACGCTGGACTGGCATGAACTTCGGTGAAAAACCGCAGCAGGGAGGCAAACAATG | |
| AATCAACAACTCTCCTGGCGCACCATCGTCGGCTACAGCCTCGGGAATTGCTACCGAGC | |
| TCttttaccgaggttgggccttgatccggagaatgaaatactgcatactctttcattct | |
| tggctggtgttatgacatggtcacagatcactgatttgccattttctttgtactcaact | |
| ttcgtgatcgagtctcggcatgggttcaacaaacaaacaatatggatgttcattaggga | |
| catgatcaaaggaacattcctctctgtcatactaggcccacccattgttgctgcgataa | |
| ttttcatagtccagaaaggaggtccttatcttgccatctatctgtgggcattcatgttt | |
| atcctgtctctagtgatgatgactatatacccggtcttgatagcaccgctcttcaacaa | |
| attcactcctcttccagatggagacctccgggagaagattgagaaacttgcttcttccc | |
| taaagtttcctttgaagaagctgtttgttgtcgatggatctacaaggtcaagccatagc | |
| aatgcttacatgtatggtttctttaagaacaaaaggattgttctttatgatacgttgat | |
| tcagcagtgcaagaatgaggatgaaattgtggcggttattgcacacgagcttggacatt | |
| gggagctcgaatttccccgatcgttcaaacatttggcaataaagtttcttaagattgaa | |
| tcctgttgccggtcttgcgatgattatcatataatttctgttgaattacgttaagcatg | |
| taataattaacatgtaatgcatgacgttatttatgagatgggtttttatgattagagtc | |
| ccgcaattatacatttaatacgcgatagaaaacaaaatatagcgcgcaaactaggataa | |
| attatcgcgcgcggtgtcatctatgttactagatcgggaattcactggccgtcgtttta | |
| caacgtcgtgactgggaaaaccctggcgttacccaacttaatcgccttgcagcacatcc | |
| ccctttcgccagctggcgtaatagcgaagaggcccgcaccgatcgcccttcccaacagt | |
| tgcgcagcctgaatggcgcccgctcctttcgctttcttcccttcctttctcgccacgtt | |
| cgccggctttccccgtcaagctctaaatcgggggctccctttagggttccgatttagtg | |
| ctttacggcacctcgaccccaaaaaacttgatttgggtgatggttcacgtagtgggcca | |
| tcgccctgatagacggtttttcgccctttgacgttggagtccacgttctttaatagtgg | |
| actcttgttccaaactggaacaacactcaaccctatctcgggctattcttttgatttat | |
| aagggattttgccgatttcggaaccaccatcaaacaggattttcgcctgctggggcaaa | |
| ccagcgtggaccgcttgctgcaactctctcagggccaggcggtgaagggcaatcagctg | |
| ttgcccgtctcactggtgaaaagaaaaaccaccccagtacattaaaaacgtccgcaatg | |
| tgttattaagttgtctaagcgtcaatttgtttacaccacaatatatcctgcca | |

SEQ ID NO:5 is the nucleic acid sequence of pBI121-HP-AtCPP. Italicized sequences are the right and left border repeats. Underlined sequence is the ³⁵S promoter and bolded sequence is the AtCPP anti-sense sequence. Sequence in upper case is the truncated GUS fragment. Sequence in bold and underlined is the AtCPP sense sequence.

| | |
|---|---|
| gtttacccgccaatatatcctgtcaaacactgatagtttaaactgaaggcgggaaacga | SEQ ID NO:35 |
| caatctgatcatgagcggagaattaagggagtcacgttatgacccccgccgatgacgcg | |
| ggacaagccgttttacgtttggaactgacagaaccgcaacgttgaaggagccactcagc | |
| cgcgggtttctggagtttaatgagctaagcacatacgtcagaaaccattattgcgcgtt | |
| caaaagtcgcctaaggtcactatcagctagcaaatatttcttgtcaaaaatgctccact | |
| gacgttccataaattcccctcggtatccaattagagtctcatattcactctcaatccaa | |
| ataatctgcaccggatctggatcgtttcgcatgattgaacaagatggattgcacgcagg | |
| ttctccggccgcttgggtggagaggctattcggctatgactgggcacaacagacaatcg | |
| gctgctctgatgccgccgtgttccggctgtcagcgcaggggcgcccggttctttttgtc | |
| aagaccgacctgtccggtgccctgaatgaactgcaggacgaggcagcgcggctatcgtg | |
| gctggccacgacgggcgttccttgcgcagctgtgctcgacgttgtcactgaagcgggaa | |
| gggactggctgctattgggcgaagtgccggggcaggatctcctgtcatctcaccttgct | |
| cctgccgagaaagtatccatcatggctgatgcaatgcggcggctgcatacgcttgatcc | |
| ggctacctgcccattcgaccaccaagcgaaacatcgcatcgagcgagcacgtactcgga | |
| tggaagccggtcttgtcgatcaggatgatctggacgaagagcatcaggggctcgcgcca | |
| gccgaactgttcgccaggctcaaggcgcgcatgcccgacggcgatgatctcgtcgtgac | |
| ccatggcgatgcctgcttgccgaatatcatggtggaaaatggccgcttttctggattca | |
| tcgactgtggccggctgggtgtggcggaccgctatcaggacatagcgttggctacccgt | |
| gatattgctgaagagcttggcggcgaatgggctgaccgcttcctcgtgctttacggtat | |
| cgccgctcccgattcgcagcgcatcgccttctatcgccttcttgacgagttcttctgag | |
| cgggactctggggttcgaaatgaccgaccaagcgacgcccaacctgccatcacgagatt | |
| tcgattccaccgccgccttctatgaaaggttgggcttcggaatcgttttccgggacgcc | |
| ggctggatgatcctccagcgcggggatctcatgctggagttcttcgcccacgggatctc | |
| tgcggaacaggcggtcgaaggtgccgatatcattacgacagcaacggccgacaagcaca | |
| acgccacgatcctgagcgacaatatgatcgggcccggcgtccacatcaacggcgtcggc | |
| ggcgactgcccaggcaagaccgagatgcaccgcgatatcttgctgcgttcggatatttt | |
| cgtggagttcccgccacagacccggatgatccccgatcgttcaaacatttggcaataaa | |
| gtttcttaagattgaatcctgttgccggtcttgcgatgattatcatataatttctgttg | |
| aattacgttaagcatgtaataattaacatgtaatgcatgacgttatttatgagatgggt | |
| ttttatgattagagtcccgcaattatacatttaatacgcgatagaaaacaaaatatagc | |
| gcgcaaactaggataaattatcgcgcgcggtgtcatctatgttactagatcgggcctcc | |
| tgtcaatgctggcggcggctctggtggtggttctggtggcggctctgagggtggtggct | |
| ctgagggtggcggttctgagggtggcggctctgagggaggcggttccggtggtggctct | |
| ggttccggtgattttgattatgaaaagatggcaaacgctaataagggggctatgaccga | |
| aaatgccgatgaaaacgcgctacagtctgacgctaaaggcaaacttgattctgtcgcta | |
| ctgattacggtgctgctatcgatggtttcattggtgacgtttccggccttgctaatggt | |
| aatggtgctactggtgattttgctggctctaattcccaaatggctcaagtcggtgacgg | |
| tgataattcacctttaatgaataatttccgtcaatatttaccttccctccctcaatcgg | |
| ttgaatgtcgcccttttgtctttggcccaatacgcaaaccgcctctccccgcgcgttgg | |
| ccgattcattaatgcagctggcacgacaggtttcccgactggaaagcgggcagtgagcg | |
| caacgcaattaatgtgagttagctcactcattaggcaccccaggctttacactttatgc | |
| ttccggctcgtatgttgtgtggaattgtgagcggataacaatttcacacaggaaacagc | |
| tatgaccatgattacgccaagcttgcatgcctgcagcccacagatggttagagaggctt | |
| acgcagcaggtctcatcaagacgatctacccgagcaataatctccaggaaatcaaatac | |
| cttcccaagaaggttaaagatgcagtcaaaagattcaggactaactgcatcaagaacac | |
| agagaaagatatatttctcaagatcagaagtactattccagtatggacgattcaaggct | |
| tgcttcacaaaccaaggcaagtaatagagattggagtctctaaaaaggtagttcccact | |
| gaatcaaaggccatggagtcaaagattcaaatagaggacctaacagaactcgccgtaaa | |
| gactggcgaacagttcatacagagtctcttacgactcaatgacaagaagaaaatcttcg | |
| tcaacatggtggagcacgacacacttgtctactccaaaaatatcaaagatacagtctca | |
| gaagaccaaagggcaattgagacttttcaacaaagggtaatatccggaaacctcctcgg | |
| attccattgcccagctatctgtcactttattgtgaagatagtggaaaaggaaggtggct | |
| cctacaaatgccatcattgcgataaaggaaaggccatcgttgaagatgcctctgccgac | |
| agtggtcccaaagatggacccccacccacgaggagcatcgtggaaaaagaagacgttcc | |
| aaccacgtcttcaaagcaagtggattgatgtgatatctccactgacgtaagggatgacg | |
| cacaatcccactatccttcgcaagacccttcctctatataaggaagttcatttcatttg | |
| gagagaacacgggggactctagaggatccttaatctgtcttcttgtcttctccatcagt | |
| GGCTCGAAGCCTTTCAACAAGAGGAGGATGTCAGTAGTGATAAGCTGAGTACAATGGAT | |
| CAGTGTTCATTGTTGATAAGTTCTCTTCCTGTAGTTTCACTAGAGCAGGACGAAGATCT | |
| TTTGCATAGTCAAGCTTCACAGCAAAAGCATCAGCCTGAAACTCAAACGCTCGACTAAC | |
| GAGGTTCAGGCCAAAGCTTACTAGATGTTGCAGTGGTATTACAGTGTGCTGAAATATGA | |
| TCAAACCAATGAGAACAGGCTGTGTATCAAATCCGAAACTCCTGAAGAGATCAGTGGAG | |
| TTTCTGAGAAGAGTGTATCCTCCAAATTGTAAGAAGGCAAGGATTTGAACTGCAATGAA | |
| CGAGTATGTAGTGTGATTCAGTTTCCAATGTCCAAGCTCGTGTGCAATAACCGCCACAA | |
| TTTCATCCTCATTCTTGCACTGCTGAATCAACGTATCATAAAGAACAATCCTTTTGTTC | |
| TTAAAGAAACCATACATGTAAGCATTGCTATGGCTTGACCTTGTAGATCCATCGACAAC | |
| AAACAGCTTCTTCAAAGGAAACTTTAGGGAAGAAGCAAGTTTCTCAATCTTCTCCCGGA | |
| GGTCTCCATCTGGAAGAGGAGTGAATTTGTTGAAGAGCGGTGCTATCAAGACCGGGTAT | |
| ATAGTCATCATCACTAGAGACAGGATAAACATGAATGCCCACAGATAGATGGCAAGATA | |
| AGGACCTCCTTTCTGGACTATGAAAATTATCGCAGCAACAATGGGTGGGCCTAGTATGA | |
| CAGAGAGGAATGTTCCTTTGATCATGTCCCTAATGAACATCCATATTGTTTGTTTGTTG | |
| AACCCATGCCGAGACTCGATCACGAAAGTTGAGTACAAAGAAAATGGCAAATCAGTGAT | |
| CTGTGACCATGTCATAACACCAGCCAAGAATGAAAGAGTATGCAGTATTTCATTCTCCG | |
| GATCAAGGCCCAACCTCGGTAAAACAGCTCCAGACATCTTCCAAAACCAAGGCAAGATC | |
| CCAAAGAACAAAATTGCAGAGTCCATAAGTATAGTTACAAACTCATGAACAAAGTGAAA | |
| ATAGCTTTTGTCAAGACTGTATGCTCGTGATTTCTCAAACTTCTCTTGGCTAATTACAC | |
| CAACCAAGGTTTTCGGGAGAGTTGGAAGCTTGAGAGCAGTGAGTTGCCTCAGATCCAAA | |
| TACGTCTCAAAAATGTACATCACTATCATAAAACCCACGACGGTTTCCATGAAAGGAAT | |
| CGCCATCCCCTCGAATTTCCCCGATCGTTCAAACATTTGGCAATAAAGTTTCTTAAGAT | |
| tgaatcctgttgccggtcttgcgatgattatcatataatttctgttgaattacgttaag | |
| catgtaataattaacatgtaatgcatgacgttatttatgagatgggtttttatgattag | |
| agtcccgcaattatacatttaatacgcgatagaaaacaaaatatagcgcgcaaactagg | |
| ataaattatcgcgcgcggtgtcatctatgttactagatcgggaattcactggccgtcgt | |
| tttacaacgtcgtgactgggaaaaccctggcgttacccaacttaatcgccttgcagcac | |
| atccccctttcgccagctggcgtaatagcgaagaggcccgcaccgatcgcccttcccaa | |
| cagttgcgcagcctgaatggcgcccgctcctttcgctttcttcccttcctttctcgcca | |
| cgttcgccggctttccccgtcaagctctaaatcgggggctccctttagggttccgattt | |
| agtgctttacggcacctcgaccccaaaaaacttgatttgggtgatggttcacgtagtgg | |
| gccatcgccctgatagacggtttttcgccctttgacgttggagtccacgttctttaata | |
| gtggactcttgttccaaactggaacaacactcaaccctatctcgggctattcttttgat | |
| ttataagggatttttgccgatttcggaaccaccatcaaacaggattttccctgctgggg | |
| caaaccagcgtggaccgcttgctgcaactctctcagggccaggcggtgaagggcaatca | |
| gctgttgcccgtctcactggtgaaaagaaaaaccaccccagtacattaaaaacgtccgc | |
| aatgtgttattaagttgtctaagcgtcaatttgtttacaccacaatatatcctgcca | |

SEQ ID NO:35 is the nucleic acid sequence of pBI121-antisense-AtCPP. Italicized sequences are the right and left border repeats. Underlined sequence is the ³⁵S promoter. Sequence in upper case is the AtCPP anti-sense sequence.

| | |
|---|---|
| gtttacccgccaatatatcctgtcaaacactgatagtttaaactgaaggcgggaaacga | SEQ ID NO:36 |
| caatctgatcatgagcggagaattaagggagtcacgttatgacccccgccgatgacgcg | |
| ggacaagccgttttacgtttggaactgacagaaccgcaacgttgaaggagccactcagc | |
| cgcgggtttctggagtttaatgagctaagcacatacgtcagaaaccattattgcgcgtt | |
| caaaagtcgcctaaggtcactatcagctagcaaatatttcttgtcaaaaatgctccact | |
| gacgttccataaattcccctcggtatccaattagagtctcatattcactctcaatccaa | |
| ataatctgcaccggatctggatcgtttcgcatgattgaacaagatggattgcacgcagg | |
| ttctccggccgcttgggtggagaggctattcggctatgactgggcacaacagacaatcg | |
| gctgctctgatgccgccgtgttccggctgtcagcgcaggggcgcccggttctttttgtc | |
| aagaccgacctgtccggtgccctgaatgaactgcaggacgaggcagcgcggctatcgtg | |
| gctggccacgacgggcgttccttgcgcagctgtgctcgacgttgtcactgaagcgggaa | |
| gggactggctgctattgggcgaagtgccggggcaggatctcctgtcatctcaccttgct | |
| cctgccgagaaagtatccatcatggctgatgcaatgcggcggctgcatacgcttgatcc | |
| ggctacctgcccattcgaccaccaagcgaaacatcgcatcgagcgagcacgtactcgga | |
| tggaagccggtcttgtcgatcaggatgatctggacgaagagcatcaggggctcgcgcca | |
| gccgaactgttcgccaggctcaaggcgcgcatgcccgacggcgatgatctcgtcgtgac | |
| ccatggcgatgcctgcttgccgaatatcatggtggaaaatggccgcttttctggattca | |
| tcgactgtggccggctgggtgtggcggaccgctatcaggacatagcgttggctacccgt | |
| gatattgctgaagagcttggcggcgaatgggctgaccgcttcctcgtgctttacggtat | |
| cgccgctcccgattcgcagcgcatcgccttctatcgccttcttgacgagttcttctgag | |
| cgggactctggggttcgaaatgaccgaccaagcgacgcccaacctgccatcacgagatt | |
| tcgattccaccgccgccttctatgaaaggttgggcttcggaatcgttttccgggacgcc | |
| ggctggatgatcctccagcgcggggatctcatgctggagttcttcgcccacgggatctc | |
| tgcggaacaggcggtcgaaggtgccgatatcattacgacagcaacggccgacaagcaca | |
| acgccacgatcctgagcgacaatatgatcgggcccggcgtccacatcaacggcgtcggc | |
| ggcgactgcccaggcaagaccgagatgcaccgcgatatcttgctgcgttcggatatttt | |
| cgtggagttcccgccacagacccggatgatccccgatcgttcaaacatttggcaataaa | |
| gtttcttaagattgaatcctgttgccggtcttgcgatgattatcatataatttctgttg | |
| aattacgttaagcatgtaataattaacatgtaatgcatgacgttatttatgagatgggt | |
| ttttatgattagagtcccgcaattatacatttaatacgcgatagaaaacaaaatatagc | |
| gcgcaaactaggataaattatcgcgcgcggtgtcatctatgttactagatcgggcctcc | |
| tgtcaatgctggcggcggctctggtggtggttctggtggcggctctgagggtggtggct | |
| ctgagggtggcggttctgagggtggcggctctgagggaggcggttccggtggtggctct | |
| ggttccggtgattttgattatgaaaagatggcaaacgctaataagggggctatgaccga | |
| aaatgccgatgaaaacgcgctacagtctgacgctaaaggcaaacttgattctgtcgcta | |
| ctgattacggtgctgctatcgatggtttcattggtgacgtttccggccttgctaatggt | |
| aatggtgctactggtgattttgctggctctaattcccaaatggctcaagtcggtgacgg | |
| tgataattcacctttaatgaataatttccgtcaatatttaccttccctccctcaatcgg | |
| ttgaatgtcgcccttttgtctttggcccaatacgcaaaccgcctctccccgcgcgttgg | |
| ccgattcattaatgcagctggcacgacaggtttcccgactggaaagcgggcagtgagcg | |
| caacgcaattaatgtgagttagctcactcattaggcaccccaggctttacactttatgc | |
| ttccggctcgtatgttgtgtggaattgtgagcggataacaatttcacacaggaaacagc | |
| tatgaccatgattacgccaagcttgcatgcctgcagggagccatagatgcaattcaatc | |
| aaactgaaatttctgcaagaatctcaaacacggagatctcaaagtttgaaagaaaattt | |
| atttcttcgactcaaaacaaacttacgaaatttaggtagaacttatatacattatattg | |
| taattttttgtaacaaaatgtttttattattattatagaattttactggttaaattaaa | |
| aatgaatagaaaaggtgaattaagaggagagaggaggtaaacattttcttctatttttt | |
| catattttcaggataaattattgtaaaagtttacaagatttccatttgactagtgtaaa | |
| tgaggaatattctctagtaagatcattatttcatctacttcttttatcttctaccagta | |
| gaggaataaacaatatttagctcctttgtaaatacaaattaattttccttcttgacatc | |
| attcaattttaattttacgtataaaataaaagatcatacctattagaacgattaaggag | |
| aaatacaattcgaatgagaaggatgtgccgtttgttataataaacagccacacgacgta | |
| aacgtaaaatgaccacatgatgggccaatagacatggaccgactactaataatagtaag | |
| ttacattttaggatggaataaatatcataccgacatcagttttgaaagaaaagggaaaa | |
| aaagaaaaaataaataaaagatatactaccgacatgagttccaaaaagcaaaaaaaaag | |
| atcaagccgacacagacacgcgtagagagcaaaatgactttgacgtcacaccacgaaaa | |
| cagacgcttcatacgtgtccctttatctctctcagtctctctataaacttagtgagacc | |
| ctcctctgttttactcacaaatatgcaaactagaaaacaatcatcaggaataaagggtt | |
| tgattacttctattggaaaggactctagaggatccatggcgattcctttcatggaaacc | |
| gtcgtgggttttatgatagtgatgtacatttttgagacgtatttggatctgaggcaact | |
| cactgctctcaagcttccaactctcccgaaaaccttggttggtgtaattagccaagaga | |
| agtttgagaaatcacgagcatacagtcttgacaaaagctattttcactttgttcatgag | |
| tttgtaactatacttatggactctgcaattttgttctttgggatcttgccttggttttg | |
| gaagatgtctggagctgttttaccgaggttgggccttgatccggagaatgaaatactgc | |
| atactctttcattcttggctggtgttatgacatggtcacagatcactgatttgccattt | |
| tctttgtactcaactttcgtgatcgagtctcggcatgggttcaacaaacaaacaatatg | |
| gatgttcattagggacatgatcaaaggaacattcctctctgtcatactaggcccaccca | |
| ttgttgctgcgataattttcatagtccagaaaggaggtccttatcttgccatctatctg | |
| tgggcattcatgtttatcctgtctctagtgatgatgactatatacccggtcttgatagc | |
| accgctcttcaacaaattcactcctcttccagatggagacctccgggagaagattgaga | |
| aacttgcttcttccctaaagtttcctttgaagaagctgtttgttgtcgatggatctaca | |
| aggtcaagccatagcaatgcttacatgtatggtttctttaagaacaaaaggattgttct | |
| ttatgatacgttgattcagcagtgcaagaatgaggatgaaattgtggcggttattgcac | |
| acgagcttggacattggaaactgaatcacactacatactcgttcattgcagttcaaatc | |
| cttgccttcttacaatttggaggatacactcttctcagaaactccactgatctcttcag | |
| gagtttcggatttgatacacagcctgttctcattggtttgatcatatttcagcacactg | |
| taataccactgcaacatctagtaagctttggcctgaacctcgttagtcgagcgtttgag | |
| tttcaggctgatgcttttgctgtgaagcttgactatgcaaaagatcttcgtcctgctct | |
| agtgaaactacaggaagagaacttatcaacaatgaacactgatccattgtactcagctt | |
| atcactactcacatcctcctcttgttgaaaggcttcgagccactgatggagaagacaag | |
| aagacagattaacccctcgaatttccccgatcgttcaaacatttggcaataaagtttct | |
| taagattgaatcctgttgccggtcttgcgatgattatcatataatttctgttgaattac | |
| gttaagcatgtaataattaacatgtaatgcatgacgttatttatgagatgggtttttat | |
| gattagagtcccgcaattatacatttaatacgcgatagaaaacaaaatatagcgcgcaa | |
| actaggataaattatcgcgcgcggtgtcatctatgttactagatcgggaattcactggc | |
| cgtcgttttacaacgtcgtgactgggaaaaccctggcgttacccaacttaatcgccttg | |
| cagcacatccccctttcgccagctggcgtaatagcgaagaggcccgcaccgatcgccct | |
| tcccaacagttgcgcagcctgaatggcgcccgctcctttcgctttcttcccttcctttc | |
| tcgccacgttcgccggctttccccgtcaagctctaaatcgggggctccctttagggttc | |
| cgatttagtgctttacggcacctcgaccccaaaaaacttgatttgggtgatggttcacg | |
| tagtgggccatcgccctgatagacggtttttcgccctttgacgttggagtccacgttct | |
| ttaatagtggactcttgttccaaactggaacaacactcaaccctatctcgggctattct | |
| tttgatttataagggattttgccgatttcggaaccaccatcaaacaggattttcgcctg | |
| ctggggcaaaccagcgtggaccgcttgctgcaactctctcagggccaggcggtgaaggg | |
| caatcagctgttgcccgtctcactggtgaaaagaaaaaccaccccagtacattaaaaac | |
| gtccgcaatgtgttattaagttgtctaagcgtcaatttgtttacaccacaatatatcct | |
| gcca | |

SEQ ID NO:36 is the nucleic acid sequence of RD29A-AtCPP. Italicized sequences are the right and left border repeats. Underlined sequence is the RD29A promoter. Sequence in bold is the AtCPP sense sequence.

| | |
|---|---|
| gtttacccgccaatatatcctgtcaaacactgatagtttaaactgaaggcgggaaacga | SEQ ID NO:37 |
| caatctgatcatgagcggagaattaagggagtcacgttatgacccccgccgatgacgcg | |
| ggacaagccgttttacgtttggaactgacagaaccgcaacgttgaaggagccactcagc | |
| cgcgggtttctggagtttaatgagctaagcacatacgtcagaaaccattattgcgcgtt | |
| caaaagtcgcctaaggtcactatcagctagcaaatatttcttgtcaaaaatgctccact | |
| gacgttccataaattcccctcggtatccaattagagtctcatattcactctcaatccaa | |
| ataatctgcaccggatctggatcgtttcgcatgattgaacaagatggattgcacgcagg | |
| ttctccggccgcttgggtggagaggctattcggctatgactgggcacaacagacaatcg | |
| gctgctctgatgccgccgtgttccggctgtcagcgcaggggcgcccggttctttttgtc | |
| aagaccgacctgtccggtgccctgaatgaactgcaggacgaggcagcgcggctatcgtg | |
| gctggccacgacgggcgttccttgcgcagctgtgctcgacgttgtcactgaagcgggaa | |
| gggactggctgctattgggcgaagtgccggggcaggatctcctgtcatctcaccttgct | |
| cctgccgagaaagtatccatcatggctgatgcaatgcggcggctgcatacgcttgatcc | |
| ggctacctgcccattcgaccaccaagcgaaacatcgcatcgagcgagcacgtactcgga | |
| tggaagccggtcttgtcgatcaggatgatctggacgaagagcatcaggggctcgcgcca | |
| gccgaactgttcgccaggctcaaggcgcgcatgcccgacggcgatgatctcgtcgtgac | |
| ccatggcgatgcctgcttgccgaatatcatggtggaaaatggccgcttttctggattca | |
| tcgactgtggccggctgggtgtggcggaccgctatcaggacatagcgttggctacccgt | |
| gatattgctgaagagcttggcggcgaatgggctgaccgcttcctcgtgctttacggtat | |
| cgccgctcccgattcgcagcgcatcgccttctatcgccttcttgacgagttcttctgag | |
| cgggactctggggttcgaaatgaccgaccaagcgacgcccaacctgccatcacgagatt | |
| tcgattccaccgccgccttctatgaaaggttgggcttcggaatcgttttccgggacgcc | |
| ggctggatgatcctccagcgcggggatctcatgctggagttcttcgcccacgggatctc | |
| tgcggaacaggcggtcgaaggtgccgatatcattacgacagcaacggccgacaagcaca | |
| acgccacgatcctgagcgacaatatgatcgggcccggcgtccacatcaacggcgtcggc | |
| ggcgactgcccaggcaagaccgagatgcaccgcgatatcttgctgcgttcggatatttt | |
| cgtggagttcccgccacagacccggatgatccccgatcgttcaaacatttggcaataaa | |
| gtttcttaagattgaatcctgttgccggtcttgcgatgattatcatataatttctgttg | |
| aattacgttaagcatgtaataattaacatgtaatgcatgacgttatttatgagatgggt | |
| ttttatgattagagtcccgcaattatacatttaatacgcgatagaaaacaaaatatagc | |
| gcgcaaactaggataaattatcgcgcgcggtgtcatctatgttactagatcgggcctcc | |
| tgtcaatgctggcggcggctctggtggtggttctggtggcggctctgagggtggtggct | |
| ctgagggtggcggttctgagggtggcggctctgagggaggcggttccggtggtggctct | |
| ggttccggtgattttgattatgaaaagatggcaaacgctaataagggggctatgaccga | |
| aaatgccgatgaaaacgcgctacagtctgacgctaaaggcaaacttgattctgtcgcta | |
| ctgattacggtgctgctatcgatggtttcattggtgacgtttccggccttgctaatggt | |
| aatggtgctactggtgattttgctggctctaattcccaaatggctcaagtcggtgacgg | |
| tgataattcacctttaatgaataatttccgtcaatatttaccttccctccctcaatcgg | |
| ttgaatgtcgcccttttgtctttggcccaatacgcaaaccgcctctccccgcgcgttgg | |
| ccgattcattaatgcagctggcacgacaggtttcccgactggaaagcgggcagtgagcg | |
| caacgcaattaatgtgagttagctcactcattaggcaccccaggctttacactttatgc | |
| ttccggctcgtatgttgtgtggaattgtgagcggataacaatttcacacaggaaacagc | |
| tatgaccatgattacgccaagcttgcatgcctgcagggagccatagatgcaattcaatc | |
| aaactgaaatttctgcaagaatctcaaacacggagatctcaaagtttgaaagaaaattt | |
| atttcttcgactcaaaacaaacttacgaaatttaggtagaacttatatacattatattg | |
| taattttttgtaacaaaatgtttttattattattatagaattttactggttaaattaaa | |
| aatgaatagaaaaggtgaattaagaggagagaggaggtaaacattttcttctatttttt | |
| catattttcaggataaattattgtaaaagtttacaagatttccatttgactagtgtaaa | |
| tgaggaatattctctagtaagatcattatttcatctacttcttttatcttctaccagta | |
| gaggaataaacaatatttagctcctttgtaaatacaaattaattttccttcttgacatc | |
| attcaattttaattttacgtataaaataaaagatcatacctattagaacgattaaggag | |
| aaatacaattcgaatgagaaggatgtgccgtttgttataataaacagccacacgacgta | |
| aacgtaaaatgaccacatgatgggccaatagacatggaccgactactaataatagtaag | |
| ttacattttaggatggaataaatatcataccgacatcagttttgaaagaaaagggaaaa | |
| aaagaaaaaataaataaaagatatactaccgacatgagttccaaaaagcaaaaaaaaag | |
| atcaagccgacacagacacgcgtagagagcaaaatgactttgacgtcacaccacgaaaa | |
| cagacgcttcatacgtgtccctttatctctctcagtctctctataaacttagtgagacc | |
| ctcctctgttttactcacaaatatgcaaactagaaaacaatcatcaggaataaagggtt | |
| tgattacttctattggaaaggactctagaggatcctcccaatgtccaagctcgtgtgca | |
| ataaccgccacaatttcatcctcattcttgcactgctgaatcaacgtatcataaagaac | |
| aatccttttgttcttaaagaaaccatacatgtaagcattgctatggcttgaccttgtag | |
| atccatcgacaacaaacagcttcttcaaaggaaactttagggaagaagcaagtttctca | |
| atcttctcccggaggtctccatctggaagaggagtgaatttgttgaagagcggtgctat | |
| caagaccgggtatatagtcatcatcactagagacaggataaacatgaatgcccacagat | |
| agatggcaagataaggacctcctttctggactatgaaaattatcgcagcaacaatgggt | |
| gggcctagtatgacagagaggaatgttcctttgatcatgtccctaatgaacatccatat | |
| tgtttgtttgttgaacccatgccgagactcgatcacgaaagttgagtacaaagaaaatg | |
| gcaaatcagtgatctgtgaccatgtcataacaccagccaagaatgaaagagtatgcagt | |
| atttcattctccggatcaaggcccaacctcggtaaaagaggatccccATCTACCCGCTT | |
| CGCGTCGGCATCCGGTCAGTGGCAGTGAAGGGCGAACAGTTCCTGATTAACCACAAACC | |
| GTTCTACTTTACTGGCTTTGGTCGTCATGAAGATGCGGACTTGCGTGGCAAAGGATTCG | |
| ATAACGTGCTGATGGTGCACGACCACGCATTAATGGACTGGATTGGGGCCAACTCCTAC | |
| CGTACCTCGCATTACCCTTACGCTGAAGAGATGCTCGACTGGGCAGATGAACATGGCAT | |
| CGTGGTGATTGATGAAACTGCTGCTGTCGGCTTTTCGCTCTCTTTAGGCATTGGTTTCG | |
| AAGCGGGCAACAAGCCGAAAGAACTGTACAGCGAAGAGGCAGTCAACGGGGAAACTCAG | |
| CAAGCGCACTTACAGGCGATTAAAGAGCTGATAGCGCGTGACAAAAACCACCCAAGCGT | |
| GGTGATGTGGAGTATTGCCAACGAACCGGATACCCGTCCGCAAGGTGCACGGGAATATT | |
| TCGCGCCACTGGCGGAAGCAACGCGTAAACTCGACCCGACGCGTCCGATCACCTGCGTC | |
| AATGTAATGTTCTGCGACGCTCACACCGATACCATCAGCGATCTCTTTGATGTGCTGTG | |
| CCTGAACCGTTATTACGGATGGTATGTCCAAAGCGGCGATTTGGAAACGGCAGAGAAGG | |
| TACTGGAAAAAGAACTTCTGGCCTGGCAGGAGAAACTGTACACCGACATGTGGAGTGAA | |
| GAGTATCAGTGTGCATGGCTGGATATGTATCACCGCGTCTTTGATCGCGTCAGCGCCGT | |
| CGTCGGTGAACAGGTATGGAATTTCGCCGATTTTGCGACCTCGCAAGGCATATTGCGCG | |
| TTGGCGGTAACAAGAAAGGGATCTTCACTCGCGACCGCAAACCGAAGTCGGCGGCTTTT | |
| CTGCTGCAAAAACGCTGGACTGGCATGAACTTCGGTGAAAAACCGCAGCAGGGAGGCAA | |
| ACAATGAATCAACAACTCTCCTGGCGCACCATCGTCGGCTACAGCCTCGGGAATTGCTA | |
| CCGAGCTCttttaccgaggttgggccttgatccggagaatgaaatactgcatactcttt | |
| cattcttggctggtgttatgacatggtcacagatcactgatttgccattttctttgtac | |
| tcaactttcgtgatcgagtctcggcatgggttcaacaaacaaacaatatggatgttcat | |
| tagggacatgatcaaaggaacattcctctctgtcatactaggcccacccattgttgctg | |
| cgataattttcatagtccagaaaggaggtccttatcttgccatctatctgtgggcattc | |
| atgtttatcctgtctctagtgatgatgactatatacccggtcttgatagcaccgctctt | |
| caacaaattcactcctcttccagatggagacctccgggagaagattgagaaacttgctt | |
| cttccctaaagtttcctttgaagaagctgtttgttgtcgatggatctacaaggtcaagc | |
| catagcaatgcttacatgtatggtttctttaagaacaaaaggattgttctttatgatac | |
| gttgattcagcagtgcaagaatgaggatgaaattgtggcggttattgcacacgagcttg | |
| gacattgggagctcgaatttccccgatcgttcaaacatttggcaataaagtttcttaag | |
| attgaatcctgttgccggtcttgcgatgattatcatataatttctgttgaattacgtta | |
| agcatgtaataattaacatgtaatgcatgacgttatttatgagatgggtttttatgatt | |
| agagtcccgcaattatacatttaatacgcgatagaaaacaaaatatagcgcgcaaacta | |
| ggataaattatcgcgcgcggtgtcatctatgttactagatcgggaattcactggccgtc | |
| gttttacaacgtcgtgactgggaaaaccctggcgttacccaacttaatcgccttgcagc | |
| acatccccctttcgccagctggcgtaatagcgaagaggcccgcaccgatcgcccttccc | |
| aacagttgcgcagcctgaatggcgcccgctcctttcgctttcttcccttcctttctcgc | |
| cacgttcgccggctttccccgtcaagctctaaatcgggggctccctttagggttccgat | |
| ttagtgctttacggcacctcgaccccaaaaaacttgatttgggtgatggttcacgtagt | |
| gggccatcgccctgatagacggtttttcgccctttgacgttggagtccacgttctttaa | |
| tagtggactcttgttccaaactggaacaacactcaaccctatctcgggctattcttttg | |
| atttataagggattttgccgatttcggaaccaccatcaaacaggattttcgcctgctgg | |
| ggcaaaccagcgtggaccgcttgctgcaactctctcagggccaggcggtgaagggcaat | |
| cagctgttgcccgtctcactggtgaaaagaaaaaccaccccagtacattaaaaacgtcc | |
| gcaatgtgttattaagttgtctaagcgtcaatttgtttacaccacaatatatcctgcca | |

SEQ ID NO:37 is the nucleic acid sequence of RD29A-HP-AtCPP. Italicized sequences are the right and left border repeats. Underlined sequence is the RD29A promoter. Sequence in bold is the AtCPP anti-sense sequence. Upper case sequence represents the truncated GUS fragment. Bold and underlined sequence represents the A. thaliana CaaX prenyl protease sense fragment.

| | |
|---|---|
| gtttacccgccaatatatcctgtcaaacactgatagtttaaactgaaggcgggaaacga | SEQ ID NO:38 |
| caatctgatcatgagcggagaattaagggagtcacgttatgacccccgccgatgacgcg | |
| ggacaagccgttttacgtttggaactgacagaaccgcaacgttgaaggagccactcagc | |
| cgcgggtttctggagtttaatgagctaagcacatacgtcagaaaccattattgcgcgtt | |
| caaaagtcgcctaaggtcactatcagctagcaaatatttcttgtcaaaaatgctccact | |
| gacgttccataaattcccctcggtatccaattagagtctcatattcactctcaatccaa | |
| ataatctgcaccggatctggatcgtttcgcatgattgaacaagatggattgcacgcagg | |
| ttctccggccgcttgggtggagaggctattcggctatgactgggcacaacagacaatcg | |
| gctgctctgatgccgccgtgttccggctgtcagcgcaggggcgcccggttctttttgtc | |
| aagaccgacctgtccggtgccctgaatgaactgcaggacgaggcagcgcggctatcgtg | |
| gctggccacgacgggcgttccttgcgcagctgtgctcgacgttgtcactgaagcgggaa | |
| gggactggctgctattgggcgaagtgccggggcaggatctcctgtcatctcaccttgct | |
| cctgccgagaaagtatccatcatggctgatgcaatgcggcggctgcatacgcttgatcc | |
| ggctacctgcccattcgaccaccaagcgaaacatcgcatcgagcgagcacgtactcgga | |
| tggaagccggtcttgtcgatcaggatgatctggacgaagagcatcaggggctcgcgcca | |
| gccgaactgttcgccaggctcaaggcgcgcatgcccgacggcgatgatctcgtcgtgac | |
| ccatggcgatgcctgcttgccgaatatcatggtggaaaatggccgcttttctggattca | |
| tcgactgtggccggctgggtgtggcggaccgctatcaggacatagcgttggctacccgt | |
| gatattgctgaagagcttggcggcgaatgggctgaccgcttcctcgtgctttacggtat | |
| cgccgctcccgattcgcagcgcatcgccttctatcgccttcttgacgagttcttctgag | |
| cgggactctggggttcgaaatgaccgaccaagcgacgcccaacctgccatcacgagatt | |
| tcgattccaccgccgccttctatgaaaggttgggcttcggaatcgttttccgggacgcc | |
| ggctggatgatcctccagcgcggggatctcatgctggagttcttcgcccacgggatctc | |
| tgcggaacaggcggtcgaaggtgccgatatcattacgacagcaacggccgacaagcaca | |
| acgccacgatcctgagcgacaatatgatcgggcccggcgtccacatcaacggcgtcggc | |
| ggcgactgcccaggcaagaccgagatgcaccgcgatatcttgctgcgttcggatatttt | |
| cgtggagttcccgccacagacccggatgatccccgatcgttcaaacatttggcaataaa | |
| gtttcttaagattgaatcctgttgccggtcttgcgatgattatcatataatttctgttg | |
| aattacgttaagcatgtaataattaacatgtaatgcatgacgttatttatgagatgggt | |
| ttttatgattagagtcccgcaattatacatttaatacgcgatagaaaacaaaatatagc | |
| gcgcaaactaggataaattatcgcgcgcggtgtcatctatgttactagatcgggcctcc | |
| tgtcaatgctggcggcggctctggtggtggttctggtggcggctctgagggtggtggct | |
| ctgagggtggcggttctgagggtggcggctctgagggaggcggttccggtggtggctct | |
| ggttccggtgattttgattatgaaaagatggcaaacgctaataagggggctatgaccga | |
| aaatgccgatgaaaacgcgctacagtctgacgctaaaggcaaacttgattctgtcgcta | |
| ctgattacggtgctgctatcgatggtttcattggtgacgtttccggccttgctaatggt | |
| aatggtgctactggtgattttgctggctctaattcccaaatggctcaagtcggtgacgg | |
| tgataattcacctttaatgaataatttccgtcaatatttaccttccctccctcaatcgg | |
| ttgaatgtcgcccttttgtctttggcccaatacgcaaaccgcctctccccgcgcgttgg | |
| ccgattcattaatgcagctggcacgacaggtttcccgactggaaagcgggcagtgagcg | |
| caacgcaattaatgtgagttagctcactcattaggcaccccaggctttacactttatgc | |
| ttccggctcgtatgttgtgtggaattgtgagcggataacaatttcacacaggaaacagc | |
| tatgaccatgattacgccaagcttgcatgcctgcagggagccatagatgcaattcaatc | |
| aaactgaaatttctgcaagaatctcaaacacggagatctcaaagtttgaaagaaaattt | |
| atttcttcgactcaaaacaaacttacgaaatttaggtagaacttatatacattatattg | |
| taattttttgtaacaaaatgtttttattattattatagaattttactggttaaattaaa | |
| aatgaatagaaaaggtgaattaagaggagagaggaggtaaacattttcttctatttttt | |
| catattttcaggataaattattgtaaaagtttacaagatttccatttgactagtgtaaa | |
| tgaggaatattctctagtaagatcattatttcatctacttcttttatcttctaccagta | |
| gaggaataaacaatatttagctcctttgtaaatacaaattaattttccttctttacatc | |
| attcaattttaattttacgtataaaataaaagatcatacctattagaacgattaaggag | |
| aaatacaattcgaatgagaaggatgtgccgtttgttataataaacagccacacgacgta | |
| aacgtaaaatgaccacatgatgggccaatagacatggaccgactactaataatagtaag | |
| ttacattttaggatggaataaatatcataccgacatcagttttgaaagaaaagggaaaa | |
| aaagaaaaaataaataaaagatatactaccgacatgagttccaaaaagcaaaaaaaaag | |
| atcaagccgacacagacacgcgtagagagcaaaatgactttgacgtcacaccacgaaaa | |
| cagacgcttcatacgtgtccctttatctctctcagtctctctataaacttagtgagacc | |
| ctcctctgttttactcacaaatatgcaaactagaaaacaatcatcaggaataaagggtt | |
| tgattacttctattggaaaggactctagaggatccTTAATCTGTCTTCTTGTCTTCTCC | |
| ATCAGTGGCTCGAAGCCTTTCAACAAGAGGAGGATGTGAGTAGTGATAAGCTGAGTACA | |
| ATGGATCAGTGTTCATTGTTGATAAGTTCTCTTCCTGTAGTTTCACTAGAGCAGGACGA | |
| AGATCTTTTGCATAGTCAAGCTTCACAGCAAAAGCATCAGCCTGAAACTCAAACGCTCG | |
| ACTAACGAGGTTCAGGCCAAAGCTTACTAGATGTTGCAGTGGTATTACAGTGTGCTGAA | |
| ATATGATCAAACCAATGAGAACAGGCTGTGTATCAAATCCGAAACTCCTGAAGAGATCA | |
| GTGGAGTTTCTGAGAAGAGTGTATCCTCCAAATTGTAAGAAGGCAAGGATTTGAACTGC | |
| AATGAACGAGTATGTAGTGTGATTCAGTTTCCAATGTCCAAGCTCGTGTGCAATAACCG | |
| CCACAATTTCATCCTCATTCTTGCACTGCTGAATCAACGTATCATAAAGAACAATCCTT | |
| TTGTTCTTAAAGAAACCATACATGTAAGCATTGCTATGGCTTGACCTTGTAGATCCATC | |
| GACAACAAACAGCTTCTTCAAAGGAAACTTTAGGGAAGAAGCAAGTTTCTCAATCTTCT | |
| CCCGGAGGTCTCCATCTGGAAGAGGAGTGAATTTGTTGAAGAGCGGTGCTATCAAGACC | |
| GGGTATATAGTCATCATCACTAGAGACAGGATAAACATGAATGCCCACAGATAGATGGC | |
| AAGATAAGGACCTCCTTTCTGGACTATGAAAATTATCGCAGCAACAATGGGTGGGCCTA | |
| GTATGACAGAGAGGAATGTTCCTTTGATCATGTCCCTAATGAACATCCATATTGTTTGT | |
| TTGTTGAACCCATGCCGAGACTCGATCACGAAAGTTGAGTACAAAGAAAATGGCAAATC | |
| AGTGATCTGTGACCATGTCATAACACCAGCCAAGAATGAAAGAGTATGCAGTATTTCAT | |
| TCTCCGGATCAAGGCCCAACCTCGGTAAAACAGCTCCAGACATCTTCCAAAACCAAGGC | |
| AAGATCCCAAAGAACAAAATTGCAGAGTCCATAAGTATAGTTACAAACTCATGAACAAA | |
| GTGAAAATAGCTTTTGTCAAGACTGTATGCTCGTGATTTCTCAAACTTCTCTTGGCTAA | |
| TTACACCAACCAAGGTTTTCGGGAGAGTTGGAAGCTTGAGAGCAGTGAGTTGCCTCAGA | |
| TCCAAATACGTCTCAAAAATGTACATCACTATCATAAAACCCACGACGGTTTCCATGAA | |
| AGGAATCGCCATcccctcgaatttccccgatcgttcaaacatttggcaataaagtttct | |
| taagattgaatcctgttgccggtcttgcgatgattatcatataatttctgttgaattac | |
| gttaagcatgtaataattaacatgtaatgcatgacgttatttatgagatgggtttttat | |
| gattagagtcccgcaattatacatttaatacgcgatagaaaacaaaatatagcgcgcaa | |
| actaggataaattatcgcgcgcggtgtcatctatgttactagatcgggaattcactggc | |
| cgtcgttttacaacgtcgtgactgggaaaaccctggcgttacccaacttaatcgccttg | |
| cagcacatccccctttcgccagctggcgtaatagcgaagaggcccgcaccgatcgccct | |
| tcccaacagttgcgcagcctgaatggcgcccgctcctttcgctttcttcccttcctttc | |
| tcgccacgttcgccggctttccccgtcaagctctaaatcgggggctccctttagggttc | |
| cgatttagtgctttacggcacctcgaccccaaaaaacttgatttgggtgatggttcacg | |
| tagtgggccatcgccctgatagacggtttttcgccctttgacgttggagtccacgttct | |
| ttaatagtggactcttgttccaaactggaacaacactcaaccctatctcgggctattct | |
| tttgatttataagggattttgccgatttcggaaccaccatcaaacaggattttcgcctg | |
| ctggggcaaaccagcgtggaccgcttgctgcaactctctcagggccaggcggtgaaggg | |
| caatcagctgttgcccgtctcactggtgaaaagaaaaaccaccccagtacattaaaaac | |
| gtccgcaatgtgttattaagttgtctaagcgtcaatttgtttacaccacaatatatcct | |
| gcca | |

SEQ ID NO:38 is the nucleic acid sequence of RD29A-antisense-AtCPP. Italicized sequences are the right and left border repeats. Underlined sequence is the RD29A promoter. Sequence in upper case sequence is the AtCPP anti-sense sequence.

| | |
|---|---|
| gtttacccgccaatatatcctgtcaaacactgatagtttaaactgaaggcgggaaacga | SEQ ID NO:39 |
| caatctgatcatgagcggagaattaagggagtcacgttatgacccccgccgatgacgcg | |
| ggacaagccgttttacgtttggaactgacagaaccgcaacgttgaaggagccactcagc | |
| caaaagtcgcctaaggtcactatcagctagcaaatatttcttgtcaaaaatgctccact | |
| gacgttccataaattcccctcggtatccaattagagtctcatattcactctcaatccaa | |
| ataatctgcaccggatctggatcgtttcgcatgattgaacaagatggattgcacgcagg | |
| ttctccggccgcttgggtggagaggctattcggctatgactgggcacaacagacaatcg | |
| gctgctctgatgccgccgtgttccggctgtcagcgcaggggcgcccggttctttttgtc | |
| aagaccgacctgtccggtgccctgaatgaactgcaggacgaggcagcgcggctatcgtg | |
| gctggccacgacgggcgttccttgcgcagctgtgctcgacgttgtcactgaagcgggaa | |
| gggactggctgctattgggcgaagtgccggggcaggatctcctgtcatctcaccttgct | |
| cctgccgagaaagtatccatcatggctgatgcaatgcggcggctgcatacgcttgatcc | |
| ggctacctgcccattcgaccaccaagcgaaacatcgcatcgagcgagcacgtactcgga | |
| tggaagccggtcttgtcgatcaggatgatctggacgaagagcatcaggggctcgcgcca | |
| gccgaactgttcgccaggctcaaggcgcgcatgcccgacggcgatgatctcgtcgtgac | |
| ccatggcgatgcctgcttgccgaatatcatggtggaaaatggccgcttttctggattca | |
| tcgactgtggccggctgggtgtggcggaccgctatcaggacatagcgttggctacccgt | |
| gatattgctgaagagcttggcggcgaatgggctgaccgcttcctcgtgctttacggtat | |
| cgccgctcccgattcgcagcgcatcgccttctatcgccttcttgacgagttcttctgag | |
| cgggactctggggttcgaaatgaccgaccaagcgacgcccaacctgccatcacgagatt | |
| tcgattccaccgccgccttctatgaaaggttgggcttcggaatcgttttccgggacgcc | |
| ggctggatgatcctccagcgcggggatctcatgctggagttcttcgcccacgggatctc | |
| tgcggaacaggcggtcgaaggtgccgatatcattacgacagcaacggccgacaagcaca | |
| acgccacgatcctgagcgacattatgatcgggcccggcgtccacatcaacggcgtcggc | |
| ggcgactgcccaggcaagaccgagatgcaccgcgatatcttgctgcgttcggatatttt | |
| cgtggagttcccgccacagacccggatgatccccgatcgttcaaacatttggcaataaa | |
| gtttcttaagattgaatcctgttgccggtcttgcgatgattatcatataatttctgttg | |
| aattacgttaagcatgtaataattaacatgtaatgcatgacgttatttatgagatgggt | |
| ttttatgattagagtcccgcaattatacatttaatacgcgatagaaaacaaaatatagc | |
| gcgcaaactaggataaattatcgcgcgcggtgtcatctatgttactagatcgggcctcc | |
| tgtcaatgctggcggcggctctggtggtggttctggtggcggctctgagggtggtggct | |
| ctgagggtggcggttctgagggtggcggctctgagggaggcggttccggtggtggctct | |
| ggttccggtgattttgattatgaaaagatggcaaacgctaataagggggctatgaccga | |
| aaatgccgatgaaaacgcgctacagtctgacgctaaaggcaaacttgattctgtcgcta | |
| ctgattacggtgctgctatcgatggtttcattggtgacgtttccggccttgctaatggt | |
| aatggtgctactggtgattttgctggctctaattcccaaatggctcaagtcggtgacgg | |
| tgataattcacctttaatgaataatttccgtcaatatttaccttccctccctcaatcgg | |
| ttgaatgtcgcccttttgtctttggcccaatacgcaaaccgcctctccccgcgcgttgg | |
| ccgattcattaatgcagctggcacgacaggtttcccgactggaaagcgggcagtgagcg | |
| caacgcaattaatgtgagttagctcactcattaggcaccccaggctttacactttatgc | |
| ttccggctcgtatgttgtgtggaattgtgagcggataacaatttcacacaggaaacagc | |
| tatgaccatgattacgccaagctGGGAAATTTTTCGCCAGTTCTAAATATCCGGAAACC | |
| TCTTGGGATGCCATTGCCCATCTATCTGTAATTTATTGACGAAATAGACGAAAAGGAAG | |
| GTGGCTCCTATAAAGCACATCATTGCGATAACAGAAAGGCCATTGTTGAAGATACCTCT | |
| GCTGACATTGGTCCCCAAGTGGAAGCACCACCCCATGAGGAGCACCGTGGAGTAAGAAG | |
| ACGTTCGAGCCACGTCGAAAAAGCAAGTGTGTTGATGTAGTATCTCCATTGACGTAAGG | |
| GATGACGCACAATCCAACTATCCATCGCAAGACCATTGCTCTATATAAGAAAGTTAATA | |
| TCATTTCGAGTGGCCACGCTGAGGGGGATCCatggcgattcctttcatggaaaccgtcg | |
| tgggttttatgatagtgatgtacatttttgagacgtatttggatctgaggcaactcact | |
| gctctcaagcttccaactctcccgaaaaccttggttggtgtaattagccaagagaagtt | |
| tgagaaatcacgagcatacagtcttgacaaaagctattttcactttgttcatgagtttg | |
| taactatacttatggactctgcaattttgttctttgggatcttgccttggttttggaag | |
| atgtctggagctgttttaccgaggttgggccttgatccggagaatgaaatactgcatac | |
| tctttcattcttggctggtgttatgacatggtcacagatcactgatttgccattttctt | |
| tgtactcaactttcgtgatcgagtctcggcatgggttcaacaaacaaacaatatggatg | |
| ttcattagggacatgatcaaaggaacattcctctctgtcatactaggcccacccattgt | |
| tgctgcgataattttcatagtccagaaaggaggtccttatcttgccatctatctgtggg | |
| cattcatgtttatcctgtctctagtgatgatgactatatacccggtcttgatagcaccg | |
| ctcttcaacaaattcactcctcttccagatggagacctccgggagaagattgagaaact | |
| tgcttcttccctaaagtttcctttgaagaagctgtttgttgtcgatggatctacaaggt | |
| caagccatagcaatgcttacatgtatggtttctttaagaacaaaaggattgttctttat | |
| gatacgttgattcagcagtgcaagaatgaggatgaaattgtggcggttattgcacacga | |
| gcttggacattggaaactgaatcacactacatactcgttcattgcagttcaaatccttg | |
| ccttcttacaatttggaggatacactcttctcagaaactccactgatctcttcaggagt | |
| ttcggatttgatacacagcctgttctcattggtttgatcatatttcagcgcgctgtaat | |
| accactgcaacatctagtaagctttggcctgaacctcgttagtcgagcgtttgagtttc | |
| aggctgatgcttttgctgtgaagcttgactatgcaaaagatcttcgtcctgctctagtg | |
| aaactacaggaagagaacttatcaacaatgaacactgatccattgtactcagcttatca | |
| ctactcacatcctcctcttgttgaaaggcttcgagccactgatggagaagacaagaaga | |
| cagattaacccctcgaatttccccgatcgttcaaacatttggcaataaagtttcttaag | |
| attgaatcctgttgccggtcttgcgatgattatcatataatttctgttgaattacgtta | |
| agcatgtaataattaacatgtaatgcatgacgttatttatgagatgggtttttatgatt | |
| agagtcccgcaattatacatttaatacgcgatagaaaacaaaatatagcgcgcaaacta | |
| ggataaattatcgcgcgcggtgtcatctatgttactagatcgggaattcactggccgtc | |
| gttttacaacgtcgtgactgggaaaaccctggcgttacccaacttaatcgccttgcagc | |
| acatccccctttcgccagctggcgtaatagcgaagaggcccgcaccgatcgcccttccc | |
| aacagttgcgcagcctgaatggcgcccgctcctttcgctttcttcccttcctttctcgc | |
| cacgttcgccggctttccccgtcaagctctaaatcgggggctccctttagggttccgat | |
| ttagtgctttacggcacctcgaccccaaaaaacttgatttgggtgatggttcacgtagt | |
| gggccatcgccctgatagacggtttttcgccctttgacgttggagtccacgttctttaa | |
| tagtggactcttgttccaaactggaacaacactcaaccctatctcgggctattcttttg | |
| atttataagggattttgccgatttcggaaccaccatcaaacaggattttcgcctgctgg | |
| ggcaaaccagcgtggaccgcttgctgcaactctctcagggccaggcggtgaagggcaat | |
| cagctgttgcccgtctcactggtgaaaagaaaaaccaccccagtacattaaaaacgtgg | |
| gcaatgtgttattaagttgtctaagcgtcaatttgtttacaccacaatatatcctgcca | |

SEQ ID NO:39 is the nucleic acid sequence of MuA-AtCPP. Italicized sequences are the right and left border repeats. Sequence in upper case is the MuA promoter. The A. thaliana CaaX prenyl protease sense sequence is in bold.

| | |
|---|---|
| gtttacccgccaatatatcctgtcaaacactgatagtttaaactgaaggcgggaaacga | SEQ ID NO:40 |
| caatctgatcatgagcggagaattaagggagtcacgttatgacccccgccgatgacgcg | |
| ggacaagccgttttacgtttggaactgacagaaccgcaacgttgaaggagccactcagc | |
| cgcgggtttctggagtttaatgagctaagcacatacgtcagaaaccattattgcgcgtt | |
| caaaagtcgcctaaggtcactatcagctagcaaatatttcttgtcaaaaatgctccact | |
| gacgttccataaattcccctcggtatccaattagagtctcatattcactctcaatccaa | |
| ataatctgcaccggatctggatcgtttcgcatgattgaacaagatggattgcacgcagg | |
| ttctccggccgcttgggtggagaggctattcggctatgactgggcacaacagacaatcg | |
| gctgctctgatgccgccgtgttccggctgtcagcgcaggggcgcccggttctttttgtc | |
| aagaccgacctgtccggtgccctgaatgaactgcaggacgaggcagcgcggctatcgtg | |
| gctggccacgacgggcgttccttgcgcagctgtgctcgacgttgtcactgaagcgggaa | |
| gggactggctgctattgggcgaagtgccggggcaggatctcctgtcatctcaccttgct | |
| cctgccgagaaagtatccatcatggctgatgcaatgcggcggctgcatacgcttgatcc | |
| ggctacctgcccattcgaccaccaagcgaaacatcgcatcgagcgagcacgtactcgga | |
| tggaagccggtcttgtcgatcaggatgatctggacgaagagcatcaggggctcgcgcca | |
| gccgaactgttcgccaggctcaaggcgcgcatgcccgacggcgatgatctcgtcgtgac | |
| ccatggcgatgcctgcttgccgaatatcatggtggaaaatggccgcttttctggattca | |
| tcgactgtggccggctgggtgtggcggaccgctatcaggacatagcgttggctacccgt | |
| gatattgctgaagagcttggcggcgaatgggctgaccgcttcctcgtgctttacggtat | |
| cgccgctcccgattcgcagcgcatcgccttctatcgccttcttgacgagttcttctgag | |
| cgggactctggggttcgaaatgaccgaccaagcgacgcccaacctgccatcacgagatt | |
| tcgattccaccgccgccttctatgaaaggttgggcttcggaatcgttttccgggacgcc | |
| ggctggatgatcctccagcgcggggatctcatgctggagttcttcgcccacgggatctc | |
| tgcggaacaggcggtcgaaggtgccgatatcattacgacagcaacggccgacaagcaca | |
| acgccacgatcctgagcgacaatatgatcgggcccggcgtccacatcaacggcgtcggc | |
| ggcgactgcccaggcaagaccgagatgcaccgcgatatcttgctgcgttcggatatttt | |
| cgtggagttcccgccacagacccggatgatccccgatcgttcaaacatttggcaataaa | |
| gtttcttaagattgaatcctgttgccggtcttgcgatgattatcatataatttctgttg | |
| aattacgttaagcatgtaataattaacatgtaatgcatgacgttatttatgagatgggt | |
| ttttatgattagagtcccgcaattatacatttaatacgcgatagaaaacaaaatatagc | |
| gcgcaaactaggataaattatcgcgcgcggtgtcatctatgttactagatcgggcctcc | |
| tgtcaatgctggcggcggctctggtggtggttctggtggcggctctgagggtggtggct | |
| ctgagggtggcggttctgagggtggcggctctgagggaggcggttccggtggtggctct | |
| ggttccggtgattttgattatgaaaagatggcaaacgctaataagggggctatgaccga | |
| aaatgccgatgaaaacgcgctacagtctgacgctaaaggcaaacttgattctgtcgcta | |
| ctgattacggtgctgctatcgatggtttcattggtgacgtttccggccttgctaatggt | |
| aatggtgctactggtgattttgctggctctaattcccaaatggctcaagtcggtgacgg | |
| tgataattcacctttaatgaataatttccgtcaatatttaccttccctccctcaatcgg | |
| ttgaatgtcgcccttttgtctttggcccaatacgcaaaccgcctctccccgcgcgttgg | |
| ccgattcattaatgcagctggcacgacaggtttcccgactggaaagcgggcagtgagcg | |
| caacgcaattaatgtgagttagctcactcattaggcaccccaggctttacactttatgc | |
| ttccggctcgtatgttgtgtggaattgtgagcggataacaatttcacacaggaaacagc | |
| tatgaccatgattacgccaagctGGGAAATTTTTCGCCAGTTCTAAATATCCGGAAACC | |
| TCTTGGGATGCCATTGCCCATCTATCTGTAATTTATTGACGAAATAGACGAAAAGGAAG | |
| GTGGCTCCTATAAAGCACATCATTGCGATAACAGAAAGGCCATTGTTGAAGATACCTCT | |
| GCTGACATTGGTCCCCAAGTGGAAGCACCACCCCATGAGGAGCACCGTGGAGTAAGAAG | |
| ACGTTCGAGCCACGTCGAAAAAGCAAGTGTGTTGATGTAGTATCTCCATTGACGTAAGG | |
| GATGACGCACAATCCAACTATCCATCGCAAGACCATTGCTCTATATAAGAAAGTTAATA | |
| TCATTTCGAGTGGCCACGCTGAGGGGGATCGGGATGGCGTTTCCCTACATGGAAGCCGT | |
| TGTCGGATTTATGATATTAATGTACATTTTTGAAACTTACTTGGATGTGCGACAACATA | |
| GGGCCCTCAAACTTCCTACTCTTCCAAAGACTTTAGAGGGTGTTATCAGCCAAGAGAAA | |
| TTTGAGAAATCTAGAGCCTATAGTCTTGATAAAAGCCACTTCCATTTTGTTCACGAGTT | |
| TGTGACAATAGTGACAGACTCTACAATTTTGTACTTTGGGGTATTGCCCTGGTTTTGGA | |
| AGAAATCAGGAGATTTTATGACAATAGCTGGTTTCAATGCTGAGAATGAAATACTGCAT | |
| ACCCTTGCCTTCTTAGCAGGGCTGATGATTTGGTCACAGATAACAGATTTGCCCTTTTC | |
| TCTGTACTCAACTTTTGTGATTGAGGCCCGTCATGGTTTTAATAAGCAAACACCATGGT | |
| TATTCTTTAGGGACATGCTTAAAGGAATTTTCCTTTCTGTAATAATTGGTCCACCTATT | |
| GTGGCTGCAATCATTGTAATAGTACAGAAAGGAGGTCCATACTTGGCCATCTATCTTTG | |
| GGTTTTTACGTTTGGTCTTTCTATTGTGATGATGACCCTTTATCCAGTACTAATAGCTC | |
| CACTCTTCAATAAGTTCACTCCACTTCCAGATGGTCAACTCAGGGAGAAAATCGAGAAA | |
| CTTGCTTCCTCCCTCAACTATCCGTTAAAGAAACTATTTGTTGTCGATGGATCCACAAG | |
| ATCAAGTCACAGCAATGCCTATATGTATGGATTCTTCAAGAACAAGAGGATTGTCCCTT | |
| ATGACACATTAATTCAACAGTGCAAAGACGATGAGGAAATTGTTGCTGTTATTGCCCAT | |
| GAGTTGGGACACTGGAAGCTCAACCATACTGTGTACACATTTGTTGCTATGCAGATTCT | |
| TACACTTCTACAATTTGGAGGATATACACTAGTGCGAAATTCAGCTGATCTGTATCGAA | |
| GCTTTGGGTTTGATACGCAGCCAGTCCTCATTGGGCTCATCATATTTCAGCATACTGTA | |
| ATCCCACTTCAGCAATTGGTCAGCTTTGGTCTGAACCTAGTCAGCCGATCATTTGAATT | |
| TCAGGCTGATGGCTTTGCCAAGAAGCTTGGATATGCATCTGGATTACGCGGTGGTCTTG | |
| TGAAACTACAGGAGGAGAATCTGTCAGCTATGAATACAGATCCTTGGTACTCTGCTTAT | |
| CACTATTCTCATCCTCCCCTTGTTGAAAGATTGGCCGCGCTGGACGAACCGGATAAGAA | |
| GGAAGACTAAgagctcgaatttccccgatcgttcaaacatttggcaataaagtttctta | |
| agattgaatcctgttgccggtcttgcgatgattatcatataatttctgttgaattacgt | |
| taagcatgtaataattaacatgtaatgcatgacgttatttatgagatgggtttttatga | |
| ttagagtcccgcaattatacatttaatacgcgatagaaaacaaaatatagcgcgcaaac | |
| taggataaattatcgcgcgcggtgtcatctatgttactagatcgggaattcactggccg | |
| tcgttttacaacgtcgtgactgggaaaaccctggcgttacccaacttaatcgccttgca | |
| gcacatccccctttcgccagctggcgtaatagcgaagaggcccgcaccgatcgcccttc | |
| ccaacagttgcgcagcctgaatggcgcccgctcctttcgctttcttcccttcctttctc | |
| gccacgttcgccggctttccccgtcaagctctaaatcgggggctccctttagggttccg | |
| atttagtgctttacggcacctcgaccccaaaaaacttgatttgggtgatggttcacgta | |
| gtgggccatcgccctgatagacggtttttcgccctttgacgttggagtccacgttcttt | |
| aatagtggactcttgttccaaactggaacaacactcaaccctatctcgggctattcttt | |
| tgatttataagggattttgccgatttcggaaccaccatcaaacaggattttcgcctgct | |
| ggggcaaaccagcgtggaccgcttgctgcaactctctcagggccaggcggtgaagggca | |
| atcagctgttgcccgtctcactggtgaaaagaaaaaccaccccagtacattaaaaacgt | |
| ccgcaatgtgttattaagttgtctaagcgtcaatttgtttacaccacaatatatcctgc | |
| ca | |

SEQ ID NO:40 is the nucleic acid sequence of MuA-GmCPP. Italicized sequences are the right and left border repeats. Sequence in upper case is the MuA promoter. The G. max CaaX prenyl protease sense sequence is in upper case and bold.

| | |
|---|---|
| gtttacccgccaatatatcctgtcaaacactgatagtttaaactgaaggcgggaaacga | SEQ ID NO:41 |
| caatctgatcatgagcggagaattaagggagtcacgttatgacccccgccgatgacgcg | |
| ggacaagccgttttacgtttggaactgacagaaccgcaacgttgaaggagccactcagc | |
| cgcgggtttctggagtttaatgagctaagcacatacgtcagaaaccattattgcgcgtt | |
| caaaagtcgcctaaggtcactatcagctagcaaatatttcttgtcaaaaatgctccact | |
| gacgttccataaattcccctcggtatccaattagagtctcatattcactctcaatccaa | |
| ataatctgcaccggatctggatcgtttcgcatgattgaacaagatggattgcacgcagg | |
| ttctccggccgcttgggtggagaggctattcggctatgactgggcacaacagacaatcg | |
| gctgctctgatgccgccgtgttccggctgtcagcgcaggggcgcccggttctttttgtc | |
| aagaccgacctgtccggtgccctgaatgaactgcaggacgaggcagcgcggctatcgtg | |
| gctggccacgacgggcgttccttgcgcagctgtgctcgacgttgtcactgaagcgggaa | |
| gggactggctgctattgggcgaagtgccggggcaggatctcctgtcatctcaccttgct | |
| cctgccgagaaagtatccatcatggctgatgcaatgcggcggctgcatacgcttgatcc | |
| ggctacctgcccattcgaccaccaagcgaaacatcgcatcgagcgagcacgtactcgga | |
| tggaagccggtcttgtcgatcaggatgatctggacgaagagcatcaggggctcgcgcca | |
| gccgaactgttcgccaggctcaaggcgcgcatgcccgacggcgatgatctcgtcgtgac | |
| ccatggcgatgcctgcttgccgaatatcatggtggaaaatggccgcttttctggattca | |
| tcgactgtggccggctgggtgtggcggaccgctatcaggacatagcgttggctacccgt | |
| gatattgctgaagagcttggcggcgaatgggctgaccgcttcctcgtgctttacggtat | |
| cgccgctcccgattcgcagcgcatcgccttctatcgccttcttgacgagttcttctgag | |
| cgggactctggggttcgaaatgaccgaccaagcgacgcccaacctgccatcacgagatt | |
| tcgattccaccgccgccttctatgaaaggttgggcttcggaatcgttttccgggacgcc | |
| ggctggatgatcctccagcgcggggatctcatgctggagttcttcgcccacgggatctc | |
| tgcggaacaggcggtcgaaggtgccgatatcattacgacagcaacggccgacaagcaca | |
| acgccacgatcctgagcgacaatatgatcgggcccggcgtccacatcaacggcgtcggc | |
| ggcgactgcccaggcaagaccgagatgcaccgcgatatcttgctgcgttcggatatttt | |
| cgtggagttcccgccacagacccggatgatccccgatcgttcaaacatttggcaataaa | |
| gtttcttaagattgaatcctgttgccggtcttgcgatgattatcatataatttctgttg | |
| aattacgttaagcatgtaataattaacatgtaatgcatgacgttatttatgagatgggt | |
| ttttatgattagagtcccgcaattatacatttaatacgcgatagaaaacaaaatatagc | |
| gcgcaaactaggataaattatcgcgcgcggtgtcatctatgttactagatcgggcctcc | |
| tgtcaatgctggcggcggctctggtggtggttctggtggcggctctgagggtggtggct | |
| ctgagggtggcggttctgagggtggcggctctgagggaggcggttccggtggtggctct | |
| ggttccggtgattttgattatgaaaagatggcaaacgctaataagggggctatgaccga | |
| aaatgccgatgaaaacgcgctacagtctgacgctaaaggcaaacttgattctgtcgcta | |
| ctgattacggtgctgctatcgatggtttcattggtgacgtttccggccttgctaatggt | |
| aatggtgctactggtgattttgctggctctaattcccaaatggctcaagtcggtgacgg | |
| tgataattcacctttaatgaataatttccgtcaatatttaccttccctccctcaatcgg | |
| ttgaatgtcgcccttttgtctttggcccaatacgcaaaccgcctctccccgcgcgttgg | |
| ccgattcattaatgcagctggcacgacaggtttcccgactggaaagcgggcagtgagcg | |
| caacgcaattaatgtgagttagctcactcattaggcaccccaggctttacactttatgc | |
| ttccggctcgtatgttgtgtggaattgtgagcggataacaatttcacacaggaaacagc | |
| tatgaccatgattacgccaagcttgcatgcctgcagcccacagatggttagagaggctt | |
| acgcagcaggtctcatcaagacgatctacccgagcaataatctccaggaaatcaaatac | |
| cttcccaagaaggttaaagatgcagtcaaaagattcaggactaactgcatcaagaacac | |
| agagaaagatatatttctcaagatcagaagtactattccagtatggacgattcaaggct | |
| tgcttcacaaaccaaggcaagtaatagagattggagtctctaaaaaggtagttcccact | |
| gaatcaaaggccatggagtcaaagattcaaatagaggacctaacagaactcgccgtaaa | |
| gactggcgaacagttcatacagagtctcttacgactcaatgacaagaagaaaatcttcg | |
| tcaacatggtggagcacgacacacttgtctactccaaaaatatcaaagatacagtctca | |
| gaagaccaaagggcaattgagacttttcaacaaagggtaatatccggaaacctcctcgg | |
| attccattgcccagctatctgtcactttattgtgaagatagtggaaaaggaaggtggct | |
| cctacaaatgccatcattgcgataaaggaaaggccatcgttgaagatgcctctgccgac | |
| agtggtcccaaagatggacccccacccacgaggagcatcgtggaaaaagaagacgttcc | |
| aaccacgtcttcaaagcaagtggattgatgtgatatctccactgacgtaagggatgacg | |
| cacaatcccactatccttcgcaagacccttcctctatataaggaagttcatttcatttg | |
| gagagaacacgggggactctagaggatccccgggatggcgtttccctacatggaagccg | |
| ttgtcggatttatgatattaatgtacatttttgaaacttacttggatgtgcgacaacat | |
| agggccctcaaacttcctactcttccaaagactttagagggtgttatcagccaagagaa | |
| atttgagaaatctagagcctatagtcttgataaaagccacttccattttgttcacgagt | |
| ttgtgacaatagtgacagactctacaattttgtactttggggtattgccctggttttgg | |
| aagaaatcaggagattttatgacaatagctggtttcaatgctgagaatgaaatactgca | |
| tacccttgccttcttagcagggctgatgatttggtcacagataacagatttgccctttt | |
| ctctgtactcaacttttgtgattgaggcccgtcatggttttaataagcaaacaccatgg | |
| ttattctttagggacatgcttaaaggaattttcctttctgtaataattggtccacctat | |
| tgtggctgcaatcattgtaatagtacagaaaggaggtccatacttggccatctatcttt | |
| gggtttttacgtttggtctttctattgtgatgatgaccctttatccagtactaatagct | |
| ccactcttcaataagttcactccacttccagatggtcaactcagggagaaaatcgagaa | |
| acttgcttcctccctcaactatccgttaaagaaactatttgttgtcgatggatccacaa | |
| gatcaagtcacagcaatgcctatatgtatggattcttcaagaacaagaggattgtccct | |
| tatgacacattaattcaacagtgcaaagacgatgaggaaattgttgctgttattgccca | |
| tgagttgggacactggaagctcaaccatactgtgtacacatttgttgctatgcagattc | |
| ttacacttctacaatttggaggatatacactagtgcgaaattcagctgatctgtatcga | |
| agctttgggtttgatacgcagccagtcctcattgggctcatcatatttcagcatactgt | |
| agctttgggtttgatacgcagccagtcctcattgggctcatcatatttcagcatactgt | |
| aatcccacttcagcaattggtcagctttggtctgaacctagtcagccgatcatttgaat | |
| ttcaggctgatggctttgccaagaagcttggatatgcatctggattacgcggtggtctt | |
| gtgaaactacaggaggagaatctgtcagctatgaatacagatccttggtactctgctta | |
| tcactattctcatcctccccttgttgaaagattggccgcgctggacgaaccggataaga | |
| aggaagactaagagctcgaatttccccgatcgttcaaacatttggcaataaagtttctt | |
| aagattgaatcctgttgccggtcttgcgatgattatcatataatttctgttgaattacg | |
| ttaagcatgtaataattaacatgtaatgcatgacgttatttatgagatgggtttttatg | |
| attagagtcccgcaattatacatttaatacgcgatagaaaacaaaatatagcgcgcaaa | |
| ctaggataaattatcgcgcgcggtgtcatctatgttactagatcgggaattcactggcc | |
| gtcgttttacaacgtcgtgactgggaaaaccctggcgttacccaacttaatcgccttgc | |
| agcacatccccctttcgccagctggcgtaatagcgaagaggcccgcaccgatcgccctt | |
| cccaacagttgcgcagcctgaatggcgcccgctcctttcgctttcttcccttcctttct | |
| cgccacgttcgccggctttccccgtcaagctctaaatcgggggctccctttagggttcc | |
| gatttagtgctttacggcacctcgaccccaaaaaacttgatttgggtgatggttcacgt | |
| agtgggccatcgccctgatagacggtttttcgccctttgacgttggagtccacgttctt | |
| taatagtggactcttgttccaaactggaacaacactcaaccctatctcgggctattctt | |
| ttgatttataagggattttgccgatttcggaaccaccatcaaacaggattttcgcctgc | |
| tggggcaaaccagcgtggaccgcttgctgcaactctctcagggccaggcggtgaagggc | |
| aatcagctgttgcccgtctcactggtgaaaagaaaaaccaccccagtacattaaaaacg | |
| tccgcaatgtgttattaagttgtctaagcgtcaatttgtttacaccacaatatatcctg | |
| cca | |

SEQ ID NO:41 is the nucleic acid sequence of pBI121-GmCPP. Italicized sequences are the right and left border repeats. Underlined sequence is the ³⁵S promoter. The G. max CaaX prenyl protease sense sequence is in bold.

| | |
|---|---|
| gtttacccgccaatatatcctgtcaaacactgatagtttaaactgaaggcgggaaacga | SEQ ID NO:42 |
| caatctgatcatgagcggagaattaagggagtcacgttatgacccccgccgatgacgcg | |
| ggacaagccgttttacgtttggaactgacagaaccgcaacgttgaaggagccactcagc | |
| cgcgggtttctggagtttaatgagctaagcacatacgtcagaaaccattattgcgcgtt | |
| caaaagtcgcctaaggtcactatcagctagcaaatatttcttgtcaaaaatgctccact | |
| gacgttccataaattcccctcggtatccaattagagtctcatattcactctcaatccaa | |
| ataatctgcaccggatctggatcgtttcgcatgattgaacaagatggattgcacgcagg | |
| ttctccggccgcttgggtggagaggctattcggctatgactgggcacaacagacaatcg | |
| gctgctctgatgccgccgtgttccggctgtcagcgcaggggcgcccggttctttttgtc | |
| aagaccgacctgtccggtgccctgaatgaactgcaggacgaggcagcgcggctatcgtg | |
| gctggccacgacgggcgttccttgcgcagctgtgctcgacgttgtcactgaagcgggaa | |
| gggactggctgctattgggcgaagtgccggggcaggatctcctgtcatctcaccttgct | |
| cctgccgagaaagtatccatcatggctgatgcaatgcggcggctgcatacgcttgatcc | |
| ggctacctgcccattcgaccaccaagcgaaacatcgcatcgagcgagcacgtactcgga | |
| tggaagccggtcttgtcgatcaggatgatctggacgaagagcatcaggggctcgcgcca | |
| gccgaactgttcgccaggctcaaggcgcgcatgcccgacggcgatgatctcgtcgtgac | |
| ccatggcgatgcctgcttgccgaatatcatggtggaaaatggccgcttttctggattca | |
| tcgactgtggccggctgggtgtggcggaccgctatcaggacatagcgttggctacccgt | |
| gatattgctgaagagcttggcggcgaatgggctgaccgcttcctcgtgctttacggtat | |
| cgccgctcccgattcgcagcgcatcgccttctatcgccttcttgacgagttcttctgag | |
| cgggactctggggttcgaaatgaccgaccaagcgcagcccaacctgccatcacgagatt | |
| tcgattccaccgccgccttctatgaaaggttgggcttcggaatcgttttccgggacgcc | |
| ggctggatgatcctccagcgcggggatctcatgctggagttcttcgcccacgggatctc | |
| tgcggaacaggcggtcgaaggtgccgatatcattacgacagcaacggccgacaagcaca | |
| acgccacgatcctgagcgacaatatgatcgggcccggcgtccacatcaacggcgtcggc | |
| ggcgactgcccaggcaagaccgagatgcaccgcgatatcttgctgcgttcggatatttt | |
| cgtggagttcccgccacagacccggatgatccccgatcgttcaaacatttggcaataaa | |
| gtttcttaagattgaatcctgttgccggtcttgcgatgattatcatataatttctgttg | |
| aattacgttaagcatgtaataattaacatgtaatgcatgacgttatttatgagatgggt | |
| ttttatgattagagtcccgcaattatacatttaatacgcgatagaaaacaaaatatagc | |
| gcgcaaactaggataaattatcgcgcgcggtgtcatctatgttactagatcgggcctcc | |
| tgtcaatgctggcggcggctctggtggtggttctggtggcggctctgagggtggtggct | |
| ctgagggtggcggttctgagggtggcggctctgagggaggcggttccggtggtggctct | |
| ggttccggtgattttgattatgaaaagatggcaaacgctaataagggggctatgaccga | |
| aaatgccgatgaaaacgcgctacagtctgacgctaaaggcaaacttgattctgtcgcta | |
| ctgattacggtgctgctatcgatggtttcattggtgacgtttccggccttgctaatggt | |
| aatggtgctactggtgattttgctggctctaattcccaaatggctcaagtcggtgacgg | |
| tgataattcacctttaatgaataatttccgtcaatatttaccttccctccctcaatcgg | |
| ttgaatgtcgcccttttgtctttggcccaatacgcaaaccgcctctccccgcgcgttgg | |
| ccgattcattaatgcagctggcacgacaggtttcccgactggaaagcgggcagtgagcg | |
| caacgcaattaatgtgagttagctcactcattaggcaccccaggctttacactttatgc | |
| ttccggctcgtatgttgtgtggaattgtgagcggataacaatttcacacaggaaacagc | |
| tatgaccatgattacgccaagcttgcatgcctgcagcccacagatggttagagaggctt | |
| acgcagcaggtctcatcaagacgatctacccgagcaataatctccaggaaatcaaatac | |
| cttcccaagaaggttaaagatgcagtcaaaagattcaggactaactgcatcaagaacac | |
| agagaaagatatatttctcaagatcagaagtactattccagtatggacgattcaaggct | |
| tgcttcacaaaccaaggcaagtaatagagattggagtctctaaaaaggtagttcccact | |
| gaatcaaaggccatggagtcaaagattcaaatagaggacctaacagaactcgccgtaaa | |
| gactggcgaacagttcatacagagtctcttacgactcaatgacaagaagaaaatcctcg | |
| tcaacatggtggagcacgacacacttgtctactccaaaaatatcaaagatacagtctca | |
| gaagaccaaagggcaattgagacttttcaacaaagggtaatatccggaaacctcctcgg | |
| attccattgcccagctatctgtcactttattgtgaagatagtggaaaaggaaggtggct | |
| cctacaaatgccatcattgcgataaaggaaaggccatcgttgaagatgcctctgccgac | |
| agtggtcccaaagatggacccccacccacgaggagcatcgtggaaaaagaagacgttcc | |
| aaccacgtcttcaaagcaagtggattgatgtgatatctccactgacgtaagggatgacg | |
| cacaatcccactatccttcgcaagacccttcctctatataaggaagttcatttcatttg | |
| gagagaacacgggggactctagaccggttcgtccagcgcggccaatctttcaacaaggg | |
| gaggatgagaatagtgataagcagagtaccaaggatctgtattcatagctgacagattc | |
| tcctcctgtagtttcacaagaccaccgcgtaatccagatgcatatccaagcttcttggc | |
| aaagccatcagcctgaaattcaaatgatcggctgactaggttcagaccaaagctgacca | |
| attgctgaagtgggattacagtatgctgaaatatgatgagcccaatgaggactggctgc | |
| gtatcaaacccaaagcttcgatacagatcagctgaatttcgcactagtgtatatcctcc | |
| aaattgtagaagtgtaagaatctgcatagcaacaaatgtgtacacagtatggttgagct | |
| tccagtgtcccaactcatgggcaataacagcaacaatttcctcatcgtctttgcactgt | |
| tgaattaatgtgtcataagggacaatcctcttgttcttgaagaatccatacatataggc | |
| attgctgtgacttgatcttgtggatccccATCTACCCGCTTCGCGTCGGCATCCGGTCA | |
| GTGGCAGTGAAGGGCGAACAGTTCCTGATTAACCACAAACCGTTCTACTTTACTGGCTT | |
| TGGTCGTCATGAAGATGCGGACTTGCGTGGCAAAGGATTCGATAACGTGCTGATGGTGC | |
| ACGACCACGCATTAATGGACTGGATTGGGGCCAACTCCTACCGTACCTCGCATTACCCT | |
| TACGCTGAAGAGATGCTCGACTGGGCAGATGAACATGGCATCGTGGTGATTGATGAAAC | |
| TGCTGCTGTCGGCTTTTCGCTCTCTTTAGGCATTGGTTTCGAAGCGGGCAACAAGCCGA | |
| AAGAACTGTACAGCGAAGAGGCAGTCAACGGGGAAACTCAGCAAGCGCACTTACAGGCG | |
| ATTAAAGAGCTGATAGCGCGTGACAAAAACCACCCAAGCGTGGTGATGTGGAGTATTGC | |
| CAACGAACCGGATACCCGTCCGCAAGGTGCACGGGAATATTTCGCGCCACTGGCGGAAG | |
| CAACGCGTAAACTCGACCCGACGCGTCCGATCACCTGCGTCAATGTAATGTTCTGCGAC | |
| GCTCACACCGATACCATCAGCGATCTCTTTGATGTGCTGTGCCTGAACCGTTATTACGG | |
| ATGGTATGTCCAAAGCGGCGATTTGGAAACGGCAGAGAAGGTACTGGAAAAAGAACTTC | |
| TGGCCTGGCAGGAGAAACTGTACACCGACATGTGGAGTGAAGAGTATCAGTGTGCATGG | |
| CTGGATATGTATCACCGCGTCTTTGATCGCGTCAGCGCCGTCGTCGGTGAACAGGTATG | |
| GAATTTCGCCGATTTTGCGACCTCGCAAGGCATATTGCGCGTTGGCGGTAACAAGAAAG | |
| GGATCTTCACTCGCGACCGCAAACCGAAGTCGGCGGCTTTTCTGCTGCAAAAACGCTGG | |
| ACTGGCATGAACTTCGGTGAAAAACCGCAGCAGGGAGGCAAACAATGAatcaacaactc | |
| tcctggcgcaccatcgtcggctacagcctcgggaattgctaccgagctcacaagatcaa | |
| gtcacagcaatgcctatatgtatggattcttcaagaacaagaggattgtcccttatgac | |
| acattaattcaacagtgcaaagacgatgaggaaattgttgctgttattgcccatgagtt | |
| gggacactggaagctcaaccatactgtgtacacatttgttgctatgcagattcttacac | |
| ttctacaatttggaggatatacactagtgcgaaattcagctgatctgtatcgaagcttt | |
| gggtttgatacgcagccagtcctcattgggctcatcatatttcagcatactgtaatccc | |
| acttcagcaattggtcagctttggtctgaacctagtcagccgatcatttgaatttcagg | |
| ctgatggctttgccaagaagcttggatatgcatctggattacgcggtggtcttgtgaaa | |
| ctacaggaggagaatctgtcagctatgaatacagatccttggtactctgcttatcacta | |
| ttctcatcctccccttgttgaaagattggccgcgctggacgaaccgggagctcgaattt | |
| ccccgatcgttcaaacatttggcaataaagtttcttaagattgaatcctgttgccggtc | |
| ttgcgatgattatcatataatttctgttgaattacgttaagcatgtaataattaacatg | |
| taatgcatgacgttatttatgagatgggtttttatgattagagtcccgcaattatacat | |
| ttaatacgcgatagaaaacaaaatatagcgcgcaaactaggataaattatcgcgcgcgg | |
| tgtcatctatgttactagatcgggaattcactggccgtcgttttacaacgtcgtgactg | |
| ggaaaaccctggcgttacccaacttaatcgccttgcagcacatccccctttcgccagct | |
| ggcgtaatagcgaagaggcccgcaccgatcgcccttcccaacagttgcgcagcctgaat | |
| ggcgcccgctcctttcgctttcttcccttcctttctcgccacgttcgccggctttcccc | |
| gtcaagctctaaatcgggggctccctttagggttccgatttagtgctttacggcacctc | |
| gaccccaaaaaacttgatttgggtgatggttcacgtagtgggccatcgccctgatagac | |
| ggtttttcgccctttgacgttggagtccacgttctttaatagtggactcttgttccaaa | |
| ctggaacaacactcaaccctatctcgggctattcttttgatttataagggattttgccg | |
| atttcggaaccaccatcaaacaggattttcgcctgctggggcaaaccagcgtggaccgc | |
| ttgctgcaactctctcagggccaggcggtgaagggcaatcagctgttgcccgtctcact | |
| ggtgaaaagaaaaaccaccccagtacattaaaaacgtccgcaatgtgttattaagttgt | |
| ctaagcgtcaatttgtttacaccacaatatatcctgcca | |

SEQ ID NO:42 is the nucleic acid sequence of pBI121-HP-GmCPP. Italicized sequences are the right and left border repeats. Underlined sequence is the ³⁵S promoter. Bold sequence is the antisense prenyl protease fragment of G. max. Bold and underlined sequence is the G. max sense prenyl protease fragment and sequence in upper case is the truncated GUS fragment.

| | |
|---|---|
| gtttacccgccaatatatcctgtcaaacactgatagtttaaactgaaggcgggaaacga | SEQ ID NO:43 |
| caatctgatcatgagcggagaattaagggagtcacgttatgacccccgccgatgacgcg | |
| ggacaagccgttttacgtttggaactgacagaaccgcaacgttgaaggagccactcagc | |
| cgcgggtttctggagtttaatgagctaagcacatacgtcagaaaccattattgcgcgtt | |
| caaaagtcgcctaaggtcactatcagctagcaaatatttcttgtcaaaaatgctccact | |
| gacgttccataaattcccctcggtatccaattagagtctcatattcactctcaatccaa | |
| ataatctgcaccggatctggatcgtttcgcatgattgaacaagatggattgcacgcagg | |
| ttctccggccgcttgggtggagaggctattcggctatgactgggcacaacagacaatcg | |
| gctgctctgatgccgccgtgttccggctgtcagcgcaggggcgcccggttctttttgtc | |
| aagaccgacctgtccggtgccctgaatgaactgcaggacgaggcagcgcggctatcgtg | |
| gctggccacgacgggcgttccttgcgcagctgtgctcgacgttgtcactgaagcgggaa | |
| gggactggctgctattgggcgaagtgccggggcaggatctcctgtcatctcaccttgct | |
| cctgccgagaaagtatccatcatggctgatgcaatgcggcggctgcatacgcttgatcc | |
| ggctacctgcccattcgaccaccaagcgaaacatcgcatcgagcgagcacgtactcgga | |
| tggaagccggtcttgtcgatcaggatgatctggacgaagagcatcaggggctcgcgcca | |
| gccgaactgttcgccaggctcaaggcgcgcatgcccgacggcgatgatctcgtcgtgac | |
| ccatggcgatgcctgcttgccgaatatcatggtggaaaatggccgcttttctggattca | |
| tcgactgtggccggctgggtgtggcggaccgctatcaggacatagcgttggctacccgt | |
| gatattgctgaagagcttggcggcgaatgggctgaccgcttcctcgtgctttacggtat | |
| cgccgctcccgattcgcagcgcatcgccttctatcgccttcttgacgagttcttctgag | |
| cgggactctggggttcgaaatgaccgaccaagcgacgcccaacctgccatcacgagatt | |
| tcgattccaccgccgccttctatgaaaggttgggcttcggaatcgttttccgggacgcc | |
| ggctggatgatcctccagcgcggggatctcatgctggagttcttcgcccacgggatctc | |
| tgcggaacaggcggtcgaaggtgccgatatcattacgacagcaacggccgacaagcaca | |
| acgccacgatcctgagcgacaatatgatcgggcccggcgtccacatcaacggcgtcggc | |
| ggcgactgcccaggcaagaccgagatgcaccgcgatatcttgctgcgttcggatatttt | |
| cgtggagttcccgccacagacccggatgatccccgatcgttcaaacatttggcaataaa | |
| gtttcttaagattgaatcctgttgccggtcttgcgatgattatcatataatttctgttg | |
| aattacgttaagcatgtaataattaacatgtaatgcatgacgttatttatgagatgggt | |
| ttttatgattagagtcccgcaattatacatttaatacgcgatagaaaacaaaatatagc | |
| gcgcaaactaggataaattatcgcgcgcggtgtcatctatgttactagatcgggcctcc | |
| tgtcaatgctggcggcggctctggtggtggttctggtggcggctctgagggtggtggct | |
| ctgagggtggcggttctgagggtggcggctctgagggaggcggttccggtggtggctct | |
| ggttccggtgattttgattatgaaaagatggcaaacgctaataagggggctatgaccga | |
| aaatgccgatgaaaacgcgctacagtctgacgctaaaggcaaacttgattctgtcgcta | |
| ctgattacggtgctgctatcgatggtttcattggtgacgtttccggccttgctaatggt | |
| aatggtgctactggtgattttgctggctctaattcccaaatggctcaagtcggtgacgg | |
| tgataattcacctttaatgaataatttccgtcaatatttaccttccctccctcaatcgg | |
| ttgaatgtcgcccttttgtctttggcccaatacgcaaaccgcctctccccgcgcgttgg | |
| ccgattcattaatgcagctggcacgacaggtttcccgactggaaagcgggcagtgagcg | |
| caacgcaattaatgtgagttagctcactcattaggcaccccaggctttacactttatgc | |
| ttccggctcgtatgttgtgtggaattgtgagcggataacaatttcacacaggaaacagc | |
| tatgaccatgattacgccaagcttgcatgcctgcagcccacagatggttagagaggctt | |
| acgcagcaggtctcatcaagacgatctacccgagcaataatctccaggaaatcaaatac | |
| cttcccaagaaggttaaagatgcagtcaaaagattcaggactaactgcatcaagaacac | |
| agagaaagatatatttctcaagatcagaagtactattccagtatggacgattcaaggct | |
| tgcttcacaaaccaaggcaagtaatagagattggagtctctaaaaaggtagttcccact | |
| gaatcaaaggccatggagtcaaagattcaaatagaggacctaacagaactcgccgtaaa | |
| gactggcgaacagttcatacagagtctcttacgactcaatgacaagaagaaaatcttcg | |
| tcaacatggtggagcacgacacacttgtctactccaaaaatatcaaagatacagtctca | |
| gaagaccaaagggcaattgagacttttcaacaaagggtaatatccggaaacctcctcgg | |
| attccattgcccagctatctgtcactttattgtgaagatagtggaaaaggaaggtggct | |
| cctacaaatgccatcattgcgataaaggaaaggccatcgttgaagatgcctctgccgac | |
| agtggtcccaaagatggacccccacccacgaggagcatcgtggaaaaagaagacgttcc | |
| aaccacgtcttcaaagcaagtggattgatgtgatatctccactgacgtaagggatgacg | |
| cacaatcccactatccttcgcaagacccttcctctatataaggaagttcatttcatttg | |
| gagagaacacgggggactctagaggatccccgggttagtcttccttcttatccggttcg | |
| tccagcgcggccaatctttcaacaaggggaggatgagaatagtgataagcagagtacca | |
| aggatctgtattcatagctgacagattctcctcctgtagtttcacaagaccaccgcgta | |
| atccagatgcatatccaagcttcttggcaaagccatcagcctgaaattcaaatgatcgg | |
| ctgactaggttcagaccaaagctgaccaattgctgaagtgggattacagtatgctgaaa | |
| tatgatgagcccaatgaggactggctgcgtatcaaacccaaagcttcgatacagatcag | |
| ctgaatttcgcactagtgtatatcctccaaattgtagaagtgtaagaatctgcatagca | |
| acaaatgtgtacacagtatggttgagcttccagtgtcccaactcatgggcaataacagc | |
| aacaatttcctcatcgtctttgcactgttgaattaatgtgtcataagggacaatcctct | |
| tgttcttgaagaatccatacatataggcattgctgtgacttgatcttgtggatccatcg | |
| acaacaaatagtttctttaacggatagttgagggaggaagcaagtttctcgattttctc | |
| cctgagttgaccatctggaagtggagtgaacttattgaagagtggagctattagtactg | |
| gataaagggtcatcatcacaatagaaagaccaaacgtaaaaacccaaagatagatggcc | |
| aagtatggacctcctttctgtactattacaatgattgcagccacaataggtggaccaat | |
| tattacagaaaggaaaattcctttaagcatgtccctaaagaataaccatggtgtttgct | |
| tattaaaaccatgacgggcctcaatcacaaaagttgagtacagagaaaagggcaaatct | |
| gttatctgtgaccaaatcatcagccctgctaagaaggcaagggtatgcagtatttcatt | |
| ctcagcattgaaaccagctattgtcataaaatctcctgatttcttccaaaaccagggca | |
| ataccccaaagtacaaaattgtagagtctgtcactattgtcacaaactcgtgaacaaaa | |
| tggaagtggcttttatcaagactataggctctagatttctcaaatttctcttggctgat | |
| aacaccctctaaagtctttggaagagtaggaagtttgagggccctatgttgtcgcacat | |
| ccaagtaagtttcaaaaatgtacattaatatcataaatccgacaacggcttccatgtag | |
| ggaaacgccatgagctcgaatttccccgatcgttcaaacatttggcaataaagtttctt | |
| aagattgaatcctgttgccggtcttgcgatgattatcatataatttctgttgaattacg | |
| ttaagcatgtaataattaacatgtaatgcatgacgttatttatgagatgggtttttatg | |
| attagagtcccgcaattatacatttaatacgcgatagaaaacaaaatatagcgcgcaaa | |
| ctaggataaattatcgcgcgcggtgtcatctatgttactagatcgggaattcactggcc | |
| gtcgttttacaacgtcgtgactgggaaaaccctggcgttacccaacttaatcgccttgc | |
| agcacatccccctttcgccagctggcgtaatagcgaagaggcccgcaccgatcgccctt | |
| cccaacagttgcgcagcctgaatggcgcccgctcctttcgctttcttcccttcctttct | |
| cgccacgttcgccggctttccccgtcaagctctaaatcgggggctccctttagggttcc | |
| gatttagtgctttacggcacctcgaccccaaaaaacttgatttgggtgatggttcacgt | |
| agtgggccatcgccctgatagacggtttttcgccctttgacgttggagtccacgttctt | |
| taatagtggactcttgttccaaactggaacaacactcaaccctatctcgggctattctt | |
| ttgatttataagggattttgccgatttcggaaccaccatcaaacaggattttcgcctgc | |
| tggggcaaaccagcgtggaccgcttgctgcaactctctcagggccaggcggtgaagggc | |
| aatcagctgttgcccgtctcactggtgaaaagaaaaaccaccccagtacattaaaaacg | |
| tccgcaatgtgttattaagttgtctaagcgtcaatttgtttacaccacaatatatcctg | |

SEQ ID NO:43 is the nucleic acid sequence of pBI121-antisense-GmCPP. Italicized sequences are the right and left border repeats. Underlined sequence is the ³⁵S promoter. Sequence in bold is the GmCPP anti-sense sequence.

| | |
|---|---|
| gtttacccgccaatatatcctgtcaaacactgatagtttaaactgaaggcgggaaacga | SEQ ID NO:44 |
| caatctgatcatgagcggagaattaagggagtcacgttatgacccccgccgatgacgcg | |
| ggacaagccgttttacgtttggaactgacagaaccgcaacgttgaaggagccactcagc | |
| cgcgggtttctggagtttaatgagctaagcacatacgtcagaaaccattattgcgcgtt | |
| caaaagtcgcctaaggtcactatcagctagcaaatatttcttgtcaaaaatgctccact | |
| gacgttccataaattcccctcggtatccaattagagtctcatattcactctcaatccaa | |
| ataatctgcaccggatctggatcgtttcgcatgattgaacaagatggattgcacgcagg | |
| ttctccggccgcttgggtggagaggctattcggctatgactgggcacaacagacaatcg | |
| gctgctctgatgccgccgtgttccggctgtcagcgcaggggcgcccggttctttttgtc | |
| aagaccgacctgtccggtgccctgaatgaactgcaggacgaggcagcgcggctatcgtg | |
| gctggccacgacgggcgttccttgcgcagctgtgctcgacgttgtcactgaagcgggaa | |
| gggactggctgctattgggcgaagtgccggggcaggatctcctgtcatctcaccttgct | |
| cctgccgagaaagtatccatcatggctgatgcaatgcggcggctgcatacgcttgatcc | |
| ggctacctgcccattcgaccaccaagcgaaacatcgcatcgagcgagcacgtactcgga | |
| tggaagccggtcttgtcgatcaggatgatctggacgaagagcatcaggggctcgcgcca | |
| gccgaactgttcgccaggctcaaggcgcgcatgcccgacggcgatgatctcgtcgtgac | |
| ccatggcgatgcctgcttgccgaatatcatggtggaaaatggccgcttttctggattca | |
| tcgactgtggccggctgggtgtggcggaccgctatcaggacatagcgttggctacccgt | |
| gatattgctgaagagcttggcggcgaatgggctgaccgcttcctcgtgctttacggtat | |
| cgccgctcccgattcgcagcgcatcgccttctatcgccttcttgacgagttcttctgag | |
| cgggactctggggttcgaaatgaccgaccaagcgacgcccaacctgccatcacgagatt | |
| tcgattccaccgccgccttctatgaaaggttgggcttcggaatcgttttccgggacgcc | |
| ggctggatgatcctccagcgcggggatctcatgctggagttcttcgcccacgggatctc | |
| tgcggaacaggcggtcgaaggtgccgatatcattacgacagcaacggccgacaagcaca | |
| acgccacgatcctgagcgacaatatgatcgggcccggcgtccacatcaacggcgtcggc | |
| ggcgactgcccaggcaagaccgagatgcaccgcgatatcttgctgcgttcggatatttt | |
| cgtggagttcccgccacagacccggatgatccccgatcgttcaaacatttggcaataaa | |
| gtttcttaagattgaatcctgttgccggtcttgcgatgattatcatataatttctgttg | |
| aattacgttaagcatgtaataattaacatgtaatgcatgacgttatttatgagatgggt | |
| ttttatgattagagtcccgcaattatacatttaatacgcgatagaaaacaaaatatagc | |
| gcgcaaactaggataaattatcgcgcgcggtgtcatctatgttactagatcgggcctcc | |
| tgtcaatgctggcggcggctctggtggtggttctggtggcggctctgagggtggtggct | |
| ctgagggtggcggttctgagggtggcggctctgagggaggcggttccggtggtggctct | |
| ggttccggtgattttgattatgaaaagatggcaaacgctaataagggggctatgaccga | |
| aaatgccgatgaaaacgcgctacagtctgacgctaaaggcaaacttgattctgtcgcta | |
| ctgattacggtgctgctatcgatggtttcattggtgacgtttccggccttgctaatggt | |
| aatggtgctactggtgattttgctggctctaattcccaaatggctcaagtcggtgacgg | |
| tgataattcacctttaatgaataatttccgtcaatatttaccttccctccctcaatcgg | |
| ttgaatgtcgcccttttgtctttggcccaatacgcaaaccgcctctccccgcgcgttgg | |
| ccgattcattaatgcagctggcacgacaggtttcccgactggaaagcgggcagtgagcg | |
| caacgcaattaatgtgagttagctcactcattaggcaccccaggctttacactttatgc | |
| ttccggctcgtatgttgtgtggaattgtgagcggataacaatttcacacaggaaacagc | |
| tatgaccatgattacgccaagcttgcatgcctgcagggagccatagatgcaattcaata | |
| aaactgaaatttctgcaagaatctcaaacacggagatctcaaagtttgaaagaaaattt | |
| atttcttcgactcaaaacaaacttacgaaatttaggtagaacttatatacattatattg | |
| taattttttgtaacaaaatgtttttattattattatagaattttactggttaaattaaa | |
| aatgaatagaaaaggtgaattaagaggagagaggaggtaaacattttcttctatttttt | |
| catattttcaggataaattattgtaaaagtttacaagatttccatttgactagtgtaaa | |
| tgaggaatattctctagtaagatcattatttcatctacttcttttatcttctaccagta | |
| gaggaataaacaatatttagctcctttgtaaatacaaattaattttccttcttgacatc | |
| attcaattttaattttacgtataaaataaaagatcatacctattagaacgattaaggag | |
| aaatacaattcgaatgagaaggatgtgccgtttgttataataaacagccacacgacgta | |
| aacgtaaaatgaccacatgatgggccaatagacatggaccgactactaataatagtaag | |
| ttacattttaggatggaataaatatcataccgacatcagttttgaaagaaaagggaaaa | |
| aaagaaaaaataaataaaagatatactaccgacatgagttccaaaaagcaaaaaaaaag | |
| atcaagccgacacagacacgcgtagagagcaaaatgactttgacgtcacaccacgaaaa | |
| cagacgcttcatacgtgtccctttatctctctcagtctctctataaacttagtgagacc | |
| tgattacttctattggaaaggactctagaggatccccgggatggcgtttccctacatgg | |
| aagccgttgtcggatttatgatattaatgtacatttttgaaacttacttggatgtgcga | |
| caacatagggccctcaaacttcctactcttccaaagactttagagggtgttatcagcca | |
| agagaaatttgagaaatctagagcctatagtcttgataaaagccacttccattttgttc | |
| acgagtttgtgacaatagtgacagactctacaattttgtactttggggtattgccctgg | |
| ttttggaagaaatcaggagattttatgacaatagctggtttcaatgctgagaatgaaat | |
| actgcatacccttgccttcttagcagggctgatgatttggtcacagataacagatttgc | |
| ccttttctctgtactcaacttttgtgattgaggcccgtcatggttttaataagcaaaca | |
| ccatggttattctttagggacatgcttaaaggaattttcctttctgtaataattggtcc | |
| acctattgtggctgcaatcattgtaatagtacagaaaggaggtccatacttggccatct | |
| atctttgggtttttacgtttggtctttctattgtgatgatgaccctttatccagtacta | |
| atagctccactcttcaataagttcactccacttccagatggtcaactcagggagaaaat | |
| cgagaaacttgcttcctccctcaactatccgttaaagaaactatttgttgtcgatggat | |
| ccacaagatcaagtcacagcaatgcctatatgtatggattcttcaagaacaagaggatt | |
| gtcccttatgacacattaattcaacagtgcaaagacgatgaggaaattgttgctgttat | |
| tgcccatgagttgggacactggaagctcaaccatactgtgtacacatttgttgctatgc | |
| agattcttacacttctacaatttggaggatatacactagtgcgaaattcagctgatctg | |
| tatcgaagctttgggtttgatacgcagccagtcctcattgggctcatcatatttcagca | |
| tactgtaatcccacttcagcaattggtcagctttggtctgaacctagtcagccgatcat | |
| ttgaatttcaggctgatggctttgccaagaagcttggatatgcatctggattacgcggt | |
| ggtcttgtgaaactacaggaggagaatctgtcagctatgaatacagatccttggtactc | |
| tgcttatcactattctcatcctccccttgttgaaagattggccgcgctggacgaaccgg | |
| ataagaaggaagactaagagctcgaatttccccgatcgttcaaacatttggcaataaag | |
| tttcttaagattgaatcctgttgccggtcttgcgatgattatcatataatttctgttga | |
| attacgttaagcatgtaataattaacatgtaatgcatgacgttatttatgagatgtgtt | |
| tttatgattagagtcccgcaattatacatttaatacgcgatagaaaacaaaacatagcg | |
| cgcaaactaggataaattatcgcgcgcggtgtcatctatgttactagatcgggaattca | |
| ctggccgtcgttttacaacgtcgtgactgggaaaaccctggcgttacccaacttaatcg | |
| ccttgcagcacatccccctttcgccagctggcgtaatagcgaagaggcccgcaccgatc | |
| gcccttcccaacagttgcgcagcctgaatggcgcccgctcctttcgctttcttcccttc | |
| ctttctcgccacgttcgccggctttccccgtcaagctctaaatcgggggctccctttag | |
| ggttccgatttagtgctttacggcacctcgaccccaaaaaacttgatttgggtgatggt | |
| tcacgtagtgggccatcgccctgatagacggtttttcgccctttgacgttggagtccac | |
| gttctttaatagtggactcttgttccaaactggaacaacactcaaccctatctcgggct | |
| attcttttgatttataagggattttgccgatttcggaaccaccatcaaacaggattttc | |
| gcctgctggggcaaaccagcgtggaccgcttgctgcaactctctcagggccaggcggtg | |
| aagggcaatcagctgttgcccgtctcactggtgaaaagaaaaaccaccccagtacatta | |
| aaaacgtccgcaatgtgttattaagttgtctaagcgtcaatttgtttacaccacaatat | |
| atcctgcca | |

SEQ ID NO:44 is the nucleic acid sequence of pRD29A-GmCPP. Italicized sequences are the right and left border repeats. Underlined sequence is the RD29A promoter. Sequence in bold is the GmCPP sense sequence.

SEQ ID NO:45

gtttacccgccaatatatcctgtcaaacactgatagtttaaactgaaggcgggaaacga
caatctgatcatgagcggagaattaagggagtcacgttatgacccccgccgatgacgcg
ggacaagccgttttacgtttggaactgacagaaccgcaacgttgaaggagccactcagc
cgcgggtttctggagtttaatgagctaagcacatacgtcagaaaccattattgcgcgtt
caaaagtcgcctaaggtcactatcagctagcaaatatttcttgtcaaaaatgctccact
gacgttccataaattcccctcggtatccaattagagtctcatattcactctcaatccaa
ataatctgcaccggatctggatcgtttcgcatgattgaacaagatggattgcacgcagg
ttctccggccgcttgggtggagaggctattcggctatgactgggcacaacagacaatcg
gctgctctgatgccgccgtgttccggctgtcagcgcaggggcgcccggttctttttgtc
aagaccgacctgtccggtgccctgaatgaactgcaggacgaggcagcgcggctatcgtg
gctggccacgacgggcgttccttgcgcagctgtgctcgacgttgtcactgaagcgggaa
gggactggctgctattgggcgaagtgccggggcaggatctcctgtcatctcaccttgct
cctgccgagaaagtatccatcatggctgatgcaatgcggcggctgcatacgcttgatcc
ggctacctgcccattcgaccaccaagcgaaacatcgcatcgagcgagcacgtactcgga
tggaagccggtcttgtcgatcaggatgatctggacgaagagcatcaggggctcgcgcca
gccgaactgttcgccaggctcaaggcgcgcatgcccgacggcgatgatctcgtcgtgac
ccatggcgatgcctgcttgccgaatatcatggtggaaaatggccgcttttctggattca
tcgactgtggccggctgggtgtggcggaccgctatcaggacatagcgttggctacccgt
gatattgctgaagagcttggcggcgaatgggctgaccgcttcctcgtgctttacggtat
cgccgctcccgattcgcagcgcatcgccttctatcgccttcttgacgagttcttctgag
cgggactctggggttcgaaatgaccgaccaagcgacgcccaacctgccatcacgagatt
tcgattccaccgccgccttctatgaaaggttgggcttcggaatcgttttccgggacgcc
ggctggatgatcctccagcgcggggatctcatgctggagttcttcgcccacgggatctc
tgcggaacaggcggtcgaaggtgccgatatcattacgacagcaacggccgacaagcaca
acgccacgatcctgagcgacaatatgatcgggcccggcgtccacatcaacggcgtcggc
ggcgactgcccaggcaagaccgagatgcaccgcgatatcttgctgcgttcggatatttt
cgtggagttcccgccacagacccggatgatccccgatcgttcaaacatttggcaataaa
gtttcttaagattgaatcctgttgccggtcttgcgatgattatcatataatttctgttg
aattacgttaagcatgtaataattaacatgtaatgcatgacgttatttatgagatgggt
ttttatgattagagtcccgcaattatacatttaatacgcgatagaaaacaaaatatagc
gcgcaaactaggataaattatcgcgcgcggtgtcatctatgttactagatcgggcctcc
tgtcaatgctggcggcggctctggtggtggttctggtggcggctctgagggtggtggct
ctgagggtggcggttctgagggtggcggctctgagggaggcggttccggtggtggctct
ggttccggtgattttgattatgaaaagatggcaaacgctaataagggggctatgaccga
aaatgccgatgaaaacgcgctacagtctgacgctaaaggcaaacttgattctgtcgcta
ctgattacggtgctgctatcgatggtttcattggtgacgtttccggccttgctaatggt
aatggtgctactggtgattttgctggctctaattcccaaatggctcaagtcggtgacgg
tgataattcacctttaatgaataatttccgtcaatatttaccttccctccctcaatcgg
ttgaatgtcgcccttttgtctttggcccaatacgcaaaccgcctctccccgcgcgttgg
ccgattcattaatgcagctggcacgacaggtttcccgactggaaagcgggcagtgagcg
caacgcaattaatgtgagttagctcactcattaggcaccccaggctttacactttatgc
ttccggctcgtatgttgtgtggaattgtgagcggataacaatttcacacaggaaacagc
tatgaccatgattacgccaagcttgcatgcctgcagggagccatagatgcaattcaatc
aaactgaaatttctgcaagaatctcaaacacggagatctcaaagtttgaaagaaaattt
atttcttcgactcaaaacaaacttacgaaatttaggtagaacttatatacattatattg
taattttttgtaacaaaatgtttttattattattatagaattttactggttaaattaaa
aatgaatagaaaaggtgaattaagaggagagaggaggtaaacattttcttctatttttt
catattttcaggataaattattgtaaaagtttacaagatttccatttgactagtgtaaa
tgaggaatattctctagtaagatcattatttcatctacttcttttatcttctaccagta
gaggaataaacaatatttagctcctttgtaaatacaaattaattttccttcttgacatc
attcaattttaattttacgtataaaataaaagatcatacctattagaacgattaaggag
aaatacaattcgaatgagaaggatgtgccgtttgttataataaacagccacacgacgta
aacgtaaaatgaccacatgatgggccaatagacatggaccgactactaataatagtaag
ttacattttaggatggaataaatatcataccgacatcagttttgaaagaaaagggaaaa
aaagaaaaaataaataaaagatatactaccgacatgagttccaaaaagcaaaaaaaaag
atcaagccgacacagacacgcgtagagagcaaaatgactttgacgtcacaccacgaaaa
cagacgcttcatacgtgtccctttatctctctcagtctctctataaacttagtgagacc
ctcctctgttttactcacaaatatgcaaactagaaaacaatcatcaggaataaagggtt
tgattacttctattggaaaggactctagaccggttcgtccagcgcggccaatctttcaa
caaggggaggatgagaatagtgataagcagagtaccaaggatctgtattcatagctgac
agattctcctcctgtagtttcacaagaccaccgcgtaatccagatgcatatccaagctt
cttggcaaagccatcagcctgaaattcaaatgatcggctgactaggttcagaccaaagc
tgaccaattgctgaagtgggattacagtatgctgaaatatgatgagcccaatgaggact
ggctgcgtatcaaacccaaagcttcgatacagatcagctgaatttcgcactagtgtata
tcctccaaattgtagaagtgtaagaatctgcatagcaacaaatgtgtacacagtatggt
tgagcttccagtgtcccaactcatgggcaataacagcaacaatttcctcatcgtctttg
cactgttgaattaatgtgtcataagggacaatcctcttgttcttgaagaatccatacat
ataggcattgctgtgacttgatcttgtggatccccATCTACCCGCTTCGCGTCGGCATC
CGGTCAGTGGCAGTGAAGGGCGAACAGTTCCTGATTAACCACAAACCGTTCTACTTTAC
TGGCTTTGGTCGTCATGAAGATGCGGACTTGCGTGGCAAAGGATTCGATAACGTGCTGA
TGGTGCACGACCACGCATTAATGGACTGGATTGGGGCCAACTCCTACCGTACCTCGCAT
TACCCTTACGCTGAAGAGATGCTCGACTGGGCAGATGAACATGGCATCGTGGTGATTGA
TGAAACTGCTGCTGTCGGCTTTTCGCTCTCTTTAGGCATTGGTTTCGAAGCGGGCAACA
AGCCGAAAGAACTGTACAGCGAAGAGGCAGTCAACGGGGAAACTCAGCAAGCGCACTTA
CAGGCGATTAAAGAGCTGATAGCGCGTGACAAAAACCACCCAAGCGTGGTGATGTGGAG
TATTGCCAACGAACCGGATACCCGTCCGCAAGGTGCACGGGAATATTTCGCGCCACTGG
CGGAAGCAACGCGTAAACTCGACCCGACGCGTCCGATCACCTGCGTCAATGTAATGTTC
TGCGACGCTCACACCGATACCATCAGCGATCTCTTTGATGTGCTGTGCCTGAACCGTTA
TTACGGATGGTATGTCCAAAGCGGCGATTTGGAAACGGCAGAGAAGGTACTGGAAAAAG
AACTTCTGGCCTGGCAGGAGAAACTGTACACCGACATGTGGAGTGAAGAGTATCAGTGT
GCATGGCTGGATATGTATCACCGCGTCTTTGATCGCGTCAGCGCCGTCGTCGGTGAACA
GGTATGGAATTTCGCCGATTTTGCGACCTCGCAAGGCATATTGCGCGTTGGCGGTAACA
AGAAAGGGATCTTCACTCGCGACCGCAAACCGAAGTCGGCGGCTTTTCTGCTGCAAAAA
CGCTGGACTGGCATGAACTTCGGTGAAAAACCGCAGCAGGGAGGCAAACAATGAatcaa
caactctcctggcgcaccatcgtcggctacagcctcgggaattgctaccgagctcacaa
gatcaagtcacagcaatgcctatatgtatggattcttcaagaacaagaggattgtccct
tatgacacattaattcaacagtgcaaagacgatgaggaaattgttgctgttattgccca
tgagttgggacactggaagctcaaccatactgtgtacacatttgttgctatgcagattc
ttacacttctacaatttggaggatatacactagtgcgaaattcagctgatctgtatcga
agctttgggtttgatacgcagccagtcctcattgggctcatcatatttcagcatactgt
aatcccacttcagcaattggtcagctttggtctgaacctagtcagccgatcatttgaat
ttcaggctgatggctttgccaagaagcttggatatgcatctggattacgcggtggtctt
gtgaaactacaggaggagaatctgtcagctatgaatacagatccttggtactctgctta
tcactattctcatcctccccttgttgaaagattggccgcgctggacgaaccgggagctc
gaatttccccgatcgttcaaacatttggcaataaagtttcttaagattgaatcctgttg
ccggtcttgcgatgattatcatataatttctgttgaattacgttaagcatgtaataatt
aacatgtaatgcatgacgttatttatgagatgggtttttatgattagagtcccgcaatt
atacatttaatacgcgatagaaaacaaaatatagcgcgcaaactaggataaattatcgc
gcgcggtgtcatctatgttactagatcgggaattcactggccgtcgttttacaacgtcg
tgactgggaaaaccctggcgttacccaacttaatcgccttgcagcacatccccctttcg
ccagctggcgtaatagcgaagaggcccgcaccgatcgcccttcccaacagttgcgcagc
ctgaatggcgcccgctcctttcgctttcttcccttcctttctcgccacgttcgccggct
ttccccgtcaagctctaaatcgggggctccctttagggttccgatttagtgctttacgg
cacctcgaccccaaaaaacttgatttgggtgatggttcacgtagtgggccatcgccctg
atagacggtttttcgccctttgacgttggagtccacgttctttaatagtggactcttgt
tccaaactggaacaacactcaaccctatctcgggctattcttttgatttataagggatt
ttgccgatttcggaaccaccatcaaacaggattttcgcctgctggggcaaaccagcgtg
gaccgcttgctgcaactctctcagggccaggcggtgaagggcaatcagctgttgcccgt
ctcactggtgaaaagaaaaaccaccccagtacattaaaaacgtccgcaatgtgttatta
agttgtctaagcgtcaatttgtttacaccacaatatatcctgcca

SEQ ID NO:45 is the nucleic acid sequence of pRD29A-HP-GmCPP. Italicized sequences are the right and left border repeats. Underlined sequence is the RD29A promoter. Sequence in bold is the GmCPP antisense sequence, bold and underlined sequence is the GmCPP sense sequence.

SEQ ID NO:46 [symbol]
gtttacccgccaatatatcctgtcaaacactgatagtttaaactgaaggcgggaaacgacaatctgatcatgagcggaga
attaagggagtcacgttatgacccccgccgatgacgcgggacaagccgttttacgtttggaactgacagaaccgcaacg
ttgaaggagccactcagccgcgggtttctggagtttaatgagctaagcacatacgtcagaaaccattattgcgcgttcaa
aagtcgcctaaggtcactatcagctagcaaatatttcttgtcaaaaatgctccactgacgttccataaattcccctcggtat
ccaattagagtctcatattcactctcaatccaaataatctgcaccggatctggatcgtttcgcatgattgaacaagatggat
tgcacgcaggttctccggccgcttgggtggagaggctattcggctatgactgggcacaacagacaatcggctgctctgat
gccgccgtgttccggctgtcagcgcaggggcgcccggttctttttgtcaagaccgacctgtccggtgccctgaatgaactg
caggacgaggcagcgcggctatcgtggctggccacgacgggcgttccttgcgcagctgtgctcgacgttgtcactgaag
cgggaagggactggctgctattgggcgaagtgccggggcaggatctcctgtcatctcaccttgctcctgccgagaaagta
tccatcatggctgatgcaatgcggcggctgcatacgcttgatccggctacctgcccattcgaccaccaagcgaaacatcg
catcgagcgagcacgtactcggatggaagccggtcttgtcgatcaggatgatctggacgaagagcatcaggggctcgc
gccagccgaactgttcgccaggctcaaggcgcgcatgcccgacggcgatgatctcgtcgtgacccatggcgatgcctgc
ttgccgaatatcatggtggaaaatggccgcttttctggattcatcgactgtggccggctgggtgtggcggaccgctatcag
gacatagcgttggctacccgtgatattgctgaagagcttggcggcgaatgggctgaccgcttcctcgtgctttacggtatc
gccgctcccgattcgcagcgcatcgccttctatcgccttcttgacgagttcttctgagcgggactctggggttcgaaatgac
cgaccaagcgacgcccaacctgccatcacgagatttcgattccaccgccgccttctatgaaaggttgggcttcggaatcg
ttttccgggacgccggctggatgatcctccagcgcggggatctcatgctggagttcttcgcccacgggatctctgcggaac
aggcggtcgaaggtgccgatatcattacgacagcaacggccgacaagcacaacgccacgatcctgagcgacaatatg
atcgggcccggcgtccacatcaacggcgtcggcggcgactgcccaggcaagaccgagatgcaccgcgatatcttgctg
cgttcggatattttcgtggagttcccgccacagacccggatgatccccgatcgttcaaacatttggcaataaagtttcttaa
gattgaatcctgttgccggtcttgcgatgattatcatataatttctgttgaattacgttaagcatgtaataattaacatgtaat
gcatgacgttatttatgagatgggtttttatgattagagtcccgcaattatacatttaatacgcgatagaaaacaaaatata
gcgcgcaaactaggataaattatcgcgcgcggtgtcatctatgttactagatcgggcctcctgtcaatgctggcggcggct
ctggtggtggttctggtggcggctctgagggtggtggctctgagggtggcggttctgagggtggcggctctgagggaggc
ggttccggtggtggctctggttccggtgattttgattatgaaaagatggcaaacgctaataagggggctatgaccgaaaa
tgccgatgaaaacgcgctacagtctgacgctaaaggcaaacttgattctgtcgctactgattacggtgctgctatcgatgg
tttcattggtgacgtttccggccttgctaatggtaatggtgctactggtgattttgctggctctaattcccaaatggctcaagt
cggtgacggtgataattcacctttaatgaataatttccgtcaatatttaccttccctccctcaatcggttgaatgtcgcccttt
tgtctttggcccaatacgcaaaccgcctctccccgcgcgttggccgattcattaatgcagctggcacgacaggtttcccga
ctggaaagcgggcagtgagcgcaacgcaattaatgtgagttagctcactcattaggcaccccaggctttacactttatgc
ttccggctcgtatgttgtgtggaattgtgagcggataacaatttcacacaggaaacagctatgaccatgattacgccaag
cttgcatgcctgcagggagccatagatgcaattcaatcaaactgaaatttctgcaagaatctcaaacacggagatctcaa
agtttgaaagaaatttatttcttcgactcaaaacaaacttacgaaatttaggtagaacttatatacattatattgtaattttt
tgtaacaaatgtttttattattattatagaattttactggttaaattaaaaatgaatagaaaaggtgaattaagaggaga
gaggaggtaaacattttcttctattttttcatattttcaggataaattattgtaaaagtttacaagatttccatttgactagtgt
aaatgaggaatattctctagtaagatcattatttcatctacttcttttatcttctaccagtagaggaataaacaatatttagct
cctttgtaaatacaaattaattttcttcttgacatcattcaattttaattttacgtataaaataaaagatcatacctattaga
acgattaaggagaaatacaattcgaatgagaaggatgtgccgtttgttataataaacagccacacgacgtaaacgtaaa
atgaccacatgatgggccaatagacatggaccgactactaataatagtaagttacattttaggatggaataaatatcata
ccgacatcagttttgaaagaaaagggaaaaaaagaaaaaataaataaaagatatactaccgacatgagttccaaaaa
gcaaaaaaaaagatcaagccgacacagacacgcgtagagagcaaaatgactttgacgtcacaccacgaaaacagac
gcttcatacgtgtccctttatctctctcagtctctctataaacttagtgagaccctcctctgttttactcacaaatatgcaaact
agaaaacaatcatcaggaataaagggtttgattacttctattggaaaggactctagaggatcccgggttagtcttccttctt
atccggttcgtccagcgcggccaatctttcaacaaggggaggatgagaatagtgataagcagagtaccaaggatctgtattcata
gctgacagattctcctcctgtagtttcacaagaccaccgcgtaatccagatgcatatccaagcttcttggcaaagccatcagcctg
aaattcaaatgatcggctgactaggttcagaccaaagctgaccaattgctgaagtgggattacagtatgctgaaatatgatgagcc
caatgaggactggctgcgtatcaaacccaaagcttcgatacagatcagctgaatttcgcactagtgtatatcctccaaattgtagaa
gtgtaagaatctgcatagcaacaaatgtgtacacagtatggttgagcttccagtgtcccaactcatgggcaataacagcaacaattt
cctcatcgtctttgcactgttgaattaatgtgtcataagggacaatcctcttgttcttgaagaatccatacatataggcattgctgtgac
ttgatcttgtggatccatcgacaacaaatagtttctttaacggatagttgagggaggaagcaagtttctcgattttctccctgagttga
ccatctggaagtggagtgaacttattgaagagtggagctattagtactggataaagggtcatcatcacaatagaaagaccaaacg
taaaaacccaaagatagatggccaagtatggacctcctttctgtactattacaatgattgcagccacaataggtggaccaattatta
cagaaaggaaaattcctttaagcatgtccctaaagaataaccatggtgtttgcttattaaaaccatgacgggcctcaatcacaaaag
ttgagtacagagaaaagggcaaatctgttatctgtgaccaaatcatcagccctgctaagaaggcaagggtatgcagtatttcattct
cagcattgaaaccagctattgtcataaaatctcctgatttcttccaaaaccagggcaataccccaaagtacaaaattgtagagtctgt
cactattgtcacaaactcgtgaacaaaatggaagtggcttttatcaagactataggctctagatttctcaaatttctcttggctgataac
accctctaaagtctttggaagagtaggaagtttgagggccctatgttgtcgcacatccaagtaagtttcaaaaatgtacattaatatc
ataaatccgacaacggcttccatgtagggaaacgccatgagctcgaatttccccgatcgttcaaacatttggcaataaagttt
cttaagattgaatcctgttgccggtcttgcgatgattatcatataatttctgttgaattacgttaagcatgtaataattaacat
gtaatgcatgacgttatttatgagatgggtttttatgattagagtcccgcaattatacatttaatacgcgatagaaaacaaa
atatagcgcgcaaactaggataaattatcgcgcgcggtgtcatctatgttactagatcgggaattcactggccgtcgtttt
acaacgtcgtgactgggaaaaccctggcgttacccaacttaatcgccttgcagcacatccccctttcgccagctggcgta
atagcgaagaggcccgcaccgatcgcccttcccaacagttgcgcagcctgaatggcgcccgctcctttcgctttcttccct
tcctttctcgccacgttcgccggctttccccgtcaagctctaaatcgggggctccctttagggttccgatttagtgctttacgg
cacctcgaccccaaaaaacttgatttgggtgatggttcacgtagtgggccatcgccctgatagacggtttttcgccctttga
cgttggagtccacgttctttaatagtggactcttgttccaaactggaacaacactcaaccctatctcgggctattcttttgatt
tataagggattttgccgatttcggaaccaccatcaaacaggattttcgcctgctggggcaaaccagcgtggaccgcttgct
gcaactctctcagggccaggcggtgaagggcaatcagctgttgcccgtctcactggtgaaaagaaaaaccaccccagt
acattaaaaacgtccgcaatgtgttattaagttgtctaagcgtcaattt [symbol] gtttacaccacaatatatcctgcca

SEQ ID NO:46 is the nucleic acid sequence of pRD29A-antisense-GmCPP. Italicized sequences are the right and left border repeats. Underlined sequence is the RD29A promoter. Sequence in bold is the GmCPP antisense sequence.

SEQ ID NO:47

gtttacccgccaatatatcctgtcaaacactgatagtttaaactgaaggcgggaaacga
caatctgatcatgagcggagaattaagggagtcacgttatgacccccgccgatgacgcg
ggacaagccgttttacgtttggaactgacagaaccgcaacgttgaaggagccactcagc
cgcgggtttctggagtttaatgagctaagcacatacgtcagaaaccattattgcgcgtt
caaaagtcgcctaaggtcactatcagctagcaaatatttcttgtcaaaaatgctccact
gacgttccataaattcccctcggtatccaattagagtctcatattcactctcaatccaa
ataatctgcaccggatctggatcgtttcgcatgattgaacaagatggattgcacgcagg
ttctccggccgcttgggtggagaggctattcggctatgactgggcacaacagacaatcg
gctgctctgatgccgccgtgttccggctgtcagcgcaggggcgcccggttctttttgtc
aagaccgacctgtccggtgccctgaatgaactgcaggacgaggcagcgcggctatcgtg
gctggccacgacgggcgttccttgcgcagctgtgctcgacgttgtcactgaagcgggaa
gggactggctgctattgggcgaagtgccggggcaggatctcctgtcatctcaccttgct
cctgccgagaaagtatccatcatggctgatgcaatgcggcggctgcatacgcttgatcc
ggctacctgcccattcgaccaccaagcgaaacatcgcatcgagcgagcacgtactcgga
tggaagccggtcttgtcgatcaggatgatctggacgaagagcatcaggggctcgcgcca
gccgaactgttcgccaggctcaaggcgcgcatgcccgacggcgatgatctcgtcgtgac
ccatggcgatgcctgcttgccgaatatcatggtggaaaatggccgcttttctggattca
tcgactgtggccggctgggtgtggcggaccgctatcaggacatagcgttggctacccgt
gatattgctgaagagcttggcggcgaatgggctgaccgcttcctcgtgctttacggtat
cgccgctcccgattcgcagcgcatcgccttctatcgccttcttgacgagttcttctgag
cgggactctggggttcgaaatgaccgaccaagcgacgcccaacctgccatcacgagatt
tcgattccaccgccgccttctatgaaaggttgggcttcggaatcgttttccgggacgcc
ggctggatgatcctccagcgcggggatctcatgctggagttcttcgcccacgggatctc
tgcggaacaggcggtcgaaggtgccgatatcattacgacagcaacggccgacaagcaca
acgccacgatcctgagcgacaatatgatcgggcccggcgtccacatcaacggcgtcggc
ggcgactgcccaggcaagaccgagatgcaccgcgatatcttgctgcgttcggatatttt
cgtggagttcccgccacagacccggatgatccccgatcgttcaaacatttggcaataaa
gtttcttaagattgaatcctgttgccggtcttgcgatgattatcatataatttctgttg
aattacgttaagcatgtaataattaacatgtaatgcatgacgttatttatgagatgggt
ttttatgattagagtcccgcaattatacatttaatacgcgatagaaaacaaaatatagc
gcgcaaactaggataaattatcgcgcgcggtgtcatctatgttactagatcgggcctcc
tgtcaatgctggcggcggctctggtggtggttctggtggcggctctgagggtggtggct
ctgagggtggcggttctgagggtggcggctctgagggaggcggttccggtggtggctct
ggttccggtgattttgattatgaaaagatggcaaacgctaataagggggctatgaccga
aaatgccgatgaaaacgcgctacagtctgacgctaaaggcaaacttgattctgtcgcta
ctgattacggtgctgctatcgatggtttcattggtgacgtttccggccttgctaatggt
aatggtgctactggtgattttgctggctctaattcccaaatggctcaagtcggtgacgg
tgataatcacctttaatgaataatttccgtcaatatttaccttccctccctcaatccgg
ttgaatgtcgcccttttgtctttggcccaatacgcaaaccgcctctccccgcgcgttgg
ccgattcattaatgcagctggcacgacaggtttcccgactggaaagcgggcagtgagcg
caacgcaattaatgtgagttagctcactcattaggcaccccaggctttacactttatgc
ttccggctcgtatgttgtgtggaattgtgagcggataacaatttcacacaggaaacagc
tatgaccatgattacgccaagcttgcatgcctgcagcccacagatggttagagaggctt
acgcagcaggtctcatcaagacgatctacccgagcaataatctccaggaaatcaaatac
cttcccaagaaggttaaagatgcagtcaaaagattcaggactaactgcatcaagaacac
agagaaagatatatttctcaagatcagaagtactattccagtatggacgattcaaggct
tgcttcacaaaccaaggcaagtaatagagattggagtctctaaaaaggtagttcccact
gaatcaaaggccatggagtcaaagattcaaatagaggacctaacagaactcgccgtaaa
gactggcgaacagttcatacagagtctcttacgactcaatgacaagaagaaaatcttcg
tcaacatggtggagcacgacacacttgtctactccaaaaatatcaaagatacagtctca
gaagaccaaagggcaattgagacttttcaacaaagggtaatatccggaaacctcctcgg
attccattgcccagctatctgtcactttattgtgaagatagtggaaaaggaaggtggct
cctacaaatgccatcattgcgataaaggaaaggccatcgttgaagatgcctctgccgac
agtggtcccaaagatggacccccacccacgaggagcatcgtggaaaaagaagacgttcc
aaccacgtcttcaaagcaagtggattgatgtgatatctccactgacgtaagggatgacg
cacaatcccactatccttcgcaagacccttcctctatataaggaagttcatttcatttg
gagagaacacgggggactctagaggatccatggcgattcctttcatggaaaccgtcgtt
ggttttatgatagtgatgtacgtttttgagacgtatttggatctgaggcaacatactgc
tctcaagcttcccactctcccaaagactttggttggagtcattagccaagagaagtttg
agaaatctcgagcttacagtcttgacaaaagccattttcactttgttcatgagtttgtt
actatacttatggactctgcgattctgttctttgggatcttgccttggttttggaagat
atctggcggctttctaccaatggtgggactcgatccagagaatgaaatcctgcacactc
tttcattcttggctggtcttatgacatggtcacagatcactgatttgccattttctttg
tactcaactttcgtgatcgagtctcggcatgggttcaacaaacaaacaatatggatgtt
cattagggacatgatcaaaggaatactcctctctgtcatacctgcccctcctatcgttg
ccgcaattattgttatagttcagaaaggaggtccttacctcgccatctatctgtgggca
ttcatgtttatcctgtctctagtgatgatgactatataccctgttttgattgcacctct
tttcaacaagttcactcctcttcctgatggagacctccgggagaagattgagaaacttg
cttcttctctaaagtttcctctgaagaagctgtttgttgtcgatggatctacaaggtca
agccatagtaatgcttacatgtatggtttcttcaagaacaaaaggattgttctttatga
cacattgattcagcagtgccagaatgagaatgaaattgtggcggttattgcacacgagc
tgggacactggaagctgaatcacactacatactcgttcattgctgttcaaatccttgcc
ttcttgcaatttggaggatacactcttgtcagaaactccactgatctcttcaggagttt
tggttttgatacacaaccagttctcattggtttgatcatatttcagcacactgtaatac
cacttcaacacctagtaagctttgacctcaaccttgttagtcgagcgtttgagtttcag
gctgatgcttttgcagtgaatcttggttatgcaaaggatctacgtcctgccctagtgaa
gctacaggaagagaacttatcagcgatgaacacagacccattgtactcagcttatcact
actcacaccctcctcttgtagagaggcttcgagccattgatggagaagacaagaagaca
gattaacccctcgaatttccccgatcgttcaaacatttggcaataaagtttcttaagat
tgaatcctgttgccggtcttgcgatgattatcatataatttctgttgaattacgttaag
catgtaataattaacatgtaatgcatgacgttatttatgagatgggtttttatgattag
agtcccgcaattatacatttaatacgcgatagaaaacaaaatatagcgcgcaaactagg
ataaattatcgcgcgcggtgtcatctatgttactagatcgggaattcactggccgtcgt
tttacaacgtcgtgactgggaaaaccctggcgttacccaacttaatcgccttgcagcac
atccccctttcgccagctggcgtaatagcgaagaggcccgcaccgatcgcccttcccaa
cagttgcgcagcctgaatggcgcccgctcctttcgctttcttcccttcctttctcgcca
cgttcgccggctttccccgtcaagctctaaatcgggggctccctttagggttccgattt
agtgctttacggcacctcgaccccaaaaaacttgatttgggtgatggttcacgtagtgg
gccatcgccctgatagacggtttttcgccctttgacgttggagtccacgttctttaata
gtggactcttgttccaaactggaacaacactcaaccctatctcgggctattcttttgat
ttataagggattttgccgatttcggaaccaccatcaaacaggattttcgcctgctgggg
caaaccagcgtggaccgcttgctgcaactctctcagggccaggcggtgaagggcaatca
gctgttgcccgtctcactggtgaaaagaaaaaccaccccagtacattaaaaacgtccgc
aatgtgttattaagttgtctaagcgtcaatttgtttacaccacaatatatcctgcca

SEQ ID NO:47 is the nucleic acid sequence of pBI121-BnCPP. Italicized sequences are the right and left border repeats. Underlined sequence is the ³⁵S promoter. Sequence in bold is the BnCPP antisense sequence.

SEQ ID NO:48

gtttacccgccaatatatcctgtcaaacactgatagtttaaactgaaggcgggaaacga
caatctgatcatgagcggagaattaagggagtcacgttatgacccccgccgatgacgcg
ggacaagccgttttacgtttggaactgacagaaccgcaacgttgaaggagccactcagc
cgcgggtttctggagtttaatgagctaagcacatacgtcagaaaccattattgcgcgtt
caaaagtcgcctaaggtcactatcagctagcaaatatttcttgtcaaaaatgctccact
gacgttccataaattcccctcggtatccaattagagtctcatattcactctcaatccaa
ataatctgcaccggatctggatcgtttcgcatgattgaacaagatggattgcacgcagg
ttctccggccgcttgggtggagaggctattcggctatgactgggcacaacagacaatcg
gctgctctgatgccgccgtgttccggctgtcagcgcaggggcgcccggttctttttgtc
aagaccgacctgtccggtgccctgaatgaactgcaggacgaggcagcgcggctatcgtg
gctggccacgacgggcgttccttgcgcagctgtgctcgacgttgtcactgaagcgggaa
gggactggctgctattgggcgaagtgccggggcaggatctcctgtcatctcaccttgct
cctgccgagaaagtatccatcatggctgatgcaatgcggcggctgcatacgcttgatcc
ggctacctgcccattcgaccaccaagcgaaacatcgcatcgagcgagcacgtactcgga
tggaagccggtcttgtcgatcaggatgatctggacgaagagcatcaggggctcgcgcca
gccgaactgttcgccaggctcaaggcgcgcatgcccgacggcgatgatctcgtcgtgac
ccatggcgatgcctgcttgccgaatatcatggtggaaaatggccgcttttctggattca
tcgactgtggccggctgggtgtggcggaccgctatcaggacatagcgttggctacccgt
gatattgctgaagagcttggcggcgaatgggctgaccgcttcctcgtgctttacggtat
cgccgctcccgattcgcagcgcatcgccttctatcgccttcttgacgagttcttctgag
cgggactctggggttcgaaatgaccgaccaagcgacgcccaacctgccatcacgagatt
tcgattccaccgccgccttctatgaaaggttgggcttcggaatcgttttccgggacgcc
ggctggatgatcctccagcgcggggatctcatgctggagttcttcgcccacgggatctc
tgcggaacaggcggtcgaaggtgccgatatcattacgacagcaacggccgacaagcaca
acgccacgatcctgagcgacaatatgatcgggcccggcgtccacatcaacggcgtcggc
ggcgactgcccaggcaagaccgagatgcaccgcgatatcttgctgcgttcggatatttt
cgtggagttcccgccacagacccggatgatccccgatcgttcaaacatttggcaataaa
gtttcttaagattgaatcctgttgccggtcttgcgatgattatcatataatttctgttg
aattacgttaagcatgtaataattaacatgtaatgcatgacgttatttatgagatgggt
ttttatgattagagtcccgcaattatacatttaatacgcgatagaaaacaaaatatagc
gcgcaaactaggataaattatcgcgcgcggtgtcatctatgttactagatcgggcctcc
tgtcaatgctggcggcggctctggtggtggttctggtggcggctctgagggtggtggct
ctgagggtggcggttctgagggtggcggctctgagggaggcggttccggtggtggctct
ggttccggtgattttgattatgaaaagatggcaaacgctaataagggggctatgaccga
aaatgccgatgaaaacgcgctacagtctgacgctaaaggcaaacttgattctgtcgcta
ctgattacggtgctgctatcgatggtttcattggtgacgtttccggccttgctaatggt
aatggtgctactggtgattttgctggctctaattcccaaatggctcaagtcggtgacgg
tgataattcacctttaatgaataatttccgtcaatatttaccttccctccctcaatcgg
ttgaatgtcgcccttttgtctttggcccaatacgcaaaccgcctctccccgcgcgttgg
ccgattcattaatgcagctggcacgacaggtttcccgactggaaagcgggcagtgagcg
caacgcaattaatgtgagttagctcactcattaggcaccccaggctttacactttatgc
ttccggctcgtatgttgtgtggaattgtgagcggataacaatttcacacaggaaacagc
tatgaccatgattacgccaagcttgcatgcctgcagcccacagatggttagagaggctt
acgcagcaggtctcatcaagacgatctacccgagcaataatctccaggaaatcaaatac
cttcccaagaaggttaaagatgcagtcaaaagattcaggactaactgcatcaagaacac
agagaaagatatatttctcaagatcagaagtactattccagtatggacgattcaaggct
tgcttcacaaaccaaggcaagtaatagagattggagtctctaaaaaggtagttcccact
gaatcaaaggccatggagtcaaagattcaaatagaggacctaacagaactcgccgtaaa
gactggcgaacagttcatacagagtctcttacgactcaatgacaagaagaaaatcttcg
tcaacatggtggagcacgacacacttgtctactccaaaaatatcaaagatacagtctca
gaagaccaaagggcaattgagacttttcaacaaagggtaatatccggaaacctcctcgg
attccattgcccagctatctgtcactttattgtgaagatagtggaaaaggaaggtggct
cctacaaatgccatcattgcgataaaggaaaggccatcgttgaagatgcctctgccgac
agtggtcccaaagatggacccccacccacgaggagcatcgtggaaaaagaagacgttcc
aaccacgtcttcaaagcaagtggattgatgtgatatctccactgacgtaagggatgacg
cacaatcccactatccttcgcaagacccttcctctatataaggaagttcatttcatttg
gagagaacacgggggactctagaccagtgtcccagctcgtgtgcaataaccgccacaat
ttcattctcattctggcactgctgaatcaatgtgtcataaagaacaatccttttgttct
tgaagaaaccatacatgtaagcattactatggcttgaccttgtagatccatcgacaaca
aacagcttcttcagaggaaactttagagaagaagcaagtttctcaatcttctcccggag
gtctccatcaggaagaggagtgaacttgttgaaaagaggtgcaatcaaaacagggtata
tagtcatcatcactagagacaggataaacatgaatgcccacagatagatggcgaggtaa
ggacctcctttctgaactataacastasttgcggcaacgataggaggggcaggtatgac
agagaggagtattcctttgatcatgtccctaatgaacatccatattgtttgtttgttga
acccatgccgagactcgatcacgaaagttgagtacaaagaaaatggcaaatcagtgatc
tgtgaccatgtcataagaccagccaagaatgaaagagtgtgcaggatttcattctctgg
atcgagtcccaccattggtagaaggatccccATCTACCCGCTTCGCGTCGGCATCCGGT
CAGTGGCAGTGAAGGGCGAACAGTTCCTGATTAACCACAAACCGTTCTACTTTACTGGC
TTTGGTCGTCATGAAGATGCGGACTTGCGTGGCAAAGGATTCGATAACGTGCTGATGGT
GCACGACCACGCATTAATGGACTGGATTGGGGCCAACTCCTACCGTACCTCGCATTACC
CTTACGCTGAAGAGATGCTCGACTGGGCAGATGAACATGGCATCGTGGTGATTGATGAA
ACTGCTGCTGTCGGCTTTTCGCTCTCTTTAGGCATTGGTTTCGAAGCGGGCAACAAGCC
GAAAGAACTGTACAGCGAAGAGGCAGTCAACGGGGAAACTCAGCAAGCGCACTTACAGG
CGATTAAAGAGCTGATAGCGCGTGACAAAAACCACCCAAGCGTGGTGATGTGGAGTATT
GCCAACGAACCGGATACCCGTCCGCAAGGTGCACGGGAATATTTCGCGCCACTGGCGGA
AGCAACGCGTAAACTCGACCCGACGCGTCCGATCACCTGCGTCAATGTAATGTTCTGCG
ACGCTCACACCGATACCATCAGCGATCTCTTTGATGTGCTGTGCCTGAACCGTTATTAC
GGATGGTATGTCCAAAGCGGCGATTTGGAAACGGCAGAGAAGGTACTGGAAAAAGAACT
TCTGGCCTGGCAGGAGAAACTGTACACCGACATGTGGAGTGAAGAGTATCAGTGTGCAT
GGCTGGATATGTATCACCGCGTCTTTGATCGCGTCAGCGCCGTCGTCGGTGAACAGGTA
TGGAATTTCGCCGATTTTGCGACCTCGCAAGGCATATTGCGCGTTGGCGGTAACAAGAA
AGGGATCTTCACTCGCGACCGCAAACCGAAGTCGGCGGCTTTTCTGCTGCAAAAACGCT
GGACTGGCATGAACTTCGGTGAAAAACCGCAGCAGGGAGGCAAACAATGAatcaacaac
tctcctggcgcaccatcgtcggctacagcctcgggaattgctaccgagctcttctacca
atggtgggactcgatccagagaatgaaatcctgcacactctttcattcttggctggtct
tatgacatggtcacagatcactgatttgccattttctttgtactcaactttcgtgatcg
agtctcggcatgggttcaacaaacaaacaatatggatgttcattagggacatgatcaaa
ggaatactcctctctgtcatacctgcccctcctatcgttgccgcaattattgttatagt
tcagaaaggaggtccttacctcgccatctatctgtgggcattcatgtttatcctgtctc
tagtgatgatgactatataccctgttttgattgcacctcttttcaacaagttcactcct
cttcctgatggagacctccgggagaagattgagaaacttgcttcttctctaaagtttcc
tctgaagaagctgtttgttgtcgatggatctacaaggtcaagccatagtaatgcttaca
tgtatggtttcttcaagaacaaaaggattgttctttatgacacattgattcagcagtgc
cagaatgagaatgaaattgtggcggttattgcacacgagctgggacactgggagctcga
atttccccgatcgttcaaacatttggcaataaagtttcttaagattgaatcctgttgcc
ggtcttgcgatgattatcatataatttctgttgaattacgttaagcatgtaataattaa
catgtaatgcatgacgttatttatgagatgggtttttatgattagagtcccgcaattat
acatttaatacgcgatagaaaacaaaatatagcgcgcaaactaggataaattatcgcgc
gcggtgtcatctatgttactagatcgggaattcactggccgtcgttttacaacgtcgtg
actgggaaaaccctggcgttacccaacttaatcgccttgcagcacatccccctttcgcc
agctggcgtaatagcgaagaggcccgcaccgatcgcccttcccaacagttgcgcagcct
gaatggcgcccgctcctttcgctttcttcccttcctttctcgccacgttcgccggcttt
ccccgtcaagctctaaatcgggggctccctttagggttccgatttagtgctttacggca
cctcgaccccaaaaaacttgatttgggtgatggttcacgtagtgggccatcgccctgat
agacggtttttcgccctttgacgttggagtccacgttctttaatagtggactcttgttc
caaactggaacaacactcaaccctatctcgggctattcttttgatttataagggatttt
gccgatttcggaaccaccatcaaacaggattttcgcctgctggggcaaaccagcgtgga
ccgcttgctgcaactctctcagggccaggcggtgaagggcaatcagctgttgcccgtct
cactggtgaaaagaaaaaccaccccagtacattaaaaacgtccgcaatgtgttattaag
ttgtctaagcgtcaatttgtttacaccacaatatatcctgcca

SEQ ID NO:48 is the nucleic acid sequence of pBI121-HP-BnCPP. Italicized sequences are the right and left border repeats. Underlined sequence is the ³⁵S promoter. Sequence in bold is the BnCPP antisense sequence, bold and underlined sequence is the BnCPP sense fragment and upper case indicates the truncated GUS fragment.

SEQ ID NO:49

gtttacccgccaatatatcctgtcaaacactgatagtttaaactgaaggcgggaaacga
caatctgatcatgagcggagaattaagggagtcacgttatgacccccgccgatgacgcg
ggacaagccgttttacgtttggaactgacagaaccgcaacgttgaaggagccactcagc
cgcgggtttctggagtttaatgagctaagcacatacgtcagaaaccattattgcgcgtt
caaaagtcgcctaaggtcactatcagctagcaaatatttcttgtcaaaaatgctccact
gacgttccataaattcccctcggtatccaattagagtctcatattcactctcaatccaa
ataatctgcaccggatctggatcgtttcgcatgattgaacaagatggattgcacgcagg
ttctccggccgcttgggtggagaggctattcggctatgactgggcacaacagacaatcg
gctgctctgatgccgccgtgttccggctgtcagcgcaggggcgcccggttctttttgtc
aagaccgacctgtccggtgccctgaatgaactgcaggacgaggcagcgcggctatcgtg
gctggccacgacgggcgttccttgcgcagctgtgctcgacgttgtcactgaagcgggaa
gggactggctgctattgggcgaagtgccggggcaggatctcctgtcatctcaccttgct
cctgccgagaaagtatccatcatggctgatgcaatgcggcggctgcatacgcttgatcc
ggctacctgcccattcgaccaccaagcgaaacatcgcatcgagcgagcacgtactcgga
tggaagccggtcttgtcgatcaggatgatctggacgaagagcatcaggggctcgcgcca
gccgaactgttcgccaggctcaaggcgcgcatgcccgacggcgatgatctcgtcgtgac
ccatggcgatgcctgcttgccgaatatcatggtggaaaatggccgcttttctggattca
tcgactgtggccggctgggtgtggcggaccgctatcaggacatagcgttggctacccgt
gatattgctgaagagcttggcggcgaatgggctgaccgcttcctcgtgctttacggtat
cgccgctcccgattcgcagcgcatcgccttctatcgccttcttgacgagttcttctgag
cgggactctggggttcgaaatgaccgaccaagcgacgcccaacctgccatcacgagatt
tcgattccaccgccgccttctatgaaaggttgggcttcggaatcgttttccgggacgcc
ggctggatgatcctccagcgcggggatctcatgctggagttcttcgcccacgggatctc
tgcggaacaggcggtcgaaggtgccgatatcattacgacagcaacggccgacaagcaca
acgccacgatcctgagcgacaatatgatcgggcccggcgtccacatcaacggcgtcggc
ggcgactgcccaggcaagaccgagatgcaccgcgatatcttgctgcgttcggatatttt
cgtggagttcccgccacagacccggatgatccccgatcgttcaaacatttggcaataaa
gtttcttaagattgaatcctgttgccggtcttgcgatgattatcatataatttctgttg
aattacgttaagcatgtaataattaacatgtaatgcatgacgttatttatgagatgggt
ttttatgattagagtcccgcaattatacatttaatacgcgatagaaaacaaaatatagc
gcgcaaactaggataaattatcgcgcgcggtgtcatctatgttactagatcgggcctcc
tgtcaatgctggcggcggctctggtggtggttctggtggcggctctgagggtggtggct
ctgagggtggcggttctgagggtggcggctctgagggaggcggttccggtggtggctct
ggttccggtgattttgattatgaaaagatggcaaacgctaataagggggctatgaccga
aaatgccgatgaaaacgcgctacagtctgacgctaaaggcaaacttgattctgtcgcta
ctgattacggtgctgctatcgatggtttcattggtgacgtttccggccttgctaatggt
aatggtgctactggtgattttgctggctctaattcccaaatggctcaagtcggtgacgg
tgataattcacctttaatgaataatttccgtcaatatttaccttccctccctcaatcgg
ttgaatgtcgcccttttgtctttggcccaatacgcaaaccgcctctccccgcgcgttgg
ccgattcattaatgcagctggcacgacaggtttcccgactggaaagcgggcagtgagcg
caacgcaattaatgtgagttagctcactcattaggcaccccaggctttacactttatgc
ttccggctcgtatgttgtgtggaattgtgagcggataacaatttcacacaggaaacagc
tatgaccatgattacgccaagcttgcatgcctgcagcccacagatggttagagaggctt
acgcagcaggtctcatcaagacgatctacccgagcaataatctccaggaaatcaaatac
cttcccaagaaggttaaagatgcagtcaaaagattcaggactaactgcatcaagaacac
agagaaagatatatttctcaagatcagaagtactattccagtatggacgattcaaggct
tgcttcacaaaccaaggcaagtaatagagattggagtctctaaaaaggtagttcccact
gaatcaaaggccatggagtcaaagattcaaatagaggacctaacagaactcgccgtaaa
gactggcgaacagttcatacagagtctcttacgactcaatgacaagaagaaaatcttcg
tcaacatggtggagcacgacacacttgtctactccaaaaatatcaaagatacagtctca
gaagaccaaagggcaattgagacttttcaacaaagggtaatatccggaaacctcctcgg
attccattgcccagctatctgtcactttattgtgaagatagtggaaaaggaaggtggct
cctacaaatgccatcattgcgataaaggaaaggccatcgttgaagatgcctctgccgac
agtggtcccaaagatggacccccacccacgaggagcatcgtggaaaaagaagacgttcc
aaccacgtcttcaaagcaagtggattgatgtgatatctccactgacgtaagggatgacg
cacaatcccactatccttcgcaagacccttcctctatataaggaagttcatttcatttg
gagagaacacgggggactctagaggatccttaatctgtcttcttgtcttctccatcaat
ggctcgaagcctctctacaagaggagggtgtgagtagtgataagctgagtacaatgggt
ctgtgttcatcgctgataagttctcttcctgtagcttcactagggcaggacgtagatcc
tttgcataaccaagattcactgcaaaagcatcagcctgaaactcaaacgctcgactaac
aaggttgaggtcaaagcttactaggtgttgaagtggtattacagtgtgctgaaatatga
tcaaaccaatgagaactggttgtgtatcaaaaccaaaactcctgaagagatcagtggag
tttctgacaagagtgtatcctccaaattgcaagaaggcaaggatttgaacagcaatgaa
cgagtatgtagtgtgattcagcttccagtgtcccagctcgtgtgcaataaccgccacaa
tttcattctcattctggcactgctgaatcaatgtgtcataaagaacaatccttttgttc
ttgaagaaaccatacatgtaagcattactatggcttgaccttgtagatccatcgacaac
aaacagcttcttcagaggaaactttagagaagaagcaagtttctcaatcttctcccgga
ggtctccatcaggaagaggagtgaacttgttgaaaagaggtgcaatcaaaacagggtat
atagtcatcatcactagagacaggataaacatgaatgcccacagatagatggcgaggta
aggacctcctttctgaactataacaataattgcggcaacgataggaggggcaggtatga
cagagaggagtattcctttgatcatgbccctaatgaacatccatattgtttgtttgttg
aacccatgccgagactcgatcacgaaagttgagtacaaagaaaatggcaaatcagtgat
ctgtgaccatgtcataagaccagccaagaatgaaagagtgtgcaggatttcattctctg
gatcgagtcccaccattggtagaaagccgccagatatcttccaaaaccaaggcaagatc
ccaaagaacagaatcgcagagtccataagtatagtaacaaactcatgaacaaagtgaaa
atggcttttgtcaagactgtaagctcgagatttctcaaacttctcttggctaatgactc
caaccaaagtctttgggagagtgggaagcttgagagcagtatgttgcctcagatccaaa
tacgtctcaaaaacgtacatcactatcataaaaccaacgacggtttccatgaaaggaat
cgccatcccctcgaatttccccgatcgttcaaacatttggcaataaagtttcttaagat
tgaatcctgttgccggtcttgcgatgattatcatataatttctgttgaattacgttaag
catgtaataattaacatgtaatgcatgacgttatttatgagatgggtttttatgattag
agtcccgcaattatacatttaatacgcgatagaaaacaaaatatagcgcgcaaactagg
ataaattatcgcgcgcggtgtcatctatgttactagatcgggaattcactggccgtcgt
tttacaacgtcgtgactgggaaaaccctggcgttacccaacttaatcgccttgcagcac
atccccctttcgccagctggcgtaatagcgaagaggcccgcaccgatcgcccttcccaa
cagttgcgcagcctgaatggcgcccgctcctttcgctttcttcccttcctttctcgcca
cgttcgccggctttccccgtcaagctctaaatcgggggctccctttagggttccgattt
agtgctttacggcacctcgaccccaaaaaacttgatttgggtgatggttcacgtagtgg
gccatcgccctgatagacggtttttcgccctttgacgttggagtccacgttctttaata
gtggactcttgttccaaactggaacaacactcaaccctatctcgggctattcttttgat
ttataagggattttgccgatttcggaaccaccatcaaacaggattttcgcctgctgggg
caaaccagcgtggaccgcttgctgcaactctctcagggccaggcggtgaagggcaatca
gctgttgcccgtctcactggtgaaaagaaaaaccaccccagtacattaaaaacgtccgc
aatgtgttattaagttgtctaagcgtcaatttgtttacaccacaatatatcctgcca

SEQ ID NO:49 is the nucleic acid sequence of pBI121-antisense-BnCPP. Italicized sequences are the right and left border repeats. Underlined sequence is the ³⁵S promoter. Sequence in bold is the BnCPP antisense sequence.

SEQ ID NO:50

gtttacccgccaatatatcctgtcaaacactgatagtttaaactgaaggcgggaaacga
caatctgatcatgagcggagaattaagggagtcacgttatgacccccgccgatgacgcg
ggacaagccgttttacgtttggaactgacagaaccgcaacgttgaaggagccactcagc
cgcgggtttctggagtttaatgagctaagcacatacgtcagaaaccattattgcgcgtt
caaaagtcgcctaaggtcactatcagctagcaaatatttcttgtcaaaaatgctccact
gacgttccataaattcccctcggtatccaattagagtctcatattcactctcaatccaa
ataatctgcaccggatctggatcgtttcgcatgattgaacaagatggattgcacgcagg
ttctccggccgcttgggtggagaggctattcggctatgactgggcacaacagacaatcg
gctgctctgatgccgccgtgttccggctgtcagcgcaggggcgcccggttctttttgtc
aagaccgacctgtccggtgccctgaatgaactgcaggacgaggcagcgcggctatcgtg
gctggccacgacgggcgttccttgcgcagctgtgctcgacgttgtcactgaagcgggaa
gggactggctgctattgggcgaagtgccggggcaggatctcctgtcatctcaccttgct
cctgccgagaaagtatccatcatggctgatgcaatgcggcggctgcatacgcttgatcc
ggctacctgcccattcgaccaccaagcgaaacatcgcatcgagcgagcacgtactcgga
tggaagccggtcttgtcgatcaggatgatctggacgaagagcatcaggggctcgcgcca
gccgaactgttcgccaggctcaaggcgcgcatgcccgacggcgatgatctcgtcgtgac
ccatggcgatgcctgcttgccgaatatcatggtggaaaatggccgcttttctggattca
tcgactgtggccggctgggtgtggcggaccgctatcaggacatagcgttggctacccgt
gatattgctgaagagcttggcggcgaatgggctgaccgcttcctcgtgctttacggtat
cgccgctcccgattcgcagcgcatcgccttctatcgccttcttgacgagttcttctgag
cgggactctggggttcgaaatgaccgaccaagcgacgcccaacctgccatcacgagatt
tcgattccaccgccgccttctatgaaaggttgggcttcggaatcgttttccgggacgcc
ggctggatgatcctccagcgcggggatctcatgctggagttcttcgcccacgggatctc
tgcggaacaggcggtcgaaggtgccgatatcattacgacagcaacggccgacaagcaca
acgccacgatcctgagcgacaatatgatcgggcccggcgtccacatcaacggcgtcggc
ggcgactgcccaggcaagaccgagatgcaccgcgatatcttgctgcgttcggatatttt
cgtggagttcccgccacagacccggatgatccccgatcgttcaaacatttggcaataaa
gtttcttaagattgaatcctgttgccggtcttgcgatgattatcatataatttctgttg
aattacgttaagcatgtaataattaacatgtaatgcatgacgttatttatgagatgggt
ttttatgattagagtcccgcaattatacatttaatacgcgatagaaaacaaaatatagc
gcgcaaactaggataaattatcgcgcgcggtgtcatctatgttactagatcgggcctcc
tgtcaatgctggcggcggctctggtggtggttctggtggcggctctgagggtggtggct
ctgagggtggcggttctgagggtggcggctctgagggaggcggttccggtggtggctct
ggttccggtgattttgattatgaaaagatggcaaacgctaataagggggctatgaccga
aaatgccgatgaaaacgcgctacagtctgacgctaaaggcaaacttgattctgtcgcta
ctgattacggtgctgctatcgatggtttcattggtgacgtttccggccttgctaatggt
aatggtgctactggtgattttgctggctctaattcccaaatggctcaagtcggtgacgg
tgataattcacctttaatgaataatttccgtcaatatttaccttccctccctcaatcgg
ttgaatgtcgcccttttgtctttggcccaatacgcaaaccgcctctccccgcgcgttgg
ccgattcattaatgcagctggcacgacaggtttcccgactggaaagcgggcagtgagcg
caacgcaattaatgtgagttagctcactcattaggcaccccaggctttacactttatgc
ttccggctcgtatgttgtgtggaattgtgagcggataacaatttcacacaggaaacagc
tatgaccatgattacgccaagcttgcatgcctgcagggagccatagatgcaattcaatc
aaactgaaatttctgcaagaatctcaaacacggagatctcaaagtttgaaagaaaattt
atttcttcgactcaaaacaaacttacgaaatttaggtagaacttatatacattatattg
taattttttgtaacaaaatgtttttattattattatagaattttactggttaaattaaa
aatgaatagaaaaggtgaattaagaggagagaggaggtaaacattttcttctatttttt
catattttcaggataaattattgtaaaagtttacaagatttccatttgactagtgtaaa
tgaggaatattctctagtaagatcattatttcatctacttcttttatcttctaccagta
gaggaataaacaatatttagctcctttgtaaatacaaattaattttccttcttgacatc
attcaattttaattttacgtataaaataaaagatcatacctattagaacgattaaggag
aaatacaattcgaatgagaaggatgtgccgtttgttataataaacagccacacgacgta
aacgtaaaatgaccacatgatgggccaatagacatggaccgactactaataatagtaag
ttacattttaggatggaataaatatcataccgacatcagttttgaaagaaaagggaaaa
aaagaaaaaataaataaaagatatactaccgacatgagttccaaaaagcaaaaaaaaag
atcaagccgacacagacacgcgtagagagcaaaatgactttgacgtcacaccacgaaaa
cagacgcttcatacgtgtccctttatctctctcagtctctctataaacttagtgagacc
ctcctctgttttactcacaaatatgcaaactagaaaacaatcatcaggaataaagggtt
tgattacttctattggaaaggactctagaggatccatggcgattcctttcatggaaacc
gtcgttggttttatgatagtgatgtacgtttttgagacgtatttggatctgaggcaaca
tactgctctcaagcttcccactctcccaaagactttggttggagtcattagccaagaga
agtttgagaaatctcgagcttacagtcttgacaaaagccattttcactttgttcatgag
tttgttactatacttatggactctgcgattctgttctttgggatcttgccttggttttg
gaagatatctggcggctttctaccaatggtgggactcgatccagagaatgaaatcctgc
acactctttcattcttggctggtcttatgacatggtcacagatcactgatttgccattt
tctttgtactcaactttcgtgatcgagtctcggcatgggttcaacaaacaaacaatatg
gatgttcattagggacatgatcaaaggaatactcctctctgtcatacctgcccctccta
tcgttgccgcaattattgttatagttcagaaaggaggtccttacctcgccatctatctg
tgggcattcatgtttatcctgtctctagtgatgatgactatataccctgttttgattgc
acctcttttcaacaagttcactcctcttcctgatggagacctccgggagaagattgaga
aacttgcttcttctctaaagtttcctctgaagaagctgtttgttgtcgatggatctaca
aggtcaagccatagtaatgcttacatgtatggtttcttcaagaacaaaaggattgttct
ttatgacacattgattcagcagtgccagaatgagaatgaaattgtggcggttattgcac
acgagctgggacactggaagctgaatcacactacatactcgttcattgctgttcaaatc
cttgccttcttgcaatttggaggatacactcttgtcagaaactccactgatctcttcag
gagttttggttttgatacacaaccagttctcattggtttgatcatatttcagcacactg
taataccacttcaacacctagtaagctttgacctcaaccttgttagtcgagcgtttgag
tttcaggctgatgcttttgcagtgaatcttggttatgcaaaggatctacgtcctgccct
agtgaagctacaggaagagaacttatcagcgatgaacacagacccattgtactcagctt
atcactactcacaccctcctcttgtagagaggcttcgagccattgatggagaagacaag
aagacagattaacccctcgaatttccccgatcgttcaaacatttggcaataaagtttct
taagattgaatcctgttgccggtcttgcgatgattatcatataatttctgttgaattac
gttaagcatgtaataattaacatgtaatgcatgacgttatttatgagatgggtttttat
gattagagtcccgcaattatacatttaatacgcgatagaaaacaaaatatagcgcgcaa
actaggataaattatcgcgcgcggtgtcatctatgttactagatcgggaattcactggc
cgtcgttttacaacgtcgtgactgggaaaaccctggcgttacccaacttaatcgccttg
cagcacatccccctttcgccagctggcgtaatagcgaagaggcccgcaccgatcgccct
tcccaacagttgcgcagcctgaatggcgcccgctcctttcgctttcttcccttcctttc
tcgccacgttcgccggctttccccgtcaagctctaaatcgggggctccctttagggttc
cgatttagtgctttacggcacctcgaccccaaaaaacttgatttgggtgatggttcacg
tagtgggccatcgccctgatagacggtttttcgccctttgacgttggagtccacgttct
ttaatagtggactcttgttccaaactggaacaacactcaaccctatctcgggctattct
tttgatttataagggattttgccgatttcggaaccaccatcaaacaggattttcgcctg
ctggggcaaaccagcgtggaccgcttgctgcaactctctcagggccaggcggtgaaggg
caatcagctgttgcccgtctcactggtgaaaagaaaaaccaccccagtacattaaaaac
gtccgcaatgtgttattaagttgtctaagcgtcaatttgtttacaccacaatatatcct
gcca

SEQ ID NO:50 is the nucleic acid sequence of pRD29A-BnCPP. Italicized sequences are the right and left border repeats. Underlined sequence is the RD29A promoter. Sequence in bold is the BnCPP sense sequence.

SEQ ID NO:51
gtttacccgccaatatatcctgtcaaacactgatagtttaaactgaaggcgggaaacga
caatctgatcatgagcggagaattaagggagtcacgttatgacccccgccgatgacgcg
ggacaagccgttttacgtttggaactgacagaaccgcaacgttgaaggagccactcagc
cgcgggtttctggagtttaatgagctaagcacatacgtcagaaaccattattgcgcgtt
caaaagtcgcctaaggtcactatcagctagcaaatatttcttgtcaaaaatgctccact
gacgttccataaattcccctcggtatccaattagagtctcatattcactctcaatccaa
ataatctgcaccggatctggatcgtttcgcatgattgaacaagatggattgcacgcagg
ttctccggccgcttgggtggagaggctattcggctatgactgggcacaacagacaatcg
gctgctctgatgccgccgtgttccggctgtcagcgcaggggcgcccggttctttttgtc
aagaccgacctgtccggtgccctgaatgaactgcaggacgaggcagcgcggctatcgtg
gctggccacgacgggcgttccttgcgcagctgtgctcgacgttgtcactgaagcgggaa
gggactggctgctattgggcgaagtgccggggcaggatctcctgtcatctcaccttgct
cctgccgagaaagtatccatcatggctgatgcaatgcggcggctgcatacgcttgatcc
ggctacctgcccattcgaccaccaagcgaaacatcgcatcgagcgagcacgtactcgga
tggaagccggtcttgtcgatcaggatgatctggacgaagagcatcaggggctcgcgcca
gccgaactgttcgccaggctcaaggcgcgcatgcccgacggcgatgatctcgtcgtgac
ccatggcgatgcctgcttgccgaatatcatggtggaaaatggccgcttttctggattca
tcgactgtggccggctgggtgtggcggaccgctatcaggacatagcgttggctacccgt
gatattgctgaagagcttggcggcgaatgggctgaccgcttcctcgtgctttacggtat
cgccgctcccgattcgcagcgcatcgccttctatcgccttcttgacgagttcttctgag
cgggactctggggttcgaaatgaccgaccaagcgacgcccaacctgccatcacgagatt
tcgattccaccgccgccttctatgaaaggttgggcttcggaatcgttttccgggacgcc
ggctggatgatcctccagcgcggggatctcatgctggagttcttcgcccacgggatctc
tgcggaacaggcggtcgaaggtgccgatatcattacgacagcaacggccgacaagcaca
acgccacgatcctgagcgacaatatgatcgggcccggcgtccacatcaacggcgtcggc
ggcgactgcccaggcaagaccgagatgcaccgcgatatcttgctgcgttcggatatttt
cgtggagttcccgccacagacccggatgatccccgatcgttcaaacatttggcaataaa
gtttcttaagattgaatcctgttgccggtcttgcgatgattatcatataatttctgttg
aattacgttaagcatgtaataattaacatgtaatgcatgacgttatttatgagatgggt
ttttatgattagagtcccgcaattatacatttaatacgcgatagaaaacaaaatatagc
gcgcaaactaggataaattatcgcgcgcggtgtcatctatgttactagatcgggcctcc
tgtcaatgctggcggcggctctggtggtggttctggtggcggctctgagggtggtggct
ctgagggtggcggttctgagggtggcggctctgagggaggcggttccggtggtggctct
ggttccggtgattttgattatgaaaagatggcaaacgctaataagggggctatgaccga
aaatgccgatgaaaacgcgctacagtctgacgctaaaggcaaacttgattctgtcgcta
ctgattacggtgctgctatcgatggtttcattggtgacgtttccggccttgctaatggt
aatggtgctactggtgattttgctggctctaattcccaaatggctcaagtcggtgacgg
tgataattcacctttaatgaataatttccgtcaatatttaccttccctccctcaatcgg
ttgaatgtcgcccttttgtctttggcccaatacgcaaaccgcctctccccgcgcgttgg
ccgattcattaatgcagctggcacgacaggtttcccgactggaaagcgggcagtgagcg
caacgcaattaatgtgagttagctcactcattaggcaccccaggctttacactttatgc
ttccggctcgtatgttgtgtggaattgtgagcggataacaatttcacacaggaaacagc
tatgaccatgattacgccaagcttgcatgcctgcagggagccatagatgcaattcaatc
aaactgaaatttctgcaagaatctcaaacacggagatctcaaagtttgaaagaaaattt
atttcttcgactcaaaacaaacttacgaaatttaggtagaacttatatacattatattg
taattttttgtaacaaaatgtttttattattattatagaattttactggttaaattaaa
aatgaatagaaaaggtgaattaagaggagagaggaggtaaacattttcttctatttttt
catattttcaggataaattattgtaaaagtttacaagatttccatttgactagtgtaaa
tgaggaatattctctagtaagatcattatttcatctacttcttttatcttctaccagta
gaggaataaacaatatttagctcctttgtaaatacaaattaattttccttcttgacatc
attcaattttaattttacgtataaaataaaagatcatacctattagaacgattaaggag
aaatacaattcgaatgagaaggatgtgccgtttgttataataaacagccacacgacgta
aacgtaaaatgaccacatgatgggccaatagacatggaccgactactaataatagtaag
ttacattttaggatggaataaatatcataccgacatcagttttgaaagaaaagggaaaa
aaagaaaaaataaataaaagatatactaccgacatgagttccaaaaagcaaaaaaaaag
atcaagccgacacagacacgcgtagagagcaaaatgactttgacgtcacaccacgaaaa
cagacgcttcatacgtgtccctttatctctctcagtctctctataaacttagtgagacc
ctcctctgttttactcacaaatatgcaaactagaaaacaatcatcaggaataaagggtt
tgattacttctattggaaaggactctagaccagtgtcccagctcgtgtgcaataaccgc
cacaatttcattctcattctggcactgctgaatcaatgtgtcataaagaacaatccttt
tgttcttgaagaaaccatacatgtaagcattactatggcttgaccttgtagatccatcg
acaacaaacagcttcttcagaggaaactttagagaagaagcaagtttctcaatcttctc
ccggaggtctccatcaggaagaggagtgaacttgttgaaaagaggtgcaatcaaaacag
ggtatatagtcatcatcactagagacaggataaacatgaatgcccacagatagatggcg
aggtaaggacctcctttctgaactataacaataattgcggcaacgataggaggggcagg
tatgacagagaggagtattcctttgatcatgtccctaatgaacatccatattgtttgtt
tgttgaacccatgccgagactcgatcacgaaagttgagtacaaagaaaatggcaaatca
gtgatctgtgaccatgtcataagaccagccaagaatgaaagagtgtgcaggatttcatt
ctctggatcgagtcccaccattggtagaaggatccccATCTACCCGCTTCGCGTCGGCA
TCCGGTCAGTGGCAGTGAAGGGCGAACAGTTCCTGATTAACCACAAACCGTTCTACTTT
ACTGGCTTTGGTCGTCATGAAGATGCGGACTTGCGTGGCAAAGGATTCGATAACGTGCT
GATGGTGCACGACCACGCATTAATGGACTGGATTGGGGCCAACTCCTACCGTACCTCGC
ATTACCCTTACGCTGAAGAGATGCTCGACTGGGCAGATGAACATGGCATCGTGGTGATT
GATGAAACTGCTGCTGTCGGCTTTTCGCTCTCTTTAGGCATTGGTTTCGAAGCGGGCAA
CAAGCCGAAAGAACTGTACAGCGAAGAGGCAGTCAACGGGGAAACTCAGCAAGCGCACT
TACAGGCGATTAAAGAGCTGATAGCGCGTGACAAAAACCACCCAAGCGTGGTGATGTGG
AGTATTGCCAACGAACCGGATACCCGTCCGCAAGGTGCACGGGAATATTTCGCGCCACT
GGCGGAAGCAACGCGTAAACTCGACCCGACGCGTCCGATCACCTGCGTCAATGTAATGT
TCTGCGACGCTCACACCGATACCATCAGCGATCTCTTTGATGTGCTGTGCCTGAACCGT
TATTACGGATGGTATGTCCAAAGCGGCGATTTGGAAACGGCAGAGAAGGTACTGGAAAA
AGAACTTCTGGCCTGGCAGGAGAAACTGTACACCGACATGTGGAGTGAAGAGTATCAGT
GTGCATGGCTGGATATGTATCACCGCGTCTTTGATCGCGTCAGCGCCGTCGTCGGTGAA
CAGGTATGGAATTTCGCCGATTTTGCGACCTCGCAAGGCATATTGCGCGTTGGCGGTAA
CAAGAAAGGGATCTTCACTCGCGACCGCAAACCGAAGTCGGCGGCTTTTCTGCTGCAAA
AACGCTGGACTGGCATGAACTTCGGTGAAAAACCGCAGCAGGGAGGCAAACAATGAATC
AACAACTCTCCTGGCGCACCATCGTCGGCTACAGCCTCGGGAATTGCTACCGAGCTCtt
ctaccaatggtgggactcgatccagagaatgaaatcctgcacactctttcattcttggc
tggtcttatgacatggtcacagatcactgatttgccattttctttgtactcaactttcg
tgatcgagtctcggcatgggttcaacaaacaaacaatatggatgttcattagggacatg
atcaaaggaatactcctctctgtcatacctgcccctcctatcgttgccgcaattattgt
tatagttcagaaaggaggtccttacctcgccatctatctgtgggcattcatgtttatcc
tgtctctagtgatgatgactatataccctgttttgattgcacctcttttcaacaagttc
actcctcttcctgatggagacctccgggagaagattgagaaacttgcttcttctctaaa
gtttcctctgaagaagctgtttgttgtcgatggatctacaaggtcaagccatagtaatg
cttacatgtatggtttcttcaagaacaaaaggattgttctttatgacacattgattcag
cagtgccagaatgagaatgaaattgtggcggttattgcacacgagctgggacactggga
gctcgaatttccccgatcgttcaaacatttggcaataaagtttcttaagattgaatcct
gttgccggtcttgcgatgattatcatataatttctgttgaattacgttaagcatgtaat
aattaacatgtaatgcatgacgttatttatgagatgggtttttatgattagagtcccgc
aattatacatttaatacgcgatagaaaacaaaatatagcgcgcaaactaggataaatta
tcgcgcgcggtgtcatctatgttactagatcgggaattcactggccgtcgttttacaac
gtcgtgactgggaaaaccctggcgttacccaacttaatcgccttgcagcacatccccct
ttcgccagctggcgtaatagcgaagaggcccgcaccgatcgcccttcccaacagttgcg
cagcctgaatggcgcccgctcctttcgctttcttcccttcctttctcgccacgttcgcc
ggctttccccgtcaagctctaaatcgggggctccctttagggttccgatttagtgcttt
acggcacctcgaccccaaaaaacttgatttgggtgatggttcacgtagtgggccatcgc
cctgatagacggtttttcgccctttgacgttggagtccacgttctttaatagtggactc
ttgttccaaactggaacaacactcaaccctatctcgggctattcttttgatttataagg
gattttgccgatttcggaaccaccatcaaacaggattttcgcctgctggggcaaaccag
cgtggaccgcttgctgcaactctctcagggccaggcggtgaagggcaatcagctgttgc
ccgtctcactggtgaaaagaaaaaccaccccagtacattaaaaacgtccgcaatgtgtt
attaagttgtctaagcgtcaatttgtttacaccacaatatatcctgcca

SEQ ID NO:51 is the nucleic acid sequence of pRD29A-HP-BnCPP. Italicized sequences are the right and left border repeats. Underlined sequence is the RD29A promoter. Sequence in bold is the BnCPP antisense sequence, bold and underlined sequence is BnCPP sense fragment and the upper case sequence represents the truncated GUS-fragment.

SEQ ID NO:52

gtttacccgccaatatatcctgtcaaacactgatagtttaaactgaaggcgggaaacga
caatctgatcatgagcggagaattaagggagtcacgttatgacccccgccgatgacgcg
ggacaagccgttttacgtttggaactgacagaaccgcaacgttgaaggagccactcagc
cgcgggtttctggagtttaatgagctaagcacatacgtcagaaaccattattgcgcgtt
caaaagtcgcctaaggtcactatcagctagcaaatatttcttgtcaaaaatgctccact
gacgttccataaattcccctcggtatccaattagagtctcatattcactctcaatccaa
ataatctgcaccggatctggatcgtttcgcatgattgaacaagatggattgcacgcagg
ttctccggccgcttgggtggagaggctattcggctatgactgggcacaacagacaatcg
gctgctctgatgccgccgtgttccggctgtcagcgcaggggcgcccggttctttttgtc
aagaccgacctgtccggtgccctgaatgaactgcaggacgaggcagcgcggctatcgtg
gctggccacgacgggcgttccttgcgcagctgtgctcgacgttgtcactgaagcgggaa
gggactggctgctattgggcgaagtgccggggcaggatctcctgtcatctcaccttgct
cctgccgagaaagtatccatcatggctgatgcaatgcggcggctgcatacgcttgatcc
ggctacctgcccattcgaccaccaagcgaaacatcgcatcgagcgagcacgtactcgga
tggaagccggtcttgtcgatcaggatgatctggacgaagagcatcaggggctcgcgcca
gccgaactgttcgccaggctcaaggcgcgcatgcccgacggcgatgatctcgtcgtgac
ccatggcgatgcctgcttgccgaatatcatggtggaaaatggccgcttttctggattca
tcgactgtggccggctgggtgtggcggaccgctatcaggacatagcgttggctacccgt
gatattgctgaagagcttggcggcgaatgggctgaccgcttcctcgtgctttacggtat
cgccgctcccgattcgcagcgcatcgccttctatcgccttcttgacgagttcttctgag
cgggactctggggttcgaaatgaccgaccaagcgacgcccaacctgccatcacgagatt
tcgattccaccgccgccttctatgaaaggttgggcttcggaatcgttttccgggacgcc
ggctggatgatcctccagcgcggggatctcatgctggagttcttcgcccacgggatctc
tgcggaacaggcggtcgaaggtgccgatatcattacgacagcaacggccgacaagcaca
acgccacgatcctgagcgacaatatgatcgggcccggcgtccacatcaacggcgtcggc
ggcgactgcccaggcaagaccgagatgcaccgcgatatcttgctgcgttcggatatttt
cgtggagttcccgccacagacccggatgatccccgatcgttcaaacatttggcaataaa
gtttcttaagattgaatcctgttgccggtcttgcgatgattatcatataatttctgttg
aattacgttaagcatgtaataattaacatgtaatgcatgacgttatttatgagatgggt
ttttatgattagagtcccgcaattatacatttaatacgcgatagaaaacaaaatatagc
gcgcaaactaggataaattatcgcgcgcggtgtcatctatgttactagatcgggcctcc
tgtcaatgctggcggcggctctggtggtggttctggtggcggctctgagggtggtggct
ctgagggtggcggttctgagggtggcggctctgagggaggcggttccggtggtggctct
ggttccggtgattttgattatgaaaagatggcaaacgctaataagggggctatgaccga
aaatgccgatgaaaacgcgctacagtctgacgctaaaggcaaacttgattctgtcgcta
ctgattacggtgctgctatcgatggtttcattggtgacgtttccggccttgctaatggt
aatggtgctactggtgattttgctggctctaattcccaaatggctcaagtcggtgacgg
tgataattcacctttaatgaataatttccgtcaatatttaccttccctccctcaatcgg
ttgaatgtcgcccttttgtctttggcccaatacgcaaaccgcctctccccgcgcgttgg
ccgattcattaatgcagctggcacgacaggtttcccgactggaaagcgggcagtgagcg
caacgcaattaatgtgagttagctcactcattaggcaccccaggctttacactttatgc
ttccggctcgtatgttgtgtggaattgtgagcggataacaatttcacacaggaaacagc
tatgaccatgattacgccaagcttgcatgcctgcagggagccatagatgcaattcaatc
aaactgaaatttctgcaagaatctcaaacacggagatctcaaagtttgaaagaaaattt
atttcttcgactcaaaacaaacttacgaaatttaggtagaacttatatacattatattg
taattttttgtaacaaaatgtttttattattattatagaattttactggttaaattaaa
aatgaatagaaaaggtgaattaagaggagagaggaggtaaacattttcttctatttttt
catattttcaggataaattattgtaaaagtttacaagatttccatttgactagtgtaaa
tgaggaatattctctagtaagatcattatttcatctacttcttttatcttctaccagta
gaggaataaacaatatttagctcctttgtaaatacaaattaattttccttcttgacatc
attcaattttaattttacgtataaaataaaagatcatacctattagaacgattaaggag
aaatacaattcgaatgagaaggatgtgccgtttgttataataaacagccacacgacgta
aacgtaaaatgaccacatgatgggccaatagacatggaccgactactaataatagtaag
ttacattttaggatggaataaatatcataccgacatcagttttgaaagaaaagggaaaa
aaagaaaaaataaataaaagatatactaccgacatgagttccaaaaagcaaaaaaaaag
atcaagccgacacagacacgcgtagagagcaaaatgactttgacgtcacaccacgaaaa
cagacgcttcatacgtgtccctttatctctctcagtctctctataaacttagtgagacc
ctcctctgttttactcacaaatatgcaaactagaaaacaatcatcaggaataaagggtt
tgattacttctattggaaaggactctagaggatccttaatctgtcttcttgtcttctcc
atcaatggctcgaagcctctctacaagaggagggtgtgagtagtgataagctgagtaca
atgggtctgtgttcatcgctgataagttctcttcctgtagcttcactagggcaggacgt
agatcctttgcataaccaagattcactgcaaaagcatcagcctgaaactcaaacgctcg
actaacaaggttgaggtcaaagcttactaggtgttgaagtggtattacagtgtgctgaa
atatgatcaaaccaatgagaactggttgtgtatcaaaaccaaaactcctgaagagatca
gtggagtttctgacaagagtgtatcctccaaattgcaagaaggcaaggatttgaacagc
aatgaacgagtatgtagtgtgattcagcttccagtgtcccagctcgtgtgcaataaccg
ccacaatttcattctcattctggcactgctgaatcaatgtgtcataaagaacaatcctt
ttgttcttgaagaaaccatacatgtaagcattactatggcttgaccttgtagatccatc
gacaacaaacagcttcttcagaggaaactttagagaagaagcaagtttctcaatcttct
cccggaggtctccatcaggaagaggagtgaacttgttgaaaagaggtgcaatcaaaaca
gggtatatagtcatcatcactagagacaggataaacatgaatgcccacagatagatggc
gaggtaaggacctcctttctgaactataacaataattgcggcaacgataggaggggcag
gtatgacagagaggagtattcctttgatcatgtccctaatgaacatccatattgtttgt
ttgttgaacccatgccgagactcgatcacgaaagttgagtacaaagaaaatggcaaatc
agtgatctgtgaccatgtcataagaccagccaagaatgaaagagtgtgcaggatttcat
tctctggatcgagtcccaccattggtagaaagccgccagatatcttccaaaaccaaggc
aagatcccaaagaacagaatcgcagagtccataagtatagtaacaaactcatgaacaaa
gtgaaaatggcttttgtcaagactgtaagctcgagatttctcaaacttctcttggctaa
tgactccaaccaaagtctttgggagagtgggaagcttgagagcagtatgttgcctcaga
tccaaatacgtctcaaaaacgtacatcactatcataaaaccaacgacggtttccatgaa
aggaatcgccatcccctcgaatttccccgatcgttcaaacatttggcaataaagtttct
taagattgaatcctgttgccggtcttgcgatgattatcatataatttctgttgaattac
gttaagcatgtaataattaacatgtaatgcatgacgttatttatgagatgggtttttat
gattagagtcccgcaattatacatttaatacgcgatagaaaacaaaatatagcgcgcaa
actaggataaattatcgcgcgcggtgtcatctatgttactagatcgggaattcactggc
cgtcgttttacaacgtcgtgactgggaaaaccctggcgttacccaacttaatcgccttg
cagcacatccccctttcgccagctggcgtaatagcgaagaggcccgcaccgatcgccct
tcccaacagttgcgcagcctgaatggcgcccgctcctttcgctttcttcccttcctttc
tcgccacgttcgccggctttccccgtcaagctctaaatcgggggctccctttagggttc
cgatttagtgctttacggcacctcgaccccaaaaaacttgatttgggtgatggttcacg
tagtgggccatcgccctgatagacggtttttcgccctttgacgttggagtccacgttct
ttaatagtggactcttgttccaaactggaacaacactcaaccctatctcgggctattct
tttgatttataagggattttgccgatttcggaaccaccatcaaacaggattttcgcctg
ctggggcaaaccagcgtggaccgcttgctgcaactctctcagggccaggcggtgaaggg
caatcagctgttgcccgtctcactggtgaaaagaaaaaccaccccagtacattaaaaac
gtccgcaatgtgttattaagttgtctaagcgtcaatttgtttacaccacaatatatcct
gcca

SEQ ID NO:52 is the nucleic acid sequence of pRD29A-antisense-BnCPP. Italicized sequences are the right and left border repeats. Underlined sequence is the RD29A promoter. Sequence in bold is the BnCPP antisense sequence.

| | |
|---|---|
| gtttacccgccaatatatcctgtcaaacactgatagtttaaactgaaggcgggaaacga | SEQ ID NO:53 |
| caatctgatcatgagcggagaattaagggagtcacgttatgacccccgccgatgacgcg | |
| ggacaagccgttttacgtttggaactgacagaaccgcaacgttgaaggagccactcagc | |
| cgcgggtttctggagtttaatgagctaagcacatacgtcagaaaccattattgcgcgtt | |
| caaaagtcgcctaaggtcactatcagctagcaaatatttcttgtcaaaaatgctccact | |
| gacgttccataaattcccctcggtatccaattagagtctcatattcactctcaatccaa | |
| ataatctgcaccggatctggatcgtttcgcatgattgaacaagatggattgcacgcagg | |
| ttctccggccgcttgggtggagaggctattcggctatgactgggcacaacagacaatcg | |
| gctgctctgatgccgccgtgttccggctgtcagcgcaggggcgcccggttctttttgtc | |
| aagaccgacctgtccggtgccctgaatgaactgcaggacgaggcagcgcggctatcgtg | |
| gctggccacgacgggcgttccttgcgcagctgtgctcgacgttgtcactgaagcgggaa | |
| gggactggctgctattgggcgaagtgccggggcaggatctcctgtcatctcaccttgct | |
| cctgccgagaaagtatccatcatggctgatgcaatgcggcggctgcatacgcttgatcc | |
| ggctacctgcccattcgaccaccaagcgaaacatcgcatcgagcgagcacgtactcgga | |
| tggaagccggtcttgtcgatcaggatgatctggacgaagagcatcaggggctcgcgcca | |
| gccgaactgttcgccaggctcaaggcgcgcatgcccgacggcgatgatctcgtcgtgac | |
| ccatggcgatgcctgcttgccgaatatcatggtggaaaatggccgcttttctggattca | |
| tcgactgtggccggctgggtgtggcggaccgctatcaggacatagcgttggctacccgt | |
| gatattgctgaagagcttggcggcgaatgggctgaccgcttcctcgtgctttacggtat | |
| cgccgctcccgattcgcagcgcatcgccttctatcgccttcttgacgagttcttctgag | |
| cgggactctggggttcgaaatgaccgaccaagcgacgcccaacctgccatcacgagatt | |
| tcgattccaccgccgccttctatgaaaggttgggcttcggaatcgttttccgggacgcc | |
| ggctggatgatcctccagcgcggggatctcatgctggagttcttcgcccacgggatctc | |
| tgcggaacaggcggtcgaaggtgccgatatcattacgacagcaacggccgacaagcaca | |
| acgccacgatcctgagcgacaatatgatcgggcccggcgtccacatcaacggcgtcggc | |
| ggcgactgcccaggcaagaccgagatgcaccgcgatatcttgctgcgttcggatatttt | |
| cgtggagttcccgccacagacccggatgatccccgatcgttcaaacatttggcaataaa | |
| gtttcttaagattgaatcctgttgccggtcttgcgatgattatcatataatttctgttg | |
| aattacgttaagcatgtaataattaacatgtaatgcatgacgttatttatgagatgggt | |
| ttttatgattagagtcccgcaattatacatttaatacgcgatagaaaacaaaatatagc | |
| gcgcaaactaggataaattatcgcgcgcggtgtcatctatgttactagatcgggcctcc | |
| tgtcaatgctggcggcggctctggtggtggttctggtggcggctctgagggtggtggct | |
| ctgagggtggcggttctgagggtggcggctctgagggaggcggttccggtggtggctct | |
| ggttccggtgattttgattatgaaaagatggcaaacgctaataagggggctatgaccga | |
| aaatgccgatgaaaacgcgctacagtctgacgctaaaggcaaacttgattctgtcgcta | |
| ctgattacggtgctgctatcgatggtttcattggtgacgtttccggccttgctaatggt | |
| aatggtgctactggtgattttgctggctctaattcccaaatggctcaagtcggtgacgg | |
| tgataattcacctttaatgaataatttccgtcaatatttaccttccctccctcaatcgg | |
| ttgaatgtcgcccttttgtctttggcccaatacgcaaaccgcctctccccgcgcgttgg | |
| ccgattcattaatgcagctggcacgacaggtttcccgactggaaagcgggcagtgagcg | |
| caacgcaattaatgtgagttagctcactcattaggcaccccaggctttacactttatgc | |
| ttccggctcgtatgttgtgtggaattgtgagcggataacaatttcacacaggaaacagc | |
| tatgaccatgattacgccaagctgggaaatttttcgccagttctaaatatccggaaacc | |
| tcttgggatgccattgcccatctatctgtaatttattgacgaaatagacgaaaaggaag | |
| gtggctcctataaagcacatcattgcgataacagaaaggccattgttgaagatacctct | |
| gctgacattggtccccaagtggaagcaccaccccatgaggagcaccgtggagtaagaag | |
| acgttcgagccacgtcgaaaaagcaagtgtgttgatgtagtatctccattgacgtaagg | |
| gatgacgcacaatccaactatccatcgcaagaccattgctctatataagaaagttaata | |
| tcatttcgagtggccacgctgagggggatccatggcgattcctttcatggaaaccgtcg | |
| ttggttttatgatagtgatgtacgtttttgagacgtatttggatctgaggcaacatact | |
| gctctcaagcttcccactctcccaaagactttggttggagtcattagccaagagaagtt | |
| tgagaaatctcgagcttacagtcttgacaaaagccattttcactttgttcatgagtttg | |
| ttactatacttatggactctgcgattctgttctttgggatcttgccttggttttggaag | |
| atatctggcggctttctaccaatggtgggactcgatccagagaatgaaatcctgcacac | |
| tctttcattcttggctggtcttatgacatggtcacagatcactgatttgccattttctt | |
| tgtactcaactttcgtgatcgagtctcggcatgggttcaacaaacaaacaatatggatg | |
| ttcattagggacatgatcaaaggaatactcctctctgtcatacctgcccctcctatcgt | |
| tgccgcaattattgttatagttcagaaaggaggtccttacctcgccatctatctgtggg | |
| cattcatgtttatcctgtctctagtgatgatgactatataccctgttttgattgcacct | |
| cttttcaacaagttcactcctcttcctgatggagacctccgggagaagattgagaaact | |
| tgcttcttctctaaagtttcctctgaagaagctgtttgttgtcgatggatctacaaggt | |
| caagccatagtaatgcttacatgtatggtttcttcaagaacaaaaggattgttctttat | |
| gacacattgattcagcagtgccagaatgagaatgaaattgtggcggttattgcacacga | |
| gctgggacactggaagctgaatcacactacatactcgttcattgctgttcaaatccttg | |
| ccttcttgcaatttggaggatacactcttgtcagaaactccactgatctcttcaggagt | |
| tttggttttgatacacaaccagttctcattggtttgatcatatttcagcacactgtaat | |
| accacttcaacacctagtaagctttgacctcaaccttgttagtcgagcgtttgagtttc | |
| aggctgatgcttttgcagtgaatcttggttatgcaaaggatctacgtcctgccctagtg | |
| aagctacaggaagagaacttatcagcgatgaacacagacccattgtactcagcttatca | |
| ctactcacaccctcctcttgtagagaggcttcgagccattgatggagaagacaagaaga | |
| cagattaacccctcgaatttccccgatcgttcaaacatttggcaataaagtttcttaag | |
| attgaatcctgttgccggtcttgcgatgattatcatataatttctgttgaattacgtta | |
| agcatgtaataattaacatgtaatgcatgacgttatttatgagatgggtttttatgatt | |
| agagtcccgcaattatacatttaatacgcgatagaaaacaaaatatagcgcgcaaacta | |
| ggataaattatcgcgcgcggtgtcatctatgttactagatcgggaattcactggccgtc | |
| gttttacaacgtcgtgactgggaaaaccctggcgttacccaacttaatcgccttgcagc | |
| acatccccctttcgccagctggcgtaatagcgaagaggcccgcaccgatcgcccttccc | |
| aacagttgcgcagcctgaatggcgcccgctcctttcgctttcttcccttcctttctcgc | |
| cacgttcgccggctttccccgtcaagctctaaatcgggggctccctttagggttccgat | |
| ttagtgctttacggcacctcgaccccaaaaaacttgatttgggtgatggttcacgtagt | |
| gggccatcgccctgatagacggtttttcgccctttgacgttggagtccacgttctttaa | |
| tagtggactcttgttccaaactggaacaacactcaaccctatctcgggctattcttttg | |
| atttataagggattttgccgatttcggaaccaccatcaaacaggattttcgcctgctgg | |
| ggcaaaccagcgtggaccgcttgctgcaactctctcagggccaggcggtgaagggcaat | |
| cagctgttgcccgtctcactggtgaaaagaaaaaccaccccagtacattaaaaacgtcc | |
| gcaatgtgttattaagttgtctaagcgtcaatttgtttacaccacaatatatcctgcca | |

SEQ ID NO:53 is the nucleic acid sequence of MuA-BnCPP. Italicized sequences are the right and left border repeats. Underlined sequence is the MuA promoter. Sequence in bold is the BnCPP sense sequence.

### Example 5

### Southern Analysis

Genomic Southern blot analysis of transgenic Arabidopsis was performed using standard techniques known to one skilled in the art. Typically, 10 μg of DNA was electrophoresed in a 0.8% agarose gel and transferred to an appropriate membrane such as Hybond N+ (Amersham Pharmacia Biotech). Pre-hybridization and hybridization conditions were as suggested by the membrane manufacturer, typically at 65° C. The final stringency wash was typically at 1×SSC and 0.1% SDS at 65° C. The NPTII coding region was typically used as the radiolabeled probe in Southern blot analysis.

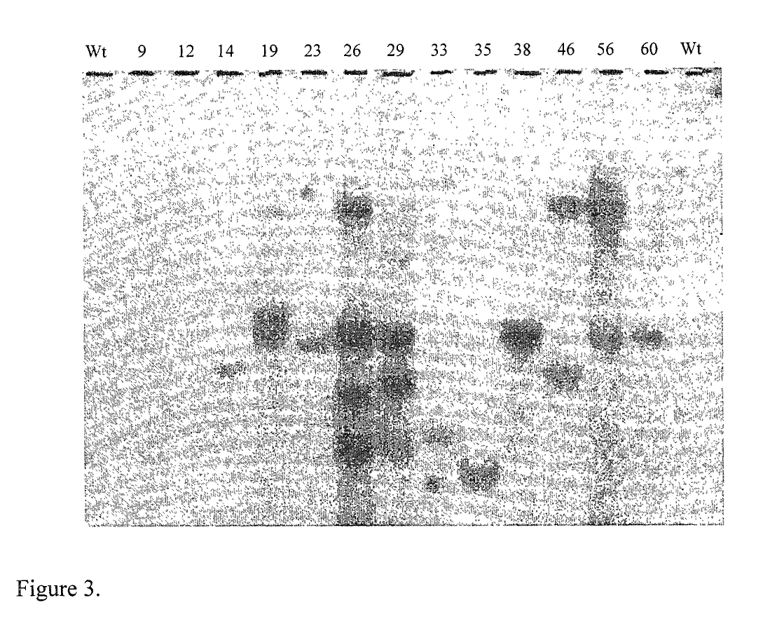

Thirty-seven Arabidopsis lines were selected as homozygous pBI121-AtCPP over-expression lines for further examination. FIG. 3 shows a representative blot confirming the presence of the pBI121-AtCPP transgene. Lines were confirmed to be transgenic by PCR analysis using transgene specific primers in the PCR assays.

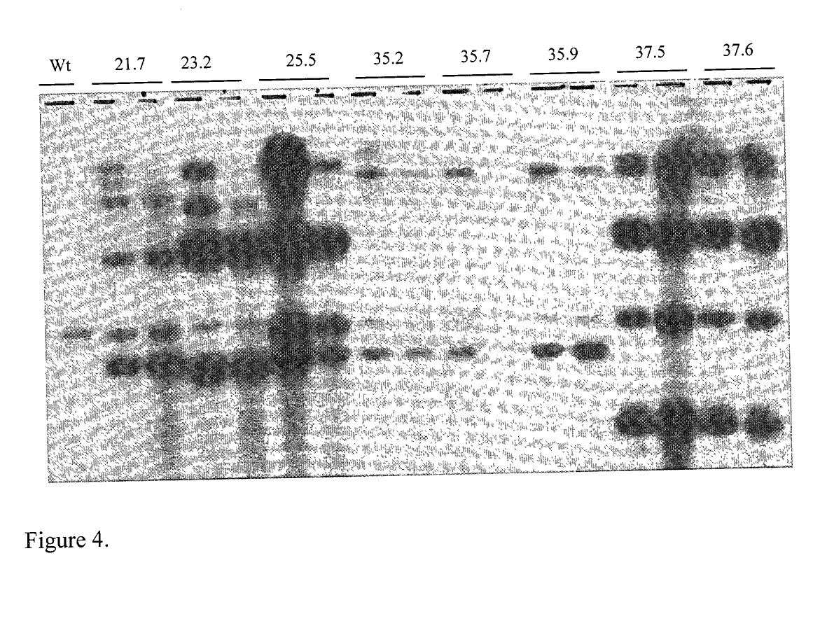

Thirty-three Arabidopsis lines were selected as homozygous pBI121-HP-AtCPP hair-pin down-regulation lines for further examination. FIG. 4 shows a representative blot confirming the presence of the pBI121-HP-AtCPP hair-pin construct. All lines were confirmed to be transgenic by PCR analysis using transgene specific primers in the PCR assays.

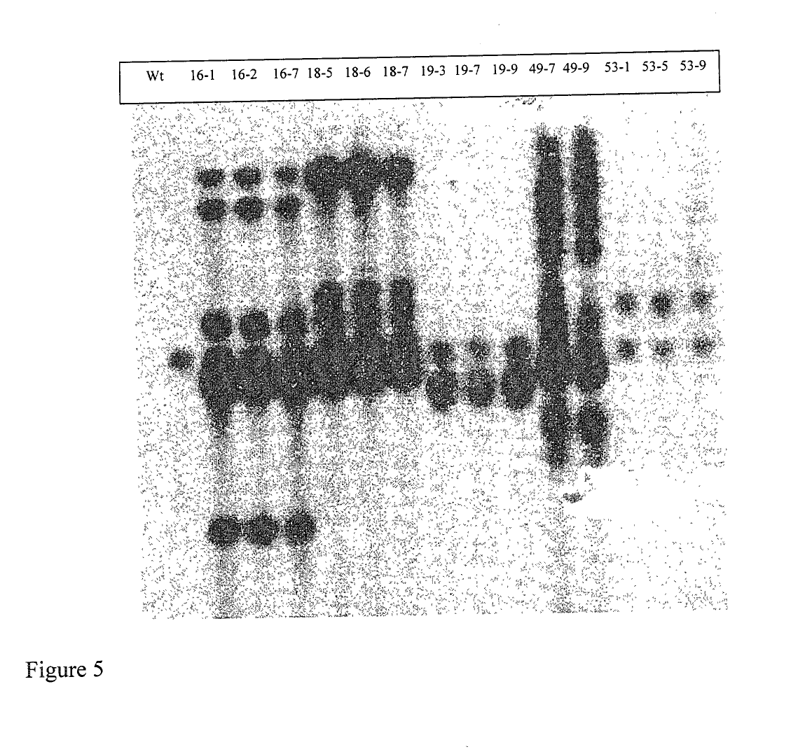

Arabidopsis lines were selected as homozygous pRD29A-AtCPP over-expression lines for further examination. FIG. 5 shows a representative blot confirming the presence of the pRD29A-AtCPP transgene. Lines were confirmed to be transgenic by PCR analysis using transgene specific primers in the PCR assays.

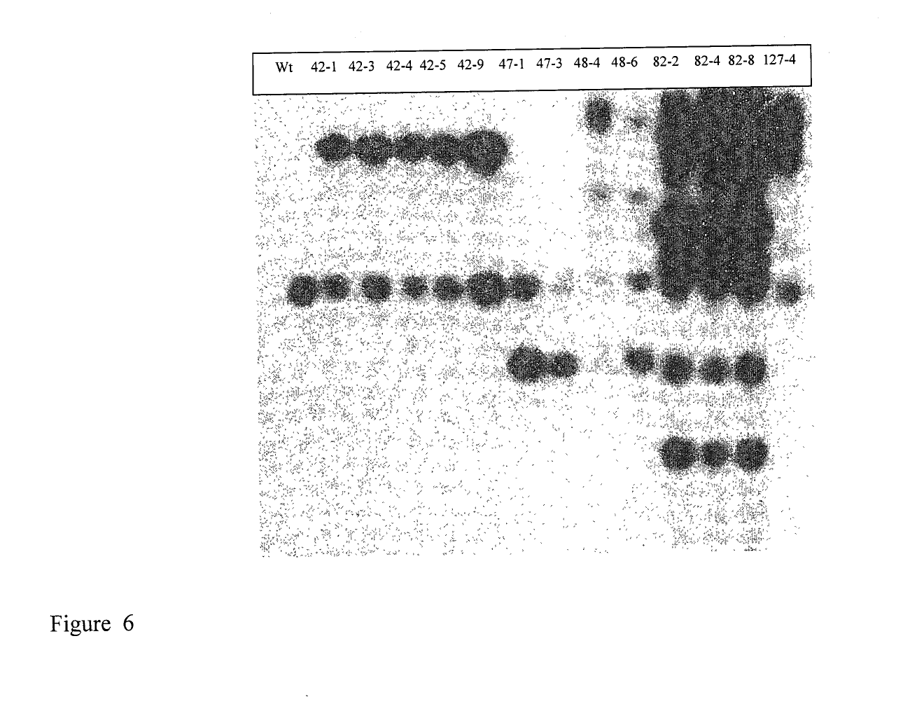

Arabidopsis lines were selected as homozygous pRD29A-HP-AtCPP lines for further examination. FIG. 6 shows a representative blot confirming the presence of the pRD29A-HP-AtCPP transgene. Lines were confirmed to be transgenic by PCR analysis using transgene specific primers in the PCR assays.

### Example 6

### PCR Analysis of Transgenic Plants

PCR was used as a method to confirm the presence of the transgene in all transgenic lines and every construct. Typical PCR mixtures contained: 1× reaction buffer (10 mM Tris-HCl pH 8.8, 1.5 mM MgCl₂, 50 mM KCl), dNTP's at 200 μM, 1 pM forward and reverse primer, 2.5U. Taq DNA polymerase, and template plus water to a final volume of 50 μL. Reactions were run at 1 minute 94° C., 1 minute 60° C., 1 minute 72° C., for 30 cycles. Primers used in the analysis of pBI121-AtCPP and pBI121-HP-AtCPP transgenic plants were as shown in Table 8. Primers used in the analysis of pRD29A-AtCPP were RD29AP1 (SEQ ID NO:66) and SEQ ID NO:7. Primers used in the analysis of pRD29A-HP-AtCPP transgenic plants were those identified as RD29APl (SEQ ID NO:66), SEQ ID NO:8 and SEQ ID NO:8, Nosterm-RV (SEQ ID NO:67).

TABLE 8

| | | |
|---|---|---|
| pBI121-AtCPP BamFW: | 5′-GCCGACAGTGGTCCCAAAGATGG-3′ | (SEQ ID NO:10) |
| p35S-AtCPP SmaRV: | 5′-AAACCCGGGTTAATCTGTCTTCTTGTCTTCTCCA-3′ | (SEQ ID NO:7) |
| p35S-AtCPP BamFW: | 5′-CTGGAGCTCTTTTACCGAGGTTGGGCCTTGATCC-3′ | (SEQ ID NO:8) |
| p35S-HP-AtCPP SmaRV: | 5′-GCAAGACCGGCAACAGGA-3′ | (SEQ ID NO:13) |
| pRD29AP1: | 5′-TTTAAGCTTGGAGCCATAGATGCAATTCAA-3′ | (SEQ ID NO:66) |
| pRD29AP1: | 5′-TTTAAGCTTGGAGCCATAGATGCAATTCAA-3′ | (SEQ ID NO:66) |
| Nosterm-RV: | 5′-GCAAGACCGGCAACAGGA-3′ | (SEQ ID NO:67) |

### Example 7

### Northern Analysis of Transgenic Plants

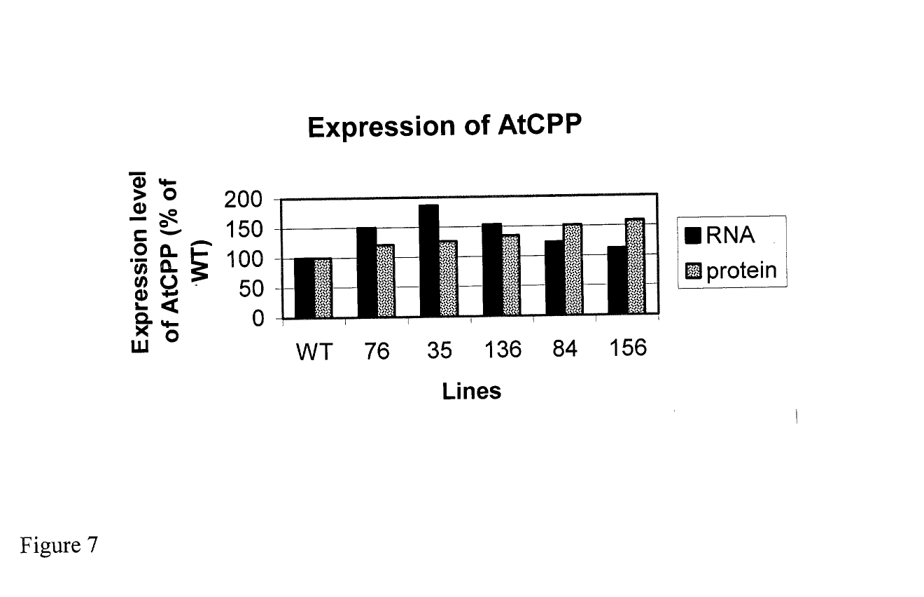

Total RNA was isolated from developing leaf tissue of 27 35S-AtCPP Arabidopsis lines (T3 plants). Approximately 10 μg of total RNA was loaded into each lane. The Northern blot was first probed with P³² labeled, single-stranded antisense transcript of AtCPP which detects sense transcript, then stripped and re-probed with cDNA of β-tubulin that was used as a reference. The hybridizing bands of AtCPP and t3-tubulin were scanned and quantified using the UN-Scan-It programme (Silk Scientific, Utah, USA), and the ratio of the two hybridizing bands for each sample was obtained. The ratio of the wild type plants was set to 100%, and was compared with those of the transgenic lines. Twenty-one out of twenty-seven lines showed higher expression of AtCPP transcript as compared to the wild type. Values ranged from 104% to 282% of wild type. The results of five lines (35, 84, 76, 136, and 156) of the 21 over-expressing lines is shown in FIG. 7.

### Example 8

### Production of Polyclonal Antibodies Against AtCPP

Anti-AtCPP antibodies were generated using AtCPP fusion protein over-expressed in E. coli. The over-expression vector, pMAL-p2, contains 1175 bp malE gene that is located upstream of AtCPP and encodes a 43 KDa maltose-binding protein (MBP). The 1275 bp BamHI/SmaI DNA fragment of AtCPP was inserted into pMAL-p2 at BamHI and SalI sites. The SalI site was converted into blunt end using Klenow fragment. The resulting fusion protein MBP-AtCPP was then over-expressed in DH50, and purified by one-step affinity for MBP as described by the manufacturer (New England Biolab). The soluble fraction of the crude bacterial extract containing the MBP-AtCPP fusion protein was loaded to a amylose column (1.5 cm×10.0 cm), and the proteins were eluted with 10 mM maltose in column buffer (50 mM Tris-HCl, pH 7.5, 1 mM EDTA, and 200 mM NaCl). Fractions containing purified MBP-AtCPP fusion protein were pooled, and concentrated with a Centriprep-30 concentrator (Amicon). All purification steps were carried out at 4° C. To generate an antibody, the purified fusion protein was further separated by SDS-PAGE and the Coomassie stained band corresponding to the fusion protein was excised. The identity of the fusion protein was confirmed by Western analysis using anti-MBP antibodies (purchased from New England Biolab). The protein was eluted from the gel slice by electroelution and then emulsified in Ribi adjuvant (Ribi Immunochem) to a final volume of 1 ml. MBP-AtCPP protein was injected into a 3 kg New Zealand rabbit on day 1 and booster injections were given on day 21 and day 35 with 175 μg of the protein each time. High-titer antisera were obtained one week after the final injection.

### Example 9

### Western Blot Analysis of ³⁵S-AtCPP Transgenic Lines Using Anti-AtCPP Antibodies

Western analysis was performed to examine expression level of AtCPP in the transgenic lines compared with that of wild type plants. Anti-Bip antibody, an ER lumenal protein (Stressgen, Victoria, B C, Canada) was used as a reference. Total proteins were extracted from developing leaf tissue of five ABAs lines and a wild type control. The antigenic protein bands of AtCPP and Bip were scanned and quantified using the UN-Scan-It programme (Silk Scientific, Utah, USA) and the ratio of the two protein bands for each sample was obtained. The ratio of the wild type plants was set to 100%, and was compared with those of the transgenic lines. Data is presented in FIG. 7 indicating that the AtCPP protein level was increased in the transgenic lines compared to the wild type plants.

### Example 10

### ABA Sensitivity of Transgenic Seedlings

Approximately 100 seeds were assessed per line per 9 cm plate. Seeds were plated on minimal medium (½ MS) supplemented with no ABA or 1.0 IM ABA. Plates were chilled for 3 days at 4° C. in the dark, and incubated for up to 21 days at 22° C. with 24 hour continuous light. Plates were assessed for germination, cotyledon expansion, true leaf development and seedling vigor. Seedlings were assessed for ABA sensitivity over 21 days of growth at which time sensitive seedlings were arrested at the cotyledon stage, lacked true leaves, and showed inhibition of root growth. Wild type control Columbia plants had two to three pairs of true leaves and a well developed root system. Lines were categorized as ABA sensitive (ABA^{S}) if less than 1% of plants looked like control, moderately ABA sensitive (ABA^{MS}) if more than 1% but less than 50% of looked like control, or ABA insensitive (ABA^{Wt}) if greater than 50% looked like control.

For example, if a plate had 20 healthy seedlings and the control plate had 60 healthy seedlings, the line would be 33% of control and categorized as moderately ABA sensitive.

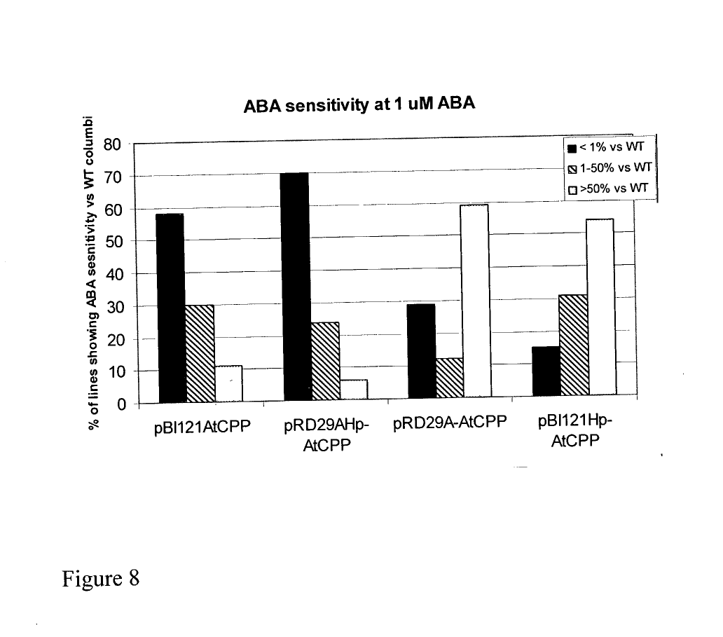

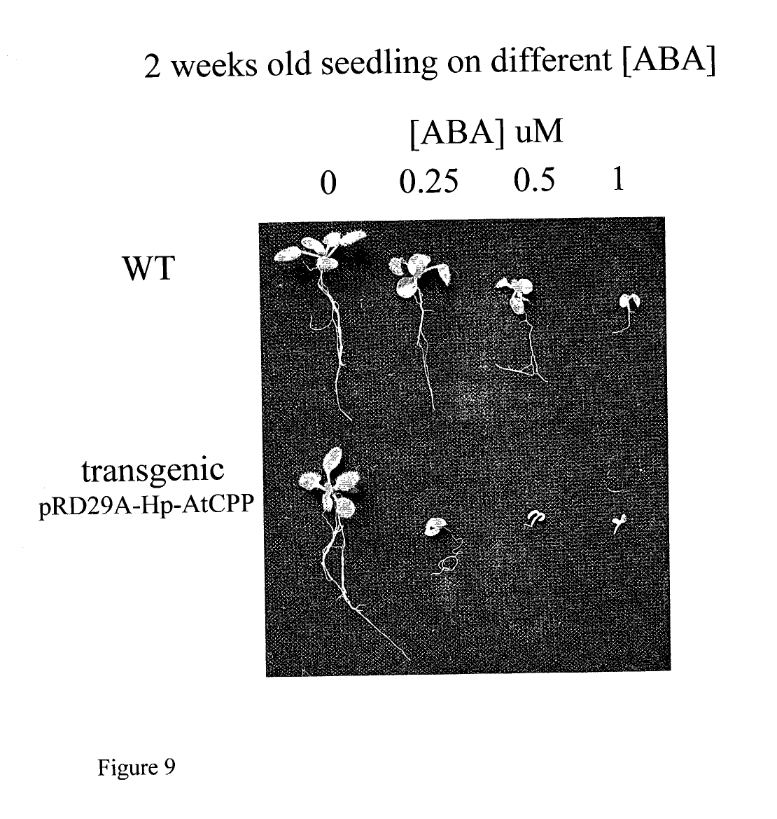

All four vector constructs (pBI121-AtCPP, pBI121Hp-AtCPP, pRD29AHp-AtCPP, pRD29A-ATCPP) have resulted in transgenic lines of Arabidopsis which have increased sensitivity to ABA which is indicative of stress tolerance. The data for all 4 constructs is shown in FIG. 8. Of the lines transformed with the pBI121-AtCPP construct to over-express the AtCPP gene, 58% (21 out of 36) were classified as sensitive and an added 30% (11 out of 36) were classified as moderately sensitive. These lines were tested again in T4 and T5 generations and their ABA sensitivity was still present indicating that ABA sensitivity is an inheritable trait. Of the lines transformed with the pBI121-HP-AtCPP construct to down-regulate the AtCPP gene by double stranded RNA-inhibition, 15% (7 out of 45) were classified as sensitive and 31% (14 out of 45) were classified as moderately sensitive. To illustrate the increased sensitivity of transgenic lines to ABA, FIG. 9 shows the results of germination and seedling development over a range of ABA concentrations. Wild type and pRD29A-HP-AtCPP are compared. Of the lines transformed with pRD29AHp-AtCPP 70% (12 out of 17) showed high sensitivity and 24% (4 out of 17) showed moderate sensitivity to ABA. Of the lines transformed with pRD29A-AtCPP 29% (5 out of 17) showed high sensitivity and 12% (2 out of 17) moderate sensitivity to ABA. Clearly all 4 transgene constructs are altering ABA sensitivity and ABA signal transduction.

### Example 11

### Drought Experiments

Arabidopsis plants were grown five plants per 4″ or 3″ pot, in a replicated water-stress experiment. All pots were filled with equal amounts of homogeneous premixed and wetted soil. Plants were grown under 16 hour daylight (150-200 pmol/m²/s) at 22° C. and 70% relative humidity. On the day that the first flower opened drought treatment was initiated. First soil water content in each pot was equalized on a weight basis and any further watering of plants was stopped. Daily measurements of soil water content were taken by recording total pot weight. At the end of the drought treatment (6 to 9 days for experiments in 4″ pots and 4-5 days for experiments in 3″ pots) plants were harvested and shoot dry weights determined. Differences in plant growth were factored into the analysis by expressing water loss on a per gram shoot dry weight basis.

11a) pBI121-AtCPP, Drought Stress Screen:

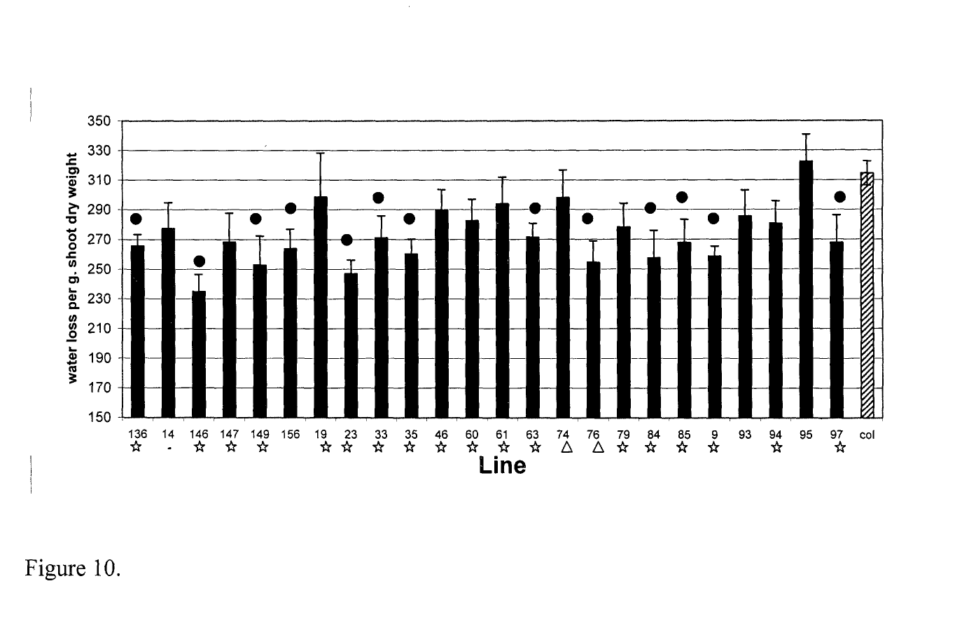

Analysis of pBI121-AtCPP transgenic lines during water-stress treatment experiments of up to an eight day period, shows a strong trend towards increased soil water content and reduced water loss per gram of shoot biomass. After three days of water-stress treatment most lines had increased soil water content relative to the wild type control with four out of twenty-four lines, 146, 149, 156 and 97, showing a statistically significant difference. The amount of water lost per gram of shoot biomass was lower for all lines except one (95), and thirteen of these lines were significantly different from the wild type Columbia control (FIG. 10). All of the lines showing a statistically significant lower water loss per gram shoot biomass also showed an increased ABA sensitivity. There is also a strong trend, for all but one line (95), which is ABA^{Wt}, towards greater shoot biomass at the end of the drought stress treatment. Seven of those lines 136, 146, 23, 46, 76, 84 and 9, were statistically significant from control at a p=0.05 value.

11b) pBI121-AtCPP, Water Loss Per Gram Shoot Biomass During Water Stress Treatment:

Lines 35, 76, 95 and a wild type control were grown and placed under a water-stress treatment as above. Plants were harvested at 2 days, 4 days and 6 days of drought treatment. The ABA^{S} lines, 35 and 76, showed a statistically significant reduction in water-loss relative to shoot dry weight at all three time points (Table 9). Additionally, the two ABA^{S} transgenic lines had increased shoot biomass, due to increased leaf biomass, and maintained higher soil water contents during drought treatment.

TABLE 9
Water loss (g) per Shoot dry weight (g) after 2, 4 and 6 days of
drought-stress treatment. Values in bold indicate statistically
significant differences from Columbia.

| | 2 days | | 4 days | | 6 days | |
|---|---|---|---|---|---|---|
| Line | Mean | Std. Error | Mean | Std. Error | Mean | Std. Error |
| 35 | 212.5 | 3.5 | 308.0 | 9.9 | 297.7 | 11.2 |
| 76 | 227.2 | 5.8 | 321.2 | 8.5 | 293.8 | 5.0 |
| 95 | 287.0 | 5.1 | 377.3 | 14.8 | 348.5 | 25.5 |
| Columbia | 265.3 | 11.8 | 408.2 | 7.7 | 345.9 | 6.7 |
| Wild type | | | | | | |

11c) pBI121-AtCPP, Drought Stress and Shoot Recovery:

Water-stress tolerance and determination of post drought-treatment recovery ability was assessed using 20 of the 24 pBI121-AtCPP transgenic lines. Drought treatment was imposed for 6 days after which the plants were watered and allowed to grow for 6 days. Recovered shoot fresh biomass was then determined. Soil water content of these plants was measured daily during the drought treatment and the results confirm previously seen trends. All ABA sensitive (ABA^{S}) lines that showed a statistically significantly reduction of water loss on a per gram dry weight basis in experiment 11a, continued to show a significant greater soil water content than control plants in this experiment (Table 10). Additionally, Table 10 shows that the recovered shoot fresh biomass after 6 days of drought treatment was significantly greater in all the ABAs lines than Columbia.

TABLE 10
Soil water content on day 3 of drought treatment and recovered
shoot fresh weight after 6 days of drought treatment (values in
bold were significantly different from Columbia at p = 0.05)

| | | soil water | | | |
|---|---|---|---|---|---|
| | | content day 3 | | recovered | |
| | ABA status | Mean | | shoot biomass | |
| Line | ABA | (% initial) | Std Error | Mean (g) | Std Error |
| 136 | ABA^{S} | 46.6 | 1.9 | 4.5 | 0.16 |
| 14 | ABA^{S} | 50.25 | 0.7 | 4.1 | 0.12 |
| 146 | ABA^{S} | 45.9 | 2.5 | 4.0 | 0.11 |
| 147 | ABA^{S} | 45.1 | 1.7 | 4.0 | 0.15 |
| 149 | ABA^{S} | 45.3 | 1.8 | 3.8 | 0.17 |
| 156 | ABA^{S} | 47.1 | 1.9 | 4.0 | 0.134 |
| 23 | ABA^{S} | 49 | 1.4 | 4.0 | 0.17 |
| 33 | ABA^{S} | 46.9 | 1.6 | 4.3 | 0.14 |
| 35 | ABA^{S} | 41.7 | 1.7 | 4.0 | 0.11 |
| 46 | ABA^{S} | 44.8 | 1.7 | 3.8 | 0.09 |
| 63 | ABA^{S} | 46.3 | 1.4 | 4.0 | 0.19 |
| 76 | ABA^{S} | 47.8 | 1.0 | 3.9 | 0.17 |
| 79 | ABA^{S} | 45.4 | 1.1 | 4.1 | 0.09 |
| 84 | ABA^{S} | 46.8 | 1.9 | 4.1 | 0.16 |
| 85 | ABA^{S} | 45.3 | 1.9 | 4.0 | 0.12 |
| 9 | ABA^{S} | 45.2 | 2.1 | 3.9 | 0.12 |
| 93 | ABA^{S} | 43.5 | 1.2 | 2.8 | 0.07 |
| 94 | ABA^{S} | 46.9 | 1.5 | 3.9 | 0.13 |
| 97 | ABA^{S} | 53 | 1.2 | 3.8 | 0.16 |
| 95 | ABA^{Wt} | 41.9 | 1.2 | 2.7 | 0.06 |
| Columbia | ABA^{Wt} | 41.3 | 1.0 | 2.7 | 0.04 |

11d) pBII21-AtCPP, Seed Yield After Drought Stress Treatment:

Seed yield after drought stress during flowering was examined using ten pBI121-AtCPPP transgenic lines, eight of which were ABA. Plants were grown one per 4″ pot and were exposed to 9 days of drought treatment as described above. A second group of plants was grown and maintained under well watered conditions as the optimal group. After 9 days of drought treatment plants were re-watered and allowed to continue growth and seed set to maturity. After drought-treatment conditions all eight ABAS lines had increased yields relative to controls, which ranged from 109% to 126% of the Columbia (Table 11). Drought-treatment resulted in a reduction of yield in all lines, including controls, relative to plants grown under optimal conditions. Expression of the seed yields obtained from drought-treated group relative to the same line under optimal conditions shows that the transgenics preserve a larger percentage of optimal seed yield than do wild type lines.

TABLE 11
Seed Yield following 9 days drought-treatment

| | ABA status | Seed Yield (g per plant) | | % | % |
|---|---|---|---|---|---|
| Line | ABA | Mean (g) | Std Error | Columbia | Optimal |
| 156 | ABA^{S} | 0.735 | 0.044 | 126.2 | 83.7 |
| 63 | ABA^{S} | 0.675 | 0.061 | 116.0 | 71.0 |
| 146 | ABA^{S} | 0.666 | 0.053 | 114.4 | 72.9 |
| 94 | ABA^{S} | 0.644 | 0.052 | 110.6 | 68.8 |
| 84 | ABA^{S} | 0.642 | 0.049 | 110.4 | 61.8 |
| 76 | ABA^{S} | 0.631 | 0.055 | 108.5 | 66.6 |
| 136 | ABA^{S} | 0.630 | 0.051 | 108.3 | 74.1 |
| 35 | ABA^{S} | 0.614 | 0.054 | 105.6 | 74.2 |
| 93 | ABA^{Wt} | 0.567 | 0.041 | 97.5 | 60.0 |
| 95 | ABA^{Wt} | 0.388 | 0.088 | 66.7 | 43.4 |
| Columbia | ABA^{Wt} | 0.582 | 0.060 | 100 | 53.8 |

11e) pBI121-AtCPP, Seed yield and Growth Under Optimal Water Conditions:

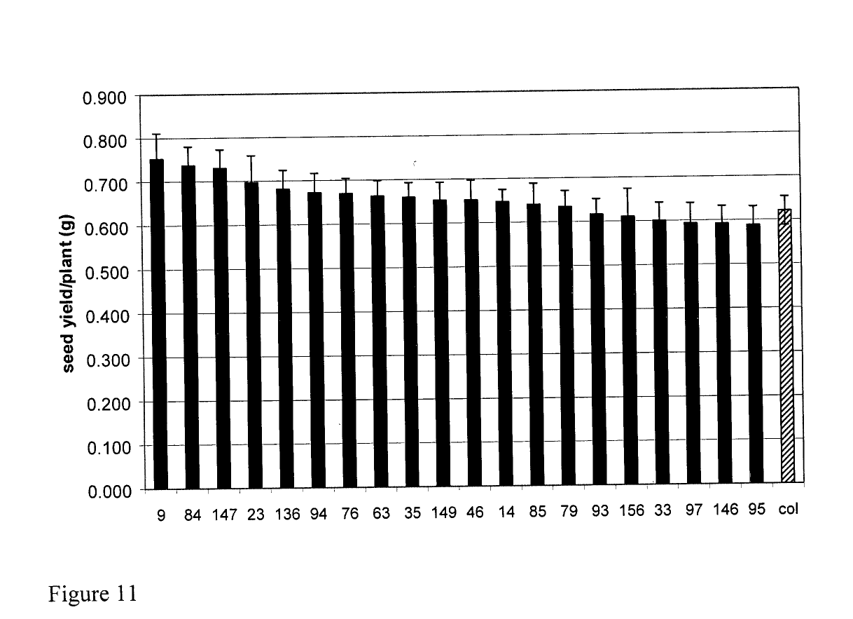

The lines evaluated above and a number of additional lines were examined in a growth and yield experiment under optimal, well-watered conditions. Results indicated that the ABAs lines were shorter at the stage of first open flower, had more rosette leaves, however, by maturity there were no differences in plant height of transgenics and Columbia. Moreover, the ABAS transgenics showed similar or higher seed yields ranging from 95% to 121% of the wild type control (FIG. 11).

11g) pRD29A-HP-AtCPP Screen for Drought Tolerant Phenotype:

Analysis of 17 transgenic lines identified 7 candidate drought tolerant lines (12, 22, 23, 47, 82, 83, 90) on the basis of higher soil water content and lower water loss per g of shoot dry weight (Tablel2). All 7 drought tolerant candidate lines showed strong ABA sensitivity and lines that did not show drought tolerance did not show ABA sensitivity.

TABLE 12
Soil water content after 3 days of drought treatment
and water lost per g shoot dry weight. Values in bold
are statistically different from those of Columbia
wild type (p = 0.05)

| | | soil water | | | |
|---|---|---|---|---|---|
| | | content day 2 | | water lost in 2days/g | |
| | ABA status | Mean | Std | shootDW | |
| Line | ABA | (% initial) | Error | Mean (g/g) | Std Error |
| 10 | ABA^{S} | 33.4 | 1.6 | 199.1 | 4.5 |
| 11 | ABA^{S} | 34.6 | 3.3 | 173.1 | 1.6 |
| 12 | ABA^{S} | 36.2 | 2.0 | 179.5 | 5.0 |
| 126 | ABA^{MS} | 32.5 | 2.6 | 199.1 | 4.1 |
| 127 | ABA^{MS} | 33.5 | 2.0 | 195.6 | 10.6 |
| 14 | ABA^{S} | 32.7 | 1.2 | 203 | 4.9 |
| 17 | ABA^{S} | 29.9 | 1.8 | 200.7 | 7.3 |
| 22 | ABA^{S} | 39.3 | 2.1 | 170.0 | 3.0 |
| 23 | ABA^{S} | 35.7 | 1.4 | 174.9 | 2.6 |
| 42 | ABA^{MS} | 28 | 0.7 | 185.4 | 5.8 |
| 47 | ABA^{S} | 35.9 | 2.2 | 181.2 | 7.7 |
| 7 | ABA^{Wt} | 35 | 1.3 | 201.8 | 5.1 |
| 82 | ABA^{S} | 36.7 | 2.2 | 178.3 | 4.0 |
| 83 | ABA^{S} | 40 | 1.4 | 180.7 | 6.9 |
| 9 | ABA^{S} | 31.4 | 1.4 | 173.8 | 8.7 |
| 90 | ABA^{S} | 38.2 | 1.3 | 177.6 | 6.2 |
| 93 | ABA^{Wt} | 30.7 | 1.8 | 175.3 | 4.6 |
| Columbia | ABA^{Wt} | 32.1 | 1.2 | 196.9 | 6.2 |

### Example 12

### Growth Analysis

The growth analysis of most promising constructs has been set up at 3 stages. Eight plants per line were grown in 3″ pots with one plant per pot at 22C, 16 hr light (150-200,Mmol/m²/s) and 70% RH. Plants were harvested at vegetative growth stage (2 week old seedlings), bolting growth stage (at first open flower) and mid-flowering growth stage (5 to 7 days from first open flower). Also, in some growth experiments additional group of plants was grown in 4″ pots (one per pot and 10 plants per line) to maturity for seed yield determinations.

12a) pBI121-AtCPP Growth Under Optimal and Biotic Stress Conditions

The growth and productivity of pBI121-AtCPP transgenic Arabidopsis lines was examined at several stages of development under optimal growth conditions. Although optimal growth conditions were maintained, plants were assessed to be under a degree of stress that was later determined to be a result of the soil properties. Soil analysis found a fungal contaminant that was believed to be responsible for the biotic stress. This stress could be negated by sterilization of the soil prior to use. Eight ABA^{S} lines, two with normal ABA sensitivity (ABA^{Wt}) and a wild type Columbia control were analyzed.

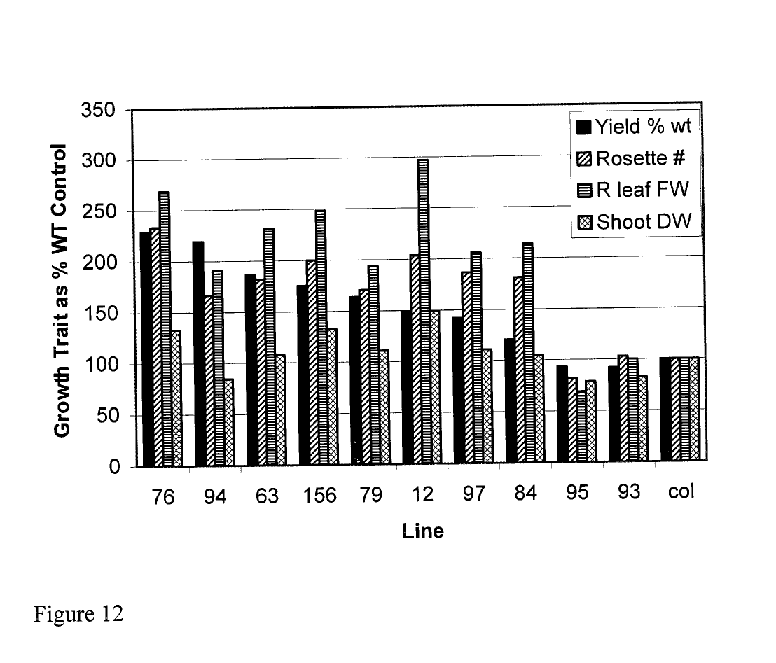

FIG. 12 presents the results of various growth (from mid-flowering stage) and yield parameters and each trait is expressed as a percentage of the Columbia control. The results strongly support an enhanced growth phenotype. This enhanced growth phenotype is present at all growth stages. At the vegetative stage, all ABA^{S} transgenic plants showed an increase in leaf number relative to that of the wild type with four of the eight lines showing a statistically significant difference. The two ABA^{Wt} lines showed the same or fewer leaves relative to wild type.

At the bolting stage ABA^{S} transgenics showed an increase in leaf number but plants were shorter at this stage (first open flower) than controls. The shoot fresh weight of transgenics was significantly increased relative to that of controls, ranging from 80% to 342% of the wild type. The ABAs transgenics displayed a delay in flowering from one to three days. The ABA^{Wt} transgenics did not show delayed flowering, increased shoot fresh weight or increased height.

At the flowering stage of development the enhanced growth phenotype is maintained (greater leaf number and fresh weight), however, there were no observable differences in plant height indicating that transgenics bolt shorter but reach same final plant height.

Of particular significance is the observation, that under these conditions (biotic stress due to presence of fungi in the soil) yields of the ABAS transgenics were significantly higher, ranging from 120% to 229% of the wild type control. The ABA^{Wt} lines showed similar or slightly reduced yields relative to the Columbia control. This finding indicates that ABA^{S} transgenic lines are affected less by the biotic stress. This observation has been confirmed, where 5 of the drought tolerant lines were grown in contaminated soil to maturity. The seed yields of transgenic lines, even though greatly reduced relative to optimal conditions, were 2.5 to 4.5 fold higher than those of Columbia wild type (Table 13).

TABLE 13
Seed yield ofpBI121-AtCPP lines grown in contaminated soil.
Values in bold indicate statistical differences at p = 0.05

| | ABA | Seed Yield per | |
|---|---|---|---|
| Line | sensitivity | plant (g) | % of Columbia |
| 156 | ABA^{S} | 0.33 ± 0.04 | 316% |
| 23 | ABA^{S} | 0.35 ± 0.05 | 336% |
| 76 | ABA^{S} | 0.31 ± 0.04 | 296% |
| 84 | ABA^{S} | 0.25 ± 0.33 | 237% |
| 9 | ABA^{S} | 0.48 ± 0.05 | 455% |
| Columbia | ABA^{Wt} | 0.11 ± 0.03 | |

12b) pBI121-AtCPP Early Seedling Growth:

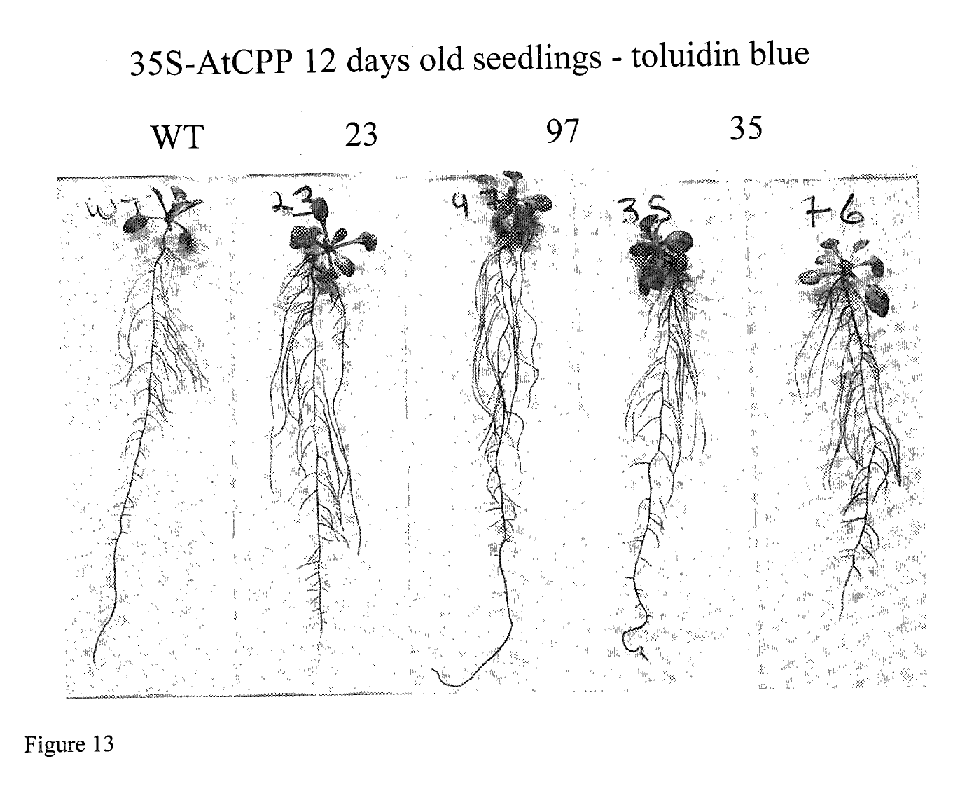

Four ABA^{S} and one ABA^{Wt} line plus Columbia were examined for early seedling growth on agar plates. Twenty seeds were plated in a line on agar plates containing 50% MS with 1% sucrose and vitamins and 6 plates per line were used. Plates were placed on slants, which allowed roots to grow downwards. Root length was measured on 7-day old seedlings and shoot and root biomass determined on 11-day old seedlings. Two of the ABA^{S} transgenic lines had significantly longer roots and all 4 ABA^{S} lines had shoot dry weights 114% to 123% of controls and root dry weights of 116% to 151% of controls. As a result, the shoot biomass to rootbiomass ratios were slightly reduced in transgenics. These results indicate that enhanced growth of these transgenics is evident in the early growth stage, shortly after germination, and the root growth is more enhanced relative to shoot growth. In a different experiment seedlings were pulled out of agar and roots were stained with toluidine blue to show their structure. FIG. 13 shows that transgenic lines had more extensive lateral root system, which would account for greater root biomass.

12c) pRD29A-HP-AtCPP Optimal Growth Characteristics

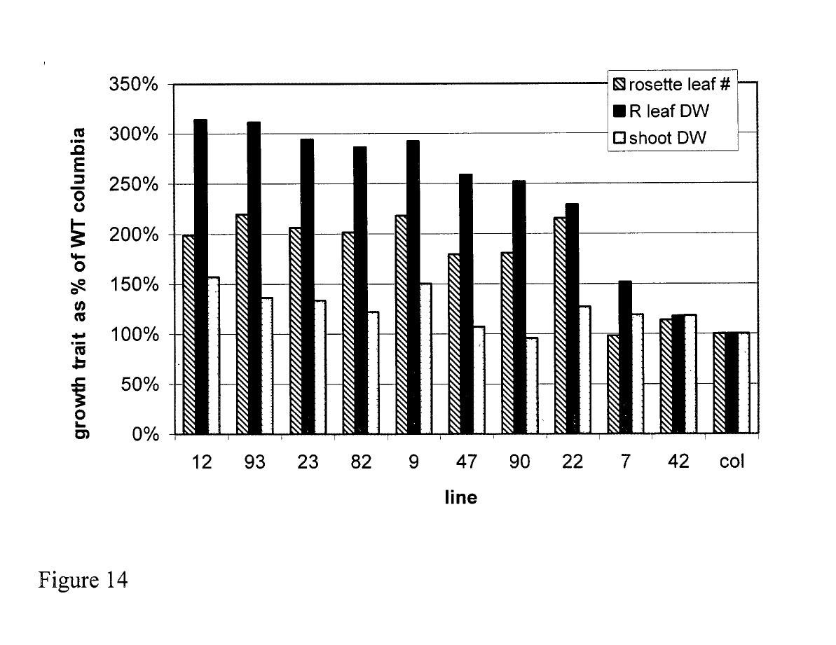

An optimal growth study has been conducted with 10 lines as described before. Vegetative growth data showed that two of the lines (12 and 9) had significantly more leaves and seven of the lines (12, 22, 23, 47, 82, 9) had significantly greater shoot biomass. Bolting data showed that eight of the lines (12, 22, 23, 47, 82, 9, 90, 93) were significantly delayed in flowering by one to two days, and seven of the lines were significantly shorter than Columbia at first open flower. All of the lines except 42 and 7 had significantly greater number of rosette leaves and shoot FW and this trend is maintained into the mid-flowering harvest (FIG. 14). The plant height, however, by mid-flowering harvest was not significantly different between the transgenic lines and control. All the lines that showed this enhanced growth also showed drought tolerance and ABA sensitivity.

### Example 13

### Ultrastructure pBII21-AtCPP

Two of the drought tolerant and ABA^{S} lines (35 and 76) plus Wt Columbia were used to examine stem and root cross-sections for any differences in ultrastructure. Free hand sections of mature stems (plants flowering for 10 days) were obtained from above the first node, stained with toluidine blue and preserved with glycerol. The stems of transgenic plants appeared to have more dense cellular structure and contain one or two more vascular bundles than those of Columbia Wt indicating more enhanced water and nutrient transport system.

Leaf disks were taken and fresh weights determined. Transgenic leaf disks were significantly heavier, 20-24% greater than corresponding wild type controls. This increase is believed to be as a result of a thicker leaf.

### Example 14

### Cold Stress Experiment pBI121-AtCPP

Four drought tolerant, ABA^{S} lines (156, 23, 35, 76) and one ABA^{Wt}(95) line plus wild type Columbia were included in a cold stress study. Plants were grown in 3″ pots one per pot) with 10 replicate pots per line at 22C for 10 days (7 days on agar plates and 4 in soil). The cold stress group was moved into 7° C. for 5 days while the optimal group was left at 22C. After 5 days in the cold both cold stress group and the optimal group were harvested for shoot biomass determination. ABA^{S} and drought tolerant lines had significantly greater shoot biomass than Columbia in both optimal (25 to 39% greater shoot fresh weight) and cold stress groups (18 to 44% greater shoot DW) (Table 14). Results of an eight-day cold stress showed that differences between the transgenic lines and Columbia were even more pronounced (53 to 61% greater shoot fresh weight). This result indicates greater plant vigor and better ability of transgenics to cope with cold stress.

TABLE 14
Shoot fresh weight of optimal and cold stressed (5C for 5d)
pBI121-AtCPP. Values in bold indicate statistical difference
at p = 0.05

| | | | | Cold stress | |
|---|---|---|---|---|---|
| | | Optimal shoot FW | | shoot FW | |
| | ABA | | % of | | % of |
| Line | sensitivity | mg | Columbia | mg | Columbia |
| 156 | ABA^{S} | 95.4 ± 3.7 | 137% | 23.1 0.7 | 118% |
| 23 | ABA^{S} | 96.3 ± 3.9 | 139% | 28.3 1.5 | 144% |
| 35 | ABA^{S} | 87.0 ± 1.7 | 125% | 25.3 1.4 | 130% |
| 76 | ABA^{S} | 94.7 ± 2.2 | 136% | 27.3 1.5 | 140% |
| 95 | ABAWt | 67 ± 2.4 | 96% | 21.4 1.0 | 109% |
| Columbia | ABAWt | 69 ± 1.9 | | 19.6 1.1 | |

### Example 15

### Drought Stress Under High Temperature pBI121-AtCPP

A drought stress experiment was conducted as described above except that day temperature of 32° C. (16 hr) and night temperature of 22° C. (8 hr) was maintained. These temperatures were achieved daily over a 2 hr ramping period. Four ABAs and one ABA^{Wt} line plus Columbia were included. Plants were monitored daily for water loss and soil water content and after 5 days of drought treatment half of the plants were harvested and the other half was re-watered and allowed to recover for four days. Shoots were harvested and shoot fresh weight determined. The results (Table 15) of this experiment showed that previously identified drought tolerant lines maintained their drought tolerant phenotype at high temperature and were able to recover well from the drought stress at high temperature

TABLE 15
Soil water content on day 2 and water lost in 2 days/final shoot
dry weight plus recovery shoot FW after 5days of drought stress
at 32 C. day and 22 C. night temperatures. Values in bold indicate
significant differences from the Columbia control.

| | ABA | soil water | water lost in | recovered shoot |
|---|---|---|---|---|
| line | sensitivity | content day 2 | 2d/shoot DW | FW (g) |
| 136 | ABA^{S} | 50.4 ± 1.1 | 485.7 ± 18.5 | 1.30 ± 0.04 |
| 146 | ABA^{S} | 52.1 ± 1.0 | 504.5 ± 7.9 | 1.15 ± 0.04 |
| 35 | ABA^{S} | 52.2 ± 0.8 | 502.8 ± 15.8 | 1.19 ± 0.02 |
| 76 | ABA^{S} | 52.1 ± 0.6 | 435.6 ± 10.5 | 1.11 ± 0.03 |
| 95 | ABAWt | 50.0 ± 0.9 | 518.2 ± 13.0 | 0.86 ± 0.03 |
| Columbia | ABAWt | 48.6 ± 0.6 | 559.7 ± 19.0 | 0.84 ± 0.03 |

### Example 16

### Heat Stress and Seed Yield pBI121-AtCPP

Two ABAS lines and one ABAWtline plus Columbia were examined for the effect of heat stress during flowering on the final seed yield. Plants were grown in 4 inch pots (one/pot) as described above and 9 days from first open flower the temperature was ramped from 22 C to 43C over 2 hours and plants were kept at 43C for 2 hr. Temperature was then ramped back to 22C over 2 hours and plants were grown under optimal conditions until maturity. The seed yields from this experiment are shown in Table 16. One of the drought tolerant lines (35) had significantly greater yield than Columbia.

TABLE 16
Seed yield of pBI121-AtCPP lines after two hour 43 C. heat
stress 9 days from first open flower. Values in bold are
statistically significant from Columbia.

| | ABA | seed yield | seed yield |
|---|---|---|---|
| line | sensitivity | (g/plant) | (% of col.) |
| 35 | ABA^{S} | 0.55 ± 0.05 | 347% |
| 76 | ABA^{S} | 0.24 ± 0.03 | 148% |
| 95 | ABAWt | 0.11 ± 0.02 | 69% |
| Columbia | ABAWt | 0.16 ± 0.03 | |

The effect of heat shock on lines of pBI121-AtCPP at the early flowering stage was assessed. Three ABAS lines (76, 136, 97) a ABAWtline (95) and a Columbia wild type control were seeded in 128 cell flats, one flat per line. At the early flowering stage flats were exposed to a temperature of 46.8° C. for 50 minutes and then returned to normal growth conditions. Lack of continued growth from main meristems was defined as main miristem death and scored for each line. Data is shown in Table 17.

TABLE 17
Meristem death due to heat shock

| Line | Wt | 95 | 76 | 136 | 97 |
|---|---|---|---|---|---|
| % Death | 91 | 97 | 79 | 59 | 18 |

### Example 17

### Stomata Density Determinations pBI121AtCPP

Two ABA^{S} lines (76 and 35) plus Columbia were examined for stomata density on the upper and lower leaf surface. Nail polish imprints of the upper and lower epidermis were obtained from a fully expanded leaf #5. These imprints were analyzed under the microscope and the number of stomata per 8.7×10⁻⁸ m² were counted. There were no significant differences found between transgenics and Columbia in the stomata of the upper or lower epidermis (Table 18). The increases seen in drought tolerance and reduced water loss is not attributable to a reduced number of leaf stomata.

TABLE 18
Stomata numbers per 8.7 × 10⁻⁸ m² of abaxial
and adaxial epidermis of fully expanded leaf #5 in pBI121AtCPP.

| | | stomata on upper | stomata on lower |
|---|---|---|---|
| line | ABA sensitivity | epidermis | epidermis |
| 35 | ABA^{S} | 68 ± 5 | 103 ± 7 |
| 76 | ABA^{S} | 58 ± 6 | 120 ± 16 |
| Columbia | ABAWt | 57 ± 6 | 116 ± 11 |

### Example 18

### CPP Consensus Sequences

Also included in the invention is the CPP consensus sequences. The consensus sequences were generated by alignment of the CPP polypeptide and nucleic acid ssequences as well as sequences homogous using the program BioEdit.

The “x” in the consensus sequence represents any amino acid or nucleotide. Preferably “x” a conservative amino acid or nucleotide substitution. More preferably, “x” is the most amino acid or nucleotide most prevalent at a given postion. For example, the amino acid at postion 145 of SEQ ID NO: 73 is a proline as it occurs 66% of the time.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

What is claimed is:

1. A method of producing a transgenic plant, comprising introducing into a plant cell a compound that increases prenyl protease expression or activity to generate a transgenic cell; and regenerating a transgenic plant from said transgenic cell.

2. The method of claim 1, wherein said plant has an altered phenotype selected from the group consisting of increased tolerance to stress, delayed senescence, increased ABA sensitivity, increased yield, increased productivity and increased biomass compared to a wild type plant.

3. The method of claim 1, wherein said compound comprises a nucleic acid sequence encoding prenyl protease.

4. The method of claim 3, wherein said nucleic acid comprises SEQ ID NO: 1, 14, 17, 68, 70, 72, 74, 21, 23, 25, 27, 29, 31, or 33.

5. The method of claim 3, wherein said nucleic acid is operably linked to a promotor.

6. The method of claim 5, wherein said promoter is selected from the group consisting of a constitutive promoter, an ABA inducible promoter, tissue specific promoters or a guard cell-specific promoter

7. The method of claim 1, wherein said compound is a prenyl protease polypeptide or fragment thereof.

8. The method of claim 7, wherein said prenyl protease polypeptide comprises the amino acid sequence of SEQ ID NO: 2, 15, 18, 22, 24, 26, 28, 30, 32, 34, 69, 71, 73, or 75.

9. The transgenic plant produced by claim 1.

10. The seed produced by the transgenic plant of claim 9, wherein said seed produces a plant that has an altered phenotype selected from the group consisting of increased tolerance to stress, delayed senescence, increased ABA sensitivity, increased yield, increased productivity and increased biomass compared to a wild type plant.

11. A method of producing a transgenic plant, comprising introducing into a plant cell a nucleic acid that inhibits prenyl protease expression or activity to generate a transgenic cell; and regenerating a transgenic plant from said transgenic cell.

12. The method of claim 11, wherein said plant has an altered phenotype selected from the group consisting of increased tolerance to stress, delayed senescence, increased ABA sensitivity, increased yield, increased productivity and increased biomass compared to a wild type plant.

13. The method of claim 11, wherein said nucleic acid comprises an antisense nucleic acid sequence encoding prenyl protease.

14. The method of claim 13, wherein said antisense nucleic acid comprises 20 or more consecutive nucleic acids complementary to SEQ ID NO: 1, 14, 17, 21, 23, 25, 27, 29, 31, 33, 68, 70, 72, or 74.

15. The method of claim 13, wherein said antisense nucleic acid comprises SEQ ID NO: 16, 19, 20, 5, 35, 37, 38, 42, 43, 45, 46, 48, 49, 51, or 52.

16 The method of claim 11, wherein said nucleic acid is operably linked to a promotor.

17. The method of claim 16, wherein said promoter is selected from the group consisting of a constitutive promoter, an ABA inducible promoter, tissue specific promoters or a guard cell-specific promoter

18. The method of claim 11, wherein the nucleic acid is an inhibitor of farnesylation.

19 The transgenic plant produced by any one of the methods of claims 11.

20. The seed produced by the transgenic plant of claim 19, wherein said seed produces a plant that has an altered phenotype selected from the group consisting of increased tolerance to stress, delayed senescence, increased ABA sensitivity, increased yield, increased productivity and increased biomass compared to a wild type plant.

21. A method of producing a transgenic plant, comprising introducing into a plant cell a nucleic acid selected from the group consisting of SEQ ID NO: 16, 19, 20, 5, 35, 37, 38, 42, 43, 45, 46, 48, 49, 51, and 52 to generate a transgenic cell; and regenerating a transgenic plant from said transgenic cell.

22. An isolated polypeptide comprising the mature form of an amino acid sequenced selected from the group consisting of SEQ ID NO: 2, 15, 18, 69, 71, 73 and 75.

23 An isolated polypeptide comprising an amino acid sequence selected from the group consisting of SEQ ID NO: 2, 15, 18, 69, 71, 73 and 75.

24. An isolated polypeptide comprising an amino acid sequence which is at least 96% identical to an amino acid sequence selected from the group consisting of SEQ ID NO: 15

25. An isolated polypeptide comprising an amino acid sequence which is at least 99% identical to an amino acid sequence selected from the group consisting of SEQ ID NO: 2, 15, 18

26 The polypeptide of claim 24, wherein said polypeptide has prenyl pretease activity.

27 An isolated polypeptide, wherein the polypeptide comprises an amino acid sequence comprising one or more conservative substitutions in the amino acid sequence selected from the group consisting of SEQ ID NO: 2, 15, and 18.

28. The polypeptide of claim 23, wherein said polypeptide is naturally occurring.

29. An isolated nucleic acid molecule comprising a nucleic acid sequence selected from the group consisting of SEQ ID NO: 1, 14, 17, 20, 16, 19, 68, 70, 72, and 74.

30. The nucleic acid molecule of claim 29, wherein the nucleic acid molecule is naturally occurring.

31. An isolated nucleic acid molecule encoding the mature form of a polypeptide having an amino acid sequence selected from the group consisting of SEQ ID NO: SEQ ID NO: 2, 15, and 18.

32. An isolated nucleic acid molecule, wherein said nucleic acid molecule hybridizes under stringent conditions to the nucleotide sequence selected from the group consisting of SEQ ID NO: 1, 14, 17, 20, 16, 19, 68, 70, 72, and 74.

33. An isolated nucleic acid molecule comprising a nucleotide sequence which is at least 94% identical to the nucleotide sequence selected from the group consisting of SEQ ID NO: 17, 18 and 19.

34. An isolated nucleic acid molecule comprising a nucleotide sequence which is at least 99% identical to the nucleotide sequence selected from the group consisting of SEQ ID NO: 1, 14, 17, 20, 16, 19.

35. A vector comprising the nucleic acid molecule of claim 29.

36. The vector of claim 35, further comprising a promoter operably linked to said nucleic acid molecule.

37. A cell comprising the vector of claim 36.

38. An antibody that immunospecifically binds to the polypeptide of claim 22.

39. The antibody of claim 38, wherein the antibody is a monoclonal antibody.

40. The antibody of claim 37, wherein the antibody is a polyclonal antibody.

41. A method of identifying an agent that binds to the polypeptide of claim 27, the method comprising:
(a) introducing said polypeptide to said agent; and
(b) determining whether said agent binds to said polypeptide.

42. The method of claim 41, wherein the agent is a farnesylation inhibitor.

43. A method for identifying farnesylation modulator, the method comprising:
(a) providing a cell expressing the polypeptide of claim 22;
(b) contacting the cell with a candidate substance; and
(c) determining whether the substance alters farnesylation activity;
whereby, if an alteration observed in the presence of the substance is not observed when the cell is contacted with a composition in the absence of the substance, the substance is identified as a farnesylation modulator.

44. A method for identifying an interacting gene of prenyl protease, the method comprising:
a) providing the transgenic plant of claim 1;
b) creating a library of mutagenized plants from (a);
c) determining whether the mutagenized plant contains an altered phenotype;
whereby, the mutagenized plant has altered the function of an interacting gene of prenyl protease which results in an altered phenotype from the transgenic plant of (a) to that of a wild type non-transgenic plant.

45. A plant, wherein a mutation has been introduced in the gene encoding prenyl protease, resulting in said plant displaying altered prenyl protease activity and an increased tolerance to stress as compared to a wild type plant.
